SEQUENCE LISTING (301 sequences)

SEQ ID NO: 1 - protein, 28 aa, Homo sapiens
SLRRSSCFGGRMDRIGAQSGLGCNSFRY

SEQ ID NO: 2 - protein, 10 aa, Homo sapiens
YGGFLRKYPK

SEQ ID NO: 3 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 4 - protein, 70 aa, Homo sapiens
MALWMRLLPLLALLALWGPDPAAAFVNQHLCGSHLVEALYLVCGERGFFYTPKTRREAEDLQVGQVELGG

SEQ ID NO: 5 - protein, 40 aa, Homo sapiens
GPGAGSLQPLALEGSLQKRGIVEQCCTSICSLYQLENYCN

SEQ ID NO: 6 - protein, 82 aa, Homo sapiens
GSGLTNIKTEEISEVKMDAEFRHDSGYEVHHQKLVFFAEDVGSNKGAIIGLMVGGVVIATVIIITLVMLKKQYTSNHHGVVE

SEQ ID NO: 7 - protein, 434 aa, Homo sapiens
MESESESGAAADTPPLETLSFHGDEEIIEVVELDPGPPDPDDLAQEMEDVDFEEEEEEEGNEEGWVLEPQEGVVGSMEGPDDSEVTFALHSASVFCVSLDPKTNTLAVTGGEDDKAFVWRLSDGELLFECAGHKDSVTCAGFSHDSTLVATGDMSGLLKVWQVDTKEEVWSFEAGDLEWMEWHPRAPVLLAGTADGNTWMWKVPNGDCKTFQGPNCPATCGRVLPDGKRAVVGYEDGTIRIWDLKQGSPIHVLKGTEGHQGPLTCVAANQDGSLILTGSVDCQAKLVSATTGKVVGVFRPETVASQPSLGEGEESESNSVESLGFCSVMPLAAVGYLDGTLAIYDLATQTLRHQCQHQSGIVQLLWEAGTAVVYTCSLDGIVRLWDARTGRLLTDYRGHTAEILDFALSKDASLVVTTSGDHKAKVFCVQRPDR

SEQ ID NO: 8 - protein, 39 aa, Artificial Sequence
  source: /note=“Description of Artificial Sequence Synthetic polypeptide”
HGEGTFTSDLSKQMEEEAVRLFIEWLKNGGPSSGAPPPS

SEQ ID NO: 9 - protein, 25 aa, Artificial Sequence
  source: /note=“Description of Artificial Sequence Synthetic peptide”
ATQRLANELVRLQTYPRTNVGSNTY

SEQ ID NO: 10 - protein, 267 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 11 - protein, 35 aa, Human immunodeficiency virus type 1
CTRPSNNTRKSIPVGPGKALYATGAIIGNIRQAHC

SEQ ID NO: 12 - protein, 508 aa, Human immunodeficiency virus type 1
MGARASVLSGGKLDKWEKIRLRPGGKKTYQLKHIVWASRELERFAVNPGLLETGGGCKQILVQLQPSLQTGSEELKSLYNAVATLYCVHQGIEVRDTKEALDKIEEEQNKSKKKAQQAAADTGNSSQVSQNYPIVQNLQGQMVHQAISPRTLNAWVKVIEEKAFSPEVIPMFSALSEGATPQDLNTMLNTVGGHQAAMQMLKETINEEAAEWDRLHPAHAGPNAPGQMREPRGSDIAGTTSTLQEQIGWMTSNPPVPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPSHKARILAEAMSQVTSPANIMMQRGNFRNQRKTIKCFNCGKEGHLARHCRAPRKKGCWKCGREGHQMKDCTERQANFLGKIWPSHKGRPGNFLQSRPEPTAPPEESFRFGEETTTPPQKQEPLPSQKQETIDKDLYPLASLKSLFGNDPSLQ

SEQ ID NO: 13 - protein, 7 aa, Artificial Sequence
  source: /note=“Description of Artificial Sequence Synthetic peptide”
NLRIALR

SEQ ID NO: 14 - protein, 7 aa, Artificial Sequence
  source: /note=“Description of Artificial Sequence Synthetic peptide”
RLAIRLN

SEQ ID NO: 15 - protein, 40 aa, Artificial Sequence
  source: /note=“Description of Artificial Sequence Synthetic polypeptide”
DAEFRHDSGYEVHHQKLVFFAEDVGSNKGAIIGLMVGGVV

SEQ ID NO: 16 - protein, 42 aa, Artificial Sequence [sequence omitted]

SEQ ID NO: 17 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 18 - protein, 382 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 19 - protein, 74 aa, Artificial Sequence
  source: /note=“Description of Artificial Sequence Synthetic polypeptide”
DTTVSEPAPSCVTLYQSWRYSQADNGCAETVTVKVVYEDDTEGLCYAVAPGQITTVGDGYIGSHGHARYLARCL

SEQ ID NO: 20 - protein, 151 aa, Homo sapiens [sequence omitted]

SEQ ID NO: 21 - protein, 8 aa, Artificial Sequence
  source: /note=“Description of Artificial Sequence Synthetic peptide”
TTSQVRPR

SEQ ID NO: 22 - protein, 10 aa, Artificial Sequence
  source: /note=“Description of Artificial Sequence Synthetic peptide”
VKTTSQVRPR

SEQ ID NO: 23 - protein, 6 aa, Artificial Sequence
  source: /note=“Description of Artificial Sequence Synthetic peptide”
SQVRPR

SEQ ID NO: 24 - protein, 4 aa, Artificial Sequence
  source: /note=“Description of Artificial Sequence Synthetic peptide”
VRPR

SEQ ID NO: 25 - protein, 11 aa, Artificial Sequence
  source: /note=“Description of Artificial Sequence Synthetic peptide”
TTSQVRPRHIT

SEQ ID NO: 26 - protein, 5 aa, Artificial Sequence
  source: /note=“Description of Artificial Sequence Synthetic peptide”
TTSQV

SEQ ID NO: 27 - protein, 5 aa, Artificial Sequence
  source: /note=“Description of Artificial Sequence Synthetic peptide”
TSQVR

SEQ ID NO: 28 - protein, 8 aa, Artificial Sequence
  source: /note=“Description of Artificial Sequence Synthetic peptide”
TTSGIHPK

SEQ ID NO: 29 - protein, 9 aa, Artificial Sequence
  source: /note=“Description of Artificial Sequence Synthetic peptide”
EGPWLEEEE

SEQ ID NO: 30 - protein, 84 aa, Hirudinaria manillensis
MFSLKLFVVFLAVCICVSQAVSYTDCTESGQNYCLCVGGNLCGGGKHCEMDGSGNKCVDGEGTPKPKSQTEGDFEEIPDEDILN

SEQ ID NO: 31 - protein, 9 aa, Artificial Sequence
  source: /note=“Description of Artificial Sequence Synthetic peptide”
YAGAVVNDL

SEQ ID NO: 32 - protein, 100 aa, Homo sapiens [sequence omitted]

SEQ ID NO: 33 - protein, 338 aa, Mycobacterium bovis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 34 - protein, 9 aa, Artificial Sequence
  source: /note=“Description of Artificial Sequence Synthetic peptide”
FPDFEPIPL

SEQ ID NO: 35 - protein, 14 aa, Artificial Sequence
  source: /note=“Description of Artificial Sequence Synthetic peptide”
EQRLGNQWAVGHLL

SEQ ID NO: 36 - protein, 4 aa, Artificial Sequence
  source: /note=”Description of Artificial Sequence Synthetic peptide“
LDVP

SEQ ID NO: 37 - protein, 14 aa, Artificial Sequence
  source: /note=”Description of Artificial Sequence Synthetic peptide“
GEPPPGKPADDAGL

SEQ ID NO: 38 - protein, 14 aa, Artificial Sequence
  source: /note=”Description of Artificial Sequence Synthetic peptide“
PQRLGNQWAVGHLM

SEQ ID NO: 39 - protein, 32 aa, Homo sapiens
SPKMVQGSGCFGRKMDRISSSSGLGCKVLRRH

SEQ ID NO: 40 - protein, 37 aa, Artificial Sequence
  source: /note=”Description of Artificial Sequence Synthetic polypeptide“
GLSKGCFGLKLDRIGSMSGLGCPSLRDPRPNAPSTSA

SEQ ID NO: 41 - protein, 31 aa, Artificial Sequence
  source: /note=”Description of Artificial Sequence Synthetic polypeptide“
EAEDLQVGQVELGGGPGAGSLQPLALEGSLQ

SEQ ID NO: 42 - protein, 4 aa, Artificial Sequence
  source: /note=”Description of Artificial Sequence Synthetic peptide“
NLGV

SEQ ID NO: 43 - protein, 6 aa, Artificial Sequence
  source: /note=”Description of Artificial Sequence Synthetic peptide“
YMGWMD

SEQ ID NO: 44 - protein, 12 aa, Artificial Sequence
  source: /note=”Description of Artificial Sequence Synthetic peptide“
TYICEVEDQKEE

SEQ ID NO: 45 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 46 - protein, 37 aa, Artificial Sequence
  source: /note=”Description of Artificial Sequence Synthetic polypeptide“
ACDTATCVTHRLAGLLSRSGGVVKNNFVPTNVGSKAF

SEQ ID NO: 47 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 48 - protein, 16 aa, Artificial Sequence
  source: /note=”Description of Artificial Sequence Synthetic peptide“
LQNRRGLDLLFLKEGG

SEQ ID NO: 49 - protein, 7 aa, Artificial Sequence
  source: /note=”Description of Artificial Sequence Synthetic peptide“
TTSQVRP

SEQ ID NO: 50 - protein, 7 aa, Artificial Sequence
  source: /note=”Description of Artificial Sequence Synthetic peptide“
FCYWRTT

SEQ ID NO: 51 - protein, 4 aa, Artificial Sequence
  source: /note=”Description of Artificial Sequence Synthetic peptide“
CVFM

SEQ ID NO: 52 - protein, 32 aa, Homo sapiens
CGNLSTCMLGTYTQDFNKFHTFPQTAIGVGAP

SEQ ID NO: 53 - protein, 32 aa, Salmo salar
  misc_feature (32)..(32): /note=”C-term amidated“
CSNLSTCVLGKLSQELHKLQTYPRTNTGSGTP

SEQ ID NO: 54 - protein, 60 aa, Dendroaspis polylepis polylepis
RICYIHKASLPRATKTCVENTCYKMFIRTQREYISERGCGCPTAMWPYQTECCKGDRCNK

SEQ ID NO: 55 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 56 - protein, 31 aa, Sus scrofa
SWLSKTAKKLENSAKKRISEGIAIAIQGGPR

SEQ ID NO: 57 - protein, 5 aa, Artificial Sequence
  source: /note=”Description of Artificial Sequence Synthetic peptide“
VVVPP

SEQ ID NO: 58 - protein, 10 aa, Artificial Sequence
  source: /note=”Description of Artificial Sequence Synthetic peptide“
EQDYTGWMDF

SEQ ID NO: 59 - protein, 41 aa, Artificial Sequence
  source: /note=”Description of Artificial Sequence Synthetic polypeptide“
SQEPPISLDLTFHLLREVLEMTKADQLAQQAHSNRKLLDIA

SEQ ID NO: 60 - protein, 5 aa, Artificial Sequence
  source: /note=”Description of Artificial Sequence Synthetic peptide“
ARGFF

SEQ ID NO: 61 - protein, 41 aa, Artificial Sequence
  source: /note=”Description of Artificial Sequence Synthetic polypeptide“
MTLTVQARQLLSQIVQQQNNLLRAIEAQQHLLQLTVWGIKQ

SEQ ID NO: 62 - protein, 4 aa, Artificial Sequence
  source: /note=”Description of Artificial Sequence Synthetic peptide“
RGDS

SEQ ID NO: 63 - protein, 17 aa, Artificial Sequence
  source: /note=”Description of Artificial Sequence Synthetic peptide“
YGGFLRRIRPKLKWDNQ

SEQ ID NO: 64 - protein, 94 aa, Homo sapiens
MRTLAILAAILLVALQAQAEPLQARADEVAAAPEQIAADIPEVVVSLAWDESLAPKHPGSRKNMDCYCRIPACIAGERRYGTCIYQGRLWAFCC

SEQ ID NO: 65 - protein, 60 aa, Ornithodoros moubata
SDDKCQGRPMYGCREDDDSVFGWTYDSNHGQCWKGSYCKHRRQPSNYFASQQECRNTCGA

SEQ ID NO: 66 - protein, 7 aa, Artificial Sequence
  source: /note=”Description of Artificial Sequence Synthetic peptide“
YGGFLRR

SEQ ID NO: 67 - protein, 60 aa, Artificial Sequence
  source: /note=”Description of Artificial Sequence Synthetic polypeptide“
EAMHSFCAFKADDGPCRAAHPRWFFNIFTRQCEEFIYGGCEGNQNRFESLEECKKMCTRD

SEQ ID NO: 68 - protein, 21 aa, Homo sapiens
MRASSFLIVVVFLIAGTLVLE

SEQ ID NO: 69 - protein, 57 aa, Homo sapiens
AQEPVKGPVSTKPGSCPIILIRCAMLNPPNRCLKDTDCPGIKKCCEGSCGMACFVPQ

SEQ ID NO: 70 - protein, 31 aa, Homo sapiens
  misc_feature (31)..(31): /note=”C-term amidated“
SNLSTNVLGKLSQELHKLQTYPRTNVGAGTP

SEQ ID NO: 71 - protein, 12 aa, Artificial Sequence
  source: /note=”Description of Artificial Sequence Synthetic peptide“
PPSKDAFAIGLM

SEQ ID NO: 72 - protein, 31 aa, Homo sapiens
YGGFMTSEKSQTPLVTLFKNAIIKNAYKKGE

SEQ ID NO: 73 - protein, 482 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ

SEQ ID NO: 74 - protein, 5 aa, Artificial Sequence
  source: /note=”Description of Artificial Sequence Synthetic peptide“
GDWPC

SEQ ID NO: 75 - protein, 5 aa, Artificial Sequence
  source: /note=”Description of Artificial Sequence Synthetic peptide“
QHNPR

SEQ ID NO: 76 - protein, 5 aa, Artificial Sequence
  source: /note=”Description of Artificial Sequence Synthetic peptide“
GRGDS

SEQ ID NO: 77 - protein, 7 aa, Artificial Sequence
  source: /note=”Description of Artificial Sequence Synthetic peptide“
YYWIGIR

SEQ ID NO: 78 - protein, 92 aa, Homo sapiens
MKPIQKLLAGLILLTWCVEGCSSQHWSYGLRPGGKRDAENLIDSFQEIVKEVGQLAETQRFECTTHQPRSPLRDLKGALESLIEEETGQKKI

SEQ ID NO: 79 - protein, 44 aa, Homo sapiens
MPLWVFFFVILTLSNSSHCSPPPPLTLRMRRYADAIFTNSYRKV

SEQ ID NO: 80 - protein, 107 aa, Homo sapiens
MPLWVFFFVILTLSNSSHCSPPPPLTLRMRRYADAIFTNSYRKVLGQLSARKLLQDIMSRQQGESNQERGARARLGRQVDSMWAEQKQMELESILVALLQKHRNSQG

SEQ ID NO: 81 - protein, 123 aa, Homo sapiens
MARGSALLLASLLLAAALSASAGLWSPAKEKRGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 82 - protein, 7 aa, Artificial Sequence
  source: /note=”Description of Artificial Sequence Synthetic peptide“
EAYGYMD

SEQ ID NO: 83 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 84 - protein, 29 aa, Homo sapiens
HSQGTFTSDYSKYLDSRRAQDFVQWLMNT

SEQ ID NO: 85 - protein, 92 aa, Homo sapiens; identical to SEQ ID NO: 78

SEQ ID NO: 86 - protein, 9 aa, Artificial Sequence
  source: /note=”Description of Artificial Sequence Synthetic peptide“
HWSYGLRPG

SEQ ID NO: 87 - protein, 16 aa, Artificial Sequence
  source: /note=”Description of Artificial Sequence Synthetic peptide“
RGGLCYCRGRFCVCVG

SEQ ID NO: 88 - protein, 4 aa, Artificial Sequence
  source: /note=”Description of Artificial Sequence Synthetic peptide“
FPRG

SEQ ID NO: 89 - protein, 140 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQAN

SEQ ID NO: 90 - protein, 5 aa, Artificial Sequence
  source: /note=”Description of Artificial Sequence Synthetic peptide“
RMFRY

SEQ ID NO: 91 - protein, 8 aa, Artificial Sequence
  source: /note=”Description of Artificial Sequence Synthetic peptide“
VHPFHXLF

SEQ ID NO: 92 - protein, 8 aa, Artificial Sequence
  source: /note=“Description of Artificial Sequence Synthetic peptide”
FLDVPAAK

SEQ ID NO: 93 - protein, 6 aa, Artificial Sequence
  source: /note=“Description of Artificial Sequence Synthetic peptide”
PVTKPQ

SEQ ID NO: 94 - protein, 6 aa, Artificial Sequence
  source: /note=“Description of Artificial Sequence Synthetic peptide”
SIKVAV

SEQ ID NO: 95 - protein, 5 aa, Artificial Sequence
  source: /note=“Description of Artificial Sequence Synthetic peptide”
YIGSR

SEQ ID NO: 96 - protein, 11 aa, Artificial Sequence
  source: /note=“Description of Artificial Sequence Synthetic peptide”
ILRWPWWPWRR

SEQ ID NO: 97 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 98 - protein, 197 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKVSSEE

SEQ ID NO: 99 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 100 - protein, 22 aa, Homo sapiens
MASQKRPSQRHGSKYLATASTM

SEQ ID NO: 101 - protein, 4 aa, Artificial Sequence
  source: /note=“Description of Artificial Sequence Synthetic peptide”
GPLA

SEQ ID NO: 102 - protein, 4 aa, Homo sapiens
NAGA

SEQ ID NO: 103 - protein, 487 aa, Homo sapiens
MRENMARGPCNAPRWASLMVLVAIGTAVTAAVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITCSSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENHHNPPPFAPPVMEFPAAHDRMVYLGLSDYFFNTAGLVYQEAGVLKMTLRDDMIPKESKFRLTTKFFGTFLPEVAKKFPNMKIQIHVSASTPPHLSVQPTGLTFYPAVDVQAFAVLPNSSLASLFLIGMHTTGSMEVSAESNRLVGELKLDRLLLELKHSNIGPFPVELLQDIMNYIVPILVLPRVNEKLQKGFPLPTPARVQLYNVVLQPHQNFLLFGADVVYK

SEQ ID NO: 104 - protein, 31 aa, Homo sapiens
  MOD_RES (3)..(3): Alpha-Glu
HAEGTFTSDVSSYLEGQAAEKEFIAWLVRGR

SEQ ID NO: 105 - protein, 70 aa, Homo sapiens
SFKIKHLGKGHYSFYSMDIREFQLPSSQISMVPNVGLKFSISNANIKISGKWKAQKRFLKMSGNFDLSIE

SEQ ID NO: 106 - protein, 70 aa, Homo sapiens
GMSISADLKLGSNPTSGKPTITCSSCSSHINSVHVHISKSKVGWLIQLFHKKIESALRNKMNSQVCEKVT

SEQ ID NO: 107 - protein, 21 aa, Homo sapiens
CLGVGSCNDFAGCGYAIVCFW

SEQ ID NO: 108 - protein, 70 aa, Homo sapiens
NSVSSELQPYFQTLPVMTKIDSVAGINYGLVAPPATTAETLDVQMKGEFYSENHHNPPPFAPPVMEFPAA

SEQ ID NO: 109 - protein, 70 aa, Homo sapiens
HDRMVYLGLSDYFFNTAGLVYQEAGVLKMTLRDDMIPKESKFRLTTKFFGTFLPEVAKKFPNMKIQIHVS

SEQ ID NO: 110 - protein, 70 aa, Homo sapiens
ASTPPHLSVQPTGLTFYPAVDVQAFAVLPNSSLASLFLIGMHTTGSMEVSAESNRLVGELKLDRLLLELK

SEQ ID NO: 111 - protein, 67 aa, Homo sapiens
HSNIGPFPVELLQDIMNYIVPILVLPRVNEKLQKGFPLPTPARVQLYNVVLQPHQNFLLFGADVVYK

SEQ ID NO: 112 - protein, 15 aa, Artificial Sequence
  source: /note=“Description of Artificial Sequence Synthetic peptide”
GEPPPGKPADDAGLV

SEQ ID NO: 113 - protein, 6 aa, Homo sapiens
DLQVFV

SEQ ID NO: 114 - protein, 5 aa, Homo sapiens
GEKKD

SEQ ID NO: 115 - protein, 5 aa, Homo sapiens
ATHES

SEQ ID NO: 116 - protein, 6 aa, Homo sapiens
CLPVSG

SEQ ID NO: 117 - protein, 6 aa, Homo sapiens
LPVSGS

SEQ ID NO: 118 - protein, 6 aa, Homo sapiens
PCHAPP

SEQ ID NO: 119 - protein, 8 aa, Homo sapiens
GGHDLESG

SEQ ID NO: 120 - protein, 6 aa, Homo sapiens
DDLQVF

SEQ ID NO: 121 - protein, 5 aa, Homo sapiens
PLTSG

SEQ ID NO: 122 - protein, 8 aa, Homo sapiens
LIHFEEGV

SEQ ID NO: 123 - protein, 7 aa, Homo sapiens
GEFSYDS

SEQ ID NO: 124 - protein, 7 aa, Homo sapiens
HAPPLTS

SEQ ID NO: 125 - protein, 7 aa, Homo sapiens
DLESGEF

SEQ ID NO: 126 - protein, 8 aa, Homo sapiens
GEFSVCDS

SEQ ID NO: 127 - protein, 10 aa, Homo sapiens
KKGEFSVADS

SEQ ID NO: 128 - protein, 9 aa, Homo sapiens
KKGEFYCSR

SEQ ID NO: 129 - protein, 13 aa, Homo sapiens
GLRVRVWNGKFPK

SEQ ID NO: 130 - protein, 16 aa, Homo sapiens
GVAFEEAPDDHSFFLF

SEQ ID NO: 131 - protein, 7 aa, Homo sapiens
GGHDLSG

SEQ ID NO: 132 - protein, 8 aa, Homo sapiens
GGHDLESG

SEQ ID NO: 133 - protein, 14 aa, Homo sapiens
GGHDLESGEFSYDS

SEQ ID NO: 134 - protein, 14 aa, Homo sapiens
GGSDLSGEFSVCDS

SEQ ID NO: 135 - protein, 15 aa, Homo sapiens
GGSDLSGGEFSVCDS

SEQ ID NO: 136 - protein, 15 aa, Homo sapiens
GGSDLSGGEFSVADS

SEQ ID NO: 137 - protein, 14 aa, Homo sapiens
GGSDLSGEFSVADS

SEQ ID NO: 138 - protein, 6 aa, Homo sapiens
ETLQFR

SEQ ID NO: 139 - protein, 8 aa, Homo sapiens
KKETLQFR

SEQ ID NO: 140 - protein, 8 aa, Homo sapiens
ETLQFRKK

SEQ ID NO: 141 - protein, 9 aa, Homo sapiens
KASTTTNYT

SEQ ID NO: 142 - protein, 13 aa, Homo sapiens
ALKRQGRTLYGFG

SEQ ID NO: 143 - protein, 8 aa, Homo sapiens
  MOD_RES (1)..(1): Sarcosine
XRVYVHPA

SEQ ID NO: 144 - protein, 60 aa, Artificial Sequence
  source: /note=“Description of Artificial Sequence Synthetic polypeptide”
RKRGRQTYTRYQTLELEKEFHFNRYLTRRRRIEIAHALCLTERQIKIWFQNRRMKWKKEN

SEQ ID NO: 145 - protein, 38 aa, Homo sapiens
RRRPRPPYLPRPRPPPFFPPRLPPRIPPGFPPRFPPRF

SEQ ID NO: 146 - protein, 14 aa, Artificial Sequence
  source: /note=“Description of Artificial Sequence Synthetic peptide”
EWQTDNCETCTCYE

SEQ ID NO: 147 - protein, 116 aa, Homo sapiens
MLSCRLQCALAALSIVLALGCVTGAPSDPRLRQFLQKSLAAAAGKQELAKYFLAELLSEPNQTENDALEPEDLSQAAEQDEMRLELQRSANSNPAMAPRERKAGCKNFFWKTFTSC

SEQ ID NO: 148 - protein, 176 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 149 - protein, 34 aa, Homo sapiens
SVSEIQLMHNLGKHLNSMERVEWLRKKLQDVHNF

SEQ ID NO: 150 - protein, 35 aa, Artificial Sequence
  source: /note=“Description of Artificial Sequence Synthetic polypeptide”
YTSLIHSLIEESQNQQEKNEQELLELDKWASLWNW

SEQ ID NO: 151 - protein, 5 aa, Homo sapiens
ARPAK

SEQ ID NO: 152 - protein, 37 aa, Artificial Sequence [sequence omitted]

SEQ ID NO: 153 - protein, 30 aa, Artificial Sequence
  source: /note=“Description of Artificial Sequence Synthetic polypeptide”
HSDAVFTENYTKLRKQLAAKKYLNDLKKGG

SEQ ID NO: 154 - protein, 5 aa, Artificial Sequence
  source: /note=“Description of Artificial Sequence Synthetic peptide”
LKRMP

SEQ ID NO: 155 - protein, 5 aa, Artificial Sequence
  source: /note=“Description of Artificial Sequence Synthetic peptide”
EEDCK

SEQ ID NO: 156 - protein, 43 aa, Artificial Sequence
  source: /note=“Description of Artificial Sequence Synthetic polypeptide”
MFLEAIPMSIPPEVKFNKPFVFLMIEQNTKSPLFMGKVVNPTQ

SEQ ID NO: 157 - protein, 7 aa, Homo sapiens
  MOD_RES (1)..(1): Isoval
VFLXAXK

SEQ ID NO: 158 - protein, 32 aa, Salmo salar; identical to SEQ ID NO: 53

SEQ ID NO: 159 - protein, 29 aa, Artificial Sequence
  source: /note=“Description of Artificial Sequence Synthetic peptide”
YADAIFTNSYRKVLGQLSARKLLQDIMSR

SEQ ID NO: 160 - protein, 6 aa, Artificial Sequence
  source: /note=“Description of Artificial Sequence Synthetic peptide”
HSDGTF

SEQ ID NO: 161 - protein, 20 aa, Artificial Sequence
  source: /note=“Description of Artificial Sequence Synthetic peptide”
KLLLLKLLLLKLLLLKLLLL

SEQ ID NO: 162 - protein, 9 aa, Artificial Sequence
  source: /note=“Description of Artificial Sequence Synthetic peptide”
WAGGDASGE

SEQ ID NO: 163 - protein, 108 aa, Homo sapiens
MPLWVFFFVILTLSNSSHCSPPPPLTLRMRRYADAIFTNSYRKVLGQLSARKLLQDIMSRQQGESNQERGARARLGRQVDSMWAEQKQMELESILVALLQKHSRNSQG

SEQ ID NO: 164 - protein, 70 aa, Homo sapiens
MPLWVFFFVILTLSNSSHCSPPPPLTLRMRRYADAIFTNSYRKVLGQLSARKLLQDIMSRQQGESNQERG

SEQ ID NO: 165 - protein, 38 aa, Homo sapiens
ARARLGRQVDSMWAEQKQMELESILVALLQKHSRNSQG

SEQ ID NO: 166 - protein, 200 aa, Homo sapiens
MASLYVGDLHPEVTEAMLYEKFSPAGPILSIRICRDKITRRSLGYAYVNYQQPVDAKRALETLNFDVIKGRPVRIMWSQRDPSLRKSGVGNVFIKNLGKTIDNKALYNIFSAFGNILSCKVACDEKGPKGYGFVHFQKQESAERAIDVMNGMFLNYRKIFVGRFKSHKEREAERGAWARQSTSADVKDFEEDTDEEATLR

SEQ ID NO: 167 - protein, 17 aa, Artificial Sequence
  source: /note=“Description of Artificial Sequence Synthetic peptide”
RRWCYRKCYKGYCYRKC

SEQ ID NO: 168 - protein, 8 aa, Homo sapiens
LEDGPKFL

SEQ ID NO: 169 - protein, 30 aa, Artificial Sequence
  source: /note=“Description of Artificial Sequence Synthetic polypeptide”
KSSQYIKANSKFIGITEAAAFLPSDFFPSV

SEQ ID NO: 170 - protein, 8 aa, Artificial Sequence
  source: /note=“Description of Artificial Sequence Synthetic peptide”
CNPRGDYR

SEQ ID NO: 171 - protein, 4 aa, Artificial Sequence
  source: /note=“Description of Artificial Sequence Synthetic peptide”
GGGK

SEQ ID NO: 172 - protein, 12 aa, Artificial Sequence
  source: /note=“Description of Artificial Sequence Synthetic peptide”
GGGCYFQNCPKG

SEQ ID NO: 173 - protein, 7 aa, Artificial Sequence
  source: /note=“Description of Artificial Sequence Synthetic peptide”
QEDLIDV

SEQ ID NO: 174 - protein, 214 aa, Mus musculus
MEPGLWLLLGLTVTSAAGLVPCPQSGDSGRASVSQGPPEAGSERGCEETVAGPGERIVSPTVALPAQPESAGQERAPGRSGKQEDKGLPAHHRPRRCTCFTYKDKECVYYCHLDIIWINTPEQTVPYGLSNYRESLRGKRSLGPVPESSQPSPWTRLRCTCMGADDKACAHFCARTRDVTSYSGRAERPAAEEMRETGGPRQRLMSRTDKAHRP

SEQ ID NO: 175 - protein, 356 aa, Homo sapiens
MAAEGWIWRWGWGRRCLGRPGLLGPGPGPTTPLFLLLLLGSVTADITDGNSEHLKREHSLIKPYQGVGSSSMPLWDFQGSTMLTSQYVRLTPDERSKEGSIWNHQPCFLKDWEMHVHFKVHGTGKKNLHGDGIALWYTRDRLVPGPVFGSKDNFHGLAIFLDTYPNDETTERVFPYISVMVNNGSLSYDHSKDGRWTELAGCTADFRNRDHDTFLAVRYSRGRLTVMTDLEDKNEWKNCIDITGVRLPTGYYFGASAGTGDLSDNHDIISMKLFQLMVEHTPDEESIDWTKIEPSVNFLKSPKDNVDDPTGNFRSGPLTGWRVFLLLLCALLGIVVCAVVGAVVFQKRQERNKRFY

SEQ ID NO: 176 - protein, 8 aa, Artificial Sequence
  source: /note=“Description of Artificial Sequence Synthetic peptide”
TDSFVGLM

SEQ ID NO: 177 - protein, 8 aa, Homo sapiens
KPGEPGPK

SEQ ID NO: 178 - protein, 4 aa, Artificial Sequence
  source: /note=“Description of Artificial Sequence Synthetic peptide”
GSHK

SEQ ID NO: 179 - protein, 4 aa, Artificial Sequence
  source: /note=“Description of Artificial Sequence Synthetic peptide”
ASHK

SEQ ID NO: 180 - protein, 4 aa, Artificial Sequence
  source: /note=“Description of Artificial Sequence Synthetic peptide”
LSHK

SEQ ID NO: 181 - protein, 4 aa, Artificial Sequence
  source: /note=“Description of Artificial Sequence Synthetic peptide”
TSHK

SEQ ID NO: 182 - protein, 4 aa, Artificial Sequence
  source: /note=“Description of Artificial Sequence Synthetic peptide”
YSHK

SEQ ID NO: 183 - protein, 4 aa, Artificial Sequence
  source: /note=“Description of Artificial Sequence Synthetic peptide”
PSHK

SEQ ID NO: 184 - protein, 4 aa, Artificial Sequence
  source: /note=“Description of Artificial Sequence Synthetic peptide”
ESHK

SEQ ID NO: 185 - protein, 4 aa, Artificial Sequence
  source: /note=“Description of Artificial Sequence Synthetic peptide”
WSHK

SEQ ID NO: 186 - protein, 5 aa, Artificial Sequence
  source: /note=“Description of Artificial Sequence Synthetic peptide”
PPGAG

SEQ ID NO: 187 - protein, 5 aa, Artificial Sequence
  source: /note=“Description of Artificial Sequence Synthetic peptide”
WASGN

SEQ ID NO: 188 - protein, 8 aa, Homo sapiens
DADPRQYA

SEQ ID NO: 189 - protein, 518 aa, Homo sapiens
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 190 - protein, 19 aa, Artificial Sequence
  source: /note=“Description of Artificial Sequence Synthetic peptide”
GYYWSWIRQPPGKGEEWIG

SEQ ID NO: 191 - protein, 82 aa, Homo sapiens; identical to SEQ ID NO: 6

SEQ ID NO: 192 - protein, 10 aa, Artificial Sequence
  source: /note=“Description of Artificial Sequence Synthetic peptide”
LRAHAVDVNG

SEQ ID NO: 193 - protein, 9 aa, Homo sapiens
LLPGGRPYR

SEQ ID NO: 194 - protein, 359 aa, Homo sapiens [sequence omitted]

SEQ ID NO: 195 - protein, 11 aa, Artificial Sequence
  source: /note=“Description of Artificial Sequence Synthetic peptide”
ILRWPWWPWRR

SEQ ID NO: 196 - protein, 109 aa, Homo sapiens
MRGALLVLALLVTQALGVKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 197 - protein, 138 aa, Homo sapiens
MNSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 198 - protein, 422 aa, Homo sapiens
MSTSYGCFWRRFIHGIGRSGDISAVQPKAAGSSLLNKITNSLVLDIIKLAGVHTVANCFVVPMATGMSLTLCFLTLRHKRPKAKYIIWPRIDQKSCFKSMITAGFEPVVIENVLEGDELRTDLKAVEAKVQELGPDCILCIHSTTSCFAPRVPDRLEELAVICANYDIPHIVNNAYGVQSSKCMHLIQQGARVGRIDAFVQSLDKNFMVPVGGAIIAGFNDSFIQEISKMYPGRASASPSLDVLITLLSLGSNGYKKLLKERKEMFSYLSNQIKKLSEAYNERLLHTPHNPISLAMTLKTLDEHRDKAVTQLGSMLFTKQVSGARVVPLGSMQTVSGYTFRGFMSHTNNYPCAYLNAASAIGMKMQDVDLFINRLDRCLKAVRKERSKESDDNYDKTEDVDIEEMALKLDNVLLDTYQDASS

SEQ ID NO: 199 - protein, 246 aa, Homo sapiens
MTLIEGVGDEVTVLFSVLACLLVLALAWVSTHTAEGGDPLPQPSGTPTPSQPSAAMAATDSMRGEAPGAETPSLRHRGQAAQPEPSTGFTATPPAPDSPQEPLVLRLKFLNDSEQVARAWPHDTIGSLKRTQFPGREQQVRLIYQGQLLGDDTQTLGSLHLPPNCVLHCHVSTRVGPPNPPCPPGSEPGPSGLEIGSLLLPLLLLLLLLLWYCQIQYRPFFPLTATLGLAGFTLLLSLLAFAMYRP

SEQ ID NO: 200 - protein, 39 aa, Homo sapiens; identical to SEQ ID NO: 8
  misc_feature (39)..(39): /note=“C-term amidated”

SEQ ID NO: 201 - protein, 177 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 202 - protein, 36 aa, Artificial Sequence
  source: /note=“Description of Artificial Sequence Synthetic polypeptide”
MCMPCFTTDHQMARKCDDCCGGKGRGKCYGPQCLCR

SEQ ID NO: 203 - protein, 12 aa, Artificial Sequence
  source: /note=“Description of Artificial Sequence Synthetic peptide”
RYYRWKKKKKKK

SEQ ID NO: 204 - protein, 10 aa, Artificial Sequence
  source: /note=“Description of Artificial Sequence Synthetic peptide”
CTTHWGFTLC

SEQ ID NO: 205 - protein, 97 aa, Homo sapiens
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 206 - protein, 8 aa, Homo sapiens
NAPVSIPQ

SEQ ID NO: 207 - protein, 711 aa, Homo sapiens
MAERESGGLGGGAASPPAASPFLGLHIASPPNFRLTHDISLEEFEDEDLSEITDECGISLQCKDTLSLRPPRAGLLSAGGGGAGSRLQAEMLQMDLIDATGDTPGAEDDEEDDDEERAARRPGAGPPKAESGQEPASRGQGQSQGQSQGPGSGDTYRPKRPTTLNLFPQVPRSQDTLNNNSLGKKHSWQDRVSRSSSPLKTGEQTPPHEHICLSDELPPQSGPAPTTDRGTSTDSPCRRSTATQMAPPGGPPAAPPGGRGHSHRDRIHYQADVRLEATEEIYLTPVQRPPDAAEPTSAFLPPTESRMSVSSDPDPAAYPSTAGRPHPSISEEEEGFDCLSSPERAEPPGGGWRGSLGEPPPPPRASLSSDTSALSYDSVKYTLVVDEHAQLELVSLRPCFGDYSDESDSATVYDNCASVSSPYESAIGEEYEEAPRPQPPACLSEDSTPDEPDVHFSKKFLNVFMSGRSRSSSAESFGLFSCIINGEEQEQTHRAIFRFVPRHEDELELEVDDPLLVELQAEDYWYEAYNMRTGARGVFPAYYAIEVTKEPEHMAALAKNSDWVDQFRVKFLGSVQVPYHKGNDVLCAAMQKIATTRRLTVHFNPPSSCVLEISVRGVKIGVKADDSQEAKGNKCSHFFQLKNISFCGYHPKNNKYFGFITKHPADHRFACHVFVSEDSTKALAESVGRAFQQFYKQFVEYTCPTEDIYLE

SEQ ID NO: 208 - protein, 710 aa, Homo sapiens
MAERESGGLGGGAASPPAASPFLGLHIASPPNFRLTHDISLEEFEDEDLSEITDECGISLQCKDTLSLRPPRAGLLSAGGGGAGSRLQAEMLQMDLIDATGDTPGAEDDEEDDDEERAARRPGAGPPKAESGQEPASRGQGQSQGQSQGPGSGDTYRPKRPTTLNLFPQVPRSQDTLNNNSLGKKHSWQDRVSRSSSPLKTGEQTPPHEHICLSDELPPQSGPAPTTDRGTSTDSPCRRSTATQMAPPGGPPAAPPGGRGHSHRDRIHYQADVRLEATEEIYLTPVQRPPDAAEPTSAFLPPTESRMSVSSDPDPAAYPSTAGRPHPSISEEEEGFDCLSSPERAEPPGGGWRGSLGEPPPPPRASLSSDTSALSYDSVKYTLVVDEHAQLELVSLRPCFGDYSDESDSATVYDNCASVSSPYESAIGEEYEEAPRPQPPACLSEDSTPDEPDVHFSKKFLNVFMSGRSRSSSAESFGLFSCIINGEEQEQTHRAIFRFVPRHEDELELEVDDPLLVELQAEDYWYEAYNMRTGARGVFPAYYAIEVTKEPEHMAALAKNSDWVDQFRVKFLGSVQVPYHKGNDVLCAAMQKIATTRRLTVHFNPPSSCVLEISVRGVKGVKADDSQEAKGNKCSHFFQLKNISFCGYHPKNNKYFGFITKHPADHRFACHVFVSEDSTKALAESVGRAFQQFYKQFVEYTCPTEDIYLE

SEQ ID NO: 209 - protein, 5 aa, Artificial Sequence
  source: /note=“Description of Artificial Sequence Synthetic peptide”
YGFGG

SEQ ID NO: 210 - protein, 9 aa, Artificial Sequence
  source: /note=“Description of Artificial Sequence Synthetic peptide”
YCWSQYLCY

SEQ ID NO: 211 - protein, 28 aa, Homo sapiens
HSDAVFTDNYTRLRKQMAVKKYLNSILN

SEQ ID NO: 212 - protein, 9 aa, Homo sapiens
SALLRSIPA

SEQ ID NO: 213 - protein, 14 aa, Artificial Sequence
  source: /note=“Description of Artificial Sequence Synthetic peptide”
LEEKKGNYVVTDHC

SEQ ID NO: 214 - protein, 6 aa, Artificial Sequence
  source: /note=“Description of Artificial Sequence Synthetic peptide”
NFGAIL

SEQ ID NO: 215 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 216 - protein, 64 aa, Homo sapiens
MAKVFSFILVTTALTMGREISALEDCAQEQMRLRAQVRLLETRVKQQQVKIKQLLQENEVQFLD

SEQ ID NO: 217 - protein, 19 aa, Vibrio cholerae
NLIDCCEICCNPACFGCLN

SEQ ID NO: 218 - protein, 83 aa, Laticauda semifasciata
MKTLLLTLVVVTIVCLDLGYTRICFNHQSSQPQTTKTCSPGESSCYNKQWSDFRGTIIERGCGCPTVKPGIKLSCCESEVCNN

SEQ ID NO: 219 - protein, 141 aa, Artificial Sequence
  source: /note=“Description of Artificial Sequence Synthetic polypeptide”
MSNKKIIKIIKLQIPGGKANPAPPIGPALGAAGVNIMGFCKEFNAATQDRPGDLLPVVITVYSDKTFSFVMKQSPVSSLIKKALGLESGSKIPNRNKVGKLTRAQITVIAEQKMKDMDVVLLESAERMVEGTARSMGVDVE

SEQ ID NO: 220 - protein, 172 aa, Mus musculus
MSVPEPPPPDGVLTGPSDSLEAGEPTPGLSDTSPDEGLIEDFPVDDRAVEHLVGGLLSHYLPDLQRSKRALQELTQNQVVLLDTLEQEISKFKECHSMLDINALFTEAKHYHAKLVTIRKEMLLLHEKTSKLKKRALKLQQKRQREELEREQQREKEFEREKQLTAKPAKRT

SEQ ID NO: 221 - protein, 90 aa, Human immunodeficiency virus type 1
KLTPLCVTLNCTDLDLRNTTNNTTTEERGEMKNCSFNITTNIRDRYQKEYALFYKLDVIPIKEDNTSDNTSYRLISCNTSVITQACPKIS

SEQ ID NO: 222 - protein, 191 aa, Bubalus bubalis
AFPAMSLSSLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKRTYDKFDTNMRSDDALLKNYGLLSCFRKDLHKTETYLRVMKCRRFGEASCAF

SEQ ID NO: 223 - protein, 14 aa, Artificial Sequence
  source: /note=“Description of Artificial Sequence Synthetic peptide”
LSENDEWTQDRAKP

SEQ ID NO: 224 - protein, 11 aa, Artificial Sequence
  source: /note=“Description of Artificial Sequence Synthetic peptide”
NPFPTWRKRPG

SEQ ID NO: 225 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 226 - protein, 87 aa, Heloderma suspectum
MKIILWLCVFGLFLATLFPISWQMPVESGLSSEDSASSESFASKIKRHGEGTFTSDLSKQMEEEAVRLFIEWLKNGGPSSGAPPPSG

SEQ ID NO: 227 - protein, 13 aa, Artificial Sequence
  source: /note=“Description of Artificial Sequence Synthetic peptide”
HGVSGHGQHGVHG

SEQ ID NO: 228 - protein, 9 aa, Artificial Sequence
  source: /note=“Description of Artificial Sequence Synthetic peptide”
KWRRWVRWI

SEQ ID NO: 229 - protein, 12 aa, Artificial Sequence
  source: /note=“Description of Artificial Sequence Synthetic peptide”
KFHEKHHSHRGY

SEQ ID NO: 230 - protein, 51 aa, Homo sapiens
MKFFVFALILALMLSMTGADSHAKRHHGYKRKFHEKHHSHRGYRSNYLYDN

SEQ ID NO: 231 - protein, 5 aa, Artificial Sequence
  source: /note=“Description of Artificial Sequence Synthetic peptide”
MQCNS

SEQ ID NO: 232 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 233 - protein, 6 aa, Artificial Sequence
  source: /note=“Description of Artificial Sequence Synthetic peptide”
CNGRCG

SEQ ID NO: 234 - protein, 4 aa, Artificial Sequence
  source: /note=“Description of Artificial Sequence Synthetic peptide”
VPVD

SEQ ID NO: 235 - protein, 5 aa, Artificial Sequence
  source: /note=“Description of Artificial Sequence Synthetic peptide”
VPDPR

SEQ ID NO: 236 - protein, 348 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIFLSNNYLPIPGIKKTDEGTYRCEGRILARGEINFNDIQVIVNVPPTIQARQNIVNATANLGQSVTLVCDAEGFPGPTMSWTKDGEQIEQEEHDEKYLFSDDSSHLTIKKVDKNHEAENICIAENKVGEQDATIHLKVFAKPQITYVEDQTAMELAEQVILTVEASGDHIPYITWWTSTWQI

SEQ ID NO: 237 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 238 - protein, 24 aa, Artificial Sequence
  source: /note=“Description of Artificial Sequence Synthetic peptide”
ELYSALANKCCHVGCTKRSLARFC

SEQ ID NO: 239 - protein, 28 aa, Artificial Sequence
  source: /note=“Description of Artificial Sequence Synthetic peptide”
DSWMEEVIKLCGRELVRAQIAICGMSTS

SEQ ID NO: 240 - protein, 31 aa, Homo sapiens; identical to SEQ ID NO: 41

SEQ ID NO: 241 - protein, 80 aa, Homo sapiens
MDKFWWHAAWGLCLVPLSLAQIDLNITCRFAGVFHVEKNGRYSISRTEAADLCKAFNSTLPTMAQMEKALSIGFETCSST

SEQ ID NO: 242 - protein, 83 aa, Bitis arietans
SPPVCGNKILEQGEDCDCGSPANCQDRCCNAATCKLTPGSQCNYGECCDQCRFKKAGTVCRIARGDWNDDYCTGKSSDCPWNH

SEQ ID NO: 243 - protein, 50 aa, Homo sapiens
MECESGPCCRNCKFLKEGTICKRARGDDLDDYCNGKTCDCPRNPHKGPAT

SEQ ID NO: 244 - protein, 82 aa, Homo sapiens
EASWKPRSQQPDAPLGTGANRDLELPWLEQQGPASHHRRQLGPQGPPHLVADPSKKQGPWLEEEEEAYGWMDFGRRSAEDEN

SEQ ID NO: 245 - protein, 23 aa, Human herpesvirus 1
MGGAAARLGAVILFVVIVGLHGV

SEQ ID NO: 246 - protein, 12 aa, Homo sapiens
LYENKPRRPYIL

SEQ ID NO: 247 - protein, 176 aa, Homo sapiens
MKVLLCDLLLLSLFSSVFSSCQRDCLTCQEKLHPALDSFDLEVCILECEEKVFPSPLWTPCTKVMARSSWQLSPAAPEHVAAALYQPRASEMQHLRRMPRVRSLFQEQEEPEPGMEEAGEMEQKQLQKRFGGFTGARKSARKLANQKRFSEFMRQYLVLSMQSSQRRRTLHQNGNV

SEQ ID NO: 248 - protein, 37 aa, Homo sapiens
HSQGTFTSDYSKYLDSRRAQDFVQWLMNTKRNRNNIA

SEQ ID NO: 249 - protein, 49 aa, Sus scrofa
GWPQAPAMDGAGKTGAEEAQPPEGKGAREHSRQEEEEETAGAPQGLFRG

SEQ ID NO: 250 - protein, 185 aa, Homo sapiens
MPRLFLFHLLEFCLLLNQFSRAVAAKWKDDVIKLCGRELVRAQIAICGMSTWSKRSLSQEDAPQTPRPVAEIVPSFINKDTETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 251 - protein, 27 aa, Homo sapiens
HSDGTFTSELSRLREGARLQRLLQGLV

SEQ ID NO: 252 - protein, 207 aa, Artificial Sequence
  source: /note=“Description of Artificial Sequence Synthetic polypeptide”
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 253 - protein, 74 aa, Artificial Sequence
  source: /note=“Description of Artificial Sequence Synthetic polypeptide”
DTTVSEPAPSCVTLYQSWRYSQADNGCAQTVTVKVVYEDDTEGLCYAVAPGQITTVGDGYIGSHGHARYLARCL

SEQ ID NO: 254 - protein, 44 aa, Homo sapiens
MSDKPDMAEIEKFDKSKLKKTETQEKNPLPSKETIEQEKQAGES

SEQ ID NO: 255 - protein, 32 aa, Rana tigrina
TAPRSLRRSSCFGGRMDRIGAQSGLGCNSFRY

SEQ ID NO: 256 - protein, 10 aa, Artificial Sequence
  source: /note=“Description of Artificial Sequence Synthetic peptide”
GSRAHSSHLK

SEQ ID NO: 257 - protein, 16 aa, Artificial Sequence
  source: /note=“Description of Artificial Sequence Synthetic peptide”
ELKCYTCKEPMTSAAC

SEQ ID NO: 258 - protein, 4 aa, Homo sapiens
DAHK

SEQ ID NO: 259 - protein, 38 aa, Artificial Sequence
  source: /note=“Description of Artificial Sequence Synthetic polypeptide”
KMVQGSGCFGRKMDISSSSGLGCPSLRDPRPNAPSTSA

SEQ ID NO: 260 - protein, 54 aa, Homo sapiens
  misc_feature (54)..(54): /note=“C-term amidated”
GTSLSPPPESSGSPQQPGLSAPHSRQIPAPQGAVLVQREKDLPNYNWNSFGLRF

SEQ ID NO: 261 - protein, 14 aa, Homo sapiens
  misc_feature (14)..(14): /note=“C-term amidated”
DLPNYNWNSFGLRF

SEQ ID NO: 262 - protein, 13 aa, Homo sapiens
  misc_feature (13)..(13): /note=“C-term amidated”
LPNYNWNSFGLRF

SEQ ID NO: 263 - protein, 10 aa, Homo sapiens
  misc_feature (10)..(10): /note=“C-term amidated”
YNWNSFGLRF

SEQ ID NO: 264 - protein, 25 aa, Artificial Sequence
  source: /note=“Description of Artificial Sequence Synthetic peptide”
CKGKGAKCSRLMYDCCTGSCRSGKC

SEQ ID NO: 265 - protein, 18 aa, Sus scrofa
  misc_feature (18)..(18): /note=“C-term amidated”
RGGRLCYCRRRFCVCVGR

SEQ ID NO: 266 - protein, 26 aa, Artificial Sequence
  source: /note=“Description of Artificial Sequence Synthetic peptide”
KWKSFLKTFKSAVKTVLHTALKAISS

SEQ ID NO: 267 - protein, 25 aa, Artificial Sequence
  source: /note=“Description of Artificial Sequence Synthetic peptide”
KWKSFLKTFKSVKTVLHTALKAISS

SEQ ID NO: 268 - protein, 26 aa, Artificial Sequence
  source: /note=“Description of Artificial Sequence Synthetic peptide”
KWKSFLKTFKSAKKTVLHTALKAISS

SEQ ID NO: 269 - protein, 26 aa, Artificial Sequence
  source: /note=“Description of Artificial Sequence Synthetic peptide”
KWKSFLKTFKSAKKKVLHTALKAISS

SEQ ID NO: 270 - protein, 33 aa, Homo sapiens
HADGSFSDEMNTILDNLAARDFINWLIQTKITD

SEQ ID NO: 271 - protein, 33 aa, Homo sapiens
HGDGSFSDEMNTILDNLAARDFINWLIQTKITD

SEQ ID NO: 272 - protein, 34 aa, Homo sapiens
HGDGSFSDEMNTILDNLAARDFINWLIQTKITDC

SEQ ID NO: 273 - protein, 16 aa, Homo sapiens
LRIVQCASVEGSCGFY

SEQ ID NO: 274 - protein, 16 aa, Homo sapiens
  misc_feature (1)..(1): /note=“N-term acetylated”
LRIVQCAKVEGSCGFY

SEQ ID NO: 275 - protein, 17 aa, Homo sapiens
  misc_feature (1)..(1): /note=“N-term acetylated”
LRIVQCASVEGSCGFYK

SEQ ID NO: 276 - protein, 31 aa, Homo sapiens; identical to SEQ ID NO: 41

SEQ ID NO: 277 - protein, 16 aa, Sus scrofa
RGGRLCYCRRRFCICV

SEQ ID NO: 278 - protein, 19 aa, Sus scrofa
RGGGLCYCRRRFCVCVGRG

SEQ ID NO: 279 - protein, 19 aa, Sus scrofa
RGGRLCYCRGWICFCVGRG

SEQ ID NO: 280 - protein, 19 aa, Sus scrofa
RGGRLCYCRPRFCVCVGRG

SEQ ID NO: 281 - protein, 149 aa, Sus scrofa
METQRASLCLGRWSLWLLLLGLVVPSASAQALSYREAVLRAVDRLNEQSSEANLYRLLELDQPPKADEDPGTPKPVSFTVKETVCPRPTRQPPELCDFKENGRVKQCVGTVTLDQIKDPLDITCNEVQGVRGGRLCYCRPRFCVCVGRG

SEQ ID NO: 282 - protein, 5 aa, Homo sapiens
EGSLC

SEQ ID NO: 283 - protein, 5 aa, Homo sapiens
YGGFM

SEQ ID NO: 284 - protein, 9 aa, Homo sapiens
  MOD_RES (3)..(3): Tyr(SO3)
RDYTGWLDF

SEQ ID NO: 285 - protein, 13 aa, Homo sapiens
YGGFLRRIRPKLK

SEQ ID NO: 286 - protein, 25 aa, Artificial Sequence
  source: /note=“Description of Artificial Sequence Synthetic peptide”
RSSCFGGRMDRIGAQSGLGCNSFRY

SEQ ID NO: 287 - protein, 37 aa, Homo sapiens
EVVPPQVLSDPNEEAGAALSPLPEVPPWTGEVSPAQR

SEQ ID NO: 288 - protein, 65 aa, Artificial Sequence
  source: /note=“Description of Artificial Sequence Synthetic polypeptide”
VVYTDCTESGQNLCLCEGSNVCGQGNKCILGSDGEKNQCVTGEGTPKPQSHNDGDFEEIPEEYLQ

SEQ ID NO: 289 - protein, 14 aa, Homo sapiens
ALKRQGRTLYGFGG

SEQ ID NO: 290 - protein, 5 aa, Homo sapiens
YGFGG

SEQ ID NO: 291 - protein, 11 aa, Homo sapiens
  misc_feature (1)..(1): /note=“N-term acetylated”
ASQKRPSQRHG

SEQ ID NO: 292 - protein, 11 aa, Homo sapiens
  misc_feature (1)..(1): /note=“N-term acetylated”
ASQYRPSQRHG

SEQ ID NO: 293 - protein, 10 aa, Artificial Sequence
  source: /note=“Description of Artificial Sequence Synthetic peptide”
EHWSYGLRPG

SEQ ID NO: 294 - protein, 15 aa, Phodopus sp.
GLHDPSHGTLPNGSG

SEQ ID NO: 295 - protein, 41 aa, Artificial Sequence
  source: /note=“Description of Artificial Sequence Synthetic polypeptide”
IVLSLDVPIGLLQILLEQARARAAREQATTNARILARVGHC

SEQ ID NO: 296 - protein, 8 aa, Artificial Sequence
  source: /note=“Description of Artificial Sequence Synthetic peptide”
KPSSPPEE

SEQ ID NO: 297 - protein, 23 aa, Homo sapiens
  misc_feature (23)..(23): /note=“C-term amidated”
FNAPFDVGIKLSGVQYQQHSQAL

SEQ ID NO: 298 - protein, 4 aa, Artificial Sequence
  source: /note=“Description of Artificial Sequence Synthetic peptide”
GKPR

SEQ ID NO: 299 - protein, 22 aa, Homo sapiens
GLSKGCFGLKLDRIGSMSGLGC

SEQ ID NO: 300 - protein, 49 aa, Homo sapiens
YLYQWLGAPVPYPDPLEPRREVCELNPDCDELADHIGFQEAYRRFYGPV

SEQ ID NO: 301 - protein, 31 aa, Artificial Sequence
  source: /note=“Description of Artificial Sequence Synthetic polypeptide”
EAEDLQVGQVELGGGPGAGCLQPLALEGSLQ

(19) United States
(12) Patent Application Publication
(10) Pub. No.: US 2011/0171312 A1
(43) Pub. Date: Jul. 14, 2011

(54) MODIFIED THERAPEUTIC PEPTIDES, METHODS OF THEIR PREPARATION AND USE

(75) Inventors: Mei-Chang Kuo, Palo Alto, CA (US); Blaine Bueche, San Jose, CA (US); Mary J. Bossard, Madison, AL (US); Cindy L. Barnes, St. Maries, ID (US)

(73) Assignee: Nektar Therapeutics, San Francisco, CA (US)

(21) Appl. No.: 13/119,208

(22) PCT Filed: Sep. 17, 2009
(86) PCT No.: PCT/US2009/005209

§ 371 Date: Mar. 16, 2011

Related U.S. Application Data
(60) Provisional application No. 61/192,673, filed on Sep. 19, 2008.

(51) Int. Cl. A61K 9/14; A61K 38/28; C07K 14/62; A61P 3/10

(52) U.S. Cl. 424/489; 514/5.9; 530/399

(57) ABSTRACT

Modified therapeutic peptide compositions comprising conjugates of therapeutic peptides covalently coupled to one or more hydrophilic polymers. Optionally, the therapeutic peptide is also covalently coupled to one or more moieties having one to ten carbon atoms. Methods of making and use are also provided. The conjugates, when administered by any of a number of administration routes, exhibit characteristics that are different from the characteristics of the peptide not attached to the water soluble oligomer and/or one or moiety having one to ten carbon atoms.

### FIELD OF THE INVENTION

Among other things, the present invention relates to conjugates comprising a therapeutic peptide moiety covalently attached to one or more water-soluble polymers. For instance, the present invention relates to modified therapeutic peptides as well as methods of their production and use. The present invention also provides pharmaceutical formulations of the modified therapeutic peptides. The modified therapeutic peptides of the invention typically exhibit surprisingly good pharmacokinetic and pharmacodynamic profiles upon administration, such as upon pulmonary administration, e.g., long-acting profiles.

### BACKGROUND OF THE INVENTION

In many ways, the chemical and biological properties of peptides make them very attractive candidates for use as therapeutic agents. Peptides are naturally occurring molecules made up of amino acid building blocks, and are involved in countless physiological processes. With 20 naturally occurring amino acids, and any number of non-naturally occurring amino acids, a nearly endless variety of peptides may be generated. Additionally, peptides display a high degree of selectivity and potency, and may not suffer from potential adverse drug-drug interactions or other negative side effects. Moreover, recent advances in peptide synthesis techniques have made the synthesis of peptides practical and economically viable. Thus peptides hold great promise as a highly diverse, highly potent, and highly selective class of therapeutic molecules with low toxicity.

A number of peptides have been identified as therapeutically promising; however, in vitro results have often not proven to bear out in vivo. Significantly, peptides suffer from a short in vivo half-life, sometimes mere minutes, making them generally impractical, in their native form, for therapeutic administration. Thus there exists a need in the art for modified therapeutic peptides having an enhanced half-life and/or reduced clearance as well as additional therapeutic advantages as compared to the therapeutic peptides in their unmodified form.

### SUMMARY OF THE INVENTION

Accordingly, the present invention provides conjugates comprising a therapeutic peptide moiety covalently attached to one or more water-soluble polymers. The water-soluble polymer may be stably bound to the therapeutic peptide moiety, or it may be releasably attached to the therapeutic peptide moiety.

The invention further provides methods of synthesizing such therapeutic peptide polymer conjugates and compositions comprising such conjugates. The invention also provides methods of treating, preventing, or ameliorating a disease, disorder or condition in a mammal comprising administering a therapeutically effective amount of a therapeutic peptide polymer conjugate of the invention.

The present invention provides advantages, including modified therapeutic peptides that exhibit a long duration of action, which further allow for less frequent administration. The modified therapeutic peptides of the invention typically exhibit surprisingly good pharmacokinetic and pharmacodynamic profiles upon administration.

The present invention relates to therapeutic peptides that are modified, including methods of their production and use. The modified therapeutic peptides of the invention may be modified, through covalent bonding, at one or more amino acid residues, to hydrophilic polymers and to moieties having one to ten carbon atoms.

The invention further provides modified therapeutic peptides having at least one amino acid residue covalently attached to a hydrophilic polymer and at least one amino acid covalently attached to a moiety having one to three carbon atoms.

The invention also provides pharmaceutical compositions comprising: a modified therapeutic peptide having at least one amino acid residue covalently attached to a hydrophilic polymer and at least one amino acid covalently attached to a moiety having from one to ten carbon atoms; and at least one pharmaceutically acceptable excipient.

The invention further provides pharmaceutical compositions comprising: a modified therapeutic peptide having at least one amino acid residue covalently attached to a moiety having from one to ten carbon atoms; and at least one pharmaceutically acceptable excipient suitable for inhalation; wherein the moiety is not a hydrophilic polymer.

Also provided are modified therapeutic peptides having at least one amino acid covalently attached to a hydrophilic polymer and at least one amino acid covalently attached to a moiety having from one to ten carbon atoms, which upon pulmonary administration to a mammal, including a human, exhibits a T_{1/2} of greater than or equal to about 4 hours.

Also provided are methods of prolonging the half-life of a pulmonarily administered therapeutic peptide comprising covalently attaching a hydrophilic polymer to at least one amino acid residue and covalently attaching a moiety having one to ten carbon atoms to at least one amino acid residue.

The invention provides aerosolized formulations comprising: a modified therapeutic peptide having at least one amino acid covalently attached to a moiety having one to ten carbon atoms; and at least one pharmaceutically acceptable excipient; wherein the moiety having one to ten carbon atoms is not a hydrophilic polymer.

The invention provides pharmaceutical formulations for inhalation, comprising particles having a mass median aerodynamic diameter (MMAD) of less than 10 μm, comprising a modified therapeutic peptide having at least one amino acid covalently attached to a hydrophilic polymer and at least one amino acid covalently attached to a moiety having one to ten carbon atoms, wherein the moiety having one to ten carbon atoms is not a hydrophilic polymer.

Also provided are methods of making a modified therapeutic peptide composition for administration, comprising reacting a therapeutic peptide with an acetylating agent, and reacting the therapeutic peptide with a reactive hydrophilic polymer; wherein an acetylated, hydrophilic polymer-conjugated therapeutic peptide is produced by the reactions; and further comprising formulating the acetylated, hydrophilic polymer-conjugated therapeutic peptide into a composition for administration.

The invention also provides compositions comprising a conjugate of therapeutic peptide covalently coupled to one or more molecules of polyethylene glycol and to one or more moieties having one to three carbon atoms.

Other features and advantages of the present invention will be set forth in the description of invention that follows, and will be apparent, in part, from the description or may be learned by practice of the invention. The invention will be realized and attained by the devices and methods particularly pointed out in the written description and claims hereof.

### BRIEF DESCRIPTION OF THE DRAWINGS

The present invention is further described in the description of invention that follows, in reference to the noted plurality of non-limiting drawings, wherein:

FIG. 1 is a plot showing acetylation reaction products at pH 8.5 for different molar ratios of acetic anhydride NHS:insulin.

FIG. 2 is a plot showing acetylation reaction products at pH 9.5 for different molar ratios of acetic anhydride NHS:insulin.

FIG. 3 is a plot showing acetylation reaction products at pH 9.7 for different molar ratios of acetic anhydride NHS:insulin.

FIG. 4 is a plot showing acetylation reaction products at pH 10.1 for different molar ratios of acetic anhydride NHS:insulin.

FIG. 5 is a plot showing acetylation reaction products at pH 9.5, 10.1, and 10.8 for different molar ratios of acetic anhydride NHS:insulin.

FIG. 6 is an HPLC chromatogram showing the composition of tri-conjugated insulin analog 750-lot 3-PEG-insulin, di-conjugated insulin analog A1-B29-PEG-Insulin (45-49%), and mono-conjugated insulin analogs, mono-A1-PEG-Insulin (25%) and mono-B29-PEG-Insulin (28%).

FIG. 7 is a plot comparing insulin binding standard curves generated using radioimmunoassay (RIA) for insulin, 550-lot 1-PEG-insulin (randomly conjugated), 750-lot 3-PEG-insulin (randomly conjugated), and 2K-lot 2-PEG-insulin (B29-mono-conjugated).

FIG. 8a is a plot illustrating the effect of PEG MW on glucose depression following intravenous administration to rats at 20 μg/animal. Values represent averages±one standard deviation of n=4 (B1-5000-PEG-ins), n=3 (750-lot 2-PEG-insulin), n=5 (2000-PEG-insulin), and n=6 (insulin control) replicates. All doses were calculated on the basis of “equivalent insulin mass.”

FIG. 8b is a plot illustrating the effect of PEG MW on glucose depression following intratracheal administration to rats at 20 μg/animal. Values represent averages±one standard deviation of n=5 (B29-750-lot 5-PEG-Insulin), n=5 (B29-2K-lot 2-PEG-Insulin), and n=4 (regular insulin) replicates. All animals were dosed at 20 μg/animal of insulin on the basis of equivalent insulin mass.

FIG. 9 (frames (a) and (b)) are plots illustrating the effect of PEG conjugation site on glucose depression following administration to rats: (a) intravenously at 80 μg insulin/animal for (i) regular insulin (n=3) and (ii) 750-PEG-insulin (n=3); (b) intratracheally at 80 μg insulin/animal for (i) regular insulin (n=4) and (iii) 550-PEG-insulin (n=4). All doses were based upon equivalent insulin mass.

FIG. 10 is a plot illustrating the effect of PEG MW on insulin absorption, as monitored by the blood glucose depression following intratracheal administration to rats. Animals were dosed (a) with 40 μg/animal of insulin control (n=7), 40 μg/animal of B1-5000-PEG-insulin (n=7), 80 μg of 750-lot 2-PEG-insulin (B1-mono-conjugated) (n=7), and 80 μg/animal of 550-lot 1-PEG-insulin (n=4). All doses were based upon equivalent insulin mass.

FIG. 11 is a plot illustrating the effect of PEGylation site on insulin pulmonary absorption in the rat. Animals were dosed with 80 μg of insulin equivalent of insulin control (n=4), 750-lot 2-PEG-insulin (n=7), 750-lot 3-PEG-insulin (n=4) and 750-lot 5-B29-PEG-insulin (n=5). All doses were based on equivalent insulin mass.

FIG. 12 is a plot illustrating the effect of acetylation with PEGylation site on insulin pulmonary absorption in the rat, as monitored by the blood glucose depression following intratracheal administration. Animals were dosed with 80 μg of insulin equivalent of insulin control (n=4), 750-lot A1-PEG-insulin (n=7) and 750-lot A2-PEG-insulin (n=4). All doses were based on equivalent insulin mass.

FIG. 13 (frames (A) and (B)) is a plot illustrating the effect of powder inhalation on pulmonary absorption of B29 2K-lot 2-PEG-insulin in dog, as monitored by (a) glucose infusion rate and (b) plasma insulin concentrations. Animals were dosed with control insulin (n=6) and 2K-2-PEG-Insulin (n=6).

FIG. 14 is a plot illustrating the group mean blood glucose concentrations following intravenous injection of 1-6 μg/rat of AKZO insulin to Sprague-Dawley rats.

FIG. 15 is a plot illustrating the group mean blood plasma insulin concentrations following intravenous injection of 1-6 μg/rat of AKZO insulin to Sprague-Dawley rats.

FIG. 16 is a plot illustrating the group mean blood glucose concentrations following subcutaneous injection of 0.4-33.3 μg/rat of HUMULIN N to Sprague-Dawley rats.

FIG. 17 is a plot illustrating the group mean blood plasma insulin concentrations following sc injection of 11 μg/rat of HUMULIN N to Sprague-Dawley. Data from IV Administration of 4 μg/rat is included for comparison.

FIG. 18 is a plot illustrating the group mean blood glucose concentrations following 15 μg intravenous injection of PEG2000-B1, PEG3000-B1, PEG5000-B1 Insulin, or PBS to Sprague-Dawley rats.

FIG. 19 is a plot illustrating the group mean plasma compound concentrations following 15 μg intravenous injection of PEG2000-B1, PEG3000-B1, PEG5000-B1 Insulin, or PBS to Sprague-Dawley rats.

FIG. 20 is a plot illustrating the group mean blood glucose concentrations following intratracheal instillation of PEG2000-B1, PEG3000-B1, PEG5000-B1 Insulin, or PBS to Sprague-Dawley rats.

FIG. 21 is a plot illustrating the group mean plasma compound concentrations following intratracheal instillation of PEG2000-B1, PEG3000-B1, PEG5000-B1 Insulin, or PBS to Sprague-Dawley rats.

FIG. 22 is a plot illustrating the group mean plasma compound concentrations following IV and IT administration of PEG5000-B1 Insulin to Sprague-Dawley rats.

FIG. 23 is a plot illustrating the mean blood glucose concentrations following 18 and 20 μg intravenous injection of PEG5000-B1 Di-acetylated Insulin to Sprague-Dawley rats.

FIG. 24 is a plot illustrating the mean blood glucose concentrations following 520 μg intratracheal instillation of PEG5000-B1 Di-acetylated Insulin to Sprague-Dawley rats.

FIG. 25 is a plot illustrating the mean plasma compound concentrations following 520 μg intratracheal instillation of PEG5000-B1 Di-acetylated Insulin to Sprague-Dawley Rats.

FIG. 26 is a plot illustrating the mean blood glucose concentrations following 200 μg intratracheal instillation of PEG2000-B1 Di-acetylated Insulin to Sprague-Dawley Rats.

FIG. 27 is a plot illustrating the mean plasma compound concentrations following 200 μg intratracheal instillation of PEG2000-B1 Di-acetylated Insulin to Sprague-Dawley Rats.

FIG. 28 is a plot illustrating the mean plasma compound concentrations following administration of 10 μg PEG2000-B1 Di-acetylated Insulin IV to Sprague-Dawley Rats. The blood glucose profile of IT Administration of 200 μg PEG2000-B1 Di-acetylated Insulin is provided for comparison.

FIG. 29 is a plot illustrating the glucose suppression in fasted rats following IT single dose of B1 PEGylated Insulins compared to NPH Profile in humans following SC administration. The dotted lines represent SC NPH profiles. The top panel corresponds to 180 μg 2K B1 PEG Insulin, the middle panel corresponds to 240 μg 3K B1 PEG Insulin, and the bottom panel corresponds to 640 μg 5K B1 PEG Insulin.

FIG. 30 is a plot illustrating the glucose suppression in fasted rats following IT single dose of B1 PEGylated diacetylated insulins compared to NPH profile in humans following SC administration. The dotted lines represent SC NPH profiles. The top panel corresponds to 200 μg 2K B1 DiAc PEG insulin, and the bottom panel corresponds to 520 μg 5K B1 DiAc PEG insulin.

FIG. 31 is a plot comparing pharmacokinetics of PEG-insulins following IT doses in rats.

FIG. 32 is a plot illustrating the cumulative absorption profiles following single IT dose of 2K, 3K, and 5K-B1 PEG insulin and 2K-B1 PEG DiAc insulin, and following single SC dose of Humulin N in rats. The absorption fraction represents cumulative dose absorbed divided by the maximum absorbable dose.

FIG. 33 is a plot illustrating the raw plasma insulin concentration-time profiles following IH dosing of placebo, insulin (0.34 mg/dog), B1-2K-PEG-DiAc-Insulin (0.6 mg/dog), and B1-5K-PEG-DiAc-Insulin (2.0 mg/dog) in beagle dogs.

FIG. 34 is a plot illustrating the raw plasma insulin concentration-time profiles following IV dosing of PBS, insulin (0.027 mg/dog), B1-2K-PEG-DiAc-Insulin (0.058 mg/dog), and B1-5K-PEG-DiAc-Insulin (0.084 mg/dog) in beagle dogs.

FIG. 35 is a plot illustrating the baseline corrected insulin concentration following IH dosing of insulin (0.34 mg/dog).

FIG. 36 is a plot illustrating the baseline corrected insulin concentration following IH dosing of B1-2K-PEG-DiAc-Insulin (0.6 mg/dog).

FIG. 37 is a plot illustrating the baseline corrected insulin concentration following IH dosing of B1-5K-PEG-DiAc-Insulin (2.0 mg/dog).

FIG. 38 is a plot illustrating the baseline corrected insulin concentration following IV dosing of insulin (0.027 mg/dog).

FIG. 39 is a plot illustrating the baseline corrected insulin concentration following IV dosing of B1-2K-PEG-DiAc-Insulin (0.058 mg/dog).

FIG. 40 is a plot illustrating the baseline corrected insulin concentration following IV dosing of B1-5K-PEG-DiAc-Insulin (0.084 mg/dog).

FIG. 41 is a plot illustrating the blood glucose profiles following IH dosing of placebo, insulin (0.34 mg/dog), B1-2K-PEG-DiAc-Insulin (0.6 mg/dog), and B1-5K-PEG-DiAc-Insulin (2.0 mg/dog) in beagle dogs.

FIG. 42 is a plot illustrating the blood glucose profiles following N dosing of PBS, insulin (0.027 mg/dog), B1-2K-PEG-DiAc-Insulin (0.058 mg/dog), and B1-5K-PEG-DiAc-Insulin (0.084 mg/dog) in beagle dogs.

FIG. 43 is a plot illustrating the PK/PD of regular insulin, 2K-PEG-DiAc-Insulin, and 5K-PEG-DiAc-Insulin following IH dosing in dogs. Each plot shows the overlay of average insulin () and glucose levels (▴) following IH dosing in dogs.

FIG. 44 shows mean blood glucose concentrations following 80-381 μg intratracheal instillation of mono-PEG2000 butyraldehyde di-acetylated insulin to Sprague-Dawley rats (Group 1—dose ranging study). (Note: Error bars indicate standard deviation; 381-μg dose animals were euthanized at 4 hr post-dose due to hypoglycemia symptoms.)

FIG. 45 shows individual blood glucose concentrations following 270 μg intratracheal instillation of mono-PEG2000 butyraldehyde di-acetylated insulin to Sprague-Dawley rats (n=9 until 8 hr, n=8 until 12 hr, n=7 thereafter) (Group 2—PK/PD study). (Note: Animal 2-1 euthanized at 12 hr due to hypoglycemia symptoms; Animal 2-4 found dead at 12 hr; Animals 2-2 and 2-9 sample at 24 hr collected via cardiac puncture due to in-operative catheter; and Animals 2-6 and 2-9 sample at 16 hr unobtainable due to in-operative catheter.)

FIG. 46 shows mean blood glucose concentrations following 270 μg intratracheal instillation of mono-PEG2000 butyraldehyde di-acetylated insulin to Sprague-Dawley rats (Group 2—PK/PD study). (Note: Error bars are standard deviation; DPBS data from a previous study.)

FIG. 47 shows mean blood glucose concentrations following subcutaneous administration of G2-PEG2-FMOC-NHS 20K or 40K insulin (random PEGylation) to diabetic mice.

FIG. 48 shows mean blood glucose concentrations following subcutaneous administration of G2-PEG2-FMOC-NHS 20K or 40K insulin (site specific PEGylation) to diabetic mice.

### DETAILED DESCRIPTION

Unless otherwise stated, a reference to a compound or component includes the compound or component by itself, as well as in combination with other compounds or components, such as mixtures of compounds.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. Unless the context clearly dictates otherwise, when the term “about” precedes a numerical value, the numerical value is understood to mean the stated numerical value and also ±10% of the stated numerical value.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

Before further discussion, a definition of the following terms will aid in the understanding of the present invention.

### Definitions

The terms used in this disclosure are defined as follows unless otherwise indicated. Standard terms are to be given their ordinary and customary meaning as understood by those of ordinary skill in the art, unless expressly defined herein.

As used in this disclosure, the singular forms “a,” “an,” and “the” include plural referents unless the context clearly dictates otherwise. Thus, for example, reference to “a polymer” includes a single polymer as well as two or more of the same or different polymers, reference to “an optional excipient” or to “a pharmaceutically acceptable excipient” refers to a single optional excipient as well as two or more of the same or different optional excipients, and the like.

“Optional” or “optionally” means that the subsequently described circumstance may or may not occur, so that the description includes instances where the circumstance occurs and instances where it does not.

“Substantially” (unless specifically defined for a particular context elsewhere or the context clearly dictates otherwise) means nearly totally or completely, for instance, satisfying one or more of the following: greater than 50%, 51% or greater, 75% or greater, 80% or greater, 90% or greater, and 95% or greater of the condition.

As used herein, the terms “therapeutic peptide” and “therapeutic peptides” mean one or more peptides having demonstrated or potential use in treating, preventing, or ameliorating one or more diseases, disorders, or conditions in a subject in need thereof, as well as related peptides. These terms may be used to refer to therapeutic peptides prior to conjugation to a water-soluble polymer as well as following the conjugation. Therapeutic peptides include, but are not limited to, those disclosed herein, including in Table A. Therapeutic peptides include peptides found to have use in treating, preventing, or ameliorating one or more diseases, disorders, or conditions after the time of filing of this application. Related peptides include fragments of therapeutic peptides, therapeutic peptide variants, and therapeutic peptide derivatives that retain some or all of the therapeutic activities of the therapeutic peptide. As will be known to one of skill in the art, as a general principle, modifications may be made to peptides that do not alter, or only partially abrogate, the properties and activities of those peptides. In some instances, modifications may be made that result in an increase in therapeutic activities. Thus, in the spirit of the invention, the terms “therapeutic peptide” or “therapeutic peptides” are meant to encompass modifications to the therapeutic peptides defined and/or disclosed herein that do not alter, only partially abrogate, or increase the therapeutic activities of the parent peptide.

The term “therapeutic activity” as used herein refers to a demonstrated or potential biological activity whose effect is consistent with a desirable therapeutic outcome in humans, or to desired effects in non-human mammals or in other species or organisms. A given therapeutic peptide may have one or more therapeutic activities, however the term “therapeutic activities” as used herein may refer to a single therapeutic activity or multiple therapeutic activities. “Therapeutic activity” includes the ability to induce a response in vitro, and may be measured in vivo or in vitro. For example, a desirable effect may be assayed in cell culture, or by clinical evaluation, EC₅₀ assays, IC₅₀ assays, or dose response curves. In vitro or cell culture assays, for example, are commonly available and known to one of skill in the art for many therapeutic peptides as defined and/or disclosed herein. Therapeutic activity includes treatment, which may be prophylactic or ameliorative, or prevention of a disease, disorder, or condition. Treatment of a disease, disorder or condition can include improvement of a disease, disorder or condition by any amount, including elimination of a disease, disorder or condition.

As used herein, the terms “peptide,” “polypeptide,” and “protein,” refer to polymers comprised of amino acid monomers linked by amide bonds. Peptides may include the standard 20 α-amino acids that are used in protein synthesis by cells (i.e., natural amino acids), as well as non-natural amino acids (non-natural amino acids may be found in nature, but not used in protein synthesis by cells, e.g., ornithine, citrulline, and sarcosine, or may be chemically synthesized), amino acid analogs, and peptidomimetics. Spatola, (1983) in Chemistry and Biochemistry of Amino Acids, Peptides, and Proteins, Weinstein, ed., Marcel Dekker, New York, p. 267. The amino acids may be D- or L-optical isomers. Peptides may be formed by a condensation or coupling reaction between the α-carbon carboxyl group of one amino acid and the amino group of another amino acid. The terminal amino acid at one end of the chain (amino terminal) therefore has a free amino group, while the terminal amino acid at the other end of the chain (carboxy terminal) has a free carboxyl group. Alternatively, the peptides may be non-linear, branched peptides or cyclic peptides. Moreover, the peptides may optionally be modified or protected with a variety of functional groups or protecting groups, including on the amino and/or carboxy terminus.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The terms “therapeutic peptide fragment” or “fragments of therapeutic peptides” refer to a polypeptide that comprises a truncation at the amino-terminus and/or a truncation at the carboxyl-terminus of a therapeutic peptide as defined herein. The terms “therapeutic peptide fragment” or “fragments of therapeutic peptides” also encompasses amino-terminal and/or carboxyl-terminal truncations of therapeutic peptide variants and therapeutic peptide derivatives. Therapeutic peptide fragments may be produced by synthetic techniques known in the art or may arise from in vivo protease activity on longer peptide sequences. It will be understood that therapeutic peptide fragments retain some or all of the therapeutic activities of the therapeutic peptides.

As used herein, the terms “therapeutic peptide variants” or “variants of therapeutic peptides” refer to therapeutic peptides having one or more amino acid substitutions, including conservative substitutions and non-conservative substitutions, amino acid deletions (either internal deletions and/or C- and/or N-terminal truncations), amino acid additions (either internal additions and/or C- and/or N-terminal additions, e.g., fusion peptides), or any combination thereof. Variants may be naturally occurring (e.g., homologs or orthologs), or non-natural in origin. The term “therapeutic peptide variants” may also be used to refer to therapeutic peptides incorporating one or more non-natural amino acids, amino acid analogs, and peptidomimetics. It will be understood that, in accordance with the invention, therapeutic peptide fragments retain some or all of the therapeutic activities of the therapeutic peptides.

The terms “therapeutic peptide derivatives” or “derivatives of therapeutic peptides” as used herein refer to therapeutic peptides, therapeutic peptide fragments, and therapeutic peptide variants that have been chemically altered other than through covalent attachment of a water-soluble polymer. It will be understood that, in accordance with the invention, therapeutic peptide derivatives retain some or all of the therapeutic activities of the therapeutic peptides.

As used herein, the terms “amino terminus protecting group” or “N-terminal protecting group,” “carboxy terminus protecting group,” “C-terminal protecting group,” or “side chain protecting group” refer to any chemical moiety capable of addition to and optionally removal from a functional group on a peptide (e.g., the N-terminus, the C-terminus, or a functional group associated with the side chain of an amino acid located within the peptide) to allow for chemical manipulation of the peptide.

“Insulin” as used herein is meant to encompass natural and synthetically-derived insulin including glycoforms thereof as well as analogs thereof including polypeptides having up to three amino acid modifications (deletion, substitution, or addition variants) to the extent that they substantially retain at least 80% (e.g., at least 85%, at least 90%, or at least 95%) of the therapeutic activity associated with full length insulin (prior to modification as described herein). The insulins of the present invention may be produced by any manner including, but not limited to, pancreatic extraction, recombinant expression, and in vitro polypeptide synthesis. Modified insulins of the present invention include, but are not limited to, insulins that are produced by modifying native or wild type insulin and compounds that are produced in any manner to provide the desired end product, regardless of whether or not insulin is itself modified. Thus, it is not necessary to begin with an “unmodified” insulin starting material, such as a native insulin; starting materials for synthesizing the modified insulin end product may be amino acids, which are modified and synthesized into a modified insulin.

“Native” or “wild type” insulin refers to human insulin having an amino acid sequence corresponding to the amino acid sequence of human insulin as found in nature. Native or wild type insulin can be natural (i.e., isolated from a natural source) or synthetically produced.

“Modified,” as in “modified therapeutic peptide,” is an adjective to describe the end product. For instance, “modified insulin” is an insulin that includes modifications as described herein. Modified therapeutic peptides, such as insulin, can be prepared by adding modifications as described herein to a complete therapeutic peptide, or by synthesizing a therapeutic peptide that includes modifications in the amino acids from which it is synthesized. Thus, for example, a modified insulin with an acetyl group attached to an amino acid residue can be made by acetylating an insulin molecule or by synthesizing an insulin molecule using at least one acetylated amino acid.

A “deletion variant” of insulin is a peptide in which up to three amino acid residues of insulin have been deleted, and the amino acid residues preceding and following the deleted amino acid residue are connected via an amide bond (except in instances where the deleted amino acid residue was located on a terminus of the peptide or protein). Deletion variants include instances where only a single amino acid residue has been deleted, as well as instances where two amino acids are deleted, or three amino acids are deleted.

A “substitution variant” of insulin is a peptide or protein in which up to three amino acid residues of insulin have been deleted, and a different amino acid residue has taken its place. Substitution variants include instances where only a single amino acid residue has been substituted, as well as instances where two amino acids are substituted, or three amino acids are substituted.

An “addition variant” of insulin is a peptide in which up to three amino acid residues of insulin have been added into an amino acid sequence, and adjacent amino acid residues are attached to the added amino acid residue by way of amide bonds (except in instances where the added amino acid residue is located on a terminus of the peptide, wherein only a single amide bond attaches the added amino acid residue). Addition variants include instances where only a single amino acid residue has been added, as well as instances where two amino acids are added, or three amino acids are added.

In the case of deletion or addition variants of insulin, the B29 Lys may no longer appear at position 29. As used herein, “B29” includes Lys that would appear at position 29 but for the deletion or addition.

“Non-naturally occurring” with respect to a polymer as described herein, means a polymer that in its entirety is not found in nature. A non-naturally occurring polymer of the invention may, however, contain one or more monomers or segments of monomers that are naturally occurring, so long as the overall polymer structure is not found in nature.

An “oligomer” is a molecule possessing from about 2 to about 30 monomers. The architecture of an oligomer can vary. Specific oligomers for use in the invention include those having a variety of geometries such as linear, branched, or forked, to be described in greater detail below. An oligomer is a type of polymer.

The term “water soluble” as in a “water-soluble polymer” is any polymer that is soluble in water at room temperature. Water-soluble polymers have a solubility of 1% (w/v) or more in water at 25° C. Typically, a water-soluble polymer will transmit at least about 75%, such as at least about 95%, of light transmitted by the same solution after filtering. On a weight basis, a water-soluble polymer will often be at least about 35% (w/v) soluble in water, such as at least about 50% (w/v) soluble in water, at least about 70% (w/v) soluble in water, or at least about 85% (w/v) soluble in water, at 25° C. Typically, the water-soluble polymer is at least about 95% (w/v) soluble in water or completely soluble in water. As used herein, the term “water-soluble polymer” refers both to a molecule as well as the residue of water-soluble polymer that has been attached to another moiety.

“Hydrophilic,” e.g., in reference to a “hydrophilic polymer,” refers to a polymer that is characterized by its solubility in and compatibility with water. In non-cross linked form, a hydrophilic polymer is able to dissolve in or be dispersed in water. Typically, a hydrophilic polymer possesses a polymer backbone composed of carbon and hydrogen, and generally possesses a high percentage of oxygen in the main polymer backbone and/or in pendent groups substituted along the polymer backbone, thereby leading to its “water-loving” nature. The water-soluble polymers of the present invention are typically hydrophilic, e.g., non-naturally occurring hydrophilic.

“Hydrophilic character” means being hydrophilic.

A “lipophilic moiety” is one that, when attached to a hydrophilic polymer in accordance with the invention, either by a degradable or non-degradable bond, is effective to substantially alter the hydrophilic nature of the polymer and thus the polymer-insulin conjugate. Typical lipophilic groups such as fatty acids will comprise from about 12-22 carbon atoms. Of course, the hydrophilic polymers of the invention may lack a lipophilic moiety.

“PEG,” “polyethylene glycol” and “poly(ethylene glycol)” as used herein, are interchangeable and encompass any nonpeptidic water-soluble poly(ethylene oxide). Typically, PEGs for use in accordance with the invention comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. Throughout the specification and claims, it should be remembered that the term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀ alkoxy group, and still more preferably a C₁₋₅ alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer (e.g., the end-capping moiety “methoxy” in CH₃O(CH₂CH₂O)ₙ— and CH₃(OCH₂CH₂)ₙ—). In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) to which the polymer is coupled can be determined by using a suitable detector. Such labels include, without limitation, fluorescers, chemiluminescers, moieties used in enzyme labeling, colorimetric (e.g., dyes), metal ions, radioactive moieties, gold particles, quantum dots, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin.

“Branched,” in reference to the geometry or overall structure of a polymer, refers to a polymer having two or more polymer “arms” extending from a branch point. A branched polymer may possess 2 polymer arms, 3 polymer arms, 4 polymer arms, 6 polymer arms, 8 polymer arms or more. One particular type of highly branched polymer is a dendritic polymer or dendrimer.

A “branch point” refers to a bifurcation point comprising one or more atoms at which a polymer branches or forks from a linear structure into one or more additional arms.

“Forked” in reference to the geometry or overall structure of a polymer, refers to a polymer having two or more functional groups (typically through one or more atoms) extending from a branch point.

A “dendrimer” or dendritic polymer is a globular, size monodisperse polymer in which all bonds emerge radially from a central focal point or core with a regular branching pattern and with repeat units that each contribute a branch point. Dendrimers exhibit certain dendritic state properties such as core encapsulation, making them unique from other types of polymers.

“Multifunctional” in the context of a polymer of the invention means a polymer having 3 or more functional groups contained therein, where the functional groups may be the same or different. Multifunctional polymers of the invention will typically contain from about 3 to 100 functional groups, such as 3 to 50, 3 to 25, or 3 to 15, or 3 to 10 functional groups, or will contain 3, 4, 5, 6, 7, 8, 9, or 10 functional groups within the polymer.

A “difunctional” polymer means a polymer having two functional groups contained therein, either the same (i.e., homodifunctional) or different (i.e., heterodifunctional).

“Monodisperse” refers to a polymer composition wherein substantially all of the polymers in the composition have a well-defined, single (i.e., the same) molecular weight and defined number of monomers, as determined by chromatography or mass spectrometry. Monodisperse polymer compositions are in one sense pure, that is, substantially having a single and definable number (as a whole number) of monomers rather than a large distribution. A monodisperse polymer composition of the invention possesses an Mw/Mn value of 1.0005 or less, such as an Mw/Mn value of 1.0000. By extension, a composition comprised of monodisperse conjugates means that substantially all polymers of all conjugates in the composition have a single and definable number (as a whole number) of monomers rather than a large distribution and would possess a Mw/Mn value of 1.0005 or less, such as a Mw/Mn value of 1.0000 if the polymer were not attached to the moiety derived from a small molecule drug. A composition comprised of monodisperse conjugates can, however, include one or more nonconjugated substances such as solvents, reagents, excipients, and so forth.

“Bimodal,” in reference to a polymer composition, refers to a polymer composition wherein substantially all polymers in the composition have one of two definable and different numbers (as whole numbers) of monomers rather than a large distribution, and whose distribution of molecular weights, when plotted as a number fraction versus molecular weight, appears as two separate identifiable peaks. Preferably, for a bimodal polymer composition as described herein, each peak is symmetric about its mean, although the size of the two peaks may differ. Ideally, the polydispersity index of each peak in the bimodal distribution, Mw/Mn, is 1.01 or less, such as 1.001 or less, 1.0005 or less, or a Mw/Mn value of 1.0000. By extension, a composition comprised of bimodal conjugates means that substantially all polymers of all conjugates in the composition have one of two definable and different numbers (as whole numbers) of monomers rather than a large distribution and would possess a Mw/Mn value of 1.01 or less, such as 1.001 or less, 1.0005 or less, or a Mw/Mn value of 1.0000. A composition comprised of bimodal conjugates can, however, include one or more nonconjugated substances such as solvents, reagents, excipients, etc.

“Polydisperse” in reference to a polymer, refers to a composition having a polymer present in a distribution of molecular weights, such as Mw/Mn greater than 1.01. The distribution generally will be a normal distribution, i.e., one that has a higher concentration of polymers with molecular weights near the mean, with a decrease in frequency as the difference from the mean molecular weight increases. The distribution may be a Gaussian distribution.

“Molecular weight” in the context of a water-soluble polymer, such as PEG, can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Unless otherwise indicated, molecular weight is determined by matrix assisted laser desorption ionization (MALDI). Other methods for measuring molecular weight values can also be used, such as the use of end-group analysis or the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 1.2, less than about 1.15, less than about 1.10, less than about 1.05, and less than about 1.03. As used herein, references will at times be made to a single hydrophilic polymer having either a weight-average molecular weight or number-average molecular weight; such references will be understood to mean that the single hydrophilic polymer was obtained from a composition of hydrophilic polymers having the stated molecular weight.

The term “reactive” or “activated” refers to a functional group that reacts readily or at a practical rate under conventional conditions of organic synthesis. This is in contrast to those groups that either do not react or require strong catalysts or impractical reaction conditions in order to react (i.e., a “nonreactive” or “inert” group).

“Not readily reactive,” with reference to a functional group present on a molecule in a reaction mixture, indicates that the group remains largely intact under conditions effective to produce a desired reaction in the reaction mixture.

The terms “linker moiety,” “spacer,” or “spacer moiety” are used herein to refer to an atom or a collection of atoms optionally used to link one moiety to another, such as a hydrophilic polymer segment to insulin. The spacer moieties of the invention may be hydrolytically stable or may include one or more physiologically hydrolyzable or enzymatically releasable linkages. Unless the context clearly dictates otherwise, a spacer moiety optionally exists between any two elements of a compound (e.g., the provided conjugates comprising a residue of a therapeutic peptide and a water-soluble polymer that can be attached directly or indirectly through a spacer moiety).

A “releasable” linkage is a relatively labile linkage or bond that breaks under physiological conditions. The tendency of a bond to break will depend not only on the general type of linkage connecting two central atoms but also on the substituents attached to these central atoms. Appropriate unstable or weak linkages include, but are not limited to, carboxylate ester, phosphate ester, anhydrides, acetals, ketals, acyloxyalkyl ether, imines, orthoesters, peptides, oligonucleotides, thioesters, thiolesters, and carbonates.

“Releasably attached,” e.g., in reference to a therapeutic peptide releasably attached to a water-soluble polymer, refers to a therapeutic peptide that is covalently attached via a linker that includes a degradable linkage as disclosed herein, wherein upon degradation (e.g., hydrolysis), the therapeutic peptide is released. The therapeutic peptide thus released will typically correspond to the unmodified parent or native therapeutic peptide, or may be slightly altered, e.g., possessing a short organic tag. Preferably, the unmodified parent therapeutic peptide is released.

An “enzymatically releasable linkage” is a linkage that is subject to degradation by one or more enzymes under physiological conditions.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks. It must be pointed out that some linkages can be hydrolytically stable or hydrolyzable, depending upon (for example) adjacent and neighboring atoms and ambient conditions. One of ordinary skill in the art can determine whether a given linkage or bond is hydrolytically stable or hydrolyzable in a given context by, for example, placing a linkage-containing molecule of interest under conditions of interest and testing for evidence of hydrolysis (e.g., the presence and amount of two molecules resulting from the cleavage of a single molecule). Other approaches known to those of ordinary skill in the art for determining whether a given linkage or bond is hydrolytically stable or hydrolyzable can also be used.

A “hydrolytically releasable” or “hydrolyzable” linkage or bond is a bond that reacts with water (i.e., is hydrolyzed) under physiological conditions. Examples include bonds that have a hydrolysis half-life at pH 8, 25° C., of less than about 30 minutes. The tendency of a bond to hydrolyze in water will depend not only on the general type of linkage connecting two given atoms but also on the substituents attached to the two given atoms. Hydrolytically unstable or releasable linkages include, but are not limited to, carbamate, carboxylate ester (referred to herein simply as “ester”), phosphate ester, anhydrides, acetals, ketals, acyloxyalkyl ether, imine, orthoester, peptide, and oligonucleotide. Hydrolytically releasable linkages exclude linkages in which release of a carrier group becomes effective only after unmasking an activating group, such as disclosed in WO 2005/099768, which is incorporated herein by reference. In other words, hydrolytically releasable linkages exclude linkages based on cascade cleavage mechanisms.

The term “protected” or “protecting group” or “protective group” refers to the presence of a moiety (i.e., the protecting group) that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule, if any. The protecting group may be removed under certain conditions. For instance, in some embodiments, at least 50% of the moiety is removed when the modified therapeutic peptide is subjected to at least one of the following conditions:

(1) 5 mg/ml of the modified therapeutic peptide is placed in trifluoroacetic acid (TFA) for 2 hours at 20° C.;

(2) 5 mg/ml of the modified therapeutic peptide is placed in water containing 2 M acetic acid for 24 hours at 20° C.;

(3) 5 mg/ml of the modified therapeutic peptide is placed in water containing 50 mM acetic acid for 24 hours at 20° C.;

(4) 5 mg/ml of the modified therapeutic peptide is placed in water containing 50 mM Tris at pH 8.5 for 24 hours at 40° C.;

(5) 5 mg/ml of the modified therapeutic peptide is placed in DMSO containing 20 (w/v) % piperidine for 5 minutes at 20° C.;

(6) 5 mg/ml of the modified therapeutic peptide is placed in a water/acetonitrile (1:1) mixture solution; the solution is bubbled with N₂ for at least 15 min; Pd/C catalyst (10 wt % on activated carbon) is then added slowly to 10 wt % of modified therapeutic peptide; then the reaction mixture is agitated; the system is evacuated and recharged with hydrogen gas under 50 psi three times (agitation is stopped during evacuation and recharging); the reaction mixture is then kept at room temperature under 50 psi for 16 hrs;

(7) 5 mg/ml of the modified therapeutic peptide is placed in ethylene glycol; 10 molar equivalents of hydrazine monohydrate 8 molar equivalents of KOH are added; the reaction mixture is heated to 100° C., under nitrogen for 30 minutes; and

(8) 5 mg/ml of the modified therapeutic peptide is placed in anhydrous HF at 0° C. for 30 minutes.

The term “blocking group” refers to the presence of a moiety (i.e., the blocking group) that prevents or blocks reaction of a particular chemically reactive functional group in a molecule under certain reaction conditions. The blocking group will vary depending upon the type of chemically reactive functional group being protected as well as the reaction conditions to be employed and the presence of additional reactive or protecting groups in the molecule, if any. Blocking groups constitute an irreversible modification.

An “irreversible modification” means that a group generally cannot be removed without breaking the amino acid chain of therapeutic peptide. For instance, acetylation is an irreversible modification. In some embodiments, less than 50% of the moiety is removed from otherwise intact modified therapeutic peptide at pH 2 to 10 or pH 3 to 8, after 24 hours at room temperature. For instance, in some embodiments, less than 50% of the moiety is removed when the modified therapeutic peptide is subjected to any one of the following conditions:

(1) 5 mg/ml of the modified therapeutic peptide is placed in trifluoroacetic acid (TFA) for 2 hours at 20° C.;

(2) 5 mg/ml of the modified therapeutic peptide is placed in water containing 2 M acetic acid for 24 hours at 20° C.;

(3) 5 mg/ml of the modified therapeutic peptide is placed in water containing 50 mM acetic acid for 24 hours at 20° C.;

(4) 5 mg/ml of the modified therapeutic peptide is placed in water containing 50 mM Tris at pH 8.5 for 24 hours at 40° C.;

(5) 5 mg/ml of the modified therapeutic peptide is placed in DMSO containing 20 (w/v) % piperidine for 5 minutes at 20° C.;

(6) 5 mg/ml of the modified therapeutic peptide is placed in a water/acetonitrile (1:1) mixture solution; the solution is bubbled with N₂ for at least 15 min; Pd/C catalyst (10 wt % on activated carbon) is then added slowly to 10 wt % of modified therapeutic peptide; then the reaction mixture is agitated; the system is evacuated and recharged with hydrogen gas under 50 psi three times (agitation is stopped during evacuation and recharging); the reaction mixture is then kept at room temperature under 50 psi for 16 hrs;

(7) 5 mg/ml of the modified therapeutic peptide is placed in ethylene glycol; 10 molar equivalents of hydrazine monohydrate 8 molar equivalents of KOH are added; the reaction mixture is heated to 100° C., under nitrogen for 30 minutes; and

(8) 5 mg/ml of the modified therapeutic peptide is placed in anhydrous HF at 0° C. for 30 minutes.

A basic or acidic reactant described herein includes neutral, charged, and any corresponding salt forms thereof.

As used herein, the term “ionizable hydrogen atom” (“H_{α}”) means a hydrogen atom that can be removed in the presence of a base, often a hydroxide or amine base. Typically, the “ionizable hydrogen atom” (“H_{α}”) will be a hydrogen atom attached to a carbon atom that, in turn, is attached to one or more aromatic moieties or another group or groups that in some way stabilize the carbanion that would form from loss of the ionizable hydrogen atom as a proton (or the transition state leading to said carbanion).

As used herein, the “halo” designator (e.g., fluoro, chloro, iodo, bromo, and so forth) is generally used when the halogen is attached to a molecule, while the suffix “ide” (e.g., fluoride, chloride, iodide, bromide, and so forth) is used when the halogen exists in its independent ionic form (e.g., such as when a leaving group leaves a molecule).

In the context of the present discussion, it should be recognized that the definition of a variable provided with respect to one structure or formula is applicable to the same variable repeated in a different structure, unless the context dictates otherwise. Thus, for example, the definition of “POLY,” “spacer moiety,” “R^{e1}” and so forth with respect to a polymeric reagent is equally applicable to a conjugate provided herein.

As used herein, the term “carboxylic acid” is a moiety having a

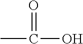

functional group (also represented as a “—COOH” or “—C(O)OH”), as well as moieties that are derivatives of a carboxylic acid, such derivatives including, for example, protected carboxylic acids. Thus, unless the context clearly dictates otherwise, the term carboxylic acid includes not only the acid form, but corresponding esters and protected forms as well. With regard to protecting groups suited for a carboxylic acid and any other functional group described herein, reference is made to Greene et al., “PROTECTIVE GROUPS IN ORGANIC SYNTHESIS,” 3^{rd} Edition, John Wiley and Sons, Inc., New York, 1999.

An “organic radical” as used herein includes, for example, alkyl, substituted alkyl, alkenyl, substituted alkenyl, alkynyl, substituted alkynyl, aryl, and substituted aryl.

The term “active” or “activated” when used in conjunction with a particular functional group refers to a reactive functional group that reacts readily with an electrophile or a nucleophile on another molecule. This is in contrast to those groups that require strong catalysts or highly impractical reaction conditions in order to react (i.e., a “non-reactive” or “inert” group).

As used herein, the term “functional group” or any synonym thereof is meant to encompass protected forms thereof as well as unprotected forms.

The terms “spacer moiety,” “linkage” and “linker” are used herein to refer to an atom or a collection of atoms optionally used to link interconnecting moieties such as a terminus of a polymer segment and a therapeutic peptide or an electrophile or nucleophile of a therapeutic peptide. The spacer moiety may be hydrolytically stable or may include a physiologically hydrolyzable or enzymatically degradable linkage.

A “monomer” or “mono-conjugate,” in reference to a polymer conjugate of a therapeutic peptide, refers to a therapeutic peptide having only one water-soluble polymer molecule covalently attached thereto, whereas a therapeutic peptide “dimer” or “di-conjugate” is a polymer conjugate of a therapeutic peptide having two water-soluble polymer molecules covalently attached thereto, and so forth.

“Alkyl” refers to hydrocarbon chains, typically ranging about 1 to 20 atoms in length, such as 1 to 15 atoms in length. The hydrocarbon chains are preferably but not necessarily saturated and may optionally contain additional functional groups attached thereto. The hydrocarbon chains may be branched or straight chain. Exemplary alkyl groups include ethyl, propyl, 1-methylbutyl, 1-ethylpropyl, and 3-methylpentyl. In one preferred embodiment of the invention, conjugates comprising an alkylated PEG, and in particular, a linear alkylated PEG, are those having an alkyl portion that is not a fatty acid or other lipophilic moiety.

“Lower alkyl” refers to an alkyl group containing from 1 to 6 carbon atoms, and may be straight chain or branched, as exemplified by methyl, ethyl, n-butyl, i-butyl, and t-butyl.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8 carbon atoms. “Cycloalkylene” refers to a cycloalkyl group that is inserted into an alkyl chain by bonding of the chain at any two carbons in the cyclic ring system.

“Alkoxy” refers to an —O—R group, wherein R is alkyl or substituted alkyl, preferably C₁₋₆ alkyl (e.g., methoxy, ethoxy, propyloxy, and so forth).

As used herein, “alkenyl” refers to a branched or unbranched hydrocarbon group of 1 to 15 atoms in length, containing at least one double bond, such as ethenyl, n-propenyl, isopropenyl, n-butenyl, isobutenyl, octenyl, decenyl, tetradecenyl, and the like.

The term “alkynyl” as used herein refers to a branched or unbranched hydrocarbon group of 2 to 15 atoms in length, containing at least one triple bond, ethynyl, n-butynyl, isopentynyl, octynyl, decynyl, and so forth.

The term “substituted” as in, for example, “substituted alkyl,” refers to a moiety (e.g., an alkyl group) substituted with one or more noninterfering substituents, such as, but not limited to: alkyl; C₃₋₈ cycloalkyl, e.g., cyclopropyl, cyclobutyl, and the like; halo, e.g., fluoro, chloro, bromo, and iodo; cyano; alkoxy, lower phenyl; substituted phenyl; and the like. “Substituted aryl” is aryl having one or more noninterfering groups as a substituent. For substitutions on a phenyl ring, the substituents may be in any orientation (i.e., ortho, meta, or para).

“Noninterfering substituents” are those groups that, when present in a molecule, are typically nonreactive with other functional groups contained within the molecule.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Heteroaryl” is an aryl group containing from one to four heteroatoms, preferably sulfur, oxygen, or nitrogen, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom that is not a carbon. Preferred heteroatoms include sulfur, oxygen, and nitrogen.

“Substituted heteroaryl” is heteroaryl having one or more noninterfering groups as substituents.

“Substituted heterocycle” is a heterocycle having one or more side chains formed from noninterfering substituents.

An “organic radical” as used herein shall include alkyl, substituted alkyl, alkenyl, substituted alkenyl, alkynyl, substituted alkynyl, aryl, and substituted aryl.

“Electrophile” and “electrophilic group” refer to an ion or atom or collection of atoms, that may be ionic, having an electrophilic center, i.e., a center that is electron seeking, capable of reacting with a nucleophile.

“Nucleophile” and “nucleophilic group” refer to an ion or atom or collection of atoms that may be ionic having a nucleophilic center, i.e., a center that is seeking an electrophilic center or with an electrophile.

A “retro-Michael type product” refers to a product arising from the reverse of a Michael-type addition reaction. A Michael addition reaction (forward direction) refers to the addition of a nucleophilic carbon species to an electrophilic double bond. Typically, but not necessarily, the nucleophile is an enolate or an enamine although the nucleophile can also be an alkoxide or an amine or other species. The electrophile is typically an alpha, beta-unsaturated ketone, ester, or nitrile, although other electron-withdrawing groups can also activate a carbon-carbon double bond to nucleophilic attack A product arising from the reverse (or backwards direction) of a Michael-type addition as described above, that is to say, an elimination reaction resulting in the loss of a nucleophilic carbon species (that may be but is not necessarily an enolate or enamine) and formation of an electrophilic double bond such as an alpha, beta unsaturated ketone or the like as described above is considered a retro-Michael type product. For example, a retro-Michael-type reaction of mPEG-propionaldehyde results in the retro-Michael type products, mPEG-OH and acrolein (CH₂═CH—CHO).

“Pharmaceutically acceptable,” as in “pharmaceutically acceptable excipient” or “pharmaceutically acceptable carrier,” refers to something can be included in the compositions of the invention and that causes no significant adverse toxicological effects to the patient.

“Pharmaceutically acceptable salts” include but are not limited to amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmitate, salicylate and stearate, as well as estolate, gluceptate, and lactobionate salts. Similarly, salts containing pharmaceutically acceptable cations include, but are not limited to, sodium, potassium, calcium, magnesium, aluminum, lithium, and ammonium (including substituted ammonium). Modified therapeutic peptides may be in the form of a pharmaceutically acceptable salt.

“Pharmacologically effective amount,” “physiologically effective amount,” and “therapeutically effective amount” are used interchangeably herein to mean the amount of a polymer-(therapeutic peptide) conjugate that is needed to provide a desired level of the conjugate (or corresponding unconjugated therapeutic peptide) in the bloodstream or in the target tissue. The precise amount will depend upon numerous factors, e.g., the particular therapeutic peptide, the components and physical characteristics of the therapeutic composition, intended patient population, individual patient considerations, and the like, and can readily be determined by one skilled in the art, based upon the information provided herein.

The terms “subject,” “individual,” or “patient” are used interchangeably herein and refer to a vertebrate, preferably a mammal. Mammals include, but are not limited to, murines, rodents, simians, humans, farm animals, sport animals, and pets.

“Glass transition temperature (Tg)”, as used herein, is the onset of a temperature range at which a composition changes from a glassy or vitreous state to a syrup or rubbery state. Generally, Tg is determined using differential scanning calorimetry (DSC). The standard designation for Tg is the temperature at which onset of the change of heat capacity (Cp) of the composition occurs upon scanning through the transition. The definition of Tg, however, can be arbitrarily defined as the onset, midpoint or endpoint of the transition. For purposes of the present invention, we will use the onset of the changes in Cp when using DSC. See “Formation of Glasses from Liquids and Biopolymers” by C. A. Angell: Science, 267, 1924-1935 (Mar. 31, 1995), and “Differential Scanning calorimetry Analysis of Glass Transitions” by Jan P. Wolanczyk: Cryo-Letters, 10, 73-76 (1989). For detailed mathematical treatment, see “Nature of the Glass Transition and the Glassy State” by Gibbs and DiMarzio: Journal of Chemical Physics, 28, No. 3, 373-383 (March, 1958). These articles are incorporated herein by reference.

A “glass-forming excipient” is an excipient that, when added to a composition, promotes glassy state formation of the composition.

The term “glass” or “glassy state,” as used herein, refers to a liquid that has lost its ability to flow, i.e., it is a liquid with a very high viscosity, wherein the viscosity ranges from 10¹⁰ to 10¹⁴ Pascal-seconds. It can be viewed as a metastable amorphous system in which the molecules have vibrational motion and reduced rotational motion, but have very slow (almost immeasurable) translational motion when compared to the liquid state. As a metastable system, it is stable for long periods of time when stored well below the glass transition temperature. Because glasses are not in a state of thermodynamic equilibrium, glasses stored at temperatures at or near the glass transition temperature relax to equilibrium upon storage and lose their high viscosity. The resultant rubbery or syrupy flowing liquid can lead to physical instability of the product. A solvent evaporation technique (U.S. Pat. No. 6,309,671) can be used to achieve a glassy state, as well as other processes that can produce a glassy state with acceptable Tg, for example, freeze drying followed by milling for micronization.

A “substantially non-immunogenic” modified therapeutic peptide of the invention possesses a reduced immunogenicity relative to native therapeutic peptide. Immunogenicity may be assessed by determining antibody titres in mice or preferably in rabbits upon administration of a PEG therapeutic peptide conjugate relative to non-modified therapeutic peptide.

A composition in “dry powder form” is a powder composition that contains less than about 20 wt % moisture, such as less than 10 wt % or less than 5 wt % moisture.

A composition that is “suitable for pulmonary delivery” refers to a composition that is capable of being aerosolized and inhaled by a subject so that at least a portion of the aerosolized particles reaches the lungs to permit penetration into the lower respiratory tract and alveoli. Such a composition is considered to be “respirable” or “inhalable.”

“Aerosolized” particles are liquid or solid particles that are suspended in a gas, typically as a result of actuation (or firing) of an inhalation device such as a dry powder inhaler, an atomizer, a metered dose inhaler, or a nebulizer.

As used herein, the term “emitted dose” or “ED” refers to an indication of the delivery of aerosolized particles from an inhaler device after an actuation or dispersion event. ED is defined as the ratio of the dose delivered by an inhaler device to the nominal dose (i.e., the mass of particles per unit dose placed into a suitable inhaler device prior to firing). The ED is an experimentally determined amount, and may be determined using an in vitro device set up which mimics patient dosing. To determine an ED value for powders, as used herein, dry powder is placed into an Exubera® inhaled insulin inhaler, described in U.S. Pat. No. 6,257,233, which is incorporated herein by reference in its entirety. The Exubera® inhaled insulin inhaler is actuated, dispersing the powder. The resulting aerosol cloud is then drawn from the device by vacuum (30 L/min) for 2.5 seconds after actuation, where it is captured on a tared glass fiber filter (Gelman, 47 mm diameter) attached to the device mouthpiece. The amount of powder that reaches the filter constitutes the delivered dose. For example, for a capsule containing 5 mg of dry powder that is placed into an inhalation device, if dispersion of the powder results in the recovery of 4 mg of powder on a tared filter as described above, then the ED for the dry powder composition is 80% (=4 mg (delivered dose)/5 mg (nominal dose)). To determine ED values for liquids, as used herein, the liquid is placed in a Salter jet nebulizer set at a driving pressure of 60 psig and an output flow rate of 15 liters/min. The resulting aerosol cloud is captured and analyzed as described above for dry powders.

As used herein, “mass median diameter” or “MMD” refers to the median diameter of a plurality of particles, typically in a polydisperse particle population, i.e., consisting of a range of particle sizes. MMD values as reported herein are determined by laser diffraction (Sympatec Helos, Clausthal-Zellerfeld, Germany), unless the context indicates otherwise. For powders, samples are added directly to the feeder funnel of the Sympatec RODOS dry powder dispersion unit. This can be achieved manually or by agitating mechanically from the end of a VIBRI vibratory feeder element. Samples are dispersed to primary particles via application of pressurized air (2 to 3 bar), with vacuum depression (suction) maximized for a given dispersion pressure. Dispersed particles are probed with a 632.8 nm laser beam that intersects the dispersed particles' trajectory at right angles. Laser light scattered from the ensemble of particles is imaged onto a concentric array of photomultiplier detector elements using a reverse-Fourier lens assembly. Scattered light is acquired in time-slices of 5 ms. Particle size distributions are back-calculated from the scattered light spatial/intensity distribution using an algorithm. As defined herein, MMD for liquids is also determined by laser diffraction.

“Mass median aerodynamic diameter,” or “MMAD,” is a measure of the aerodynamic size of a dispersed particle. The aerodynamic diameter is used to describe an aerosolized particle in terms of its settling behavior, and is the diameter of a unit density sphere having the same settling velocity, in air, as the particle. The aerodynamic diameter encompasses particle shape, density, and physical size of a particle. As used herein, for powders, MMAD refers to the midpoint or median of the aerodynamic particle size distribution of an aerosolized powder determined by cascade impaction at 28 LPM, 20° C., and 40% RH using an Exubera® inhaled insulin inhaler, described in U.S. Pat. No. 6,257,233, which is incorporated herein by reference in its entirety, unless otherwise indicated. As defined herein, MMAD for liquids is also determined by cascade impaction.

“Fine particle fraction” is the fraction of particles with an aerodynamic diameter that is less than 5 microns (μm). Where specified, the fine particle fraction may also refer to the fraction of particles with an aerodynamic diameter that is less than 3.3 microns.

“Absolute pulmonary bioavailability” is the percentage of a drug dose (e.g., of a modified therapeutic peptide in accordance with the invention) delivered to the lungs that is absorbed and enters the blood circulation of a human relative to a subcutaneous dose of the same amount of native therapeutic peptide. The inhalable therapeutic peptide compositions of the invention are, in one aspect, characterized by an absolute pulmonary bioavailability of at least about 20% in plasma or blood, with absolute pulmonary bioavailabilities generally ranging from about 10% to 30% or more, such as from 30% to 60% or from 40% to 50%. Generally, depending upon the specific nature of the modified therapeutic peptide, a conjugate of the invention will possess an absolute pulmonary bioavailability of at least about one of the following: 10%, 12%, 15%, 18%, 20%, 22%, 25%, 30%, 32%, 35%, or greater.

“Equivalent therapeutic peptide mass” means the mass of therapeutic peptide present, which is obtained by subtracting the mass of the non-therapeutic peptide portion, e.g., PEG and acetyl groups, from the overall mass.

“Residence time” means the amount of time a substance remains in a compartment—measured by half-life of elimination from that compartment.

“Rate of systemic absorption” means the rate at which a molecule crosses an epithelial layer to enter the systemic circulation.

“Distribution phase,” in reference to the half-life of a modified therapeutic peptide of the invention, refers to the initial rapid phase during which therapeutic peptide disappears from the plasma. The terminal slow or elimination phase half-life refers to the slow phase during which therapeutic peptide is eliminated from the body.

“Prolonged effect” of therapeutic peptide refers to therapeutic peptide having a duration of effect (i.e., elevated blood levels above baseline) of at least about 6 hours, preferably of at least about 8 hours.

“Glucose levels that are suppressed” refers to blood levels of glucose (e.g., after administration of a modified insulin of the invention) that are suppressed below baseline or basal levels.

“Measurable reduction in blood glucose level” refers to a statistically significant (p<0.05) reduction in blood glucose when measured with a plasma glucometer, e.g., Ascensia Elite XL (Bayer Corporation, Mishawaka, Ind.), with at least 6 measurements at each time point.

“Treating or ameliorating” a disease or medical condition means reducing or eliminating the symptoms, or effecting a desirable change in an underlying cause of the disease or medical condition. Thus, in some embodiments, “treating or ameliorating” a disease or medical condition will be directed at addressing the cause of the disease or medical condition. In some instances, addressing an underlying cause of a disease, such as abnormal insulin levels, may result in improvements in symptoms of the disease. Treating a disease may result in cure of the disease.

The term “diabetes and related conditions” refers to diseases or medical conditions caused by the reduction, lack, or inaction of, or inability to utilize, insulin. Diabetes and related conditions include type I and type II diabetes, particularly type I diabetes.

### Modified Therapeutic Peptides

Turning now to one or more aspects of the invention, conjugates are provided, the conjugates comprising a therapeutic peptide covalently attached (either directly or through a spacer moiety or linker) to a water-soluble polymer. The conjugates generally have the following formula:

PEP-[—X-POLY]ₖ

wherein PEP is a therapeutic peptide as defined herein, X is a covalent bond or is a spacer moiety or linker, POLY is a water-soluble polymer, and k in an integer ranging from 1-10, preferably 1-5, and more preferably 1-3.

The modified therapeutic peptides, e.g., modified insulin, may comprise at least one amino acid residue covalently attached to a hydrophilic polymer and/or at least one amino acid covalently attached to a moiety having one to ten carbon atoms. In some embodiments, the moiety having one to ten carbon atoms is not a hydrophilic polymer, e.g., not a PEG. As used in the following discussion, “modification” can refer to the covalent addition of a hydrophilic polymer and/or of a moiety having one to ten carbon atoms.

In other embodiments, the therapeutic peptides are selected from the group consisting of peptide G, OTS-102, Angiocol (antiangiogenic peptide group), ABT-510 (antiangiogenic peptide group), A6 (antiangiogenic peptide group), islet neogenesis gene associated protein (INGAP), tendamistat, recombinant human carperitide (alpha-atrial natriuretic peptide) (natriuretic peptide group), urodilatin (natriuretic peptide group), desirudin, Obestatin, ITF-1697, oxyntomodulin, cholecystokinin, bactericidal permeability increasing (BPI) protein, C-peptide, Prosaptide TX14(A), sermorelin acetate (GHRFA group), pralmorelin (GHRFA group), growth hormone releasing factor (GHRFA group), examorelin (GHRFA group), gonadorelin (LH-related peptide group), corticoliberin, atrial natriuretic peptide (natriuretic peptide group), anergix, somatostatin (GHRFA group), 29-amino-acid peptide growth hormone releasing hormone (GHRH) analogue (GHRFA group), bremelanotide (melanocortin agonist group), melanocortin peptidomimetic compound (melanocortin agonist group), antiprogestogens-GnRH antagonists (LH-related peptide group), recombinant LH (luteinizing hormone) (LH-related peptide group), terlipressin, Ecallantide-60-amino-acid recombinant peptide kallikrein inhibitor, calphobindin I, tiplimotide, osteogenic growth peptide, myelin basic protein, dynorphin A, anaritide (natriuretic peptide group), secretin, GLP-2, and gastrin.

The therapeutic peptides of the invention may comprise any of the 20 natural amino acids, and/or non-natural amino acids, amino acid analogs, and peptidomimetics, in any combination. The peptides may be composed of D-amino acids or L-amino acids, or a combination of both in any proportion. In addition to natural amino acids, the therapeutic peptides may contain, or may be modified to include, 1, 2, 3, 4, 5, 6, 7, 8, 9, 10, 15, 20, 25, or more non-natural amino acids. Exemplary non-natural amino acids and amino acid analogs that can be use with the invention include, but are not limited to, 2-aminobutyric acid, 2-aminoisobutyric acid, 3-(1-naphthyl)alanine, 3-(2-naphthyl)alanine, 3-methylhistidine, 3-pyridylalanine, 4-chlorophenylalanine, 4-fluorophenylalanine, 4-hydroxyproline, 5-hydroxylysine, alloisoleucine, citrulline, dehydroalanine, homoarginine, homocysteine, homoserine, hydroxyproline, N-acetylserine, N-formylmethionine, N-methylglycine, N-methylisoleucine, norleucine, N-α-methylarginine, O-phosphoserine, ornithine, phenylglycine, pipecolinic acid, piperazic acid, pyroglutamine, sarcosine, valanine, β-alanine, and β-cyclohexylalanine.

As noted above, “insulin” as used herein is meant to encompass any purified isolated polypeptide having part or all of the primary structural conformation (that is to say, contiguous series of amino acid residues) and at least one of the biological properties of naturally occurring insulin. In some embodiments, the insulin is an insulin occurring in nature, for example human, bovine or porcine insulin, or the insulin of another animal or mammal. In some embodiments, the insulin comprises an insulin analog, such as at least one of Gly(A21)-Arg(B31)-Arg(B32) human insulin; Lys(B3)-Glu(B29) human insulin; Lys^{B28}Pro^{B29} human insulin, B28 Asp human insulin, human insulin, in which proline in position B28 has been substituted by Asp, Lys, Leu, Val or Ala and where in position B29 Lys can be substituted by Pro; AlaB26 human insulin; des(B28-B30) human insulin; des(B27) human insulin or des(B30) human insulin. In additional embodiments, the polypeptide of the preparation comprises an insulin derivative selected from at least one of B29-N-myristoyl-des(B30) human insulin, B29-N-palmitoyl-des(B30) human insulin, B29-N-myristoyl human insulin, B29-N-palmitoyl human insulin, B28-N-myristoyl Lys^{B28}Pro^{B29} human insulin, B28-N-palmitoyl-Lys^{B28}Pro^{B29} human insulin, B30-N-myristoyl-Thr^{B29}Lys^{B30} human insulin, B30-N-palmitoyl-Thr^{B29}Lys^{B30} human insulin, B29-N-(N-palmitoyl-γ-glutamyl)-des (B30) human insulin, B29-N-(N-lithocholyl-γ-glutamyl)-des(B30) human insulin, B29-N-(ω-carboxyheptadecanoyl)-des(B30) human insulin, and B29-N-(ω-carboxyheptadecanoyl) human insulin. Some embodiments comprise preparations containing mixtures of one or more of insulin, an insulin analog, and an insulin derivative, for example, selected from those described above.

TABLE A
This table lists the SEQ ID NOs., names, sequences, and known or suspected therapeutic activities of various peptides described herein.
The SEQ ID NOs. 1-301 describe sequences that are required to be provided with the Sequence Listing and are therefore appended with the instant
Specification. In some instances, these peptides contain features that are either inconsistent with or not amenable to inclusion in the Sequence
Listing. For example, a sequence with less than four-amino acids; a sequence with a D-amino acid; or certain modification that cannot be
described in the Sequence Listing presently, and therefore are not provided in the Sequence Listing. However, for the ease of use and
description, a SEQ ID NO. has been provided to these peptides. (—NH₂ indicates amidation at the C-terminus; Ac indicates acetylation; other
modifications are as described herein and in the specification; SIN indicates Sequence Identification Number)

| SEQ ID | | | | |
|---|---|---|---|---|
| NO: | Name | Family | Sequence and/or other Identifying Information | Therapeutic Activity |
| 1 | carperitide | ANP | SLRRSSCFGGRMDRIGAQSGLGCNSFRY; | Cardiostimulant |
| | | | human alpha-atrial natriuretic peptide; Atriopeptin-28 | Respiratory |
| | | | (human); | |
| 2 | alpha- | Endorphin | H-Tyr-Gly-Gly-Phe-Leu-Arg-Lys-Tyr-Pro-Lys-OH | Analgesic, other |
| | neoendorphin | | | |
| 3 | A-3847 | Insulin | gi\|386828\|gb\|AAA59172.1\|insulin [Homo sapiens] | Antidiabetic |
| | | | MALWMRLLPLLALLALWGPDPAAAFVNQHLCGSHL | |
| | | | VEALYLVCGERGFFYTPKTRREAEDLQVGQVELGGG | |
| | | | PGAGSLQPLALEGSLQKRGIVEQCCTSICSLYQLENYC | |
| | | | N | |
| 4 | A-4114 | Insulin | MALWMRLLPLLALLALWGPDPAAAFVNQHLCGSHL | Antidiabetic |
| | | | VEALYLVCGERGFFYTPKTRREAEDLQVGQVELGG | |
| 5 | A-68552 | | GPGAGSLQPLALEGSLQKRGIVEQCCTSICSLYQLENY | Anorectic/Antiobesity |
| | | | CN | |
| 302 and | A-75998 | | [Ac—D-2Nal1-D-4ClPhe2-D-3Pal3-NMeTyr5-D-Lys(Nic)6- | Releasing hormone |
| 303 | | | Lys(Isp)8-D-Ala10]GnRH; | Reproductive/gonadal, |
| | | | N-acetyl-D-2-naphthylalanyl-D-4-chlorophenylalanyl-D-3- | general |
| | | | pyridylalanyl-seryl-N-methyltyrosyl-D-N(epsilon)- | |
| | | | nicotinyllysyl-leucyl-N(epsilon)-isopropyllysyl-prolyl- | |
| | | | alaninamide acetate | |
| 6 | AN-1792 | beta-amyloid | gi\|8176533\|gb\|AAB26264.2\| beta-amyloid peptide precursor; | Cognition enhancer |
| | | peptide | beta APP [Homo sapiens] | |
| | | | GSGLTNIKTEEISEVKMDAEFRHDSGYEVHHQKLVFF | |
| | | | AEDVGSNKGAIIGLMVGGVVIATVIIITLVMLK | |
| | | | KQYTSNHHGVVE | |
| 7 | AAMP-1 | | MESESESGAAADTPPLETLSFHGDEEIIEVVELDPGPPD | Anticoagulant |
| | | | PDDLAQEMEDVDFEEEEEEEGNEEGWVLEPQEGVVG | Anti-inflammatory |
| | | | SMEGPDDSEVTFALHSASVFCVSLDPKTNTLAVTGGE | Immunological |
| | | | DDKAFVWRLSDGELLFECAGHKDSVTCAGFSHDSTL | Anticancer, other |
| | | | VATGDMSGLLKVWQVDTKEEVWSFEAGDLEWMEW | Vulnerary |
| | | | HPRAPVLLAGTADGNTWMWKVPNGDCKTFQGPNCP | |
| | | | ATCGRVLPDGKRAVVGYEDGTIRIWDLKQGSPIHVLK | |
| | | | GTEGHQGPLTCVAANQDGSLILTGSVDCQAKLVSATT | |
| | | | GKVVGVFRPETVASQPSLGEGEESESNSVESLGFCSV | |
| | | | MPLAAVGYLDGTLAIYDLATQTLRHQCQHQSGIVQLL | |
| | | | WEAGTAVVYTCSLDGIVRLWDARTGRLLTDYRGHTA | |
| | | | EILDFALSKDASLVVTTSGDHKAKVFCVQRPDR | |
| 8 | Exenatide | GLP-1 | HGEGTFTSDLSKQMEEEAVRLFIEWLKNGGPSSGAPP | Antidiabetic |
| | | | PS | Anorectic/Antiobesity |
| 9 | AC-625 | | Acetyl-ATQRLANELVRLQTYPRTNVGSNTY-NH₂ | Antihypertensive, renin |
| | | | | system |
| | | | | Symptomatic antidiabetic |
| 10 | ACTH | | gi\|80861463\|ref\|NP_001030333.1\| proopiomelanocortin | Adrenal and pituitary |
| | | | preproprotein [Homo sapiens] | disorders |
| | | | MPRSCCSRSGALLLALLLQASMEVRGWCLESSQCQD | |
| | | | LTTESNLLECIRACKPDLSAETPMFPGNGDEQPLTENP | |
| | | | RKYVMGHFRWDRFGRRNSSSSGSSGAGQKREDVSAG | |
| | | | EDCGPLPEGGPEPRSDGAKPGPREGKRSYSMEHFRWG | |
| | | | KPVGKKRRPVKVYPNGAEDESAEAFPLEFKRELTGQR | |
| | | | LREGDGPDGPADDGAGAQADLEHSLLVAAEKKDEGP | |
| | | | YRMEHFRWGSPPKDKRYGGFMTSEKSQTPLVTLFKN | |
| | | | AIIKNAYKKGE | |
| 11 | AIDS | | gi\|288842\|emb\|CAA78890.1\|V3 loop [Human | Therapeutic vaccine |
| | therapeutic | | immunodeficiency virus type 1] | |
| | vaccine | | CTRPSNNTRKSIPVGPGKALYATGAIIGNIRQAHC | |
| 12 | AIDS therapy | | gi\|5081475\|gb\|AAD39400.1\|AF128998_1 gag [Human | Antiviral, anti-HIV |
| | | | immunodeficiency virus type 1] | |
| | | | MGARASVLSGGKLDKWEKIRLRPGGKKTYQLKHIVW | |
| | | | ASRELERFAVNPGLLETGGGCKQILVQLQPSLQTGSEE | |
| | | | LKSLYNAVATLYCVHQGIEVRDTKEALDKIEEEQNKS | |
| | | | KKKAQQAAADTGNSSQVSQNYPIVQNLQGQMVHQAI | |
| | | | SPRTLNAWVKVIEEKAFSPEVIPMFSALSEGATPQDLN | |
| | | | TMLNTVGGHQAAMQMLKETINEEAAEWDRLHPAHA | |
| | | | GPNAPGQMREPRGSDIAGTTSTLQEQIGWMTSNPPVP | |
| | | | VGEIYKRWIILGLNKIVRMYSPVSILDIRQGPKEPFRDY | |
| | | | VDRFYKTLRAEQASQDVKNWMTETLLVQNANPDCK | |
| | | | TILKALGPAATLEEMMTACQGVGGPSHKARILAEAMS | |
| | | | QVTSPANIMMQRGNFRNQRKTIKCFNCGKEGHLARH | |
| | | | CRAPRKKGCWKCGREGHQMKDCTERQANFLGKIWP | |
| | | | SHKGRPGNFLQSRPEPTAPPEESFRFGEETTTPPQKQEP | |
| | | | LPSQKQETIDKDLYPLASLKSLFGNDPSLQ | |
| 13 and | Allotrap-2702 | | Allotrap 1258; Allotrap 2702; Allotrap E; Allotrap G; | Immunosuppressant |
| 14 | | | RDP58; peptide Bc-1nl; | |
| | | | NLRIALR/RLAIRLN | |
| 15 and | Alzheimer's | | H— | Imaging agent |
| 16 | imaging agent | | DAEFRHDSGYEVHHQKLVFFAEDVGSNKGAIIGLMV | |
| | | | GGVV-OH; or | |
| | | | H— | |
| | | | DAEFRHDSGYEVHHQKLVFFAEDVGSNKGAIIGLMV | |
| | | | GGVVIA-OH | |
| 17 | AM-425 | | gi\|4504991\|ref\|NP_002300.1\| leukemia inhibitory factor | Antiarthritic, |
| | | | (cholinergic differentiation factor) [Homo sapiens] | immunological |
| | | | MKVLAAGVVPLLLVLHWKHGAGSPLPITPVNATCAIR | |
| | | | HPCHNNLMNQIRSQLAQLNGSANALFILYYTAQGEPF | |
| | | | PNNLDKLCGPNVTDFPPFHANGTEKAKLVELYRIVVY | |
| | | | LGTSLGNITRDQKILNPSALSLHSKLNATADILRGLLSN | |
| | | | VLCRLCSKYHVGHVDVTYGPDTSGKDVFQKKKLGCQ | |
| | | | LLGKYKQIIAVLAQAF | |
| 304 | AN-238 | | L-Threoninamide, N-[5-[2-[(2S,4S)-1,2,3,4,6,11-hexahydro- | Somatostatin |
| | | | 2,5,12-trihydroxy-7-methoxy-6,11-dioxo-4-[[2,3,6-trideoxy- | Anticancer, hormonal |
| | | | 3-(2,3-dihydro-1H-pyrrol-1-yl)-alpha-L-lyxo- | |
| | | | hexopyranosyl]oxy]-2-naphthacenyl]-2-oxoethoxy]-1,5- | |
| | | | dioxopentyl]-D-phenylalanyl-L-cysteinyl-L-tyrosyl-D- | |
| | | | tryptophyl-L-lysyl-L-valyl-L-cysteinyl-, cyclic (2-7)- | |
| | | | disulfide | |
| 305 | AV-9 | | [D-Arg]9-NH₂ | Antiviral, other |
| 8 | AZM-134 | GLP-1 | HGEGTFTSDLSKQMEEEAVRLFIEWLKNGGPSSGAPP | Anorectic/Antiobesity |
| | | | PS | Antidiabetic |
| 18 | Addressin | | gi\|109633022\|ref\|NP_570116.2\| mucosal vascular addressin | Recombinant, other |
| | | | cell adhesion molecule 1 isoform a precursor [Homo | Anti-inflammatory |
| | | | sapiens] | |
| | | | MDFGLALLLAGLLGLLLGQSLQVKPLQVEPPEPVVAV | |
| | | | ALGASRQLTCRLACADRGASVQWRGLDTSLGAVQSD | |
| | | | TGRSVLTVRNASLSAAGTRVCVGSCGGRTFQHTVQLL | |
| | | | VYAFPDQLTVSPAALVPGDPEVACTAHKVTPVDPNAL | |
| | | | SFSLLVGGQELEGAQALGPEVQEEEEEPQGDEDVLFR | |
| | | | VTERWRLPPLGTPVPPALYCQATMRLPGLELSHRQAI | |
| | | | PVLHSPTSPEPPDTTSPESPDTTSPESPDTTSQEPPDTTS | |
| | | | PEPPDKTSPEPAPQQGSTHTPRSPGSTRTRRPEISQAGP | |
| | | | TQGEVIPTGSSKPAGDQLPAALWTSSAVLGLLLLALPT | |
| | | | YHLWKRCRHLAEDDTHPPASLRLLPQVSAWAGLRGT | |
| | | | GQVGISPS | |
| 306 | ambamustine | | L-Methionine, N-[3-[bis(2-chloroethyl)amino]-N-(4-fluoro- | Anticancer, alkylating |
| | | | L-phenylalanyl)-L-phenylalanyl]-, ethyl ester | Anticancer, antimetabolite |
| 19 | amylin | | DTTVSEPAPSCVTLYQSWRYSQADNGCAETVTVKVV | Antidiabetic |
| | antagonists | | YEDDTEGLCYAVAPGQITTVGDGYIGSHGHARYLAR | |
| | | | CL | |
| 20 | anaritide | ANP | gi\|178638\|gb\|AAA35529.1\| atrial natriuretic peptide | Antihypertensive, diuretic |
| | analogues | | MSSFSTTTVSFLLLLAFQLLGQTRANPMYNAVSNADL | |
| | | | MDFKNLLDHLEEKMPLEDEVVPPQVLSDPNEEAGAA | |
| | | | LSPLPEVPPWTGEVSPAQRDGGALGRGPWDSSDRSAL | |
| | | | LKSKLRALLTAPRSLRRSSCFGGRMDRIGAQSGLGCN | |
| | | | SFRY | |
| 21-28 | anti- | | As disclosed in U.S. Pat. No. 5,470,831: | Anti-inflammatory |
| | inflammatory | | Thr-Thr-Ser-Gln-Val-Arg-Pro-Arg | Immunosuppressant |
| | peptide | | Val-Lys-Thr-Thr-Ser-Gln-Val-Arg-Pro-Arg. | Multiple sclerosis |
| | | | Ser-Gln-Val-Arg-Pro-Arg | treatment |
| | | | Val-Arg-Pro-Arg | Antiarthritic, other |
| | | | Thr-Thr-Ser-Gln-Val-Arg-Pro-Arg-His-Ile-Thr. | Stomatological |
| | | | Thr-Thr-Ser-Gln-Val | Dermatological |
| | | | Thr-Ser-Gln-Val-Arg | |
| | | | Thr-Thr-Ser-Gly-Ile-His-Pro-Lys | |
| 307 | antiflammins | | L-Leucine, N-[N-[N-[N-[N2-[N2-[N-(N-L-histidyl-L-alpha- | Anti-inflammatory |
| | | | aspartyl)-L-methionyl]-L-asparaginyl]-L-lysyl]-L-valyl]-L- | |
| | | | leucyl]-L-alpha-aspartyl]- | |
| 308 | antifungal | | tripeptides of N3-4-methoxyfumanyl | Antifungal |
| | tripeptides | | and di- and tripeptides of N3-D-trans 2,3-epoxysuccinamoyl- | |
| | | | L-2,3-diaminopropanoic acid | |
| 29 | Gastrimmune | | G17-DT; G17DT (vaccine); Gastrimmune; Glu-Gly-Pro- | Anticancer, immunological |
| | | | Trp-Leu-Glu-Glu-Glu-Glu -diphtheria toxoid; anti-gastrin 17 | |
| | | | immunogen; gastrin 17 vaccine; gastrin-17-diphtheria toxoid | |
| | | | conjugate | |
| 30 | antithrombin | | gi\|312673\|emb\|CAA51292.1\| Hirudin [Hirudinaria | Antithrombotic |
| | polypeptides | | manillensis] | Anticoagulant |
| | | | MFSLKLFVVFLAVCICVSQAVSYTDCTESGQNYCLCV | |
| | | | GGNLCGGGKHCEMDGSGNKCVDGEGTPKPKSQT | |
| | | | EGDFEEIPDEDILN | |
| 31 | antiviral | | NH₂-Tyr-Ala-Gly-Ala-Val-Val-Asn-Asp-Leu-COOH | Antiviral, other |
| | peptides | | | |
| 32 | apolipoprotein | | gi\|671882\|emb\|CAA28583.1\| apolipoprotein [Homo sapiens] | Hypolipaemic/Antiathero- |
| | | | MKLLAATVLLLTICSLEGALVRRQAKEPCVESLVSQY | sclerosis |
| | | | FQTVTDYGKDLMEKVKSPELQAEAKSYFEKSKE | |
| | | | QLTPLIKKAGTELVNFLSYFVELGTHPATQ | |
| 33 | arthritis antigen | | gi\|46369603\|gb\|AAS89650.1\| secreted antigen 85A precursor | Recombinant, other |
| | | | [Mycobacterium bovis BCG] | Antiarthritic, |
| | | | MQLVDRVRGAVTGMSRRLVVGAVGAALVSGLVGAV | immunological |
| | | | GGTATAGAFSRPGLPVEYLQVPSPSMGRDIKVQFQSG | Immunosuppressant |
| | | | GANSPALYLLDGLRAQDDFSGWDINTPAFEWYDQSG | |
| | | | LSVVMPVGGQSSFYSDWYQPACGKAGCQTYKWETF | |
| | | | LTSELPGWLQANRHVKPTGSAVVGLSMAASSALTLAI | |
| | | | YHPQQFVYAGAMSGLLDPSQAMGPTLIGLAMGDAGG | |
| | | | YKASDMWGPKEDPAWQRNDPLLNVGKLIANNTRVW | |
| | | | VYCGNGKPSDLGGNNLPAKFLEGFVRTSNIKFQDAYN | |
| | | | AGGGHNGVFDFPDSGTHSWEYWGAQLNAMKPDLQR | |
| | | | ALGATPNTGPAPQGA | |
| 309 | Avorelin | | 5-Oxo-L-prolyl-L-histidyl-L-tryptophyl-L-seryl-L-tyrosyl-2- | Releasing hormone |
| | | | methyl-D-tryptophyl-L-leucyl-L-arginyl-N-ethyl-L- | Anticancer, hormonal |
| | | | prolinamide | Menstruation disorders |
| 310 | B-956 | | N-[8(R)-Amino-2(S)-benzyl-5(S)-isopropyl-9-sulfanyl- | Anticancer, other |
| | | | 3(Z),6(E)-nonadienoyl]-L-methionine | |
| 311 | BCH-2687 | | L-Tyrosyl-D-arginyl-L-phenylalanyl-L-phenylalaninamide | Analgesic, other |
| 34 | BCH-2763 | | L-Leucine, D-phenylalanyl-L-prolyl-5-aminopentanoyl-5- | Antithrombotic |
| | | | aminopentanoyl-L-alpha-aspartyl-L-phenylalanyl-L-alpha- | Anticoagulant |
| | | | glutamyl-L-prolyl-L-isoleucyl-L-prolyl-; BCH-2763; Phe- | |
| | | | Pro-(NH(CH₂)4CO)₂-Asp-Phe-Glu-Pro-Ile-Pro-Leu; | |
| | | | phenylalanyl-proly1-(NH(CH₂)4CO)₂-aspartyl-phenylalanyl- | |
| | | | glutamyl-prolyl-isoleucyl-prolyl-leucine | |
| 312 | frakefamide | | L-phenylalaninamide, L-tyrosyl-D-alanyl-4-fluoro-L- | Analgesic, other |
| | | | phenylalanyl- | |
| 313 | BIM-22015 | | Glycinamide, D-alanyl-L-glutaminyl-L-tyrosyl-L- | ACTH |
| | | | phenylalanyl-L-arginyl-L-tryptophyl- | Neurological |
| 35 | BIM-26028 | | Pyroglutamyl-glutaminyl-arginyl-leucyl-glycyl-asparaginyl- | Releasing hormone |
| | | | glutaminyl-tryptyl-alanyl-valyl-glycyl-histidinyl-leucyl- | Respiratory |
| | | | leucyl-NH₂ | Anorectic/Antiobesity |
| | | | | Anticancer, hormonal |
| 314 | BIM-44002 | | L-Tyrosinamide, L-phenylalanyl-L-norleucyl-L-histidyl-L- | Hormone |
| | | | asparaginyl-L-leucyl-D-tryptophyl-L-lysyl-L-histidyl-L- | Osteoporosis treatment |
| | | | leucyl-L-seryl-L-seryl-L-norleucyl-L-alpha-glutamyl-L- | |
| | | | arginyl-L-valyl-L-.alpha.-glutamyl-L-tryptophyl-L-leucyl-L- | |
| | | | arginyl-L-lysyl-L-lysyl-L-leucyl-L-glutaminyl-L-alpha- | |
| | | | aspartyl-L-valyl-L-histidyl-L-asparaginyl- | |
| 36 | BIO-1211 | | L-Proline, N-((4-((((2- | Antiasthma |
| | | | methylphenyl)aminocarbonyl)amino)phenyl)acetyl)-L- | GI inflammatory/bowel |
| | | | leucyl-Lalpha-aspartyl-L-valyl-; BIO-1211; N-((4-((((2- | disorders |
| | | | methylphenyl)amino)carbonyl)amino)phenyl)acetyl)-leucyl- | Multiple sclerosis |
| | | | aspartyl-valyl-proline | treatment |
| 37 | BPC-15 | | BPC 15; BPC-15; BPC-157; booly protection compound 15; | Anti-inflammatory |
| | | | L-Valine, glycyl-L-alpha-glutamyl-L-prolyl-L-prolyl-L- | |
| | | | prolylglycyl-L-lysyl-L-prolyl-L-alanyl-L-alpha-aspartyl-L- | |
| | | | alpha-aspartyl-L-alanylglycyl-L-leucyl- | |
| 315 | bivalirudin | | L-Leucine, D-phenylalanyl-L-prolyl-L-arginyl-L- | Anticoagulant |
| | | | prolylglycylglycylglycylglycyl-L-asparaginylglycl-L-alpha- | Antianginal |
| | | | aspartyl-L-phenylalanyl-L-alpha-glutamyl-L-alpha- | |
| | | | glutamyl-L-isoleucyl-L-prolyl-L-alpha-glutamyl-L-tyrosyl-; | |
| | | | D-phenylalanyl-L-prolyl-L-arginyl-L-prolyl-glycylglycyl- | |
| | | | glycyl-glycyl-L-asparagyl-glycyl-L-aspartyl-L-phenylalanyl- | |
| | | | L-glutamyl-L-glutamyl-L-isoleucyl-Lprolyl-L-glutamyl-L- | |
| | | | glutamyl-L-tyrosyl-L-leucine trifluoroacetate (salt) hydrate | |
| 38 | bombesin | | 5-oxoPro-Gln-Arg-Leu-Gly-Asn-Gln-Trp-Ala-Val-Gly-His- | Anticancer, other |
| | antagonist | | Leu-MetNH₂ [CAS], Bombesin 14; Bombesin | |
| | | | Dihydrochloride; Dihydrochloride, Bombesin | |
| 39 | brain natriuretic | BNP | SPKMVQGSGCFGRKMDRISSSSGLGCKVLRRH | COPD treatment, cardiac |
| | peptide | | | |
| 41 | C-peptide | C-peptide | Glu-Ala-Glu-Asp-Leu-Gln-Val-Gly-Gln-Val-Glu-Leu-Gly- | Symptomatic antidiabetic |
| | analogues | | Gly-Gly-Pro-Gly-Ala-Gly-Ser-Leu-Gln-Pro-Leu-Ala-Leu- | Ophthalmological |
| | | | Glu-Gly-Ser-Leu-Gln | Neurological |
| 316 | C5a antagonist | | Me-Phe-Lys-Pro-D-Cha-L-Cha-D-Phe | Anti-inflammatory |
| 42 | CBT-101 | | L-Cysteinamide, L-asparaginyl-L-leucylglycyl-L-valyl-S- | Antiglaucoma |
| | | | [(acetylamino)methyl]-, monoacetate | |
| 43 | CCK(27-32) | | Tyr(SO₃)-Met-Gly-Trp-Met-Asp; CBZ CCK (27-32)-NH₂; | Analgesic, obesity, other |
| | | | cholecystokinin (27-32) amide, benzoyloxycarbonyl-, D-Trp | |
| 44 | CD4 | | CD4 (81-92), D-Ile; CD4 (81-92), D-Tyr; CD4 (81-92), D- | Antiviral, anti-HIV |
| | | | Tyr, D-Cys, D-Glu(5); CD4(81-92); TYICEVEDQKEE; Thr- | |
| | | | Tyr-Ile-Cys-Glu-Val-Glu-Asp-Gln-Lys-Glu-Glu; | |
| | | | threonyl-tyrosyl-isoleucyl-cysteinyl-glutamyl-valyl- | |
| | | | glutamyl-aspartyl-glutaminyl-lysyl-glutamyl-glutamic acid | |
| 317 | CEE-04-420 | | Lys-D-Pro-Thr and Lys-D-Pro-Val | Analgesic, other |
| 45 | CEP-079 | | gi\|108796063\|ref\|NP_001007140.2\| insulin-like growth | Ophthalmological |
| | | | factor 2 isoform 1 precursor [Homo sapiens] | |
| | | | MGIPMGKSMLVLLTFLAFASCCIAAYRPSETLCGGEL | |
| | | | VDTLQFVCGDRGFYFSRPASRVSRRSRGIVEEC | |
| | | | CFRSCDLALLETYCATPAKSERDVSTPPTVLPDNFPRY | |
| | | | PVGKFFQYDTWKQSTQRLRRGLPALLRARRGH | |
| | | | VLAKELEAFREAKRHRPLIALPTQDPAHGGAPPEMAS | |
| | | | NRK | |
| 318 | mifamurtide | | L-Alaninamide, N-(N-acetylmuramoyl)-L-alanyl-D-alpha- | Anticancer, immunological |
| | | | glutaminyl-N-[4-hydroxy-10-oxo-7-[(1-oxohexadecyl)oxy]- | |
| | | | 3,5,9-trioxa-4-phosphapentacos-1-yl]-, P-oxide, | |
| | | | monosodium salt, (R)— | |
| 46 | CGRP | CGRP | ACDTATCVTHRLAGLLSRSGGVVKNNFVPTNVGSKA | Hormone |
| | analogues | | F—NH₂ | Cardiovascular |
| 47 | rusalatide | | gi\|4503635\|ref\|NP_000497.1\| coagulation factor II | Musculoskeletal |
| | acetate | | preproprotein [Homo sapiens] | Vulnerary |
| | | | MAHVRGLQLPGCLALAALCSLVHSQHVFLAPQQARS | Symptomatic antidiabetic |
| | | | LLQRVRRANTFLEEVRKGNLERECVEETCSYEEAFEA | Cardiovascular |
| | | | LESSTATDVFWAKYTACETARTPRDKLAACLEGNCA | Anti-infective, other |
| | | | EGLGTNYRGHVNITRSGIECQLWRSRYPHKPEINSTTH | Ophthalmological |
| | | | PGADLQENFCRNPDSSTTGPWCYTTDPTVRRQECSIPV | |
| | | | CGQDQVTVAMTPRSEGSSVNLSPPLEQCVPDRGQQY | |
| | | | QGRLAVTTHGLPCLAWASAQAKALSKHQDFNSAVQL | |
| | | | VENFCRNPDGDEEGVWCYVAGKPGDFGYCDLNYCE | |
| | | | EAVEEETGDGLDEDSDRAIEGRTATSEYQTFFNPRTFG | |
| | | | SGEADCGLRPLFEKKSLEDKTERELLESYIDGRIVEGS | |
| | | | DAEIGMSPWQVMLFRKSPQELLCGASLISDRWVLTAA | |
| | | | HCLLYPPWDKNFTENDLLVRIGKHSRTRYERNIEKISM | |
| | | | LEKIYIHPRYNWRENLDRDIALMKLKKPVAFSDYIHP | |
| | | | VCLPDRETAASLLQAGYKGRVTGWGNLKETWTANV | |
| | | | GKGQPSVLQVVNLPIVERPVCKDSTRIRITDNMFCAG | |
| | | | YKPDEGKRGDACEGDSGGPFVMKSPFNNRWYQMGI | |
| | | | VSWGEGCDRDGKYGFYTHVFRLKKWIQKVIDQFGE | |
| 48 | CKS-17 | | L-Leucine, L-leucyl-L-glutaminyl-L-asparaginyl-L-arginyl- | Immunosuppressant |
| | | | L-arginylglycyl-L-leucyl-L-alpha-aspartyl-L-leucyl-L- | Anticancer, immunological |
| | | | leucyl-L-phenylalanyl-L-leucyl-L-lysyl-L-alpha- | |
| | | | glutamylglycylglycyl-; CKS-17; CKS-17 peptide | |
| 10 | corticorelin | corticotropin | gi\|80861463\|ref\|NP_001030333.1\| proopiomelanocortin | Neuroprotective |
| | acetate | | preproprotein [Homo sapiens] | Antiasthma |
| | | | MPRSCCSRSGALLLALLLQASMEVRGWCLESSQCQD | Anti-inflammatory |
| | | | LTTESNLLECIRACKPDLSAETPMFPGNGDEQPLTENP | |
| | | | RKYVMGHFRWDRFGRRNSSSSGSSGAGQKREDVSAG | |
| | | | EDCGPLPEGGPEPRSDGAKPGPREGKRSYSMEHFRWG | |
| | | | KPVGKKRRPVKVYPNGAEDESAEAFPLEFKRELTGQR | |
| | | | LREGDGPDGPADDGAGAQADLEHSLLVAAEKKDEGP | |
| | | | YRMEHFRWGSPPKDKRYGGFMTSEKSQTPLVTLFKN | |
| | | | AIIKNAYKKGE | |
| 49 | CT-112 | | L-Arginine, L-threonyl-L-threonyl-L-seryl-L-glutaminyl-L- | Antiarthritic, |
| | | | valyl-L-arginyl-L-prolyl-; 5-(3-ethoxy-4-pentyloxyphenyl)- | immunological |
| | | | 2,4-thiazolidinedione | |
| 50 | CTAP-III | | phenylalanyl-cysteinyl-tyrosyl-tryptophyl-arginyl-threonyl- | Vulnerary |
| | | | penicillaminyl-threoninamide; rCTAP-III-Leu-21 (des 1-15); | Antiarthritic, other |
| | | | somatostatin analog CTAP | Musculoskeletal |
| | | | | Recombinant, other |
| 51 | CVFM | | Cys-Val-Phe-Met | Anticancer, other |
| 52 and | calcitonin | calcitonin | CGNLSTCMLGTYTQDFNKFHTFPQTAIGVGAP | Formulation, oral, other |
| 53 | | | (human) | Hormone |
| | | | H-Cys-Ser-Asn-Leu-Ser-Thr-Cys-Val-Leu-Gly-Lys-Leu- | Osteoporosis treatment |
| | | | Ser-Gln-Glu-Leu-His-Lys-Leu-Gln-Thr-Tyr-Pro-Arg-Thr- | |
| | | | Asn-Thr-Gly-Ser-Gly-Thr-Pro-NH₂ (salmon) | |
| 54 | calciseptine | | sp\|P22947\|TXCAS_DENPO Calciseptin OS = Dendroaspis | Antihypertensive, other |
| | | | polylepis polylepis PE = 1 SV = 1 | |
| | | | RICYIHKASLPRATKTCVENTCYKMFIRTQREYISERG | |
| | | | CGCPTAMWPYQTECCKGDRCNK | |
| 52 and | calcitonin | calcitonin | CGNLSTCMLGTYTQDFNKFHTFPQTAIGVGAP | Hormone |
| 53 | analogues | | (human) | Osteoporosis treatment |
| | | | H-Cys-Ser-Asn-Leu-Ser-Thr-Cys-Val-Leu-Gly-Lys-Leu- | |
| | | | Ser-Gln-Glu-Leu-His-Lys-Leu-Gln-Thr-Tyr-Pro-Arg-Thr- | |
| | | | Asn-Thr-Gly-Ser-Gly-Thr-Pro-NH₂ (salmon) | |
| 55 | calphobindin I | | gi\|186680508\|ref\|NM_001154.3\| Homo sapiens annexin A5 | Ophthalmological, |
| | | | (ANXA5), | Vulnerary |
| | | | MAQVLRGTVTDFPGFDERADAETLRKAMKGLGTDEE | |
| | | | SILTLLTSRSNAQRQEISAAFKTLFGRDLLDDLKSELTG | |
| | | | KFEKLIVALMKPSRLYDAYELKHALKGAGTNEKVLT | |
| | | | EIIASRTPEELRAIKQVYEEEYGSSLEDDVVGDTSGYY | |
| | | | QRMLVVLLQANRDPDAGIDEAQVEQDAQALFQAGEL | |
| | | | KWGTDEEKFITIFGTRSVSHLRKVFDKYMTISGFQIEE | |
| | | | TIDRETSGNLEQLLLAVVKSIRSIPAYLAETLYYAMKG | |
| | | | AGTDDHTLIRVMVSRSEIDLFNIRKEFRKNFATSLYSM | |
| | | | IKGDTSGDYKKALLLLCGEDD | |
| 319 | cargutocin | | 1,6-Dicarbaoxytocin, 1-butanoic acid-7-glycine- | Labour inducer |
| 320 | casokefamide | | L-Tyrosinamide, L-tyrosyl-D-alanyl-L-phenylalanyl-D- | Antidiarrhoeal |
| | | | alanyl- | |
| 56 | cekropin-P | | sp\|P14661\|CECP1_PIG Cecropin-P1 OS = Sus scrofa PE = 1 | Antibacterial, other |
| | | | SV = 1 | |
| | | | SWLSKTAKKLENSAKKRISEGIAIAIQGGPR | |
| 57 | tasidotin | | N,N-Dimethyl-L-valyl-L-valyl-N-methyl-L-valyl-L-propyl- | Anticancer, other |
| | hydrochloride | | L-proline-tert-butylamide | |
| 58 | ceruletide | | Pyr-Gln-Asp-Tyr(SO3H)-Thr-Gly-Trp-Met-Asp-Phe-C(O)—NH₂ | Analgesic, other |
| | diethylamine | | | Gastroprokinetic |
| 321 | cetrorelix | | D-Alaninamide, N-acetyl-3-(2-naphthalenyl)-D-alanyl-4- | Fertility enhancer |
| | acetate | | chloro-D-phenylalanyl-3-(3-pyridinyl)-D-alanyl-L-seryl-L- | Prostate disorders |
| | | | tyrosyl-N5-(aminocarbonyl)-D-ol-L-leucyl-L-arginyl-L- | Menstruation disorders |
| | | | prolyl- | Anticancer, hormonal |
| 59 | corticoliberin | corticoliberin | SQEPPISLDLTFHLLREVLEMTKADQLAQQAHSNRKL | Releasing hormone |
| | | | LDIA | |
| 322 | D-22213 | | L-Histidinamide, N2-[(2,3,4,9-tetrahydro-1H-pyrido[3,4- | Anticancer, other |
| | | | b]indo1-3-yl)carbonyl]-L-glutaminyl-L-tryptophyl-L-alanyl- | |
| | | | L-valylglycyl-N-[1-[[[1-(aminocarbony1)-3- | |
| | | | methylbutyl]amino]methyl]-3-methylbutyl]-, [1(R),6[S- | |
| | | | (R*,R*)]]-, monoacetate | |
| 323 | DAP inhibitors | | L-AP-L-Ala and L-Ala-L-Ala-DL-AP; | Antibacterial, other |
| 60 | DP-640 | insulin | L-Tyrosinamide, β-alanyl-L-arginylglycyl-L-phenylalanyl- | Insulin |
| | | | L-phenylalanyl-, diacetate (salt) | Antidiabetic |
| 61 | DP-107 | | L-Leucine, L-methionyl-L-threonyl-L-leucyl-L-threonyl-L- | Antiviral, anti-HIV |
| | | | valyl-L-glutaminyl-L-alanyl-L-arginyl-L-glutaminyl-L- | |
| | | | leucyl-L-leucyl-L-seryl-L-glutaminyl-L-isoleucyl-L-valyl-L- | |
| | | | glutaminyl-L-glutaminyl-L-glutaminyl-L-asparaginyl-L- | |
| | | | asparaginyl-L-leucyl-L-leucyl-L-arginyl-L-alanyl-L- | |
| | | | isoleucyl-L-.alpha.-glutamyl-L-alanyl-L-glutaminyl-L- | |
| | | | glutaminyl-L-histidyl-L-leucyl-L-leucyl-L-glutaminyl-L- | |
| | | | leucyl-L-threonyl-L-valyl-L-tryptophylglycyl-L-isoleucyl-L- | |
| | | | lysyl-L-glutaminyl- | |
| 62 | DU-728 | | Arg-Gly-Asp-Ser | Antithrombotic |
| 63 | Dynorphin A | | H-Tyr-Gly-Gly-Phe-Leu-Arg-Arg-Ile-Arg-Pro-Lys-Leu-Lys- | Analgesic, other |
| | | | Trp-Asp-Asn-Gln-OH | Neuroprotective |
| | | | | Dependence treatment |
| 64 | defensins | | gi\|181535\|gb\|AAA52304.1\| defensin precursor | Antibiotic, other |
| | | | MRTLAILAAILLVALQAQAEPLQARADEVAAAPEQIA | Antifungal |
| | | | ADIPEVVVSLAWDESLAPKHPGSRKNMDCYCRIPACI | Vulnerary |
| | | | AGERRYGTCIYQGRLWAFCC | |
| 324 | detirelix | | D-Alaninamide, N-acetyl-3-(2-naphthalenyl)-D-alanyl-4- | Releasing hormone |
| | | | chloro-D-phenylalanyl-D-tryptophyl-L-seryl-L-tyrosyl-N6- | Abortifacient |
| | | | [bis(ethylamino)methylene]-D-lysyl-L-leucyl-L-arginyl-L- | Male contraceptive |
| | | | prolyl- | |
| 65 | disagregin | | gi\|545738\|gb\|AAB30092.1\| disagregin = fibrinogen receptor | Antithrombotic |
| | | | antagonist [Ornithodoros moubata = tick, salivary gland, | Cardiovascular |
| | | | Peptide, 60 aa] | |
| | | | SDDKCQGRPMYGCREDDDSVFGWTYDSNHGQCWKG | |
| | | | SYCKHRRQPSNYFASQQECRNTCGA | |
| 66 and | E-2078 | | D-Leucinamide, N-methyl-L-tyrosylglycylglycyl-L- | Analgesic, other |
| 65 | | | phenylalanyl-L-leucyl-L-arginyl-N2-methyl-L-arginyl-N- | |
| | | | ethyl- | |
| | | | SDDKCQGRPMYGCREDDDSVFGWTYDSNHGQCWKG | |
| | | | SYCKHRRQPSNYFASQQECRNTCGA | |
| | ELS-1 | | Arg-Lys-Glu | Immunostimulant, other |
| 67 | ecallantide | | Glu-Ala-Met-His-Ser-Phe-Cys-Ala-Phe-Lys-Ala-Asp-Asp- | Angioedema, Anti- |
| | | | Gly-Pro-Cys-Arg-Ala-Ala-His-Pro-Arg-Trp-Phe-Phe-Asn- | inflammatory, |
| | | | Ile-Phe-Thr-Arg-Gln-Cys-Glu-Glu-Phe-Ile-Tyr-Gly-Gly- | Haemostatic, Antiarthritic, |
| | | | Cys-Glu-Gly-Asn-Gln-Asn-Arg-Phe-Glu-Ser-Leu-Glu-Glu- | other |
| | | | Cys-Lys-Lys-Met-Cys-Thr-Arg-Asp | |
| 325 | ES-1005 | | bis-(l-naphthyl)methylacetyl-His-Sta-Leu-E-Lys diHCl | Antihypertensive, renin |
| | | | | system |
| 326 | efegatran | | L-prolinamide, N-methyl-D-phenylalanyl-n-(4- | Antithrombotic |
| | | | ((aminoiminomethypamino)-1-formylbutyl), (S)- | Antianginal |
| 68 and | elafin | | gi\|999146\|gb\|AAB34627.1\| elafin [Homo sapiens] | Respiratory |
| 69 | derivatives | | MRASSFLIVVVFLIAGTLVLE | COPD treatment |
| | | | H-Ala-Gln-Glu-Pro-Val-Lys-Gly-Pro-Val-Ser-Thr-Lys-Pro- | Antiarthritic, other |
| | | | Gly-Ser-Cys-Pro-Ile-Ile-Leu-Ile-Arg-Cys-Ala-Met-Leu-Asn- | |
| | | | Pro-Pro-Asn-Arg-Cys-Leu-Lys-Asp-Thr-Asp-Cys-Pro-Gly- | |
| | | | Ile-Lys-Lys-Cys-Cys-Glu-Gly-Ser-Cys-Gly-Met-Ala-Cys- | |
| | | | Phe-Val-Pro-Gln-OH (Disufide bonds between Cys16- | |
| | | | Cys45, Cys23-Cys49, Cys32-Cys44, Cys38-Cys53) | |
| 70 and | elcatonin | calcitonin | Ser-Asn-Leu-Ser-Thr-Asn-Val-Leu-Gly-Lys-Leu-Ser-Gln- | Hormone |
| 52 | | | Glu-Leu-His-Lys-Leu-Gln-Thr-Tyr-Pro-Arg-Thr-Asn-Val- | Osteoporosis treatment |
| | | | Gly-Ala-Gly-Thr-Pro-NH₂ | Analgesic, other |
| | | | CGNLSTCMLGTYTQDFNKFHTFPQTAIGVGAP | |
| | | | (human) | |
| 71 | eledoisin | | 5-oxo-L-Pro-L-Pro-L-Ser-L-Lys-L-Asp-L-Ala-L-Phe-L-Ala- | Ophthalmological |
| | | | L-isoleucylglycyl-L-Leu-L-methionin-amide | |
| 3 | encapsulated | insulin | gi\|386828\|gb\|AAA59172.1\| insulin [Homo sapiens] | Formulation, optimized, |
| | insulin | | MALWMRLLPLLALLALWGPDPAAAFVNQHLCGSHLV | nanoparticles |
| | | | EALYLVCGERGFFYTPKTRREAEDLQVGQVELGGGPG | Insulin |
| | | | AGSLQPLALEGSLQKRGIVEQCCTSICSLYQLENYCN | Antidiabetic |
| 72 | endorphin, β- | | YGGFMTSEKSQTPLVTLFKNAIIKNAYKKGE | Analgesic, other |
| 72 | endorphin, | | YGGFMTSEKSQTPLVTLFKNAIIKNAYKKGE | Analgesic, other |
| | pancreatic | | | |
| 73 | endothelial cell | | gi\|89701\|gb\|AAA60043.1\| endothelial cell growth factor | Cardiovascular |
| | growth factor | | MAALMTPGTGAPPAPGDFSGEGSQGLPDPSPEPKQLP | |
| | | | ELIRMKRDGGRLSEADIRGFVAAVVNGSAQGAQIGA | |
| | | | MLMAIRLRGMDLEETSVLTQALAQSGQQLEWPEAWR | |
| | | | QQLVDKHSTGGVGDKVSLVLAPALAACGCKVPMISG | |
| | | | RGLGHTGGTLDKLESIPGFNVIQSPEQMQVLLDQAGC | |
| | | | CIVGQSEQLVPADGILYAARDVTATVDSLPLITASILSK | |
| | | | KLVEGLSALVVDVKFGGAAVFPNQEQARELAKTLVG | |
| | | | VGASLGLRVAAALTAMDKPLGRCVGHALEVEEALLC | |
| | | | MDGAGPPDLRDLVTTLGGALLWLSGHAGTQAQGAA | |
| | | | RVAAALDDGSALGRFERMLAAQGVDPGLARALCSGS | |
| | | | PAERRQLLPRAREQEELLAPADGTVELVRALPLALVL | |
| | | | HELGAGRSRAGEPLRLGVGAELLVDVGQRLRRGTPW | |
| | | | LRVHRDGPALSGPQSRALQEALVLSDRAPFAAPSPFA | |
| | | | ELVLPPQQ | |
| 74 | eptifibatide | | MAP-HAR-GLY-ASP-TRP-PRO-CYS-NH₂ | Antianginal |
| | | | | Cardiovascular |
| 327 | examorelin | GHRP | L-Lysinamide, L-histidyl-2-methyl-D-tryptophyl-L-alanyl- | Releasing hormone |
| | | | L-tryptophyl-D-phenylalanyl- | Vulnerary |
| | | | | Cardiovascular |
| 75 | FG-005 | | SMR1-QHNPR | Male sexual dysfunction |
| 328 | FR-113680 | | L-Phenylalaninamide, N-acetyl-L-threonyl-1-formyl-D- | Antiasthma |
| | | | tryptophyl-N-methyl-N-(phenylmethyl)- | |
| 76 | fibronectin- | | Gly-Arg-Gly-Asp-Ser | Anticancer, other |
| | related peptide | | | |
| 329 | G-4120 | | L-Cysteine, N-(mercaptoacetyl)-D-tyrosyl-L-arginylglycyl- | Antithrombotic |
| | | | L-alpha-aspartyl-, cyclic (1-5)-sulfide, S-oxide | |
| 330 | EP-51216 | | 2S)-6-amino-2-[[(2S)-2-[[(2R)-2-[[(2R)-2-(4- | GH Releasing hormone |
| | | | aminobutanoylamino)-3-(2-methyl-1H-indo1-3- | Vulnerary, endocrine |
| | | | yl)propanoyl]amino]-3-(2-methyl-1H-indo1-3- | |
| | | | yl)propanoyl]amino]-3-(2-methy1-1H-indo1-3- | |
| | | | yl)propanoyl]amino]hexanamide | |
| 8 | GLP-1 + | GLP-1 | HGEGTFTSDLSKQMEEEAVRLFIEWLKNGGPSSGAPP | Antidiabetic |
| | exendin-4 | | PS | |
| 77 | GM-1986 | | YYWIGIR | Anti-inflammatory |
| 78 | GnRH- | GnRH | gi\|133908612\|ref\|NP_001076580.1\| gonadotropin-releasing | Antiprolactin |
| | associated | | hormone 1 precursor [Homo sapiens] | Menstruation disorders |
| | peptide | | MKPIQKLLAGLILLTWCVEGCSSQHWSYGLRPGGKR | Fertility enhancer |
| | | | DAENLIDSFQEIVKEVGQLAETQRFECTTHQPRSPLRD | |
| | | | LKGALESLIEEETGQKKI | |
| 79 | GRF1-44 | | gi\|11034841\|ref\|NP_066567.1\| growth hormone releasing | Musculoskeletal |
| | | | hormone preproprotein [Homo sapiens] | |
| | | | MPLWVFFFVILTLSNSSHCSPPPPLTLRMRRYADAIFT | |
| | | | NSYRKV | |
| 80 | GRF | GHRF | gi\|337133\|gb\|AAA52609.1\| growth hormone releasing factor | Idiopathic growth hormone |
| | | | MPLWVFFFVILTLSNSSHCSPPPPLTLRMRRYADAIFT | deficiency; cachexia |
| | | | NSYRKVLGQLSARKLLQDIMSRQQGESNQERGARAR | |
| | | | LGRQVDSMWAEQKQMELESILVALLQKHRNSQG | |
| 331 | GYKI-14451 | | L-Prolinamide, N-[(1,1-dimethylethoxy)carbonyl]-D- | Antithrombotic |
| | | | phenylalanyl-N-[4-[(aminoiminomethyl)amino]-1- | |
| | | | formylbutyl]-, (S)- | |
| 81 | galanin | | gi\|1247490\|emb\|CAA01907.1\| galanin [Homo sapiens] | Releasing hormone |
| | | | MARGSALLLASLLLAAALSASAGLWSPAKEKRGWTL | |
| | | | NSAGYLLGPHAVGNHRSFSDKNGLTSKRELRPEDDM | |
| | | | KPGSFDRSIPENNIMRTIIEFLSFLHLKEAGALDRLLDL | |
| | | | PAAASSEDIERS | |
| 82 | gastrin | | (benzyloxycarbonyl)-L-Glu-L-Ala-L-Tyr-Gly-L-Tyr-L-Met- | Antiulcer |
| | antagonists | | L-aspartic acid amide | |
| 332 | glaspimod | | N2,N2′-[2,7-Bis(pyroglutamyl-glutamyl-aspartylamino)- | Immunomodulator, anti- |
| | | | octanediolyl]bis(lysine) | infective |
| | | | | Immunostimulant, other |
| | | | | Radio/chemoprotective |
| 83 | glicentin | | gi\|125987831\|sp\|P01275.3\|GLUC_HUMAN Glucagon | Insulin |
| | | | precursor [Contains: Glicentin; Glicentin-related polypeptide | Antiulcer |
| | | | (GRPP); Oxyntomodulin (OXY) (OXM); Glucagon; | Antidiabetic |
| | | | Glucagon-like peptide 1 (GLP-1); Glucagon-like peptide | |
| | | | 1(7-37) (GLP-1(7-37)); Glucagon-like peptide 1(7-36) | |
| | | | (GLP-1(7-36)); Glucagon-like peptide 2 (GLP-2)] | |
| | | | MKSIYFVAGLFVMLVQGSWQRSLQDTEEKSRSFSASQ | |
| | | | ADPLSDPDQMNEDKRHSQGTFTSDYSKYLDSRRAQD | |
| | | | FVQWLMNTKRNRNNIAKRHDEFERHAEGTFTSDVSS | |
| | | | YLEGQAAKEFIAWLVKGRGRRDFPEEVAIVEELGRRH | |
| | | | ADGSFSDEMNTILDNLAARDFINWLIQTKITDRK | |
| 84 | glucagon | | H₂N-His-Ser-Gln-Gly-Thr-Phe-Thr-Ser-Asp-Tyr-Ser-Lys- | hypoglycemia |
| | | | Tyr-Leu-Asp-Ser-Arg-Arg-Ala-Gln-Asp-Phe-Val-Gln-Trp- | Diagnostic |
| | | | Leu-Met-Asn-Thr-COOH | |
| 84 | glucagon | glucagon | His-Ser-Gln-Gly-Thr-Phe-Thr-Ser-Asp-Tyr-Ser-Lys-Tyr- | Hypoglycemia |
| | | | Leu-Asp-Ser-Arg-Arg-Ala-Gln-Asp-Phe-Val-Gln-Trp-Leu- | |
| | | | Met-Asn-Thr | |
| 85 | gonadorelin | gonadorelin | gi\|121522\|sp\|P01148.1\|GON1_HUMAN Progonadoliberin-1 | Female contraceptive; |
| | analogues | | precursor (Progonadoliberin I) [Contains: Gonadoliberin-1 | enometiosis, uterine |
| | | | (Gonadoliberin I) (Luteinizing hormone-releasing hormone | leiomyoma, precocious |
| | | | I) (LH-RH I) (Gonadotropin-releasing hormone I) (GnRH-I) | puberty, prostate and breast |
| | | | (Luliberin I) (Gonadorelin); GnRH-associated peptide 1 | cancer |
| | | | (GnRH-associated peptide I)] | |
| | | | MKPIQKLLAGLILLTWCVEGCSSQHWSYGLRPGGKR | |
| | | | DAENLIDSFQEIVKEVGQLAETQRFECTTHQPRSPLRD | |
| | | | LKGALESLIEEETGQKKI | |
| 333 | gonadorelin | | [Ac-DNAL1(2),4FDPhe2,D-Trp3,D-Arg6]-LHRH | Female contraceptive; |
| | antagonist | | | enometiosis, uterine |
| | | | | leiomyoma, precocious |
| | | | | puberty, prostate and breast |
| | | | | cancer |
| 86 | gonadorelin | gonadorelin | 5-oxo-L-His-L-Trp-L-Ser-L-Tyr-Gly-L-Leu-L-Arg-L-Pro- | Female contraceptive; |
| | | | glycinamide | enometiosis, uterine |
| | | | | leiomyoma, precocious |
| | | | | puberty, prostate and breast |
| | | | | cancer |
| 334 | goralatide | | L-Proline, 1-[N2-[N-(N-acetyl-L-seryl)-L-α-aspartyl]-L- | Haematological |
| | | | lysyl]- | Immunological |
| | | | | Radio/chemoprotective |
| 335 | H-142 | | L-Lysine, N2-[N-[N-[N-[4-methyl-2-[[N-[N-[1-(N-L-prolyl- | Antihypertensive, renin |
| | | | L-histidyl)-L-prolyl]-L-phenylalanyl]-L- | system |
| | | | histidyl]amino]pentyl]-L-valyl]-L-isoleucyl]-L-histidyl]-, | |
| | | | (S)- | |
| 336 | I5B2 | | L-Tyrosinamide, N-methyl-L-valyl-N-[2-(4-hydroxyphenyl)- | Antihypertensive, renin |
| | | | 1-phosphonoethyl]- | system |
| 87 | iseganan | | L-Argininamide, L-arginylglycylglycyl-L-leucyl-L- | Antibacterial, other |
| | hydrochloride | | cysteinyl-L-tyrosyl-L-cysteinyl-L-arginylglycyl-L-arginyl-L- | Antifungal |
| | | | phenylalanyl-L-cysteinyl-L-valyl-L-cysteinyl-L-valylglycyl-, | Antiviral, other |
| | | | cyclic (5-14),(7-12)-bis(disulfide) hydrochloride | |
| 88 | netamiftide | | L-Tryptophanamide, 4-fluoro-L-phenylalanyl-(4R)-4- | Antidepressant |
| | | | hydroxy-L-prolyl-L-arginylglycyl-, bis(trifluoroacetate) | Anxiolytic |
| | | | (salt) | |
| 337 | icrocaptide | | L-Arginine, glycyl-N2-ethyl-L-lysyl-L-prolyl- | Cardiovascular |
| | | | | Septic shock treatment |
| 338 | icatibant | | L-Arginine, D-arginyl-L-arginyl-L-prolyl-trans-4-hydroxy- | Cardiovascular |
| | | | L-prolylglycyl-3-(2-thienyl)-L-alanyl-L-seryl-D-1,2,3,4- | Hepatoprotective |
| | | | tetrahydro-3-isoquinolinecarbany-L-(2α,3aβ,7aβ)-octahydro- | Vulnerary |
| | | | 1H-indole-2-carbonyl- | |
| 339 | AG-1776 | | 3-[2(S)-Hydroxy-3(S)-(3-hydroxy-2-methylbenzamido)-4- | Antiviral, anti-HIV |
| | | | phenylbutanoyl]-5,5-dimethyl-N-(2- | |
| | | | methylbenzyl)thiazolidine-4(R)-carboxamide | |
| 340 | pralmorelin | | L-Lysinamide, D-alanyl-3-(2-naphthalenyl)-D-alanyl-L- | Diagnostic |
| | | | alanyl-L-tryptophyl-D-phenylalanyl- | Releasing hormone |
| 89 | katacalcin | calcitonin | gi\|115478\|sp\|P01258.1\|CALC_HUMAN Calcitonin | Osteoporosis treatment |
| | | | precursor [Contains: Calcitonin; Katacalcin (Calcitonin | Hormone |
| | | | carboxyl-terminal peptide) (CCP) (PDN-21)] | Recombinant, other |
| | | | MGFQKFSPFLALSILVLLQAGSLHAAPFRSALESSPAD | |
| | | | PATLSEDEARLLLAALVQDYVQMKASELEQEQEREGS | |
| | | | SLDSPRSKRCGNLSTCMLGTYTQDFNKFHTFPQTAIG | |
| | | | VGAPGKKRDMSSDLERDHRPHVSMPQAN | |
| 341 | ketomethylureas | | N-[N-[3-benzoylamino-4-phenyl-2-oxobutyl]-N- | Antihypertensive, renin |
| | | | methylaminocarbonyl]proline | system |
| 90 | L-346670 | | N-L-arginyl-8-L-methionine-21a-L-phenylalanine-21b-L- | Antihypertensive, diuretic |
| | | | arginine-21c-L-tyrosine- | |
| 91 | L-364210 | | N-isovaleryl-L-histidyl-L-prolyl-L-phenylalanyl-L-histidyl- | Antihypertensive, renin |
| | | | (3S,4S)-4-amino-5-cyclohexyl-3-hydroxypentanoic acid)-L- | system |
| | | | leucyl-L-phenylalanylamide | |
| 342 | L-659837 | | L-Phenylalanine, N-[2-(3-amino-2-oxo-1-pyrrolidinyl)-4- | Analgesic, other |
| | | | methyl-1-oxopentyl]-L-methionyl-L-glutaminyl-L- | |
| | | | tryptophyl-, cyclic (4-1)-peptide, [S-(R*,S*)]- | |
| 343 | L-693549 | | 5(S)-(tert-butoxycarbonylamino)-4(S)-hydroxy-N-[2(R)- | Antiviral, anti-HIV |
| | | | hydroxyindan-l(S)-yl]-2(R)-[4-(3-hydroxypropyl)benzyl]-6- | |
| | | | phenylhexamide | |
| 344 | L-709049 | | L-Alaninamide, N-acetyl-L-tyrosyl-L-valyl-N-(2-carboxy-1- | Anti-inflammatory |
| | | | formylethyl)-, (S)- | |
| 92 | LDV- | | 4-((N′-2-methylphenyl)ureido)phenylalanyl-leucyl-alpha- | Anticancer, other |
| | containing | | aspartyl-valyl-prolyl-alanyl-alanyl-lysine | |
| | peptides | | | |
| | Lys-Phe | | L-Phenylalanine, N-L-lysyl- | Haematological |
| | | | | Antisickling |
| 93 | lagatide | | D-Alaninamide, L-prolyl-L-valyl-L-threonyl-L-lysyl-L- | Antidiarrhoeal |
| | | | prolyl-L-glutaminyl- | |
| 94 | laminin A | | seryl-isoleucyl-lysyl-valyl-alanyl-valinamide | Anticancer, other |
| | peptide | | | Neurological |
| 95 | laminin | | tyrosyl-isoleucyl-glycyl-serylarginine | Anticancer, other |
| 345 | lanreotide | somatostatin | L-Threoninamide, 3-(2-naphthalenyl)-D-alanyl-L-cysteinyl- | Acromegaly |
| | | | L-tyrosyl-D-tryptophyl-L-lysyl-L-valyl-L-cysteinyl-, cyclic | Anticancer, hormonal |
| | | | (2-7)-disulfide; L-Threoninamide, 3-(1-naphthalenyl)-D- | Cardiovascular |
| | | | alanyl-L-cysteinyl-L-tyrosyl-D-tryptophyl-L-lysyl-L-valyl- | Antidiarrhoeal |
| | | | L-cysteinyl-, cyclic (2-7)-disulfide | |
| 346 | leuprolide | | Luteinizing hormone-releasing factor (pig), 6-D-leucine-9- | Formulation, implant |
| | acetate | | (N-ethyl-L-prolinamide)-10-deglycinamide- | Anticancer, hormonal |
| | | | | Menstruation disorders |
| 347 | MCI-826 | | Butanoic acid, 2,2-diethyl-4-[[3-[2-[4-(1-methylethyl)-2- | Antiasthma |
| | | | thiazolyl]ethenyl]phenyl]amino]-4-oxo-, (E)- | |
| 96 | omiganan | | L-lysinamide, L-isoleucyl-L-leucyl-L-arginyl-L-tryptophyl- | Peptide antibiotic |
| | pentahydrochloride | | L-prolyl-L-tryptophyl-L-tryptophyl-L-prolyl-L-tryptophyl- | |
| | | | L-arginyl-L-arginyl, pentahydrochloride | |
| 97-100 | MBP | | gi\|68509940\|ref\|NP_001020272.1\| Golli-mbp isoform 1 | Multiple sclerosis |
| | | | [Homo sapiens] | treatment |
| | | | MGNHAGKRELNAEKASTNSETNRGESEKKRNLGELS | Immunosuppressant |
| | | | RTTSEDNEVFGEADANQNNGTSSQDTAVTDSKRTAD | |
| | | | PKNAWQDAHPADPGSRPHLIRLFSRDAPGREDNTFKD | |
| | | | RPSESDELQTIQEDSAATSESLDVMASQKRPSQRHGSK | |
| | | | YLATASTMDHARHGFLPRHRDTGILDSIGRFFGGDRG | |
| | | | APKRGSGKDSHHPARTAHYGSLPQKSHGRTQDENPV | |
| | | | VHFFKNIVTPRTPPPSQGKGRGLSLSRFSWGAEGQRPG | |
| | | | FGYGGRASDYKSAHKGFKGVDAQGTLSKIFKLGGRD | |
| | | | SRSGSPMARR | |
| | | | gi\|68509938\|ref\|NP_001020271.1\| Golli-mbp isoform 2 | |
| | | | [Homo sapiens] | |
| | | | MGNHAGKRELNAEKASTNSETNRGESEKKRNLGELS | |
| | | | RTTSEDNEVFGEADANQNNGTSSQDTAVTDSKRTAD | |
| | | | PKNAWQDAHPADPGSRPHLIRLFSRDAPGREDNTFKD | |
| | | | RPSESDELQTIQEDSAATSESLDVMASQKRPSQRHGSK | |
| | | | YLATASTMDHARHGFLPRHRDTGILDSIGRFFGGDRG | |
| | | | APKRGSGKVSSEE | |
| | | | gi\|68509930\|ref\|NP_001020252.1\| myelin basic protein | |
| | | | isoform 1 [Homo sapiens] | |
| | | | MASQKRPSQRHGSKYLATASTMDHARHGFLPRHRDT | |
| | | | GILDSIGRFFGGDRGAPKRGSGKVPWLKPGRSPLPSHA | |
| | | | RSQPGLCNMYKDSHHPARTAHYGSLPQKSHGRTQDE | |
| | | | NPVVHFFKNIVTPRTPPPSQGKGRGLSLSRFSWGAEGQ | |
| | | | RPGFGYGGRASDYKSAHKGFKGVDAQGTLSKIFKLG | |
| | | | GRDSRSGSPMARR | |
| | | | gi\|4505123\|ref\|NP_002376.1\| myelin basic protein isoform 2 | |
| | | | [Homo sapiens] | |
| | | | MASQKRPSQRHGSKYLATASTM | |
| 348 | MDL-104238 | | N-[4-(4-morpholinylcarbonyl)benzoyl]-L-valyl-N′- | Anti-inflammatory |
| | | | [3,3,4,4,4-pentafluoro-1-(1-methylethyl)-2-oxobutyl]-L-2- | |
| | | | azetamide | |
| 349 | MDL-28050 | | D-Glutamic acid, N-[N-[N-[N-[N-[1-[N-[1-[N-[N-(3- | Antithrombotic |
| | | | carboxy-1-oxopropyl)-L-tyrosyl]-L-alpha-glutamyl]-L- | Anticoagulant |
| | | | prolyl]-L-isoleucyl]-prolyl]-alpha-glutamyl]-L-alpha- | |
| | | | glutamyl]-L-alanyl]-3-cyclohexyl-L-alanyl]- | |
| 101 | MMP inhibitors | | FN 439; FN-439; H2N—C6H4—CO-Gly-Pro-Leu-Ala-NHOH; | Antiarthritic, Anticancer, |
| | | | MMP-inhibitor I; p-NH2-Bz-Gly-Pro-D-Leu-D-Ala-NHOH | Anti-inflammatory |
| 350 | MR-988 | | N-pivaloyl-leucyl-gamma-aminobutyric acid | Antiepileptic |
| 351 | mertiatide | | Glycine, N-[N-[N-(mercaptoacetyl)glycyl]glycyl]- | Diagnostic |
| 352 | metkephamide | | L-Methioninamide, L-tyrosyl-D-alanylglycyl-L- | Analgesic, other |
| | | | phenylalanyl-N2-methyl-, monoacetate (salt) | |
| 353 | murabutide | | D-Glutamine, N2-[N-(N-acetylmuramoyl)-L-alanyl]-, butyl | Immunomodulator, anti- |
| | | | ester | infective |
| | | | | Anticancer, immunological |
| | | | | Immunostimulant, other |
| 354 | muramyl | | D-alpha-Glutamine, N2-[N-(N-acetylmuramoyl)-L-alanyl]- | Immunomodulator, anti- |
| | dipeptide | | | infective |
| | derivatives | | | Anticancer, immunological |
| | | | | Immunostimulant, other |
| 355 | NPY24-36 | | N-acetyl[Leu-28Leu-31]NPY24-36 | Antihypotensive |
| 102 | NAGA | | Asn-Ala-Gly-Ala | Analgesic, other |
| 356 | tiplimotide | | L-Proline, D-alanyl-L-lysyl-L-prolyl-L-valyl-L-valyl-L- | Multiple sclerosis |
| | | | histidyl-L-leucyl-L-phenylalanyl-L-alanyl-L-asparaginyl-L- | treatment |
| | | | isoleucyl-L-valyl-L-threonyl-L-prolyl-L-arginyl-L-threonyl- | |
| 103 | opebecan | | gi\|157276599\|ref\|NP_001716.2\| bactericidal/permeability- | Recombinant, other |
| | | | increasing protein precursor [Homo sapiens] | Antibacterial, other |
| | | | MRENMARGPCNAPRWASLMVLVAIGTAVTAAVNPG | GI inflammatory/bowel |
| | | | VVVRISQKGLDYASQQGTAALQKELKRIKIPDYSDSF | disorders |
| | | | KIKHLGKGHYSFYSMDIREFQLPSSQISMVPNVGLKFS | Vulnerary |
| | | | ISNANIKISGKWKAQKRFLKMSGNFDLSIEGMSISADL | Anti-inflammatory |
| | | | KLGSNPTSGKPTITCSSCSSHINSVHVHISKSKVGWLIQ | Symptomatic antidiabetic |
| | | | LFHKKIESALRNKMNSQVCEKVTNSVSSELQPYFQTL | Ophthalmological |
| | | | PVMTKIDSVAGINYGLVAPPATTAETLDVQMKGEFYS | |
| | | | ENHHNPPPFAPPVMEFPAAHDRMVYLGLSDYFFNTA | |
| | | | GLVYQEAGVLKMTLRDDMIPKESKFRLTTKFFGTFLP | |
| | | | EVAKKFPNMKIQIHVSASTPPHLSVQPTGLTFYPAVDV | |
| | | | QAFAVLPNSSLASLFLIGMHTTGSMEVSAESNRLVGE | |
| | | | LKLDRLLLELKHSNIGPFPVELLQDIMNYIVPILVLPRV | |
| | | | NEKLQKGFPLPTPARVQLYNVVLQPHQNFLLFGADV | |
| | | | VYK | |
| 104 and | liraglutide | GLP-1 | Glycine, L-histidyl-L-alanyl-L-alpha-glutamylglycyl-L- | Antidiabetic |
| 105 | | | threonyl-L-phenylalanyl-L-threonyl-L-seryl-L-alpha- | Anorectic/Antiobesity |
| | | | aspartyl-L-valyl-L-seryl-L-seryl-L-tyrosyl-L-leucyl-L-alpha- | |
| | | | glutamylglycyl-L-glutaminyl-L-alanyl-L-alanyl-N6-[N-(1- | |
| | | | oxohexadecyl)-L-gamma-glutamyl]-L-lysyl-L-alpha- | |
| | | | glutamyl-L-phenylalanyl-L-isoleucyl-L-alanyl-L-tryptophyl- | |
| | | | L-leucyl-L-valyl-L-arginylglycyl-L-arginyl- | |
| | | | SFKIKHLGKGHYSFYSMDIREFQLPSSQISMVPNVGLK | |
| | | | FSISNANIKISGKWKAQKRFLKMSGNFDLSIE | |
| 106 | Nona CCK | | GMSISADLKLGSNPTSGKPTITCSSCSSHINSVHVHISK | Diagnostic |
| | | | SKVGWLIQLFHKKIESALRNKMNSQVCEKVT | Neuroleptic |
| | | | | Anorectic/Antiobesity |
| | | | | Antidepressant |
| 107 and | NP-06 | | Cysteinyl-leucyl-glycyl-valyl-glycyl-seryl-cysteinyl- | Antiviral, anti-HIV |
| 108 | | | asparaginyl-aspartyl-phenylalanyl-alanyl-glycyl-cysteinyl- | |
| | | | glycyl-tyrosyl-alanyl-isoleucyl-valyl-cysteinyl-phenylalanyl- | |
| | | | tryptophan S-3.1-S-3.13: S-3.7-S-3.19-bis(disulfide)N-2.1-C- | |
| | | | 4.9-lactam | |
| | | | NSVSSELQPYFQTLPVMTKIDSVAGINYGLVAPPATTA | |
| | | | ETLDVQMKGEFYSENHHNPPPFAPPVMEFPAA | |
| 109 | NPC-18545 | | Bradykinin, N2-D-arginyl-3-(trans-4-hydroxy-L-proline)-7- | Anti-inflammatory |
| | | | (D-1,2,3,4-tetrahydro-3-isoquinolinecarboxylic acid)-8-[L- | |
| | | | (2alpha,3aβ,7a.beta.)-octahydro-1H-indole-2-carboxylic | |
| | | | acid]- | |
| | | | HDRMVYLGLSDYFFNTAGLVYQEAGVLKMTLRDDM | |
| | | | IPKESKFRLTTKFFGTFLPEVAKKFPNMKIQIHVS | |
| 110 | Nva-FMDP | | Nva-N3-4-methoxyfumaroyl-L-2,3-diaminopropanoic acid | Antifungal |
| | | | ASTPPHLSVQPTGLTFYPAVDVQAFAVLPNSSLASLFL | |
| | | | IGMHTTGSMEVSAESNRLVGELKLDRLLLELK | |
| 111 | nacartocin | | 6-Carbaoxytocin, 1-(3-mercaptopropanoic acid)-2-(4-ethyl- | Hormone |
| | | | L-phenylalanine)- | Labour inducer |
| | | | HSNIGPFPVELLQDIMNYIVPILVLPRVNEKLQKGFPLP | Antihypertensive, diuretic |
| | | | TPARVQLYNVVLQPHQNFLLFGADVVYK | |
| 112 | natural peptide | | U.S. Pat. No. 5,288,708 | Antiulcer |
| | | | Partial N terminal sequence: H₂N-Gly-Glu-Pro-Pro-Pro-Gly- | Hepatoprotective |
| | | | Lys-Pro-Ala-Asp-Asp-Ala-Gly-Leu-Val-- . . . --COOH | Vulnerary |
| | | | | Anti-inflammatory |
| | | | | Antiparkinsonian |
| | | | | Urological |
| 39 | nesiritide citrate | BNP | SPKMVQGSGCFGRKMDRISSSSGLGCKVLRRH | Cardiostimulant |
| | | | | Vasodilator, coronary |
| 113-141 | neurotrophic | | U.S. Pat. No. 5,545,719: | Cognition enhancer |
| | factors | | AspLeuGlnValPheVal; GlyGluLysLysAsp; | Neuroprotective |
| | | | AlaThrHisGluSer; | |
| | | | CysLeuProValSerGly; LeuProValSerGlySer; | |
| | | | ProCysHisAlaProPro; GlyGlyHisAspLeuGluSerGly; | |
| | | | AspAspLeuGlnValPhe 15 ProLeuThrSerGly 15 | |
| | | | LeuIleHisPheGluGluGlyVal 15 (2) INFORMATION FOR | |
| | | | SEQ ID NO: 11: (i) SEQUENCE CHARACTERISTICS: (A) | |
| | | | LENGTH: 7 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO:11: GlyGluPheSerTyrAspSer | |
| | | | 15 (2) INFORMATION FOR SEQ ID NO: 12: (i) | |
| | | | SEQUENCE CHARACTERISTICS: (A) LENGTH: 7 amino | |
| | | | acids (B) TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 12: | |
| | | | HisAlaProProLeuThrSer 15 (2) INFORMATION FOR SEQ | |
| | | | ID NO:13: (i) SEQUENCE CHARACTERISTICS: (A) | |
| | | | LENGTH: 7 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 13: AspLeuGluSerGlyGluPhe | |
| | | | 15 (2) INFORMATION FOR SEQ ID NO: 14: (i) | |
| | | | SEQUENCE CHARACTERISTICS: (A) LENGTH: 8 amino | |
| | | | acids (B) TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 14: | |
| | | | GlyGluPheSerValCysAspSer 15 (2) INFORMATION FOR | |
| | | | SEQ ID NO: 15: (i) SEQUENCE CHARACTERISTICS: (A) | |
| | | | LENGTH: 10 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 15: | |
| | | | LysLysGlyGluPheSerValAlaAspSer 1510 (2) | |
| | | | INFORMATION FOR SEQ ID NO: 16: (i) SEQUENCE | |
| | | | CHARACTERISTICS: (A) LENGTH: 9 amino acids (B) | |
| | | | TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 16: | |
| | | | LysLysGlyGluPheTyrCysSerArg 15 (2) INFORMATION | |
| | | | FOR SEQ ID NO: 17: (i) SEQUENCE | |
| | | | CHARACTERISTICS: (A) LENGTH: 13 amino acids (B) | |
| | | | TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 17: | |
| | | | GlyLeuArgValArgValTrpAsnGlyLysPheProLys 1510 (2) | |
| | | | INFORMATION FOR SEQ ID NO: 18: (i) SEQUENCE | |
| | | | CHARACTERISTICS: (A) LENGTH: 16 amino acids (B) | |
| | | | TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO:18: | |
| | | | GlyValAlaPheGluGluAlaProAspAspHisSerPhePheLeuPhe | |
| | | | 151015 (2) INFORMATION FOR SEQ ID NO: 19: (i) | |
| | | | SEQUENCE CHARACTERISTICS: (A) LENGTH: 7 amino | |
| | | | acids (B) TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 19: | |
| | | | GlyGlyHisAspLeuSerGly 15 (2) INFORMATION FOR | |
| | | | SEQ ID NO: 20: (i) SEQUENCE CHARACTERISTICS: (A) | |
| | | | LENGTH: 8 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 20: | |
| | | | GlyGlyHisAspLeuGluSerGly 15 (2) INFORMATION FOR | |
| | | | SEQ ID NO: 21: (i) SEQUENCE CHARACTERISTICS: (A) | |
| | | | LENGTH: 14 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 21: | |
| | | | GlyGlyHisAspLeuGluSerGlyGluPheSerTyrAspSer 1510 (2) | |
| | | | INFORMATION FOR SEQ ID NO: 22: (i) SEQUENCE | |
| | | | CHARACTERISTICS: (A) LENGTH: 14 amino acids (B) | |
| | | | TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 22: | |
| | | | GlyGlySerAspLeuSerGlyGluPheSerValCysAspSer 1510 (2) | |
| | | | INFORMATION FOR SEQ ID NO: 23: (i) SEQUENCE | |
| | | | CHARACTERISTICS: (A) LENGTH: 15 amino acids (B) | |
| | | | TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 23: | |
| | | | GlyGlySerAspLeuSerGlyGlyGluPheSerValCysAspSer | |
| | | | 151015 (2) INFORMATION FOR SEQ ID NO: 24: (i) | |
| | | | SEQUENCE CHARACTERISTICS: (A) LENGTH: 15 | |
| | | | amino acids (B) TYPE: amino acid (D) TOPOLOGY: linear | |
| | | | (ii) MOLECULE TYPE: peptide (v) FRAGMENT TYPE: | |
| | | | internal (xi) SEQUENCE DESCRIPTION: SEQ ID NO: 24: | |
| | | | GlyGlySerAspLeuSerGlyGlyGluPheSerValAlaAspSer | |
| | | | 151015 (2) INFORMATION FOR SEQ ID NO: 25: (i) | |
| | | | SEQUENCE CHARACTERISTICS: (A) LENGTH: 14 | |
| | | | amino acids (B) TYPE: amino acid (D) TOPOLOGY: linear | |
| | | | (ii) MOLECULE TYPE: peptide (v) FRAGMENT TYPE: | |
| | | | internal (xi) SEQUENCE DESCRIPTION: SEQ ID NO: 25: | |
| | | | GlyGlySerAspLeuSerGlyGluPheSerValAlaAspSer 1510 (2) | |
| | | | INFORMATION FOR SEQ ID NO: 26: (i) SEQUENCE | |
| | | | CHARACTERISTICS: (A) LENGTH: 6 amino acids (B) | |
| | | | TYPE: amino acid (D) TOPOLOGY: linear (ii) | |
| | | | MOLECULE TYPE: peptide (v) FRAGMENT TYPE: | |
| | | | internal (xi) SEQUENCE DESCRIPTION: SEQ ID NO: 26: | |
| | | | GluThrLeuGlnPheArg 15 (2) INFORMATION FOR SEQ | |
| | | | ID NO: 27: (i) SEQUENCE CHARACTERISTICS: (A) | |
| | | | LENGTH: 8 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 27: | |
| | | | LysLysGluThrLeuGlnPheArg 15 (2) INFORMATION FOR | |
| | | | SEQ ID NO: 28: (i) SEQUENCE CHARACTERISTICS: (A) | |
| | | | LENGTH: 8 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 28: | |
| | | | GluThrLeuGlnPheArgLysLys 15 (2) INFORMATION FOR | |
| | | | SEQ ID NO: 29: (i) SEQUENCE CHARACTERISTICS: (A) | |
| | | | LENGTH: 9 amino acids (B) TYPE: amino acid (D) | |
| | | | TOPOLOGY: linear (ii) MOLECULE TYPE: peptide (v) | |
| | | | FRAGMENT TYPE: internal (xi) SEQUENCE | |
| | | | DESCRIPTION: SEQ ID NO: 29: | |
| | | | LysAlaSerThrThrThrAsnTyrThr 15 | |
| 357 | nifalatide | | L-Prolinamide, L-tyrosyl-4-(methylsulfinyl)-D-2- | Antidiarrhoeal |
| | | | aminobutanoylglycyl-4-nitro-L-phenylalanyl- | Analgesic, other |
| 358 | Org-2766 | | L-Phenylalanine, 4-(methylsulfonyl)-L-2-aminobutanoyl-L- | ACTH |
| | | | alpha-glutamyl-L-histidyl-L-phenylalanyl-D-lysyl- | Symptomatic antidiabetic |
| | | | | Radio/chemoprotective |
| | | | | Neurological |
| 359 | Org-30035 | | L-Phenylalanine, glycylglycyl-L-phenylalanyl-4- | Neuroleptic |
| | | | (methylsulfonyl)-L-2-aminobutanoyl-D-lysyl- | Anxiolytic |
| 360 | octreotide | somatostatin | L-Cysteinamide, D-phenylalanyl-L-cysteinyl-L- | Acromegaly |
| | | | phenylalanyl-D-tryptophyl-L-lysyl-L-threonyl-N-[2- | Antidiarrhoeal |
| | | | hydroxy-1-(hydroxymethyl)propyl]-, cyclic (2-7)-disulfide, | Anticancer, hormonal |
| | | | [R-(R*,R*)]-; L-Cysteinamide, D-phenylalanyl-L-cysteinyl- | |
| | | | L-phenylalanyl-D-tryptophyl-L-lysyl-L-threonyl-N-(2- | |
| | | | hydroxy-1-(hydroxymethyl)propyl)-, cyclic (2-7)-disulfide, | |
| | | | (R-(R*,R*))- | |
| 142 | osteogenic | | Glycine, L-alanyl-L-leucyl-L-lysyl-L-arginyl-L- | Osteoporosis treatment |
| | growth peptide | | glutaminylglycyl-L-arginyl-L-threonyl-L-leucyl-L- | |
| | | | tyrosylglycyl-L-phenylalanylglycyl- | |
| 143 | P-113 | | Angiotensin II, 1-(N-methylglycine)-5-L-valine-8-L-alanine- | Stomatological |
| | | | [CAS]; (Sar(1),Ala(8))ANGII; (Sar1,Val5,Ala8)Angiotensin | Antibacterial, other |
| | | | II; 1 Sar 8 Ala Angiotensin II; 1 Sarcosine 8 Alanine | Antifungal |
| | | | Angiotensin II; 1-Sar-8-Ala Angiotensin II; 1-Sar-8-Ala- | |
| | | | angiotensin II; 1-Sarcosine-8-Alanine Angiotensin II; | |
| | | | Acetate, Hydrated Saralasin; Angiotensin II, 1-Sar-8-Ala; | |
| | | | Angiotensin II, 1-Sarcosine-8-Alanine; Anhydrous Saralasin | |
| | | | Acetate; Hydrated Saralasin Acetate; P-113; P-113 Acetate; | |
| | | | Sar Arg Val Tyr Val His Pro Ala; Sar-Arg-Val-Tyr-Val-His- | |
| | | | Pro-Ala; Saralasin Acetate; Saralasin Acetate, Anhydrous; | |
| | | | Saralasin Acetate, Hydrated; angiotensin II, Sar(1)-Ala(8)-; | |
| | | | angiotensin II, sarcosyl(1)-alanine(8)- | |
| 361 | PACAP 27 | | Pituitary adenylate cyclase-activating peptide-27 | Antiviral, anti-HIV |
| 362 | PAPP | | N-(dibenzyloxyphosphophionyl)-L-alanyl-L-prolyl-L- | Antihypertensive, other |
| | | | proline | |
| 363 | PD-83176 | | CBZ-his-tyr(OBn)-ser(OBn)-trp-D-ala-NH₂ | Anticancer, other |
| 364 | PD-122264 | | N-[(1,1-dimethylethoxy)carbonyl]-alpha-methyltryptophyl- | Anorectic/Antiobesity |
| | | | L-phenylalaninamide | Analgesic, other |
| 365 | PD-132002 | | DL-Serinamide, N-(4-morpholinylsulfonyl)-L-phenylalanyl- | Antihypertensive, renin |
| | | | N-[1-(cyclohexylmethyl)-2,3-dihydroxy-5-methylhexyl]-O- | system |
| | | | methyl-3-oxo-, [1S-(1R*,2S*,3R*)]- | |
| 144 | Penetratin | | U.S. Pat. Nos. 5,888,762 and 6,080,762; | Formulation technology |
| | | | PCT Pub. Nos. WO/2000/29427 and WO/2000/01417: | |
| | | | NH2-Arg Lys Arg Gly Arg Gln Thr Tyr Thr Arg Tyr Gln | |
| | | | Thr Leu Glu Leu Glu Lys Glu Phe His Phe Asn Arg Tyr Leu | |
| | | | Thr Arg Arg Arg Arg Ile Glu Ile Ala His Ala Leu Cys Leu | |
| | | | Thr Glu Arg Gln Ile Lys Ile Trp Phe Gln Asn Arg Arg Met | |
| | | | Lys Trp Lys Lys Glu Asn-COOH. | |
| 366 | PL-030 | | Glycinamide, L-tyrosyl-L-prolyl-N-methyl-L-phenylalanyl- | Analgesic, other |
| | | | D-prolyl- | |
| 367 | POL-443 | | Z-prolyl-leucyl-tryptophan | Antihypertensive, renin |
| | | | | system |
| 368 | POL-509 | | L-Tryptophan, N-[N-(5-oxo-L-prolyl)-L-leucyl]-, methyl | Immunostimulant, other |
| | | | ester- | |
| 369 | PPA | | D-phenylalanine-L-proline-L-arginylchloromethane | Anticoagulant |
| | | | | Diagnostic |
| | | | | Antithrombotic |
| 145 | PR-39 | | L-Prolinamide, L-arginyl-L-arginyl-L-arginyl-L-prolyl-L- | Antibacterial, other |
| | | | arginyl-L-prolyl-L-prolyl-L-tyrosyl-L-leucyl-L-prolyl-L- | |
| | | | arginyl-L-prolyl-L-arginyl-L-prolyl-L-prolyl-L-prolyl-L- | |
| | | | phenylalanyl-L-phenylalanyl-L-prolyl-L-prolyl-L-arginyl-L- | |
| | | | leucyl-L-prolyl-L-prolyl-L-arginyl-L-isoleucyl-L-prolyl-L- | |
| | | | prolylglycyl-L-phenylalanyl-L-prolyl-L-prolyl-L-arginyl-L- | |
| | | | phenylalanyl-L-prolyl-L-prolyl-L-arginyl-L-phenylalanyl- | |
| 146 | tigapotide | | L-Threonine, L-alpha-glutamyl-L-tryptophyl-L-glutaminyl- | Anticancer, other |
| | triflutate | | L-threonyl-L-alpha-aspartyl-L-asparaginyl-S- | |
| | | | [(acetylamino)methyl]-L-cysteinyl-L-alpha-glutamyl-L- | |
| | | | threonyl-S-Racetylamino)methyl]-cysteinyl-L-threonyl-S- | |
| | | | [(acetylamino)methyl]-L-cysteinyl-L-tyrosyl-L-alpha- | |
| | | | glutamyl-, mono(trifluoroacetate) | |
| 370 | PT-14 | | L-Lysinamide, N-acetyl-L-norleucyl-L-alpha-aspartyl-L- | Male sexual dysfunction |
| | | | histidyl-D-phenylalanyl-L-arginyl-L-tryptophyl-, cyclic (2- | Female sexual dysfunction |
| | | | 7)-peptide | |
| 147 | PT-5 | somatostatin | gi\|21619156\|gb\|AAH32625.1\| Somatostatin [Homo sapiens] | Anticancer, other |
| | | | MLSCRLQCALAALSIVLALGCVTGAPSDPRLRQFLQK | |
| | | | SLAAAAGKQELAKYFLAELLSEPNQTENDALEPEDLS | |
| | | | QAAEQDEMRLELQRSANSNPAMAPRERKAGCKNFF | |
| | | | WKTFTSC | |
| 148 | semparatide | PTHrP | gi\|131542\|sp\|P12272.1\|PTHR_HUMAN Parathyroid | Hormone |
| | | | hormone-related protein precursor (PTH-rP) (PTHrP) | Osteoporosis treatment |
| | | | [Contains: PTHrP[1-36]; PTHrP[38-94]; Osteostatin | |
| | | | (PTHrP[107-139])] | |
| | | | MQRRLVQQWSVAVFLLSYAVPSCGRSVEGLSRRLKR | |
| | | | AVSEHQLLHDKGKSIQDLRRRFFLHHLIAEIHTAEIRA | |
| | | | TSEVSPNSKPSPNTKNHPVFGSDDEGRYLTQETNKVE | |
| | | | TYKEQPLKTPGKKKKGKPGKRKEQEKKKRRTRSAWL | |
| | | | DSGVTGSGLEGDHLSDTSTTSLELDSRRH | |
| 149 | parathyroid | PTH | SVSEIQLMHNLGKHLNSMERVEWLRKKLQDVHNF | Osteoporosis treatment |
| | hormone | | | |
| | fragments | | | |
| 150 | enfuvirtide | | L-Phenylalaninamide, N-acetyl-L-tyrosyl-L-threonyl-L- | Antiviral, anti-HIV |
| | | | seryl-L-leucyl-L-isoleucyl-L-histidyl-L-seryl-L-leucyl-L- | |
| | | | isoleucyl-L-alpha-glutamyl-L-alpha-glutamyl-L-seryl-L- | |
| | | | glutaminyl-L-asparaginyl-L-glutaminyl-L-glutaminyl-L- | |
| | | | alpha-glutamyl-L-lysyl-L-asparaginyl-L-alpha-glutamyl-L- | |
| | | | glutaminyl-L-alpha-glutamyl-L-leucyl-L-leucyl-L-alpha- | |
| | | | glutamyl-L-leucyl-L-alpha-aspartyl-L-lysyl-L-tryptophyl-L- | |
| | | | alanyl-L-seryl-L-leucyl-L-tryptophyl-L-asparaginyl-L- | |
| | | | tryptophyl- | |
| 151 | pentapeptide | | Ala-Arg-Pro-Ala-Lys | Vasodilator, coronary |
| | 6A | | | |
| 371 | pentigetide | | L-Arginine, N2-[1-[N-(N-L-alpha-aspartyl-L-seryl)-L-alpha- | Antiallergic, non-asthma |
| | | | aspartyl]-L-prolyl]- | Ophthalmological |
| | | | | Antiasthma |
| 372 | peptide | | N1,N3-bis(2,3-dihydroxypropyl)-2,4,6-triiodo-5-(2- | Ophthalmological |
| | analogues | | methoxyacetamido)-N1-methylisophthalamide | Antiarthritic, other |
| | | | | Antiulcer |
| | | | | Antihypertensive, other |
| | | | | Multiple sclerosis |
| | | | | treatment |
| | | | | COPD treatment |
| 373 | peptide G | | [Arg(6),D-Trp(7,9),MePhe(8)]substance P | Anticancer, other |
| 374 | peptide T | | D-Ala1-peptide T | Antiviral, anti-HIV |
| | analogue | | | |
| 375 | peptide T | | L-Threonine, N-[N-[N2-[N-[N-[N-(N-L-alanyl-L-seryl)-L- | Analgesic, other |
| | | | threonyl]-L-threonyl]-L-threonyl]-L-asparaginyl]-L-tyrosyl]- | Antiviral, other |
| | | | | Antiarthritic, other |
| | | | | GI inflammatory/bowel |
| | | | | disorders |
| | | | | Anti-inflammatory |
| 152 | pramlintide | | 1,2-Dithia-5,8,11,14,17-pentaazacycloeicosane, cyclic | Antidiabetic |
| | | | peptide derivative | Anorectic/Antiobesity |
| | | | U.S. Pat. No. 5,998,367 | |
| | | | gi\|10066209\|gb\|AAE39671.1\| Sequence 1 from U.S. Pat. No. | |
| | | | 5,998,367 | |
| | | | KCNTATCATQRLANFLVHSSNNFGAILSSTNVGSNTY; | |
| 376 | pranlukast | | Benzamide, N-[4-oxo-2-(1H-tetrazol-5-yl)-4H-1- | Antiasthma |
| | | | benzopyran-8-yl]-4-(4-phenylbutoxy)-; 8-(4(4- | Antiallergic, non-asthma |
| | | | phenylbutoxy)benzoyl)amino-2-(tetrazol-5′-yl)-4-oxo-4H-1- | |
| | | | benzopyran | |
| 3 | proinsulin | proinsulin | gi\|59036749\|gb\|AAW83741.1\| proinsulin [Homo sapiens] | Antidiabetic |
| | | | MALWMRLLPLLALLALWGPDPAAAFVNQHLCGSHL | |
| | | | VEALYLVCGERGFFYTPKTRREAEDLQVGQVELGGG | |
| | | | PGAGSLQPLALEGSLQKRGIVEQCCTSICSLYQLENYC | |
| | | | N | |
| 377 | protirelin | TRH | L-Prolinamide, 5-oxo-L-prolyl-L-histidyl-; 2-Nle-3-Prot- | Releasing hormone |
| | | | protirelin; TRH, Nle(2)-Prot(3)-; pyroglutamyl-norleucyl- | Diagnostic |
| | | | proline thioamide | |
| 378 | protirelin | TRH | prolinamide, 5-oxo-L-prolyl-L-histidyl- | Releasing hormone |
| | | | | Cognition enhancer |
| 153 | Ro-25-1553 | | L-Threoninamide, N-acetyl-L-histidyl-L-seryl-L-alpha- | Antiasthma |
| | | | aspartyl-L-alanyl-L-valyl-L-phenylalanyl-L-threonyl-L- | Anti-inflammatory |
| | | | alpha-glutamyl-L-asparaginyl-L-tyrosyl-L-threonyl-L-lysyl- | |
| | | | L-leucyl-L-arginyl-L-lysyl-L-glutaminyl-L-norleucyl-L- | |
| | | | alanyl-L-alanyl-L-lysyl-L-lysyl-L-tyrosyl-L-leucyl-L- | |
| | | | asparaginyl-L-alpha-aspartyl-L-leucyl-L-lysyl-L- | |
| | | | lysylglycylglycyl-, (25-21)-lactam | |
| 379 | RWJ-51438 | | N-methylphenylalanyl-N-(4-((aminoiminomethyl)amino)-1- | Antithrombotic |
| | | | ((6-carboxy-2-benzothiazolyl)carbonyl)butyl)prolinamide | |
| 380 | TRH | TRH | L-Prolinamide, 5-oxo-L-prolyl-L-histidyl-3,3-dimethyl-; | Diagnostic |
| | | | pyroGlu-His-Pro-NH₂ (or 5-oxo-L-prolyl-L-histidyl-L- | Thyroid hormone |
| | | | prolinamide) | Releasing hormone |
| 154 | renin inhibitors | | Boc-Leu-Lys-Arg-Met-Pro-OMe | Antihypertensive, |
| 381 | romurtide | | L-Lysine, N2-[N2-[N-(N-acetylmuramoyl)-L-alanyl]-D- | Radio/chemoprotective |
| | | | alpha-glutaminyl]N6-(1-oxooctadecyl)-; L-Lysine, N2- | Immunostimulant, other |
| | | | (N2-(N-(N-acetylmuramoyl)-L-alanyl)-D-alpha-glutaminyl)- | |
| | | | N6-(1-oxooctadecyl)- | |
| 382 | S-17162 | | L-Tryptophan, N-[(2,3- | Urological |
| | | | dihydroxypropoxy)hydroxyphosphinyl]-L-leucyl-, disodium | |
| | | | salt | |
| 383 | S-2441 | | L-Argininamide, D-prolyl-L-phenylalanyl-N-heptyl- | Antimigraine |
| | | | | Antigout |
| | | | | Septic shock treatment |
| 384 | SDZ-CO-611 | somatostatin | L-Cysteinamide, N-(1-deoxy-4-0-.alpha.-D-glucopyranosyl- | Somatostatin |
| | | | D-fructopyranos-1-yl)-D-phenylalanyl-L-cysteinyl-L- | |
| | | | phenylalanyl-D-tryptophyl-L-lysyl-L-threonyl-N-[2- | |
| | | | hydroxy-1-(hydroxymethyl)propyl]-, cyclic (2.fwdarw.7)- | |
| | | | disulfide, [R-(R*,R*)]- | |
| 385 | SK & F-101926 | | L-Argininamide, O-ethyl-N-[(1-mercaptocyclohexyl)acetyl]- | Antihypertensive, diuretic |
| | | | D-tyrosyl-L-phenylalanyl-L-valyl-L-asparaginyl-L- | |
| | | | cysteinyl-L-prolyl-, cyclic (1-5)-disulfide | |
| 386 | SK & F-110679 | | His-D-Trp-Ala-Trp-D-Phe-LysNH₂ | Releasing hormone |
| | | | | Vulnerary |
| 387 | edotreotide | | [N-[2-[4,7-Bis[(carboxy-kappaO)methyl]-10- | Anticancer, hormonal |
| | | | (carboxymethyl)-1,4,7,10-tetraazacyclododec-1-yl- | |
| | | | kappaN1, kappaN4, kappaN10]acetyl]-D-phenylalanyl-L- | |
| | | | cysteinyl-L-tyrosyl-D-tryptophyl-L-lysyl-L-threonyl-L- | |
| | | | cysteinyl-L-threoninol cyclic (2-7)-disulfidato(3-)]yttrium | |
| 155 | SP-1 | | pGlu-Glu-Asp-Cys-Lys | Anticancer, other |
| 156 | SPAAT | | L-Lysine, L-methionyl-L-phenylalanyl-L-leucyl-L-alpha- | COPD treatment |
| | | | glutamyl-L-alanyl-L-isoleucyl-L-prolyl-L-methionyl-L- | |
| | | | seryl-L-isoleucyl-L-prolyl-L-prolyl-L-alpha-glutamyl-L- | |
| | | | valyl-L-lysyl-L-phenylalanyl-L-asparaginyl-L-lysyl-L- | |
| | | | prolyl-L-phenylalanyl-L-valyl-L-phenylalanyl-L-leucyl-L- | |
| | | | methionyl-L-isoleucyl-L-alpha-glutamyl-L-glutaminyl-L- | |
| | | | asparaginyl-L-threonyl-L-lysyl-L-seryl-L-prolyl-L-leucyl-L- | |
| | | | phenylalanyl-L-methionylglycyl-L-lysyl-L-valyl-L-valyl-L- | |
| | | | asparaginyl-L-prolyl-L-threonyl-L-glutaminyl- | |
| 388 | SR-41476 | | Z-Tyr-Val-Sta-Ala-Sta-OMe | Antiviral, anti-HIV |
| 389 | SR-42128 | | 1-[N-(3-methyl-l-oxobutyl)-L-phenylalanine]-2-L- | Antihypertensive, renin |
| | | | norleucine- | system |
| 157 | SR-42654 | | isoval-phe-norleu-sta-ala-sta-lys | Antihypertensive, renin |
| | | | | system |
| 147 | SRIF-A | somatostatin | gi\|21619156\|gb\|AAH32625.1\| Somatostatin [Homo sapiens] | Somatostatin |
| | | | MLSCRLQCALAALSIVLALGCVTGAPSDPRLRQFLQK | Haemostatic |
| | | | SLAAAAGKQELAKYFLAELLSEPNQTENDALEPEDLS | Alimentary/Metabolic, |
| | | | QAAEQDEMRLELQRSANSNPAMAPRERKAGCKNFF | other |
| | | | WKTFTSC | |
| | | | 8-D-tryptophan-14-D-cysteinesomatostatin (sheep) | |
| 158 | calcitonin | calcitonin | CSNLSTCVLGKLSQELHKLQTYPRTNTGSGTP | Osteoporosis treatment |
| 390 | salmon | calcitonin | 11,18-Arg-14-Lys-salmon calcitonin; 11,18-arginyl-14- | Osteoporosis treatment |
| | calcitonin | | lysine-salmon calcitonin; Arg-Lys-Arg-CT; calcitonin, | |
| | | | salmon, arginyl(11,18)-lysine(14)- | |
| 159 | sermorelin | | Tyr-Ala-Asp-Ala-Ile-Phe-Thr-Asn-Ser-Tyr-Arg-Lys-Val- | Idiopathic growth hormone |
| | | | Leu-Gly-Gln-Leu-Ser-Ala-Arg-Lys-Leu-Leu-Gln-Asp-Ile- | deficiency |
| | | | Met-Ser-Arg-NH₂ | Imaging agent |
| 391 | saralasin acetate | | 1-Sar-8-Ala-angiotensin; Angiotensin II, 1-(N- | Antihypertensive, renin |
| | | | methylglycine)-5-L-valine-8-L-alanine- | system |
| 160 | secretin | | His-Ser-Asp-Gly-Thr-Phe-OMe; histidyl-seryl-aspartyl- | Haemostatic; pancreatic |
| | | | glycyl-threonyl-phenylalanine-O-methyl- | dysfunction (diagnostic), |
| | | | | asthma, COPD, others |
| 159 | sermorelin | | Tyr-Ala-Asp-Ala-Ile-Phe-Thr-Asn-Ser-Tyr-Arg-Lys-Val- | Releasing hormone |
| | acetate | | Leu-Gly-Gln-Leu-Ser-Ala-Arg-Lys-Leu-Leu-Gln-Asp-Ile- | Diagnostic |
| | | | Met-Ser-Arg-NH₂ | |
| 159 | sermorelin | | Tyr-Ala-Asp-Ala-Ile-Phe-Thr-Asn-Ser-Tyr-Arg-Lys-Val- | |
| | | | Leu-Gly-Gln-Leu-Ser-Ala-Arg-Lys-Leu-Leu-Gln-Asp-Ile- | |
| | | | Met-Ser-Arg-NH₂ | |
| 161 | sinapultide | | L-Lysine, L-lysyl-L-leucyl-L-leucyl-L-leucyl-L-leucyl-L- | Lung Surfactant |
| | | | lysyl-L-leucyl-L-leucyl-L-leucyl-L-leucyl-L-lysyl-L-leucyl- | |
| | | | L-leucyl-L-leucyl-L-leucyl-L-lysyl-L-leucyl-L-leucyl-L- | |
| | | | leucyl-L-leucyl- | |
| 162 | sleep inducing | | Trp-Ala-Gly-Gly-Asp-Ala-Ser-Gly-Glu | Hypnotic/Sedative |
| | peptide | | | Dependence treatment |
| 163 | somatoliberin | | gi\|11034841\|ref\|NP_066567.1\| growth hormone releasing | Growth hormone |
| | | | hormone preproprotein [Homo sapiens] | Releasing hormone |
| | | | MPLWVFFFVILTLSNSSHCSPPPPLTLRMRRYADAIFT | |
| | | | NSYRKVLGQLSARKLLQDIMSRQQGESNQERGARAR | |
| | | | LGRQVDSMWAEQKQMELESILVALLQKHSRNSQG | |
| 164 | PTR-3173 | somatostatin | Cyclic[(R)-βMeNphe-Phe-DTrp-Lys-Thr-Phe], | Acoegaly |
| | | | MPLWVFFFVILTLSNSSHCSPPPPLTLRMRRYADAIFT | Symptomatic antidiabetic |
| | | | NSYRKVLGQLSARKLLQDIMSRQQGESNQERG | Ophthalmological |
| | | | | Urological |
| | | | | Anticancer, hormonal |
| 165 | somatostatin | somatostatin | des-(Ala1,G1y2)-(D-Trp8,D-Asu(3,14))-somatostatin, | Acromegaly |
| | analogue | | ARARLGRQVDSMWAEQKQMELESILVALLQKHSRNS | Antidiabetic |
| | | | QG | Diagnostic |
| 392 | somatostatin | somatostatin | cyclo-(N-Me-Ala-Tyr-D-Trp-Lys-Val-Phe)-somatostatin | Acromegaly |
| | analogues | | | Antidiabetic |
| 393 | somatostatin | somatostatin | 3,14-Dicarbasomatostatin, 1-de-L-alanine-2-deglycine-3- | Acromegaly |
| | | | butanoic acid-11-L-tyrosine- | |
| 394 | somatostatin | somatostatin | 3,14-Dicarbasomatostatin, 1-de-L-alanine-2-deglycine-3- | Acromegaly |
| | | | butanoic acid-11-L-tyrosine- | |
| 395 | syndyphalin | | Glycinamide, L-tyrosyl-4-(methylsulfinyl)-D-2- | Analgesic, other |
| | | | aminobutanoyl-N-methyl-N-(2-phenylethyl)- | |
| 166 | synthetic | | gi\|109948285\|ref\|NP_001035971.1\| poly(A) binding protein, | Antiulcer |
| | peptide BPC | | cytoplasmic 1-like 2B [Homo sapiens] | Hepatoprotective |
| | | | MASLYVGDLHPEVTEAMLYEKFSPAGPILSIRICRDKI | Vulnerary |
| | | | TRRSLGYAYVNYQQPVDAKRALETLNFDVIKG | Anti-inflammatory |
| | | | RPVRIMWSQRDPSLRKSGVGNVFIKNLGKTIDNKALY | Antiparkinsonian |
| | | | NIFSAFGNILSCKVACDEKGPKGYGFVHFQKQE | Musculoskeletal |
| | | | SAERAIDVMNGMFLNYRKIFVGRFKSHKEREAERGA | |
| | | | WARQSTSADVKDFEEDTDEEATLR | |
| 167 | T22 | | L-Argininamide, L-arginyl-L-arginyl-L-tryptophyl-L- | Antiviral, anti-HIV |
| | | | cysteinyl-L-tyrosyl-L-arginyl-L-lysyl-L-cysteinyl-L-tyrosyl- | |
| | | | L-lysylglycyl-L-tyrosyl-L-cysteinyl-L-tyrosyl-L-arginyl-L- | |
| | | | lysyl-L-cysteinyl-, cyclic (4-17),(8-13)-bis(disulfide) | |
| 396 | Tc-99m | | Technetium-99Tc, (cyclo(L-homocysteinyl-N-methyl-L- | Imaging agent |
| | depreotide | | phenylalanyl-L-tyrosyl-D-tryptophyl-L-lysyl-L-valyl) (1-1′)- | |
| | | | thioether with 3-((mercaptoacetyl)amino)-L-alanyl-L-lysyl- | |
| | | | L-cysteinyl-L-lysinamidato(3-))oxo-, (SP-5-24)- | |
| 397 | Tc-99m-P280 | | 13,13′-[Oxybis[methylene(2,5-dioxo-1,3- | Imaging agent |
| | | | pyrrolidinediyl)]]bis[N-(mercaptoacetyl)-D-tyrosyl-S-(3- | Antithrombotic |
| | | | aminopropyl)-L-cysteinylglycyl-L-alpha-aspartyl-L- | |
| | | | cysteinylglycylglycyl-S-[(acetylamino)mehtyl]-L- | |
| | | | cysteinylglycyl-S-[(acetylamino)methyl-L- | |
| | | | cysteinylglycylglycyl-L-cysteinamide], cyclic (1 --> 5), | |
| | | | (1′ --> 5′), -bis(sulfide) | |
| 398 | TEI-1345 | | (7E)-8-(2-naphthyl)-5,6-trans-5,6-methano-7-octenyl 3-(3,4- | Anti-inflammatory |
| | | | dimethoxyphenyl)-2-propenoate | |
| 168 | THF | | Leu-Glu-Asp-Gly-Pro-Lys-Phe-Leu; leucyl-glutamyl- | Immunomodulator, anti- |
| | | | aspartyl-glycyl-proly-lysyl-phenylalanyl-leucine | infective, |
| | | | | Immunostimulant, anti- |
| | | | | AIDS |
| 169 | Theradigm- | | Dipalmitoyl-Lys-Ser-Ser-Gln-Tyr-Ile-Lys-Ala-Asn-Ser-Lys- | Immunomodulator, anti- |
| | HBV | | Phe-Ile-Gly-Ile-Thr-Glu-Ala-Ala-Ala-Phe-Leu-Pro-Ser-Asp- | infective |
| | | | Phe-Phe-Pro-Ser-Val-OH | Immunostimulant |
| 80 | tesamorelin | GHRF | gi\|337133\|gb\|AAA52609.1\| growth hormone releasing factor | Musculoskeletal, COPD, |
| | acetate | | MPLWVFFFVILTLSNSSHCSPPPPLTLRMRRYADAIFT | Hypnotic/Sedative, |
| | | | NSYRKVLGQLSARKLLQDIMSRQQGESNQERGARAR | Immunostimulant, |
| | | | LGRQVDSMWAEQKQMELESILVALLQKHRNSQG | Antidiabetic, Anabolic, |
| | | | (3E)-Hex-3-enoylsomatoliberin (human) acetate (salt) | Symptomatic antidiabetic, |
| | | | | Vulnerary |
| 170 | TP-9201 | | L-Cysteinamide, N-acetyl-L-cysteinyl-L-asparaginyl-L- | Neuroprotective, |
| | | | prolyl-L-arginylglycyl-L-alpha-aspartyl-O-methyl-L-tyrosyl- | Antithrombotic, |
| | | | L-arginyl-, cyclic (1-9)-disulfide | Antianginal, |
| | | | | Cardiovascular |
| 399 | TRH analogues | TRH | pyroGlu-His-Pro-NH₂ (or 5-oxo-L-prolyl-L-histidyl-L- | Cognition enhancer |
| | | | prolinamide) | |
| 400 | TT-235 | | [β,β-(3-Thiapentamethylene)-β-sulfanylpropionic acid, D- | Labour inhibitor |
| | | | Trp2,Pen6,Arg8]-oxytocin acetate | |
| 401 | tabilautide | | L-Lysinamide, 6-carboxy-N6-[N-[N-(1-oxododecyl)-L- | Immunomodulator, anti- |
| | | | alanyl]-D-gamma-glutamyl]-, (S)- | infective |
| | | | | Radio/chemoprotective |
| | | | | Immunostimulant, other |
| 171 and | terlipressin | | N-[N-(N-glycylglycyl)glycyl]-8-L-lysine-; Gly-Gly-Gly-8- | Haemostatic; GI bleeding |
| 172 | | | Lys-vasopressin; N-(alpha)-glycyl-glycyl-glycyl-8-lysine | |
| | | | vasopressin; Gly-Gly-Gly-c[Cys-Tyr-Phe-Gln-Asn-Cys]- | |
| | | | Pro-Lys-Gly-NH₂; N-(N-(N-glycylglycyl)glycyl)-8-L- | |
| | | | lysinevasopressin | |
| 171 and | terlipressin | | N-[N-(N-glycylglycyl)glycyl]-8-L-lysine-; Gly-Gly-Gly-8- | Haemostatic; GI bleeding |
| 172 | | | Lys-vasopressin; N-(alpha)-glycyl-glycyl-glycyl-8-lysine | |
| | | | vasopressin; Gly-Gly-Gly-c[Cys-Tyr-Phe-Gln-Asn-Cys]- | |
| | | | Pro-Lys-Gly-NH₂ | |
| 402 | teverelix | | D-Alaninamide, N-acetyl-3-(2-naphthalenyl)-D-alanyl-4- | Anticancer, hormonal |
| | | | chloro-D-phenylalanyl-3-(3-pyridinyl)-D-alanyl-L-seryl-L- | Prostate disorders |
| | | | tyrosyl-N6-(aminocarbonyl)-D-lysyl-L-leucyl-N6-(1- | Menstruation disorders |
| | | | methylethyl)-L-lysyl-L-prolyl- | Fertility enhancer |
| | | | | Male contraceptive |
| 403 | thymopentin | | L-Tyrosine, N-[N-[N-(N2-L-arginyl-L-lysyl)-L-alpha- | Immunostimulant, other |
| | | | aspartyl]-L-valyl]-; L-Tyrosine, N-(N-(N-(N2-L-arginyl-L- | Immunomodulator, anti- |
| | | | lysyl)-L-alpha-aspartyl)-L-valyl)- | infective |
| 404 | triletide | | L-Histidine, N-[N-(N-acetyl-L-phenylalanyl)-L- | Antiulcer |
| | | | phenylalanyl]-, methylester | |
| 405 | tuftsin | | L-Arginine, N2-[1-(N2-L-threonyl-L-lysyl)-L-prolyl)- | Anticancer, immunological |
| | | | | Immunostimulant, other |
| 173 | Uroguanylin | | Guanylin (rat reduced), 1-L-glutamine-2-L-glutamic acid-3- | Alimentary/Metabolic, |
| | | | L-aspartic acid-6-L-leucine-8-L-isoleucine-9-L-asparagine- | other |
| | | | 10-L-valine- | Antidiarrhoeal |
| | | | | Diagnostic |
| 174 | VIC | | gi\|6681267\|ref\|NP_031929.1\| endothelin 3 [Mus musculus] | Gastroprokinetic |
| | | | MEPGLWLLLGLTVTSAAGLVPCPQSGDSGRASVSQGP | |
| | | | PEAGSERGCEETVAGPGERIVSPTVALPAQPESAGQER | |
| | | | APGRSGKQEDKGLPAHHRPRRCTCFTYKDKECVYYC | |
| | | | HLDIIWINTPEQTVPYGLSNYRESLRGKRSLGPVPESSQ | |
| | | | PSPWTRLRCTCMGADDKACAHFCARTRDVTSYSGRA | |
| | | | ERPAAEEMRETGGPRQRLMSRTDKAHRP | |
| 175 | VIP derivative | | gi\|5803023\|ref\|NP_3006807.1\| lectin, mannose-binding 2 | Antiasthma |
| | | | [Homo sapiens] | Vasodilator, peripheral |
| | | | MAAEGWIWRWGWGRRCLGRPGLLGPGPGPTTPLFLL | |
| | | | LLLGSVTADITDGNSEHLKREHSLIKPYQGVGSSSMPL | |
| | | | WDFQGSTMLTSQYVRLTPDERSKEGSIWNHQPCFLKD | |
| | | | WEMHVHFKVHGTGKKNLHGDGIALWYTRDRLVPGP | |
| | | | VFGSKDNFHGLAIFLDTYPNDETTERVFPYISVMVNN | |
| | | | GSLSYDHSKDGRWTELAGCTADFRNRDHDTFLAVRY | |
| | | | SRGRLTVMTDLEDKNEWKNCIDITGVRLPTGYYFGAS | |
| | | | AGTGDLSDNHDIISMKLFQLMVEHTPDEESIDWTKIEP | |
| | | | SVNFLKSPKDNVDDPTGNFRSGPLTGWRVFLLLLCAL | |
| | | | LGIVVCAVVGAVVFQKRQERNKRFY | |
| 147 | vapreotide, | somatostatin | gi\|21619156\|gb\|AAH32625.1\| Somatostatin [Homo sapiens] | Formulation, modified- |
| | immediate- | | MLSCRLQCALAALSIVLALGCVTGAPSDPRLRQFLQK | release, immediate |
| | release | | SLAAAAGKQELAKYFLAELLSEPNQTENDALEPEDLS | Somatostatin |
| | | | QAAEQDEMRLELQRSANSNPAMAPRERKAGCKNFF | Haemostatic |
| | | | WKTFTSC | Anticancer, hormonal |
| | | | L-Tryptophanamide, D-phenylalanyl-L-cysteinyl-L-tyrosyl- | Antidiarrhoeal |
| | | | D-tryptophyl-L-lysyl-L-valyl-L-cysteinyl-, cyclic (2-7)- | GI inflammatory/bowel |
| | | | disulfide- | disorders |
| 406 | Pharmaprojects | | L-Proline, 1-[N-[N-[1-[4-(4-hydroxyphenyl)-1-oxobutyl]-L- | Vasodilator, renal |
| | No. 1269 | | prolyl]-.alpha.-methyl-DL-phenylalanyl]glycyl]- | |
| 407 | Pharmaprojects | | N(α)-((3S)-1-oxo-1,2,3,4-tetrahydroisoquinoline-3- | Neuroleptic |
| | No. 1583 | | carbonyl)-L-histidyl-L-prolinamide | Antiparkinsonian |
| 408 | Pharmaprojects | | D-2-phenylglycyl-D-2-phenylglycine | Anticancer, immunological |
| | No. 1626 | | | Immunostimulant, other |
| 409 | Pharmaprojects | | N-acyl-D-glutamyl-1-meso-diaminopimelyl-1-lysine | Immunomodulator, anti- |
| | No. 1779 | | tripeptide derivatives | infective |
| | | | | Immunostimulant, other |
| 176 | Pharmaprojects | | Thr-Asp-Ser-Phe-Val-Gly-Leu-Methionylamide | Antihypertensive, other |
| | No. 1876 | | | |
| 410 | Pharmaprojects | | L-leucyl-D-methionyl-glucyl-N-(2-adamantyl)-L- | Antihypertensive, renin |
| | No. 1913 | | phenylalanylamide | system |
| 177 | Pharmaprojects | | Lys-Pro-Gly-Glu-Pro-Gly-Pro-Lys | Anticoagulant |
| | No. 1939 | | | |
| 178-182, | Pharmaprojects | | U.S. Pat. No. 4,461,724 and European Patent No. | Antiulcer |
| 178, | No. 2063 | | EP0078228: GSHK; ASHK; A_{D}SHK; LSHK; TSHK; | Antithrombotic |
| 183-185 | | | YSHK; GSHKCH₃COOH•H₂O; SAR-SHK; PSHK; | |
| and 178 | | | (PYR)ESHK; WSHK; GSHK•2TosOH | |
| 411 | Pharmaprojects | | N-methyl-D-Phe-Pro-Arg-H | Antithrombotic |
| | No. 2363 | | | |
| 186 | Pharmaprojects | | N-3-(4-hydroxyphenyl)propionyl-Pro-Hyp-Gly-Ala-Gly | Antiarrhythmic |
| | No. 2388 | | | |
| 412 | Pharmaprojects | | Glp-lys-NH₂-L-mandelate | Anticancer, immunological |
| | No. 2425 | | | Immunostimulant, other |
| 413 | Pharmaprojects | | D-1-Tiq-Pro-Arg-H-sulfate | Antithrombotic |
| | No. 3341 | | | |
| 414 | Pharmaprojects | | (2R,4S,5S,1′S)-5-(t-butoxycarbonyl)amino-4-hydroxy-N-[1′- | Antiviral, anti-HIV |
| | No. 3415 | | isopropy1-1′-(4-isopropylcarbonylimidazol-2-yl)]methyl-6- | |
| | | | phenyl-2-phenylmethyl-hexanamide | |
| 415 | Pharmaprojects | | Piv-1-Ser-Leu-GABA, and Piv-Ser-Leu-GABA | Neurological |
| | No. 4004 | | | |
| 416 | Pharmaprojects | | (1R,4aR,8aR)-1,2,3,4,5,6,7,8-perhydroisoquinolin-1- | Antithrombotic |
| | No. 4323 | | carbonyl-(L)-prolinyl-(L)-arinine aldehyde | Anticoagulant |
| 187, and | Pharmaprojects | | H-Trp-Ala-Ser-Gly-L-Asn-OH & H-Trp-D-Ala-Ser-Gly- | Hypnotic/Sedative |
| 417 | No. 491 | | Asp(OH)₂ | Antidepressant |
| | | | | Neuroprotective |
| 188 | Pharmaprojects | | H₂N-Asp-Ala-Asp-Pro-Arg-Gln-Tyr-Ala-COOH | Anti-inflammatory |
| | No. 4975 | | | |
| 418 | Pharmaprojects | | 2-Amino-N-{1-(R)-benzyloxymethyl-2-[4-(morpholine-4- | Osteoporosis treatment |
| | No. 5200 | | carbonyl)-4-phenyl-piperidin-1-yl]-2-oxo-ethyl}- | |
| | | | isobutyramide | |
| 419 | Pharmaprojects | | 4-chloro-phenylcarbamoyl-thienylalanyl-leucyl- | Anti-inflammatory |
| | No. 5356 | | phenylalanine | Anti-infective, other |
| 420 | DMP-444 | | synthetic cyclic pentapeptide (cyclo(D-Val-NMeArg-Gly- | Imaging agent |
| | | | Asp-Mamb))with a tethered hydrazinonicotinyl (HYNIC) | |
| | | | chelator for radiolabelling with 99mTc | |
| 189 | RIP-3 | | MSCVKLWPSGAPAPLVSIEELENQELVGKGGFGTVFR | Anticancer, other |
| | | | AQHRKWGYDVAVKIVNSKAISREVKAMASLDNEFVL | |
| | | | RLEGVIEKVNWDQDPKPALVTKFMENGSLSGLLQSQ | |
| | | | CPRPWPLLCRLLKEVVLGMFYLHDQNPVLLHRDLKPS | |
| | | | NVLLDPELHVKLADFGLSTFQGGSQSGTGSGEPGGTL | |
| | | | GYLAPELFVNVNRKASTASDVYSFGILMWAVLAGRE | |
| | | | VELPTEPSLVYEAVCNRQNRPSLAELPQAGPETPGLEG | |
| | | | LKELMQLCWSSEPKDRPSFQECLPKTDEVFQMVENN | |
| | | | MNAAVSTVKDFLSQLRSSNRRFSIPESGQGGTEMDGF | |
| | | | RRTIENQHSRNDVMVSEWLNKLNLEEPPSSVPKKCPS | |
| | | | LTKRSRAQEEQVPQAWTAGTSSDSMAQPPQTPETSTF | |
| | | | RNQMPSPTSTGTPSPGPRGNQGAERQGMNWSCRTPEP | |
| | | | NPVTGRPLVNIYNCSGVQVGDNNYLTMQQTTALPTW | |
| | | | GLAPSGKGRGLQHPPPVGSQEGPKDPEAWSRPQGWY | |
| | | | NHSGK | |
| 421 | Pharmaprojects | | N-(N-acetyl-1-isoleucyl-L-tyrosyl)-(−)-1-amino-2-(4- | Antihypertensive, other |
| | No. 955 | | hydroxyphenyl)ethylphosphonic acid | |
| 422 | leuprolide | | 6-D-leucine-9-(N-ethyl-L-prolinamide)-10-deglycinamide- | Formulation, modified- |
| | | | | release, Anticancer |
| 190 | edratide | | L-glycyl-L-tyrosyl-L-tyrosyl-L-tryptophyl-L-seryl-L- | Immunosuppressant |
| | | | tryptophyl-L-isoleucyl-L-arginyl-L-glutaminyl-Lprolyl-L- | |
| | | | prolyl-L-glycyl-L-lysyl-L-glycyl-L-glutamyl-L-glutamyl-L- | |
| | | | tryptophyl-L-isoleucyl-L-glycine | |
| 423 | Prosaptide | | H-Thr-D-Ala-Leu-Ile-Asp-Asn-Asn-Ala-Thr-Glu-Glu-Ile- | Symptomatic antidiabetic |
| | TX14(A) | | Leu-Tyr-OH | Neurological |
| | | | | Analgesic, other |
| 8 | GLP-1 | GLP-1 | HGEGTFTSDLSKQMEEEAVRLFIEWLKNGGPSSGAPP | Antidiabetic |
| | | | PS | |
| 160 | secretin | | His-Ser-Asp-Gly-Thr-Phe-OMe; histidyl-seryl-aspartyl- | Hormone, Diagnostic, GI |
| | | | glycyl-threonyl-phenylalanine-O-methyl- | inflammatory/bowel |
| | | | | disorders, Neurological, |
| | | | | Neuroleptic |
| 147 | BIM-23190 | somatostatin | gi\|21619156\|gb\|AAH32625.1\| Somatostatin [Homo sapiens] | Acromegaly |
| | | | MLSCRLQCALAALSIVLALGCVTGAPSDPRLRQFLQK | Antidiabetic |
| | | | SLAAAAGKQELAKYFLAELLSEPNQTENDALEPEDLS | |
| | | | QAAEQDEMRLELQRSANSNPAMAPRERKAGCKNFF | |
| | | | WKTFTSC | |
| | | | L-Threoninamide, N-[[4-(2-hydroxyethyl)-1- | |
| | | | piperazinyl]acetyl]-D-phenylalanyl-L-cysteinyl-L-tyrosyl-D- | |
| | | | tryptophyl-L-lysyl-(2S)-2-aminobutanoyl-L-cysteinyl-, | |
| | | | cyclic (2-7)-disulfide | |
| 424 | leuprorelin | | 6-D-leucine-9-(N-ethyl-L-prolinamide)-10-deglycinamide- | Formulation, Anticancer |
| 191 | β-amyloid | beta-amyloid | gi\|8176533\|gb\|AAB26264.2\| beta-amyloid peptide precursor; | Cognition enhancer |
| | peptides | peptide | beta APP [Homo sapiens] | |
| | | | GSGLTNIKTEEISEVKMDAEFRHDSGYEVHHQKLVFF | |
| | | | AEDVGSNKGAIIGLMVGGVVIATVIIITLVMLK | |
| | | | KQYTSNHHGVVE | |
| 425 | oglufanide | | L-tryptophan, L-alpha-glutamyl-, disodium salt | Immunomodulator, anti- |
| | disodium | | | infective |
| | | | | Anticancer, immunological |
| 192 | HAV peptide | | leucyl-arginyl-alanyl-histidyl-alanyl-valyl-aspartyl-valyl- | Neurological |
| | matrix | | asparaginyl-glycinamide | |
| 149 | PTH 1-34 | PTH | SVSEIQLMHNLGKHLNSMERVEWLRKKLQDVHNF | Hormone |
| | leuprorelin | | 6-D-leucine-9-(N-ethyl-L-prolinamide)-10-deglycinamide- | Anticancer |
| 193 | TRP-2 | | H-Leu-Leu-Pro-Gly-Gly-Arg-Pro-Tyr-Arg-OH | Anticancer, immunological |
| 426 | golotimod | | (2R)-2-amino-5-[[(1S)-1-carboxy-2-(1H-indol-3- | Immunostimulant, other |
| | | | yl)ethyl]amino]-5-oxopentanoic acid | Immunomodulator, anti- |
| | | | | infective |
| | | | | Anticancer, immunological |
| | | | | Stomatological |
| 194 | angiotensin-II | Angiotensin | gi\|28710\|emb\|CAA77513.1\| angiotensin II [Homo sapiens] | Vulnerary |
| | | II | MILNSSTEDGIKRIQDDCPKAGRHNYIFVMIPTLYSIIF | Symptomatic antidiabetic |
| | | | VVGIFGNSLVVIVIYFYMKLKTVASVFLLNLALADLCF | |
| | | | LLTLPLWAVYTAMEYRWPFGNYLCKIASASVSFNLY | |
| | | | ASVFLLTCLSIDRYLAIVHPMKSRLRRTMLVAKVTCIII | |
| | | | WLLAGLASLPAIIHRNVFFIENTNITVCAFHYESQNSTL | |
| | | | PIGLGLTKNILGFLFPFLIILTSYTLIWKALKKAYEIQKN | |
| | | | KPRNDDIFKIIMAIVLFFFFSWIPHQIFTFLDVLIQLGIIR | |
| | | | DCRIADIVDTAMPITICIAYFNNCLNPLFYGFLGKKFKR | |
| | | | YFLQLLKYIPPKAKSHSNLSTKMSTLSYRPSDNVSSST | |
| | | | KKPAPCFEVE | |
| 195 | omiganan | | L-lysinamide, L-isoleucyl-L-leucyl-L-arginyl-L-tryptophyl- | Formulation, dermal, |
| | | | L-prolyl-L-tryptophyl-L-tryptophyl-L-prolyl-L-tryptophyl- | topical |
| | | | L-arginyl-L-arginyl, pentahydrochloride | Peptide antibiotic |
| | | | | Antiacne |
| 427 | leuprorelin | | 6-D-leucine-9-(N-ethyl-L-prolinamide)-10-deglycinamide- | Transmucosal, nasal, |
| | | | | Menstruation disorders, |
| | | | | Anticancer, hormonal, |
| | | | | Fertility enhancer |
| 428 | delmitide | | D-Tyrosinamide, D-arginyl-D-norleucyl-D-norleucyl-D- | GI inflammatory/bowel |
| | acetate | | norleucyl-D-arginyl-D-norleucyl-D-norleucyl-D- | disorders, |
| | | | norleucylglycyl-, monoacetate | Radio/chemoprotective, |
| | | | | Antipsoriasis, |
| | | | | Antipruritic/inflamm, |
| | | | | allergic, Multiple sclerosis |
| | | | | treatment, Alimentary/ |
| | | | | Metabolic, other, |
| | | | | Antiviral, anti-HIV, |
| | | | | Antiasthma, COPD |
| | | | | treatment, Respiratory |
| | | | | Stomatological |
| 196 | cat PAD | | MRGALLVLALLVTQALGVKMAETCPIFYDVFFAVAN | Antiasthma |
| | | | GNELLLDLSLTKVNATEPERTAMKKIQDCYVENGLIS | Antiallergic, non-asthma |
| | | | RVLDGLVMTTISSSKDCMGEAVQNTVEDLKLNTLGR | |
| 429 | NOV-002 | | bis-(gamma-L-glutamyl)-L-cysteinyl-bis-glycin disodium | Anticancer, immunological |
| | | | salt | Radio/chemosensitizer |
| | | | | Antidote |
| 430 | GPG-NH2 | | glycyl-prolyl-glycine amide | Antiviral, anti-HIV |
| 431 | ABT-510 | | NAc-Sar-Gly-ValDalloleThrNValleArgProNHE | Anticancer, other |
| 8 | CJC-1131 | GLP-1 | HGEGTFTSDLSKQMEEEAVRLFIEWLKNGGPSSGAPP | Antidiabetic |
| | | | PS | |
| 432 | desmopressin | | Vasopressin, 1-(3-mercaptopropanoic acid)-8-D-arginine- | Formulation, oral, |
| | | | | Hormone, Antidiabetic, |
| | | | | Urological |
| 197 | metastin | | MNSLVSWQLLLFLCATHFGEPLEKVASVGNSRPTGQQ | Anticancer, other |
| | | | LESLGLLAPGEQSLPCTERKPAATARLSRRGTSLSPPPE | |
| | | | SSGSPQQPGLSAPHSRQIPAPQGAVLVQREKDLPNYN | |
| | | | WNSFGLRFGKREAAPGNHGRSAGRG | |
| 433 | leuprorelin | | 5-Oxo-L-prolyl-L-histidyl-L-tryptophyl-L-seryl-L-tyrosyl- | Anticancer |
| | | | D-leucyl-L-leucyl-L-arginyl-N-ethyl-L-prolinamide acetate | |
| | | | (salt) | |
| 434 | SGS-111 | | N-phenylacetylprolylglycine ethyl ester | Cognition enhancer |
| | | | | Neuroprotective |
| 435 | taltobulin | | (4S)-4-[[(2S)-3,3-dimethyl-2-[[(2S)-3-methyl-2- | Anticancer, other |
| | | | (methylamino)-3- | |
| | | | phenylbutanoyl]amino]butanoyl]methylamino]-2,5- | |
| | | | dimethylhex-2-enoic acid | |
| 436 | leuprolide | | 6-D-leucine-9-(N-ethyl-L-prolinamide)-10-deglycinamide- | inhalable, systemic, |
| | | | | Anticancer , Menstruation |
| | | | | disorders |
| 103 | XOMA-629 | | gi\|157276599\|ref\|NP_001716.2\| bactericidal/permeability- | Antiacne |
| | | | increasing protein precursor [Homo sapiens] | Anti-infective, other |
| | | | MRENMARGPCNAPRWASLMVLVAIGTAVTAAVNPG | |
| | | | VVVRISQKGLDYASQQGTAALQKELKRIKIPDYSDSF | |
| | | | KIKHLGKGHYSFYSMDIREFQLPSSQISMVPNVGLKFS | |
| | | | ISNANIKISGKWKAQKRFLKMSGNFDLSIEGMSISADL | |
| | | | KLGSNPTSGKPTITCSSCSSHINSVHVHISKSKVGWLIQ | |
| | | | LFHKKIESALRNKMNSQVCEKVTNSVSSELQPYFQTL | |
| | | | PVMTKIDSVAGINYGLVAPPATTAETLDVQMKGEFYS | |
| | | | ENHHNPPPFAPPVMEFPAAHDRMVYLGLSDYFFNTA | |
| | | | GLVYQEAGVLKMTLRDDMIPKESKFRLTTKFFGTFLP | |
| | | | EVAKKFPNMKIQIHVSASTPPHLSVQPTGLTFYPAVDV | |
| | | | QAFAVLPNSSLASLFLIGMHTTGSMEVSAESNRLVGE | |
| | | | LKLDRLLLELKHSNIGPFPVELLQDIMNYIVPILVLPRV | |
| | | | NEKLQKGFPLPTPARVQLYNVVLQPHQNFLLFGADV | |
| | | | VYK | |
| 198 | synthetic | | gi\|8393713\|ref\|INP_058651.1\| Sep (O-phosphoserine) | Antianaemic |
| | erythropoiesis | | tRNA: Sec (selenocysteine) tRNA synthase isoform 1 [Homo | Radio/chemoprotective |
| | pro | | sapiens] | |
| | | | MSTSYGCFWRRFIHGIGRSGDISAVQPKAAGSSLLNKI | |
| | | | TNSLVLDIIKLAGVHTVANCFVVPMATGMSLTLCFLT | |
| | | | LRHKRPKAKYIIWPRIDQKSCFKSMITAGFEPVVIENV | |
| | | | LEGDELRTDLKAVEAKVQELGPDCILCIHSTTSCFAPR | |
| | | | VPDRLEELAVICANYDIPHIVNNAYGVQSSKCMHLIQ | |
| | | | QGARVGRIDAFVQSLDKNFMVPVGGAIIAGFNDSFIQE | |
| | | | ISKMYPGRASASPSLDVLITLLSLGSNGYKKLLKERKE | |
| | | | MFSYLSNQIKKLSEAYNERLLHTPHNPISLAMTLKTLD | |
| | | | EHRDKAVTQLGSMLFTKQVSGARVVPLGSMQTVSGY | |
| | | | TFRGFMSHTNNYPCAYLNAASAIGMKMQDVDLFINR | |
| | | | LDRCLKAVRKERSKESDDNYDKTEDVDIEEMALKLD | |
| | | | NVLLDTYQDA | |
| | | | SS | |
| 191 | β-amyloid | beta-amyloid | gi\|8176533\|gb\|AAB26264.2\| beta-amyloid peptide precursor; | Cognition enhancer |
| | vaccine | peptide | beta APP [Homo sapiens] | |
| | | | GSGLTNIKTEEISEVKMDAEFRHDSGYEVHHQKLVFF | |
| | | | AEDVGSNKGAIIGLMVGGVVIATVIIITLVMLKKQYTS | |
| | | | NHHGVVE | |
| 437 | sincalide | | 1-De-(5-oxo-L-proline)-2-de-L-glutamine-5-L- | Imaging agent |
| | | | methioninecaerulein | Alimentary/Metabolic |
| 438 | albiglutide | | ([8-glycine]human glucagon-like peptide 1-(7-36)- | Antidiabetic |
| | | | peptidyl)([8-glycine]human glucagon-like peptide 1-(7-36)- | Anorectic/Antiobesity |
| | | | peptidyl)(human serum albumin (585 residues)) | |
| 199 | SB-144 | | gi\|13899257\|ref\|NP_113622.1\| transmembrane and ubiquitin- | Anticancer, other |
| | | | like domain containing 1 [Homo sapiens] | Radio/chemosensitizer |
| | | | MTLIEGVGDEVTVLFSVLACLLVLALAWVSTHTAEG | |
| | | | GDPLPQPSGTPTPSQPSAAMAATDSMRGEAPGAETPS | |
| | | | LRHRGQAAQPEPSTGFTATPPAPDSPQEPLVLRLKFLN | |
| | | | DSEQVARAWPHDTIGSLKRTQFPGREQQVRLIYQGQL | |
| | | | LGDDTQTLGSLHLPPNCVLHCHVSTRVGPPNPPCPPGS | |
| | | | EPGPSGLEIGSLLLPLLLLLLLLLWYCQIQYRPFFPLTA | |
| | | | TLGLAGFTLLLSLLAFAMYRP | |
| 200 | exenatide LAR | | L-histidylglycyl-L-glutamylglycyl-L-threonyl-L- | Antidiabetic |
| | | | phenylalanyl-L-threonyl-L-seryl-L-aspartyl-L-leucyl-L- | |
| | | | seryl-L-lysyl-L-glutaminyl-L-methionyl-L-glutamyl-L- | |
| | | | glutamyl-L-glutamyl-L-alanyl-L-valyl-L-arginyl-L-leucyl-L- | |
| | | | phenylalanyl-L-isoleucyl-L-glutamyl-L-tryptophyl-L-leucyl- | |
| | | | L-lysyl-L-asparaginylglycylglycyl-L-prolyl-L-seryl-L- | |
| | | | serylglycyl-L-alanyl-L-prolyl-L-prolyl-L-prolyl-L- | |
| | | | serinamide | |
| 201 | BA-058 | PTHrP | gi\|131542\|sp\|P12272.1\|PTHR_HUMAN Parathyroid | Osteoporosis treatment |
| | | | hormone-related protein precursor (PTH-rP) (PTHrP) | |
| | | | [Contains: PTHrP[1-36]; PTHrP[38-94]; Osteostatin | |
| | | | (PTHrP[107-139])] | |
| | | | MQRRLVQQWSVAVFLLSYAVPSCGRSVEGLSRRLKR | |
| | | | AVSEHQLLHDKGKSIQDLRRRFFLHHLIAEIHTAEIRA | |
| | | | TSEVSPNSKPSPNTKNHPVRFGSDDEGRYLTQETNKV | |
| | | | ETYKEQPLKTPGKKKKGKPGKRKEQEKKKRRTRSAW | |
| | | | LDSGVTGSGLEGDHLSDTSTTSLELDSRRH | |
| 8 | BIM-51077 | GLP-1 | HGEGTFTSDLSKQMEEEAVRLFIEWLKNGGPSSGAPP | Antidiabetic |
| | | | PS | |
| | | | [[(aminoisobutyric acid) 8,35]hGLP-1(1-36)NH₂, has the | |
| | | | same amino acid sequence as human GLP-1(7-36 amide) | |
| | | | except for the replacement of amino acids 8 and 35 with α- | |
| | | | aminoisobutyric acid (Aib) to reduce protease susceptibility. | |
| 202 | TM-701 | | H-Met-Cys-Met-Pro-Cys-Phe-Thr-Thr-Asp-His-Gln-Met- | Anticancer, other |
| | | | Ala-Arg-Lys-Cys-Asp-Asp-Cys-Cys-Gly-Gly-Lys-Gly-Arg- | Radio/chemosensitizer |
| | | | Gly-Lys-Cys-Tyr-Gly-Pro-Gln-Cys-Leu-Cys-Arg-NH₂ | |
| | | | (Disulfide bridge: 2-19, 5-28, 16-33, 20-35) | |
| 439 | CZEN-002 | | [dNal(2′)-7,Phe-12]-α-MSH 6-13 | Antifungal, Antibacterial, |
| | | | | other, Antiviral, anti-HIV, |
| | | | | Immunosuppressant, |
| | | | | Metabolic and enzyme |
| | | | | disorders, Anti- |
| | | | | inflammatory, |
| | | | | Antiarthritic, otherGI |
| | | | | inflammatory/bowel |
| | | | | disorders |
| 203 | ZP-120 | | Ac-RYYRWKKKKKKK-NH₂ | Cardiostimulant |
| 204 | CTT | | H-Cys-Thr-Thr-His-Trp-Gly-Phe-Thr-Leu-Cys-OH | Formulation technology |
| 205 | PYY3-36 | | gi\|71361686\|ref\|NP_004151.2\| peptide YY [Homo sapiens] | Anorectic/Antiobesity |
| | | | MVFVRRPWPALTTVLLALLVCLGALVDAYPIKPEAPR | |
| | | | EDASPEELNRYYASLRHYLNLVTRQRYGKRDGPDTLL | |
| | | | SKTFFPDGEDRPVRSRSEGPDLW | |
| | AEZS-130 | | EP1572 UMV1843 [Aib-DTrp-DgTrp-CHO] | Growth hormone |
| | | | | Anabolic |
| | | | | Musculoskeletal |
| 206 | AL-108 | | H-Asn-Ala-Pro-Val-Ser-Ile-Pro-Gln-OH | Neuroprotective |
| | | | | Cognition enhancer |
| | | | | Antiparkinsonian |
| | | | | Ophthalmological |
| 202 | TM-801 | | H-Met-Cys-Met-Pro-Cys-Phe-Thr-Thr-Asp-His-Gln-Met- | Imaging agent |
| | | | Ala-Arg-Lys-Cys-Asp-Asp-Cys-Cys-Gly-Gly-Lys-Gly-Arg- | |
| | | | Gly-Lys-Cys-Tyr-Gly-Pro-Gln-Cys-Leu-Cys-Arg-NH₂ | |
| | | | (Disulfide bridge: 2-19, 5-28, 16-33, 20-35) | |
| 202 | TM-901 | | H-Met-Cys-Met-Pro-Cys-Phe-Thr-Thr-Asp-His-Gln-Met- | Anticancer, other |
| | | | Ala-Arg-Lys-Cys-Asp-Asp-Cys-Cys-Gly-Gly-Lys-Gly-Arg- | Imaging agent |
| | | | Gly-Lys-Cys-Tyr-Gly-Pro-Gln-Cys-Leu-Cys-Arg-NH₂ | |
| | | | (Disulfide bridge: 2-19, 5-28, 16-33, 20-35) | |
| 440 | S-0373 | TRH | pyroGlu-His-Pro-NH₂ (or 5-oxo-L-prolyl-L-histidyl-L- | Neurological |
| | | | prolinamide) | Psychostimulant |
| | | | | Antiparkinsonian |
| 205 | PYY3-36 | | gi\|71361686\|ref\|NP_004151.2\| peptide YY [Homo sapiens] | Formulation, oral, other |
| | | | MVFVRRPWPALTTVLLALLVCLGALVDAYPIKPEAPR | Anorectic/Antiobesity |
| | | | EDASPEELNRYYASLRHYLNLVTRQRYGKRDGPDTLL | |
| | | | SKTFFPDGEDRPVRSRSEGPDLW | |
| 207 | XG-101 | | gi\|4885433\|ref\|NP_005447.1\| mitogen-activated protein | Immunological |
| | | | kinase 8 interacting protein 1 [Homo sapiens] | Cardiovascular |
| | | | MAERESGGLGGGAASPPAASPFLGLHIASPPNFRLTHD | Neuroprotective |
| | | | ISLEEFEDEDLSEITDECGISLQCKDTLSLRPPRAGLLSA | Immunosuppressant |
| | | | GGGGAGSRLQAEMLQMDLIDATGDTPGAEDDEEDDD | |
| | | | EERAARRPGAGPPKAESGQEPASRGQGQSQGQSQGPG | |
| | | | SGDTYRPKRPTTLNLFPQVPRSQDTLNNNSLGKKHSW | |
| | | | QDRVSRSSSPLKTGEQTPPHEHICLSDELPPQSGPAPTT | |
| | | | DRGTSTDSPCRRSTATQMAPPGGPPAAPPGGRGHSHR | |
| | | | DRIHYQADVRLEATEEIYLTPVQRPPDAAEPTSAFLPP | |
| | | | TESRMSVSSDPDPAAYPSTAGRPHPSISEEEEGFDCLSS | |
| | | | PERAEPPGGGWRGSLGEPPPPPRASLSSDTSALSYDSV | |
| | | | KYTLVVDEHAQLELVSLRPCFGDYSDESDSATVYDNC | |
| | | | ASVSSPYESAIGEEYEEAPRPQPPACLSEDSTPDEPDVH | |
| | | | FSKKFLNVFMSGRSRSSSAESFGLFSCIINGEEQEQTHR | |
| | | | AIFRFVPRHEDELELEVDDPLLVELQAEDYWYEAYN | |
| | | | MRTGARGVFPAYYAIEVTKEPEHMAALAKNSDWVD | |
| | | | QFRVKFLGSVQVPYHKGNDVLCAAMQKIATTRRLTV | |
| | | | HFNPPSSCVLEISVRGVKIGVKADDSQEAKGNKCSHFF | |
| | | | QLKNISFCGYHPKNNKYFGFITKHPADHRFACHVFVS | |
| | | | EDSTKALAESVGRAFQQFYKQFVEYTCPTEDIYLE | |
| 208 | XG-102 | | gi\|4885433\|ref\|NP_005447.1\| mitogen-activated protein | Neuroprotective |
| | | | kinase 8 interacting protein 1 [Homo sapiens] | Cardiovascular |
| | | | MAERESGGLGGGAASPPAASPFLGLHIASPPNFRLTHD | Otological |
| | | | ISLEEFEDEDLSEITDECGISLQCKDTLSLRPPRAGLLSA | Ophthalmological |
| | | | GGGGAGSRLQAEMLQMDLIDATGDTPGAEDDEEDDD | Antiparkinsonian |
| | | | EERAARRPGAGPPKAESGQEPASRGQGQSQGQSQGPG | Immunosuppressant |
| | | | SGDTYRPKRPTTLNLFPQVPRSQDTLNNNSLGKKHSW | |
| | | | QDRVSRSSSPLKTGEQTPPHEHICLSDELPPQSGPAPTT | |
| | | | DRGTSTDSPCRRSTATQMAPPGGPPAAPPGGRGHSHR | |
| | | | DRIHYQADVRLEATEEIYLTPVQRPPDAAEPTSAFLPP | |
| | | | TESRMSVSSDPDPAAYPSTAGRPHPSISEEEEGFDCLSS | |
| | | | PERAEPPGGGWRGSLGEPPPPPRASLSSDTSALSYDSV | |
| | | | KYTLVVDEHAQLELVSLRPCFGDYSDESDSATVYDNC | |
| | | | ASVSSPYESAIGEEYEEAPRPQPPACLSEDSTPDEPDVH | |
| | | | FSKKFLNVFMSGRSRSSSAESFGLFSCIINGEEQEQTHR | |
| | | | AIFRFVPRHEDELELEVDDPLLVELQAEDYWYEAYN | |
| | | | MRTGARGVFPAYYAIEVTKEPEHMAALAKNSDWVD | |
| | | | QFRVKFLGSVQVPYHKGNDVLCAAMQKIATTRRLTV | |
| | | | HFNPPSSCVLEISVRGVKGVKADDSQEAKGNKCSHFF | |
| | | | QLKNISFCGYHPKNNKYFGFITKHPADHRFACHVFVS | |
| | | | EDSTKALAESVGRAFQQFYKQFVEYTCPTEDIYLE | |
| 441 | lanreotide SR | | L-Threonamide,3-(2-naphthaleny1)-D-alanyl-L-cysteinyl-L- | Formulation, modified- |
| | | | tyrosyl-D-tryptophyl-L-lysyl-L-valyl-L-cysteinyl-, cyclic (2- | release, other |
| | | | 7)-disulfide | Somatostatin |
| | | | | Antihypertensive, other |
| 209 | OGP-(10-14)-L | | H-Tyrosine-Glycine-Phenylalanine-Glycine-Glycine-OH | Haematological |
| | | | | Musculoskeletal |
| 210 | WP9QY | | cyclo(Tyr-Cys-Trp-Ser-Gln-Tyr-Leu-Cys-Tyr); | Antiarthritic, other |
| | | | cyclo(tyrosyl-cysteinyl-tryptophyl-seryl-glutaminyl-tyrosyl- | Anti-inflammatory |
| | | | leucyl-cysteinyl-tyrosyl) | |
| 211 | aviptadil | | His-Ser-Asp-Ala-Val-Phe-Thr-Asp-Asn-Tyr-Thr-Arg-Leu- | Antihypertensive, other |
| | | | Arg-Lys-Gln-Met-Ala-Val-Lys-Lys-Tyr-Leu-Asn-Ser-Ile- | Respiratory |
| | | | Leu-Asn | Immunosuppressant |
| 212 | AL-209 | | Ser-Ala-Leu-Leu-Arg-Ser-Ile-Pro-Ala | Neuroprotective |
| | | | | Cognition enhancer |
| | | | | Ophthalmological |
| 442 | octreotide | | L-Cysteinamide, D-phenylalanyl-L-cysteinyl-L- | Formulation, implant |
| | | | phenylalanyl-D-tryptophyl-L-lysyl-L-threonyl-N-[2- | Formulation, modified- |
| | | | hydroxy-1-(hydroxymethyl)propyl]-, cyclic (2-7)-disulfide, | release, >24 hr |
| | | | [R-(R*,R*)]- | Somatostatin |
| 213 | CDX-110 | | Leu-Glu-Glu-Lys-Lys-Gly-Asn-Tyr-Val-Val-Thr-Asp-His- | Recombinant vaccine |
| | | | Cys-KLH | Anticancer, immunological |
| 444 | desmopressin | | Vasopressin, 1-(3-mercaptopropanoic acid)-8-D-arginine- | Hormone, Urological, |
| | | | | Reproductive/gonadal, |
| | | | | general |
| 445 | obinepitide | | [34-L-glutamine]pancreatic hormone (human) | Anorectic/Antiobesity |
| | Insulin | | Insulin (ox), 8A-L-threonine-10A-L-isoleucine-30B-L- | solubility-enhanced |
| | | | threonine- | Insulin |
| 171 | terlipressin | | N-(N-(N-glycylglycyl)glycyl)-8-L-lysinevasopressin | Hepatoprotective, |
| | | | [CAS}; Gly-Gly-Gly-8-Lys-vasopressin; N-(alpha)-glycyl- | Urological, Gi bleeding |
| | | | glycyl-glycyl-8-lysine vasopressin; Remestyp; TGLVP; | |
| | | | glipressin; glycylpressin; glypressin; terlypressin; triglycyl | |
| | | | lysine vasopressin; triglycyl-(8-lysine)vasopressin; | |
| | | | triglycylvasopressin; vasopressin, tri-Gly-8-Lys- | |
| 214 | ZT-153 | | Asn-Phe-Gly-Ala-Ile-Leu; NFGAIL; asparagyl- | Antidiabetic |
| | | | phenylalanyl-glycyl-alanyl-isoleucyl-leucine; islet amyloid | |
| | | | polypeptide (22-27) | |
| 215, 215 | FGLL | | gi\|42544189\|ref\|NP_004458.3\| fibrinogen-like 1 precursor | Cognition enhancer |
| and 216 | | | [Homo sapiens] | Neurological |
| | | | MAKVFSFILVTTALTMGREISALEDCAQEQMRLRAQV | |
| | | | RLLETRVKQQQVKIKQLLQENEVQFLDKGDENTVIDL | |
| | | | GSKRQYADCSEIFNDGYKLSGFYKIKPLQSPAEFSVYC | |
| | | | DMSDGGGWTVIQRRSDGSENFNRGWKDYENGFGNF | |
| | | | VQKHGEYWLGNKNLHFLTTQEDYTLKIDLADFEKNS | |
| | | | RYAQYKNFKVGDEKNFYELNIGEYSGTAGDSLAGNF | |
| | | | HPEVQWWASHQRMKFSTWDRDHDNYEGNCAEEDQS | |
| | | | GWWFNRCHSANLNGVYYSGPYTAKTDNGIVWYTWH | |
| | | | GWWYSLKSVVMKIRPNDFIPNVI | |
| | | | gi\|42544200\|ref\|NP_963846.1\| fibrinogen-like 1 precursor | |
| | | | [Homo sapiens] | |
| | | | MAKVFSFILVTTALTMGREISALEDCAQEQMRLRAQV | |
| | | | RLLETRVKQQQVKIKQLLQENEVQFLDKGDENTVIDL | |
| | | | GSKRQYADCSEIFNDGYKLSGFYKIKPLQSPAEFSVYC | |
| | | | DMSDGGGWTVIQRRSDGSENFNRGWKDYENGFGNF | |
| | | | VQKHGEYWLGNKNLHFLTTQEDYTLKIDLADFEKNS | |
| | | | RYAQYKNFKVGDEKNFYELNIGEYSGTAGDSLAGNF | |
| | | | HPEVQWWASHQRMKFSTWDRDHDNYEGNCAEEDQS | |
| | | | GWWFNRCHSANLNGVYYSGPYTAKTDNGIVWYTWH | |
| | | | GWWYSLKSVVMKIRPNDFIPNVI | |
| | | | gi\|42544198\|ref\|NP_671736.2\| fibrinogen-like 1 precursor | |
| | | | [Homo sapiens] | |
| | | | MAKVFSFILVTTALTMGREISALEDCAQEQMRLRAQV | |
| | | | RLLETRVKQQQVKIKQLLQENEVQFLD | |
| 217 | ST-03 | | gi\|386634\|gb\|AAB27460.1\| 01-ST- = heat-stable enterotoxin | Recombinant growth factor |
| | | | [Vibrio cholerae, 01, Peptide, 19 aa] | Musculoskeletal |
| | | | NLIDCCEICCNPACFGCLN | Osteoporosis treatment |
| 446 | cetrorelix | | D-Alaninamide, N-acetyl-3-(2-naphthalenyl)-D-alanyl-4- | Formulation, modified- |
| | acetate | | chloro-D-phenylalanyl-3-(3-pyridinyl)-D-alanyl-L-seryl-L- | release, >24 hr |
| | | | tyrosyl-N5-(aminocarbonyl)-D-ol-L-leucyl-L-arginyl-L- | Menstruation disorders |
| | | | prolyl- | |
| 218 | neurodegenerative | | alpha toxin, Naja; cobra alpha toxin; cobra toxin alpha; toxin | Cognition enhancer |
| | ther | | alpha, cobra; | |
| | | | gi\|64054\|emb\|CAA26373.1\| unnamed protein product | |
| | | | [Laticauda semifasciata] | |
| | | | MKTLLLTLVVVTIVCLDLGYTRICFNHQSSQPQTTKTC | |
| | | | SPGESSCYNKQWSDFRGTIIERGCGCPTVKPGIKLSCC | |
| | | | ESEVCNN | |
| | | | gi\|4519816\|dbj\|BAA75752.1\| short chain neurotoxin | |
| | | | [Laticauda semifasciata] | |
| | | | MKTLLLTLVVVTIVCLDLGYTRICFNHQSSQPQTTKTC | |
| | | | SPGESSCYNKQWSDFRGTIIERGCGCPTVKPGIKLSCC | |
| | | | ESEVCNN | |
| | | | gi\|3214056\|dbj\|BAC78199.1\| erabutoxin a [Laticauda | |
| | | | semifasciata] | |
| | | | MKTLLLTLVVVTIVCLDLGYTRICFNHQSSQPQTTKTC | |
| | | | SPGESSCYNKQWSDFRGTIIERGCGCPTVKPGIKLSCC | |
| | | | ESEVCNN | |
| | | | gi\|32140563\|dbj\|BAC782OO.1\| erabutoxin a [Laticauda | |
| | | | semifasciata] | |
| | | | MKTLLLTLVVVTIVCLDLGYTRICFNHQSSQPQTTKTC | |
| | | | SPGESSCYNKQWSDFRGTIIERGCGCPTVKPGIKLSCC | |
| | | | ESEVCNN | |
| 219 | CT-319 | | MSNKKIIKIIKLQIPGGKANPAPPIGPALGAAGVNIMGF | Antiviral, anti-HIV |
| | | | CKEFNAATQDRPGDLLPVVIT | |
| | | | VYSDKTFSFVMKQSPVSSLIKKALGLESGSKIPNRNKV | |
| | | | GKLTRAQITVIAEQKMKDMDVV | |
| | | | LLESAERMVEGTARSMGVDVE | |
| 447 | Peptide T | | L-Threonine, N-(N-(N2-(N-(N-(N-(N-D-alanyl-L-seryl)-L- | Antipsoriasis |
| | | | threonyl)-L-threonyl)-L-threonyl)-L-asparaginyl)-L-tyrosyl)- | Multiple sclerosis |
| | | | [CAS]; HIV Peptide T; Peptide T, HIV | treatment |
| | | | | Cognition enhancer |
| | | | | Musculoskeletal |
| 220 and | APP-018 | | pallidin [Mus musculus] | Hypolipaemic/Antiathero- |
| 221 | | | gi\|80790039\|ref\|NP_062762.1\|[9790039] | sclerosis |
| | | | MSVPEPPPPDGVLTGPSDSLEAGEPTPGLSDTSPDEGLI | |
| | | | EDFPVDDRAVEHLVGGLLSHY | |
| | | | LPDLQRSKRALQELTQNQVVLLDTLEQEISKFKECHS | |
| | | | MLDINALFTEAKHYHAKLVTIRK | |
| | | | EMLLLHEKTSKLKKRALKLQQKRQREELEREQQREK | |
| | | | EFEREKQLTAKPAKRT | |
| | | | envelope glycoprotein [Human immunodeficiency virus type | |
| | | | 1] | |
| | | | gi\|14205319\|gb\|AAD11044.1\|[4205319] | |
| | | | KLTPLCVTLNCTDLDLRNTTNNTTTEERGEMKNCSFN | |
| | | | ITTNIRDRYQKEYALFYKLDVIP | |
| | | | IKEDNTSDNTSYRLISCNTSVITQACPKIS | |
| 222 | somatropin | | gi\|60651145\|gb\|AAX31661.1\| somatotropin [Bubalus | Formulation, transmucosal, |
| | | | bubalis] | nasal |
| | | | AFPAMSLSSLFANAVLRAQHLHQLAADTFKEFERTYI | Growth hormone |
| | | | PEGQRYSIQNTQVAFCFSETIPA | Anabolic |
| | | | PTGKNEAQQKSDLELLRISLLLIQSWLGPLQFLSRVFT | Reproductive/gonadal, |
| | | | NSLVFGTSDRVYEKLKDLEEGI | general |
| | | | LALMRELEDGTPRAGQILKRTYDKFDTNMRSDDALL | |
| | | | KNYGLLSCFRKDLHKTETYLRVMKCRRFGEASCAF | |
| 448 | heparin | | 6-[5-acetamido-4,6-dihydroxy-2-(sulfooxymethyl)oxan-3- | Formulation, transmucosal, |
| | | | yl]oxy-3-[5-(6-carboxy-4,5-dihydroxy- | nasal |
| | | | 3-sulfooxyoxan-2-yl)oxy-6-(hydroxymethyl)-3- | Anticoagulant |
| | | | (sulfoamino)-4-sulfooxyoxan-2-yl]oxy- | |
| | | | 4-hydroxy-5-sulfooxyoxane-2-carboxylic acid | |
| 46 | CGRP | CGRP | ACDTATCVTHRLAGLLSRSGGVVKNNFVPTNVGSKA | Cardiovascular |
| | | | F—NH₂ | Cardiostimulant |
| 449 | YM-216391 | | A concise total synthesis of the unusual oxazole-based | Anticancer, other |
| | | | cyclopeptide structure YM-216391, which also establishes | |
| | | | the stereochemistry of the natural product i.e. 1, is described. | |
| | | | The unusual polyoxazole-thiazole-based cyclopeptide 1, | |
| | | | designated YM-216391, was recently isolated from | |
| | | | Streptomyces nobilis.1 It shares both a structural and | |
| | | | biological homology with the potent telomerase inhibitor | |
| | | | telomestatin 2 which is showing promise in cancer | |
| | | | chemotherapy. 2 The structure of YM-216391 comprises a | |
| | | | continuum of five azoles which have their origins in serine, | |
| | | | cysteine and phenylalanine, linked via a glycine- valine- | |
| | | | isoleucine tripeptide tether. The complete stereochemical | |
| | | | assignment of YM-216391 has not been established. In this | |
| | | | communication we describe a concise total synthesis of the | |
| | | | cyclopeptide, which not only confirms its unique structure | |
| | | | but also allows the assignment of its stereochemistry, shown | |
| | | | in formula 1. Thus, the 2,4-disubstituted oxazoles 3 and 4 | |
| | | | and the trisubstituted oxazole 5 were first elaborated | |
| 223 | FGLm | | LSENDEWTQDRAKP | Cognition enhancer |
| | | | | Neurological |
| 224 | prohanin | | NPFPTWRKRPG | Analgesic, other |
| 225 | heart failure | NP | gi\|189079\|gb\|AAA36355.1\| natriuretic peptide | Cardiostimulant |
| | therapy | | MDPQTAPSRALLLLLFLHLAFLGGRSHPLGSPGSASDL | |
| | | | ETSGLQEQRNHLQGKLSELQVEQTSLEPLQES | |
| | | | PRPTGVWKSREVATEGIRGHRKMVLYTLRAPRSPKM | |
| | | | VQGSGCFGRKMDRISSSSGLGCKVLRRH | |
| 450 | SEN-304 | | D-[(chG)Y-(chG)(chG)(MeL)]-NH₂, where chG is R- | Cognition enhancer |
| | | | cyclohexylglycine | Anti-inflammatory |
| 451 | Primacoll | | Synthetic growth factor | Musculoskeletal |
| 452 | Octreotide | | L-Cysteinamide, D-phenylalanyl-L-cysteinyl-L- | Formulation, modified- |
| | | | phenylalanyl-D-tryptophyl-L-lysyl-L-threonyl-N-[2- | release, >24 hr |
| | | | hydroxy-1-(hydroxymethyl)propyl]-, cyclic (2-7)-disulfide, | Symptomatic antidiabetic |
| | | | [R-(R*,R*)]- | Ophthalmological |
| | | | | Somatostatin |
| 453 | ALS-02 | | Glycine, N-(aminoiminomethyl)-N-methyl- | Neuroprotective |
| 200 | exendin-4, PC- | GLP-1 | L-histidylglycyl-L-glutamylglycyl-L-threonyl-L- | Antidiabetic |
| | DAC | | phenylalanyl-L-threonyl-L-seryl-L-aspartyl-L-leucyl-L- | |
| | | | seryl-L-lysyl-L-glutaminyl-L-methionyl-L-glutamyl-L- | |
| | | | glutamyl-L-glutamyl-L-alanyl-L-valyl-L-arginyl-L-leucyl-L- | |
| | | | phenylalanyl-L-isoleucyl-L-glutamyl-L-tryptophyl-L-leucyl- | |
| | | | L-lysyl-L-asparaginylglycylglycyl-L-prolyl-L-seryl-L- | |
| | | | serylglycyl-L-alanyl-L-prolyl-L-prolyl-L-prolyl-L- | |
| | | | serinamide | |
| 226 | Exenatide | | gi\|1916067\|gb\|AAB51130.1\| exendin 4 [Heloderma | Formulation, transmucosal, |
| | | | suspectum] | nasal |
| | | | MKIILWLCVFGLFLATLFPISWQMPVESGLSSEDSASS | Antidiabetic |
| | | | ESFASKIKRHGEGTFTSDLSKQMEEEAVRLFIEWLKNG | |
| | | | GPSSGAPPPSG | |
| 225 | Cardeva | BNP | gi\|113836\|sp\|P16860.1\|ANFB_HUMAN Natriuretic peptides | Cardiostimulant |
| | | | B precursor [Contains: Gamma-brain natriuretic peptide; | |
| | | | Brain natriuretic peptide 32 (BNP-32)] | |
| | | | MDPQTAPSRALLLLLFLHLAFLGGRSHPLGSPGSASDL | |
| | | | ETSGLQEQRNHLQGKLSELQVEQTSLEPLQES | |
| | | | PRPTGVWKSREVATEGIRGHRKMVLYTLRAPRSPKM | |
| | | | VQGSGCFGRKMDRISSSSGLGCKVLRRH | |
| 227 | Alloferon | | H-His-Gly-Val-Ser-Gly-His-Gly-Gln-His-Gly-Val-His-Gly- | Immunomodulator, anti- |
| | | | OH | infective |
| 454 | PAC-G31P | | AMCF-I; Alveolar Macrophage Chemotactic Factor I; | Recombinant interleukin |
| | | | Alveolar Macrophage Chemotactic Factor-I; Anionic | Respiratory |
| | | | Neutrophil Activating Peptide; Anionic Neutrophil- | Antiasthma |
| | | | Activating Peptide; CXCL8 Chemokine; CXCL8 | COPD treatment |
| | | | Chemokines; CXCL8, Chemokine; Chemokine CXCL8; | |
| | | | Chemokine, CXCL8; Chemokines, CXCL8; Chemotactic | |
| | | | Factor, Macrophage Derived; Chemotactic Factor, | |
| | | | Macrophage-Derived; Chemotactic Factor, Neutrophil; | |
| | | | Chemotactic Factor, Neutrophil, Monocyte-Derived; | |
| | | | Chemotactic Peptide-Interleukin-8, Granulocyte; | |
| | | | Granulocyte Chemotactic Peptide Interleukin 8; Granulocyte | |
| | | | Chemotactic Peptide-Interleukin-8; IL-8; IL8; Interleukin 8; | |
| | | | Lymphocyte-Derived Neutrophil-Activating Peptide; | |
| | | | Macrophage-Derived Chemotactic Factor; Monocyte- | |
| | | | Derived Neutrophil Chemotactic Factor; Monocyte-Derived | |
| | | | Neutrophil-Activating Peptide; Neutrophil Activating | |
| | | | Peptide, Lymphocyte Derived; Neutrophil Activating | |
| | | | Peptide, Monocyte Derived; Neutrophil Activation Factor; | |
| | | | Neutrophil Chemotactic Factor; Neutrophil-Activating | |
| | | | Peptide, Anionic; Neutrophil-Activating Peptide | |
| 228 | PAC-525 | | Ac-KWRRWVRWI-NH₂ | Antibacterial, other |
| 229, 229 | PAC-113 | | Lys-Phe-His-Glu-Lys-His-His-Ser-His-Arg-Gly-Tyr | Antifungal |
| and 230 | | | histatin 10, human; histatin 11, human; histatin 12, human; | |
| | | | histatin 3, human; histatin 4, human; histatin 5, human; | |
| | | | histatin 6, human; histatin 7, human; histatin 8, human; | |
| | | | histatin 9, human; histatin-3 (1-24), human; histatin-3 (1-25), | |
| | | | human; histatin-3 (12-24), human; histatin-3 (12-25), human; | |
| | | | histatin-3 (12-32), human; histatin-3 (13-25), human; | |
| | | | histatin-3 (5-11), human; histatin-3 (5-12), human; lysyl- | |
| | | | phenylalanyl-histidyl-glutamyl-lysyl-histidyl-histidyl-seryl- | |
| | | | histidyl-arginyl-glycyl-tyrosine | |
| | | | gi\|4557653\|ref\|NP_000191.1\| histatin 3 [Homo sapiens] | |
| | | | MKFFVFALILALMLSMTGADSHAKRHHGYKRKFHEK | |
| | | | HHSHRGYRSNYLYDN | |
| 231 | MLIF | | Met-Gln-Cys-Asn-Ser | Anti-inflammatory |
| | | | U.S. Pat. No. 6,524,591 | |
| 454 | carfilzomib | | L-Phenylalaninamide, (alphaS)-alpha-[(4- | Anticancer, other |
| | | | morpholinylacetyl)amino]benzenebutanoyl-L-leucyl-N- | |
| | | | [(1S)-3-methyl-1-[[(2R)-2-methyloxiranyl]carbonyl]butyl]- | |
| 232 | NAFB001 | | gi\|63025222\|ref\|NP_000651.3\| transforming growth factor, | Ophthalmological |
| | | | beta 1 [Homo sapiens] | Hepatoprotective |
| | | | MPPSGLRLLPLLLPLLWLLVLTPGRPAAGLSTCKTIDM | |
| | | | ELVKRKRIEAIRGQILSKLRLASPPSQGEVPP | |
| | | | GPLPEAVLALYNSTRDRVAGESAEPEPEPEADYYAKE | |
| | | | VTRVLMVETHNEIYDKFKQSTHSIYMFFNTSEL | |
| | | | REAVPEPVLLSRAELRLLRLKLKVEQHVELYQKYSNN | |
| | | | SWRYLSNRLLAPSDSPEWLSFDVTGVVRQWLSR | |
| | | | GGEIEGFRLSAHCSCDSRDNTLQVDINGFTTGRRGDL | |
| | | | ATIHGMNRPFLLLMATPLERAQHLQSSRHRRAL | |
| | | | DTNYCFSSTEKNCCVRQLYIDFRKDLGWKWIHEPKG | |
| | | | YHANFCLGPCPYIWSLDTQYSKVLALYNQHNPGA | |
| | | | SAAPCCVPQALEPLPIVYYVGRKPKVEQLSNMIVRSC | |
| | | | KCS [PIR] | |
| 233 | IL12-NGR | | H-Cys-Asn-Gly-Arg-Cys-Gly-OH (Disulfide bridge: 1-5) | Recombinant, other |
| | | | | Cytokine |
| | | | | Anticancer, immunological |
| 234 and | enterostatin | | Val-Pro-Val-Asp; Val-Pro-Asp-Pro-Arg | Anorectic/Antiobesity |
| 235 | | | | |
| 455 | octreotide | | L-Cysteinamide, D-phenylalanyl-L-cysteinyl-L- | Formulation, modified- |
| | | | phenylalanyl-D-tryptophyl-L-lysyl-L-threonyl-N-[2- | release, >24 hr |
| | | | hydroxy-1-(hydroxymethyl)propyl]-, cyclic (2-7)-disulfide, | Somatostatin |
| | | | [R-(R*,R*)]- | |
| 150 | enfuvirtide | | L-Phenylalaninamide, N-acetyl-L-tyrosyl-L-threonyl-L- | Formulation, parenteral, |
| | | | seryl-L-leucyl-L-isoleucyl-L-histidyl-L-seryl-L-leucyl-L- | needle-free |
| | | | isoleucyl-L-alpha-glutamyl-L-alpha-glutamyl-L-seryl-L- | Antiviral, anti-HIV |
| | | | glutaminyl-L-asparaginyl-L-glutaminyl-L-glutaminyl-L- | |
| | | | alpha-glutamyl-L-lysyl-L-asparaginyl-L-alpha-glutamyl-L- | |
| | | | glutaminyl-L-alpha-glutamyl-L-leucyl-L-leucyl-L-alpha- | |
| | | | glutamyl-L-leucyl-L-alpha-aspartyl-L-lysyl-L-tryptophyl-L- | |
| | | | alanyl-L-seryl-L-leucyl-L-tryptophyl-L-asparaginyl-L- | |
| | | | tryptophyl- | |
| 236 | PR-21 | | gi\|2213924\|gb\|AAB61615.1\| neural cell adhesion molecule | Neurological |
| | | | [Homo sapiens] | Cognition enhancer |
| | | | MLQTKDLIWTLFFLGTAVSLQVDIVPSQGEISVGESKF | |
| | | | FLCQVAGDAKDKDISWFSPNGEKLTPNQQRIS | |
| | | | VVWNDDSSSTLTIYNANIDDAGIYKCVVTGEDGSESE | |
| | | | ATVNVKIFQKLMFKNAPTPQEFREGEDAVIVCD | |
| | | | VVSSLPPTIIWKHKGRDVILKKDVRFIFLSNNYLPIPGI | |
| | | | KKTDEGTYRCEGRILARGEINFNDIQVIVNV | |
| | | | PPTIQARQNIVNATANLGQSVTLVCDAEGFPGPTMSW | |
| | | | TKDGEQIEQEEHDEKYLFSDDSSHLTIKKVDKN | |
| | | | HEAENICIAENKVGEQDATIHLKVFAKPQITYVEDQTA | |
| | | | MELAEQVILTVEASGDHIPYITWWTSTWQI | |
| 237 | AC-163794 | GIP | gi\|183221\|gb\|AAA53192.1\| gastric inhibitory polypeptide | Antidiabetic |
| | | | precursor | |
| | | | MVATKTFALLLLSLFLAVGLGEKKEGHFSALPSLPVG | |
| | | | SHAKVSSPQPRGPRYAEGTFISDYSIAMDKIHQ | |
| | | | QDFVNWLLAQKGKKNDWKHNITQREARALELASQA | |
| | | | NRKEEEAVEPQSSPAKNPSDEDLLRDLLIQELLAC | |
| | | | LLDQTNLCRLRSR; | |
| 456 | glucagon | | Glucagon (1-29); Glukagon; HG Factor; HG-Factor; | Formulation, transdermal, |
| | | | Hyperglycemic Glycogenolytic Factor; Hyperglycemic- | systemic |
| | | | Glycogenolytic Factor; Proglucagon (33-61) | hypoglycemia |
| 457 | Insulin | | Insulin (ox), 8A-L-threonine-10A-L-isoleucine-30B-L- | Formulation, oral, other |
| | | | threonine- | Formulation, optimized, |
| | | | | nanoparticles |
| | | | | Antidiabetic |
| 458 | Dekafin-2 | | DNA Synthesis Factor; Fibroblast Growth Factor; Fibroblast | Anticancer, other |
| | | | Growth Regulatory Factor; Growth Factor, Fibroblast; | |
| | | | Growth Factors, Fibroblast | |
| 238 and | relaxin | | (1) Glu-Leu-Tyr-Ser-Ala-Leu-Ala.Asn-Lys-Cys-Cys-His- | Recombinant hormone |
| 239 | | | Val-Gly-Cys-Thr-Lys-Arg-Ser-Leu-Ala-Arg-Phe-Cys | Hormone |
| | | | (2) H-Asp-Ser-Trp-Met-Glu-Glu-Val-Ile-Lys-Leu-Cys-Gly- | Labour inducer |
| | | | Arg-Glu-Leu-Val-Arg-Ala-Gln-Ile-Ala-Ile-Cys-Gly-Met- | Antihypertensive, other |
| | | | Ser-Thr-Ser | |
| | | | Cys 11 of each chain form disulfide bond; cys 24 of the first | |
| | | | chain forms disulfide bond with cys 23 of chain 2 | |
| 459 | rhNRG-1 | | Differentiation Factor, neu; GGF Protein; Glial Growth | Recombinant, other |
| | | | Factor; Heregulin; NDF Protein; NRG1 Protein; Neuregulin | Cardiostimulant |
| | | | 1; neu Differentiation Factor | |
| 240 | c-peptide | C-peptide | Glu-Ala-Glu-Asp-Leu-Gln-Val-Gly-Gln-Val-Glu-Leu-Gly- | Symptomatic antidiabetic |
| | analogue | | Gly-Gly-Pro-Gly-Ala-Gly-Ser-Leu-Gln-Pro-Leu-Ala-Leu- | |
| | | | Glu-Gly-Ser-Leu-Gln | |
| 241 | SB-101 | | gi\|30353933\|gb\|AAH52287.1\| CD44 protein [Homo sapiens] | Recombinant, other |
| | | | MDKFWWHAAWGLCLVPLSLAQIDLNITCRFAGVFHV | Anticancer, other |
| | | | EKNGRYSISRTEAADLCKAFNSTLPTMAQMEKAL | |
| | | | SIGFETCSST | |
| 242 | Britistatin | | gi\|66270695\|gb\|AAY43681.1\| disintegrin isoform D-1 [Bitis | Antithrombotic |
| | | | arietans] | |
| | | | SPPVCGNKILEQGEDCDCGSPANCQDRCCNAATCKLT | |
| | | | PGSQCNYGECCDQCRFKKAGTVCRIARGDWNDDYCT | |
| | | | GKSSDCPWNH | |
| 243 | echistatin | | gi\|208338\|gb\|AAA72777.1\| echistatin | Antithrombotic |
| | | | MECESGPCCRNCKFLKEGTICKRARGDDLDDYCNGK | |
| | | | TCDCPRNPHKGPAT | |
| 244 | gastrin | | gi\|4503923: 20-101 gastrin preproprotein [Homo sapiens] | diabetes |
| | | | EASWKPRSQQPDAPLGTGANRDLELPWLEQQGPASH | |
| | | | HRRQLGPQGPPHLVADPSKKQGPWLEEEEEAYGWM | |
| | | | DFGRRSAEDEN | |
| 245 | herpes simplex | | gi\|9629447: 1-23 envelope glycoprotein D [Human | Prophylactic vaccine |
| | vaccine | | herpesvirus 1] | |
| | | | MGGAAARLGAVILFVVIVGLHGV | |
| 246 | neurotensin | | gi\|5453816: 152-163 neurotensin/neuromedin N | Analgesic, other |
| | | | preproprotein [Homo sapiens] | |
| | | | LYENKPRRPYIL | |
| 247 | nociceptin | | gi\|5453922\|ref\|NP_006219.1\| prepronociceptin [Homo | Neurological |
| | | | sapiens] | Cognition enhancer |
| | | | MKVLLCDLLLLSLFSSVFSSCQRDCLTCQEKLHPALDS | Analgesic, other |
| | | | FDLEVCILECEEKVFPSPLWTPCTKVMARSSWQLSPA | |
| | | | APEHVAAALYQPRASEMQHLRRMPRVRSLFQEQEEP | |
| | | | EPGMEEAGEMEQKQLQKRFGGFTGARKSARKLANQ | |
| | | | KRFSEFMRQYLVLSMQSSQRRRTLHQNGNV | |
| 248 | oxyntomodulin | | sp\|P01275.3\|GLUC_HUMAN: 53-89 Glucagon precursor | Obesity; Antiulcer |
| | | | [Contains: Glicentin; Glicentin-related polypeptide (GRPP); | |
| | | | Oxyntomodulin (OXY) (OXM)] | |
| | | | HSQGTFTSDYSKYLDSRRAQDFVQWLMNTKRNRNNI | |
| | | | A | |
| 249 | pancreastatin | | gi\|164417: 256-304 chromogranin A precursor | Antidiabetic |
| | | | GWPQAPAMDGAGKTGAEEAQPPEGKGAREHSRQEEE | |
| | | | EETAGAPQGLFRG | |
| 250 | relaxin | Relaxin | gi\|5902052\|ref\|NP_008842.1\| relaxin 1 preproprotein [Homo | Recombinant hormone |
| | | | sapiens] | Hormone |
| | | | MPRLFLFHLLEFCLLLNQFSRAVAAKWKDDVIKLCGR | Labour inducer |
| | | | ELVRAQIAICGMSTWSKRSLSQEDAPQTPRPVAEIVPS | |
| | | | FINKDTETIIIMLEFIANLPPELKAALSERQPSLPELQQY | |
| | | | VPALKDSNLSFEEFKKLIRNRQSEAADSNPSELKYLGL | |
| | | | DTHSQKKRRPYVALFEKCCLIGCTKRSLAKYC | |
| 251 | secretin | | gi\|11345450: 28-54 secretin preproprotein [Homo sapiens] | Haemostatic; diagnostic of |
| | | | HSDGTFTSELSRLREGARLQRLLQGLV | pancreatic dysfunction, |
| | | | | asthma, COPD, others |
| 252 | TIMP | | MAPFEPLASGILLLLWLIAPSRACTCVPPHPQTAFCNS | Recombinant, other |
| | | | DLVIRAKFVGTPEVNQTTLYQRYEIKMTKMYKGFQA | Vulnerary |
| | | | LGDAADIRFVYTPAMESVCGYFHRSHNRSEEFLIAGK | Antiarthritic, other |
| | | | LQDGLLHITTCSFVAPWNSLSLAQRRGFTKTYTVGCE | Stomatological |
| | | | ECTVFPCLSIPCKLQSGTHCLWTDQLLQGSEKGFQSRH | |
| | | | LACLPREPGLCTWQSLRSQIA | |
| 252 | TIMP | | MAPFEPLASGILLLLWLIAPSRACTCVPPHPQTAFCNS | Recombinant, other |
| | | | DLVIRAKFVGTPEVNQTTLYQRYEIKMTKMYKGFQA | Antiarthritic, other |
| | | | LGDAADIRFVYTPAMESVCGYFHRSHNRSEEFLIAGK | Stomatological |
| | | | LQDGLLHITTCSFVAPWNSLSLAQRRGFTKTYTVGCE | |
| | | | ECTVFPCLSIPCKLQSGTHCLWTDQLLQGSEKGFQSRH | |
| | | | LACLPREPGLCTWQSLRSQIA | |
| 253 | tendamistat | | Asp-Thr-Thr-Val-Ser-Glu-Pro-Ala-Pro-Ser-Cys-Val-Thr- | Antidiabetic |
| | | | Leu-Tyr-Gln-Ser-Trp-Arg-Tyr-Ser-Gln-Ala-Asp-Asn-Gly- | |
| | | | Cys-Ala-Gln-Thr-Val-Thr-Val-Lys-Val-Val-Tyr-Glu-Asp- | |
| | | | Asp-Thr-Glu-Gly-Leu-Cys-Tyr-Ala-Val-Ala-Pro-Gly-Gln- | |
| | | | Ile-Thr-Thr-Val-Gly-Asp-Gly-Tyr-Ile-Gly-Ser-His-Gly-His- | |
| | | | Ala-Arg-Tyr-Leu-Ala-Arg-Cys-Leu | |
| 254 | thymosin β4 | | gi\|11056061\|ref\|NP_066932.1\| thymosin, beta 4 [Homo | Vulnerary |
| | | | sapiens] | Ophthalmological |
| | | | MSDKPDMAEIEKFDKSKLKKTETQEKNPLPSKETIEQE | Symptomatic antidiabetic |
| | | | KQAGES | Dermatological |
| | | | | Cardiovascular |
| | | | | Septic shock treatment |
| | | | | Antiasthma |
| 255 | urodilatin | | Thr-Ala-Pro-Arg-Ser-Leu-Arg-Arg-Ser-Ser-Cys-Phe-Gly- | Cardiostimulant |
| | | | Gly-Arg-Met-Asp-Arg-Ile-Gly-Ala-Gln-Ser-Gly-Leu-Gly- | Urological |
| | | | Cys-Asn-Ser-Phe-Arg-Tyr | Antiasthma |
| 256 | Pharmaprojects | | Gly-Ser-Arg-Ala-His-Ser-Ser-His-Leu-Lys | Anticancer, other |
| | No. 6236 | | | Antiarrhythmic |
| | | | | Antiparkinsonian |
| | | | | Cognition enhancer |
| | | | | Neuroprotective |
| 257 | ANUP-1 | | Glu-Leu-Lys-Cys-Tyr-Thr-Cys-Lys-Glu-Pro-Met-Thr-Ser- | Anticancer, other |
| | | | Ala-Ala-Cys | |
| 258 | DMI-4983 | | Asp-Ala-His-Lys | Cardiovascular |
| 460 | Glypromate | | Gly-Pro-Glu | Neuroprotective |
| 259 | CD-NP | | Lys Met Val Gln Gly Ser Gly Cys Phe Gly Arg Lys Met Asp | Cardiostimulant |
| | | | Ile Ser Ser Ser Ser Gly Leu Gly Cys Pro Ser Leu Arg Asp | |
| | | | Pro Arg Pro Asn Ala Pro Ser Thr Ser Ala | |
| 260 | Kisspeptin-54 | | GTSLSPPPESSGSPQQPGLSAPHSRQIPAPQGAVLVQRE | Cancer metastasis, |
| | | | KDLPNYNWNSFGLRF-NH2 | angiogenesis |
| 261 | Kisspeptin-14 | | DLPNYNWNSFGLRF-NH2 | Cancer metastasis, |
| | | | | angiogenesis |
| 262 | Kisspeptin-13 | | LPNYNWNSFGLRF-NH2 | Cancer metastasis, |
| | | | | angiogenesis |
| 263 | Kisspeptin-10 | | YNWNSFGLRF-NH2 | Cancer metastasis, |
| | | | | angiogenesis |
| 264 | Ziconotide | | CKGKGAKCSRLMYDCCTGSCRSGKC | |
| 461 | Biphalin | | Tyr-D-Ala-Gly-Phe-NH—NH-Phe-Gly-D-Ala-Tyr | |
| 39 | Nesiritide | Brain | SPKMVQGSGCFGRKMDRISSSSGLGCKVLRRH | |
| | | Netriuritic | | |
| | | peptide | | |
| | | (BNP) | | |
| 40 | CD-NP | | GLSKGCFGLKLDRIGSMSGLGCPSLRDPRPNAPSTSA | |
| 265 | Protegrin-1 | Cytolytic | RGGRLCYCRRRFCVCVGR-NH2 | antibiotic |
| 266 | V681 | | Ac-KWKSFLKTFKSAVKTVLHTALKAISS-NH2 | |
| 462 | V681 (V13A_{D}) | | Ac-KWKSFLKTFKSA(AD)KTVLHTALKAISS-NH2 | ‘(AD)’ discloses the D- |
| | | | | configuration of Alanine |
| 267 | V681 des Al2 | | KWKSFLKTFKSVKTVLHTALKAISS | |
| 268 | V681 V13K | | KWKSFLKTFKSAKKTVLHTALKAISS | |
| 269 | V681 V13K, | | KWKSFLKTFKSAKKKVLHTALKAISS | |
| | T15K | | | |
| 270 | GLP-2 | GLP | HADGSFSDEMNTILDNLAARDFINWLIQTKITD | |
| 271 | GLP-2 (A2G) | GLP | HGDGSFSDEMNTILDNLAARDFINWLIQTKITD | |
| 272 | GLP-2 | GLP | HGDGSFSDEMNTILDNLAARDFINWLIQTKITDC | |
| | (A2G/C34) | | | |
| 273 | AOD-9604 | Human | LRIVQCASVEGSCGFY | Musculoskeletal, COPD, |
| | | Growth | | Hypnotic/Sedative, |
| | | Hormone | | Immunostimulant, |
| | | | | Antidiabetic, Anabolic, |
| | | | | Symptomatic antidiabetic, |
| | | | | Vulnerary |
| 274 | Ac-AOD- | Human | Ac-LRIVQCAKVEGSCGFY | Musculoskeletal, COPD, |
| | 9604(S8K) | Growth | | Hypnotic/Sedative, |
| | | Hormone | | Immunostimulant, |
| | | | | Antidiabetic, Anabolic, |
| | | | | Symptomatic antidiabetic, |
| | | | | Vulnerary |
| 275 | Ac-AOD- | Human | Ac-LRIVQCASVEGSCGFYK | Musculoskeletal, COPD, |
| | 9604(K17) | Growth | | Hypnotic/Sedative, |
| | | Hormone | | Immunostimulant, |
| | | | | Antidiabetic, Anabolic, |
| | | | | Symptomatic antidiabetic, |
| | | | | Vulnerary |
| 276 | C-peptide | Insulin | EAEDLQVGQVELGGGPGAGSLQPLALEGSLQ | |
| 463 | CR845 | Opioids | 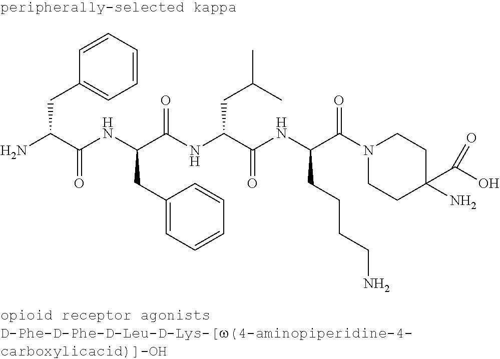 | acute and chronic pain of visceral, inflammatory and neuropathic origin, and for the treatment of pruritis (itch) |
| 277 | Protegrin-2 | Cytolytic | RGGRLCYCRRRFCICV | antibiotic |
| 278 | Protegrin-3 | Cytolytic | RGGGLCYCRRRFCVCVGRG | antibiotic |
| 279 | Protegrin-4 | Cytolytic | RGGRLCYCRGWICFCVGRG | antibiotic |
| 280 | Protegrin-5 | Cytolytic | RGGRLCYCRPRFCVCVGRG | antibiotic |
| 281 | Preprotegrin | Cytolytic | METQRASLCLGRWSLWLLLLGLVVPSASAQALSYRE | antibiotic |
| | | | AVLRAVDRLNEQSSEANLYRLLELDQPPKADEDPGT | |
| | | | PKPVSFTVKETVCPRPTRQPPELCDFKENGRVKQCV | |
| | | | GTVTLDQIKDPLDITCNEVQGVRGGRLCYCRPRFCV | |
| | | | CVGRG | |
| 248 | Oxyntomodulin | | HSQGTFTSDYSKYLDSRRAQDFVQWLMNTKRNRNNI | |
| | | | A | |
| 276 | C-peptide | | EAEDLQVGQVELGGGPGAGSLQPLALEGSLQ | |
| 282 | C-peptide | | EGSLC | |
| | mutant | | | |
| 283 | Human Opioid | Enkephalin | Tyr-Gly-Gly-Phe-Met | |
| | Growth Factor | | | |
| 284 | cholecystokinin | | RDY(S03—)TGW(Nle)DF | |
| 285 | Dynorphin A | | YGGFLRRIRPKLK | |
| | (1-13) | | | |
| 464 | Pralmorelin | | D-Ala-D-2-Nal-Ala-Trp-D-Phe-Lys-NH₂ | |
| | (GHRFA) | | | |
| 286 | Aniritide | | RSSCFGGRMDRIGAQSGLGCNSFRY | |
| 287 | Vessel dilator | | EVVPPQVLSDPNEEAGAALSPLPEVPPWTGEVSPAQR | |
| | proANP31-67 | | | |
| 465 | Peptide G | | Arg-Pro-Lys-Pro-Gln-Arg-D-Trp-MePhe-D-Trp-Leu-Met | |
| 466 | Tiplimotide | | D-Ala-lys-pro-val-val-his-leu-phe-ala-asp-ile- | |
| | | | val-thr-pro-arg-thr-pro | |
| 288 | Desirudin (63- | | VVYTDCTESGQNLCLCEGSNVCGQGNKCILGSDGEK | |
| | desulfohirudin) | | NQCVTGEGTPKPQSHNDGDFEEIPEEYLQ | |
| 467 | Examorelin | | His-DTrp(2-Me)-Ala-Trp-DPhe-Lys-NH2 | |
| 172 | Terlipressin | Vesopressin | Gly-Gly-Gly-c[Cys-Tyr-Phe-Gln-Asn-Cys]-Pro-Lys-Gly- | |
| | | | NH2 | |
| 289 | Osteogenic | | ALKRQGRTLYGFGG | |
| | Growth Factor | | | |
| | (WT) | | | |
| 290 | Osteogenic | | YGFGG | |
| | Growth Factor | | | |
| | (10-14) | | | |
| 291 | Myelin Basic | | Ac-ASQKRPSQRHG | |
| | Protein peptide | | | |
| 292 | Myelin Basic | | Ac-ASQYRPSQRHG | |
| | Protein peptide | | | |
| | Ac1-11[4Y] | | | |
| 293 | Gonadorelin | | pyroGlu-His-Trp-Ser-Tyr-Gly-Leu-Arg-Pro-Gly CONH2 | |
| | (24-33) | | | |
| 468 | Bremelanotide | Alpha-MSH | Ac-Nle-cyclo[Asp-His-D-Phe-Arg-Trp-Lys]-OH | |
| 294 | Islet | | GLHDPSHGTLPNGSG | Diabetes |
| | Neogenesis | | | |
| | associated | | | |
| | peptide | | | |
| | (INGAP) | | | |
| 295 | Urocortin II | | IVLSLDVPIGLLQILLEQARARAAREQATTNARILARV | |
| | | | GHC | |
| 296 | A6 (anti- | | CH3CO—NH2-KPSSPPEE-CONH2 | |
| | angiogenic | | | |
| | peptide) | | | |
| 297 | Obestatin | | H-Phe-Asn-Ala-Pro-Phe-Asp-Val-Gly-Ile-Lys-Leu-Ser-Gly- | |
| | | | Val-Gln-Tyr-Gln-Gln-His-Ser-Gln-Ala-Leu-NH2 | |
| 298 | ITF-1697 | | Gly-Lys(Et)-Pro-Arg | |
| 299 | CNP (C-type | | GLSKGCFGLKLDRIGSMSGLGC | |
| | netriuretic | | | |
| | peptide | | | |
| 300 | Osteocalcin | | YLYQWLGAPVPYPDPLEPRREVCELNPDCDELADHIG | Diabetes |
| | | | FQEAYRRFYGPV | |
| 301 | | | EAEDLQVGQVELGGGPGAGCLQPLALEGSLQ | |
| 469 | D4F-APO1 | | Ac-D-W-F-K-A-F-Y-D-K-V-A-E-K-F-K-E-A-F-NH₂ | |
| | mimetic peptide | | | |

The therapeutic peptides may be, or may be modified to be, linear, branched, or cyclic, with our without branching.

As discussed in more detail below, the therapeutic peptides may optionally be modified or protected with a variety of functional groups or protecting groups, including amino terminus protecting groups and/or carboxy terminus protecting groups. Protecting groups, and the manner in which they are introduced and removed are described, for example, in “Protective Groups in Organic Chemistry,” Plenum Press, London, N.Y. 1973; and Greene et al., “PROTECTIVE GROUPS IN ORGANIC SYNTHESIS” 3^{rd} Edition, John Wiley and Sons, Inc., New York, 1999. Numerous protecting groups are known in the art. An illustrative, non-limiting list of protecting groups includes methyl, formyl, ethyl, acetyl, t-butyl, anisyl, benzyl, trifluoroacetyl, N-hydroxysuccinimide, t-butoxycarbonyl, benzoyl, 4-methylbenzyl, thioanizyl, thiocresyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl, triphenylmethyl, and 2,2,5,7,8-pentamethyl-chroman-6-sulphonyl. For discussions of various different types of amino- and carboxy-protecting groups, see, for example, U.S. Pat. No. 5,221,736 (issued Jun. 22, 1993); U.S. Pat. No. 5,256,549 (issued Oct. 26, 1993); U.S. Pat. No. 5,049,656 (issued Sep. 17, 1991); and U.S. Pat. No. 5,521,184 (issued May 28, 1996).

The therapeutic peptides contain, or may be modified to contain, functional groups to which a water-soluble polymer may be attached, either directly or through a spacer moiety or linker. Functional groups include, but are not limited to, the N-terminus of the therapeutic peptide, the C-terminus of the therapeutic peptide, and any functional groups on the side chain of an amino acid, e.g., lysine, cysteine, histidine, aspartic acid, glutamic acid, tyrosine, arginine, serine, methionine, and threonine, present in the therapeutic peptide.

The therapeutic peptides can be prepared by any means known in the art, including non-recombinant and recombinant methods, or they may, in some instances, be commercially available. Chemical or non-recombinant methods include, but are not limited to, solid phase peptide synthesis (SPPS), solution phase peptide synthesis, native chemical ligation, intein-mediated protein ligation, and chemical ligation, or a combination thereof. In a preferred embodiment, the therapeutic peptides are synthesized using standard SPPS, either manually or by using commercially available automated SPPS synthesizers.

SPPS has been known in the art since the early 1960's (Merrifield, R. B., J. Am. Chem. Soc., 85:2149-2154 (1963)), and is widely employed (see also, Bodanszky, Principles of Peptide Synthesis, Springer-Verlag, Heidelberg (1984)). There are several known variations on the general approach (see, for example, “Peptide Synthesis, Structures, and Applications” ©1995 by Academic Press, Chapter 3; and White (2003) Fmoc Solid Phase Peptide Synthesis, A practical Approach, Oxford University Press, Oxford). Very briefly, in solid phase peptide synthesis, the desired C-terminal amino acid residue is coupled to a solid support. The subsequent amino acid to be added to the peptide chain is protected on its amino terminus with Boc, Fmoc, or other suitable protecting group, and its carboxy terminus is activated with a standard coupling reagent. The free amino terminus of the support-bound amino acid is allowed to react with the carboxy-terminus of the subsequent amino acid, coupling the two amino acids. The amino terminus of the growing peptide chain is deprotected, and the process is repeated until the desired polypeptide is completed. Side chain protecting groups may be utilized as needed.

Alternatively, the therapeutic peptides may be prepared recombinantly. Exemplary recombinant methods used to prepare therapeutic peptides include the following, among others, as will be apparent to one skilled in the art. Typically, a therapeutic peptide as defined and/or described herein is prepared by constructing the nucleic acid encoding the desired peptide or fragment, cloning the nucleic acid into an expression vector, transforming a host cell (e.g., plant, bacteria such as Escherichia coli, yeast such as Saccharomyces cerevisiae, or mammalian cell such as Chinese hamster ovary cell or baby hamster kidney cell), and expressing the nucleic acid to produce the desired peptide or fragment. The expression can occur via exogenous expression or via endogenous expression (when the host cell naturally contains the desired genetic coding). Methods for producing and expressing recombinant polypeptides in vitro and in prokaryotic and eukaryotic host cells are known to those of ordinary skill in the art. See, for example, U.S. Pat. No. 4,868,122; and Sambrook et al., Molecular Cloning—A Laboratory Manual, Cold Spring Harbor Laboratory Press (1989).

To facilitate identification and purification of the recombinant peptide, nucleic acid sequences that encode an epitope tag or other affinity binding sequence can be inserted or added in-frame with the coding sequence, thereby producing a fusion peptide comprised of the desired therapeutic peptide and a peptide suited for binding. Fusion peptides can be identified and purified by first running a mixture containing the fusion peptide through an affinity column bearing binding moieties (e.g., antibodies) directed against the epitope tag or other binding sequence in the fusion peptide, thereby binding the fusion peptide within the column. Thereafter, the fusion peptide can be recovered by washing the column with the appropriate solution (e.g., acid) to release the bound fusion peptide. Optionally, the tag may subsequently be removed by techniques known in the art. The recombinant peptide can also be identified and purified by lysing the host cells, separating the peptide, e.g., by size exclusion chromatography, and collecting the peptide. These and other methods for identifying and purifying recombinant peptides are known to those of ordinary skill in the art.

### Related Peptides

It will be appreciated and understood by one of skill in the art that certain modifications can be made to the therapeutic peptides defined and/or disclosed herein that do not alter, or only partially abrogate, the properties and activities of these therapeutic peptides. In some instances, modifications may be made that result in an increase in therapeutic activities. Additionally, modifications may be made that increase certain biological and chemical properties of the therapeutic peptides in a beneficial way, e.g., increased in vivo half-life, increased stability, decreased susceptibility to proteolytic cleavage, etc. Thus, in the spirit and scope of the invention, the term “therapeutic peptide” is used herein in a manner to include not only the therapeutic peptides defined and/or disclosed herein, but also related peptides, i.e., peptides that contain one or more modifications relative to the therapeutic peptides defined and/or disclosed herein, wherein the modification(s) do not alter, only partially abrogate, or increase the therapeutic activities as compared to the parent peptide.

Related peptides include, but are not limited to, fragments of therapeutic peptides, therapeutic peptide variants, and therapeutic peptide derivatives. Related peptides also include any and all combinations of these modifications. In a non-limiting example, a related peptide may be a fragment of a therapeutic peptide as disclosed herein having one or more amino acid substitutions. Thus, it will be understood that any reference to a particular type of related peptide is not limited to a therapeutic peptide having only that particular modification, but rather encompasses a therapeutic peptide having that particular modification and optionally any other modification.

Related peptides may be prepared by action on a parent peptide or a parent protein (e.g., proteolytic digestion to generate fragments) or through de novo preparation (e.g., solid phase synthesis of a peptide having a conservative amino acid substitution relative to the parent peptide). Related peptides may arise by natural processes (e.g., processing and other post-translational modifications) or may be made by chemical modification techniques. Such modifications are known to those of skill in the art.

A related peptide may have a single alteration or multiple alterations relative to the parent peptide. Where multiple alterations are present, the alterations may be of the same type or a given related peptide may contain different types of modifications. Furthermore, modifications can occur anywhere in a polypeptide, including the peptide backbone, the amino acid side-chains, and the N- or C-termini.

As previously noted, related peptides include fragments of the therapeutic peptides defined and/or disclosed herein, wherein the fragment retains some of or all of at least one therapeutic activity of the parent peptide. The fragment may also exhibit an increase in at least one therapeutic activity of the parent peptide. In certain embodiments of the invention, therapeutic peptides include related peptides having at least 2, 3, 4, 5, 6, 7, 8, 9, 10, 11, 12, 13, 14, 15, 20, 25, 30, 35, 40, 45, 50, 60, 70, 80, 90, or 100 contiguous amino acid residues, or more than 125 contiguous amino acid residues, of any of the therapeutic peptides disclosed, herein, including in Table A. In other embodiments of the invention, therapeutic peptides include related peptides having 0, 1, 2, 3, 4, 5, 6, 7, 8, 9, 10, 15, 20, 25, 30, 35, 40, 45, or 50 amino acid residues deleted from the N-terminus and/or having 0, 1, 2, 3, 4, 5, 6, 7, 8, 9, 10, 15, 20, 25, 30, 35, 40, 45, or 50 amino acid residues deleted from the C-terminus of any of the therapeutic peptides disclosed herein, including in Table A.

Related peptides also include variants of the therapeutic peptides defined and/or disclosed herein, wherein the variant retains some of or all of at least one therapeutic activity of the parent peptide. The variant may also exhibit an increase in at least one therapeutic activity of the parent peptide. In certain embodiments of the invention, therapeutic peptides include variants having 1, 2, 3, 4, 5, 6, 7, 8, 9, 10, 15, 20, 25, 30, 35, 40, 45, or 50 conservative and/or non-conservative amino acid substitutions relative to the therapeutic peptides disclosed herein, including in Table A. Desired amino acid substitutions, whether conservative or non-conservative, can be determined by those skilled in the art.

In certain embodiments of the invention, therapeutic peptides include variants having conservative amino substitutions; these substitutions will produce a therapeutic peptide having functional and chemical characteristics similar to those of the parent peptide. In other embodiments, therapeutic peptides include variants having non-conservative amino substitutions; these substitutions will produce a therapeutic peptide having functional and chemical characteristics that may differ substantially from those of the parent peptide. In certain embodiments of the invention, therapeutic peptide variants have both conservative and non-conservative amino acid substitutions. In other embodiments, each amino acid residue may be substituted with alanine.

Natural amino acids may be divided into classes based on common side chain properties: nonpolar (Gly, Ala, Val, Leu, Ile, Met); polar neutral (Cys, Ser, Thr, Pro, Asn, Gln); acidic (Asp, Glu); basic (His, Lys, Arg); and aromatic (Trp, Tyr, Phe). By way of example, non-conservative amino acid substitutions may involve the substitution of an amino acid of one class for that of another, and may be introduced in regions of the peptide not critical for therapeutic activity.

Preferably, amino acid substitutions are conservative. Conservative amino acid substitutions may involve the substitution of an amino acid of one class for that of the same class. Conservative amino acid substitutions may also encompass non-natural amino acid residues, including peptidomimetics and other atypical forms of amino acid moieties, and may be incorporated through chemical peptide synthesis,

Amino acid substitutions may be made with consideration to the hydropathic index of amino acids. The importance of the hydropathic amino acid index in conferring interactive biological function on a protein is generally understood in the art (Kyte et al., 1982, J. Mol. Biol. 157:105-31). Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); praline (−1.6); histidine (−3.2); glutamate (−3.5); glutamine (−3.5); aspartate (−3.5); asparagine (−3.5); lysine (−3.9); and arginine (−4.5).

It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. The greatest local average hydrophilicity of a protein, as governed by the hydrophilicity of its adjacent amino acids, correlates with its biological properties. According to U.S. Pat. No. 4,554,101, incorporated herein by reference, the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred.

In certain embodiments of the invention, therapeutic peptides include variants having 1, 2, 3, 4, 5, 6, 7, 8, 9, 10, 15, 20, 25, 30, 35, 40, 45, or 50 amino acid deletions relative to the therapeutic peptides disclosed herein, including in Table A. The deleted amino acid(s) may be at the N- or C-terminus of the peptide, at both termini, at an internal location or locations within the peptide, or both internally and at one or both termini. Where the variant has more than one amino acid deletion, the deletions may be of contiguous amino acids or of amino acids at different locations within the primary amino acid sequence of the parent peptide.

In other embodiments of the invention, therapeutic peptides include variants having 1, 2, 3, 4, 5, 6, 7, 8, 9, 10, 15, 20, 25, 30, 35, 40, 45, or 50 amino acid additions relative to the therapeutic peptides disclosed herein, including in Table A. The added amino acid(s) may be at the N- or C-terminus of the peptide, at both termini, at an internal location or locations within the peptide, or both internally and at one or both termini. Where the variant has more than one amino acid addition, the amino acids may be added contiguously, or the amino acids may be added at different locations within the primary amino acid sequence of the parent peptide.

Addition variants also include fusion peptides. Fusions can be made either at the N-terminus or at the C-terminus of the therapeutic peptides disclosed herein, including in Table A. In certain embodiments, the fusion peptides have 1, 2, 3, 4, 5, 6, 7, 8, 9, 10, 15, 20, 25, 30, 35, 40, 45, or 50 amino acid additions relative to the therapeutic peptides disclosed herein, including in Table A. Fusions may be attached directly to the therapeutic peptide with no connector molecule or may be through a connector molecule. As used in this context, a connector molecule may be an atom or a collection of atoms optionally used to link a therapeutic peptide to another peptide. Alternatively, the connector may be an amino acid sequence designed for cleavage by a protease to allow for the separation of the fused peptides.

The therapeutic peptides of the invention may be fused to peptides designed to improve certain qualities of the therapeutic peptide, such as therapeutic activity, circulation time, or reduced aggregation. Therapeutic peptides may be fused to an immunologically active domain, e.g., an antibody epitope, to facilitate purification of the peptide, or to increase the in vivo half-life of the peptide. Additionally, therapeutic peptides may be fused to known functional domains, cellular localization sequences, or peptide permeant motifs known to improve membrane transfer properties.

In certain embodiments of the invention, therapeutic peptides also include variants incorporating one or more non-natural amino acids, amino acid analogs, and peptidomimetics. Thus, the present invention encompasses compounds structurally similar to the therapeutic peptides defined and/or disclosed herein, which are formulated to mimic the key portions of the therapeutic peptides of the present invention. Such compounds may be used in the same manner as the therapeutic peptides of the invention. Certain mimetics that mimic elements of protein secondary and tertiary structure have been previously described. Johnson et al., Biotechnology and Pharmacy, Pezzuto et al. (Eds.), Chapman and Hall, NY, 1993. The underlying rationale behind the use of peptide mimetics is that the peptide backbone of proteins exists chiefly to orient amino acid side chains in such a way as to facilitate molecular interactions. A peptide mimetic is thus designed to permit molecular interactions similar to the parent peptide. Mimetics can be constructed to achieve a similar spatial orientation of the essential elements of the amino acid side chains. Methods for generating specific structures have been disclosed in the art. For example, U.S. Pat. Nos. 5,446,128; 5,710,245; 5,840,833; 5,859,184; 5,440,013; 5,618,914; 5,670,155; 5,475,085; 5,929,237; 5,672,681; and 5,674,976, the contents of which are hereby incorporated by reference, all disclose peptidomimetic structures that may have improved properties over the parent peptide, for example, they may be conformationally restricted, be more thermally stable, exhibit increased resistance to degradation, etc.

In another embodiment, related peptides comprise or consist of a peptide sequence that is at least 70% identical to any of the therapeutic peptides disclosed herein, including in Table A. In additional embodiments, related peptides are at least 75% identical, at least 80% identical, at least 85% identical, 90% identical, at least 91% identical, at least 92% identical, 93% identical, at least 94% identical, at least 95% identical, 96% identical, at least 97% identical, at least 98% identical, or at least 99% identical to any of the therapeutic peptides disclosed herein, including in Table A.

Sequence identity (also known as % homology) of related polypeptides can be readily calculated by known methods. Such methods include, but are not limited to those described in Computational Molecular Biology (A. M. Lesk, ed., Oxford University Press 1988); Biocomputing: Informatics and Genome Projects (D. W. Smith, ed., Academic Press 1993); Computer Analysis of Sequence Data (Part 1, A. M. Griffin and H. G. Griffin, eds., Humana Press 1994); G. von Heinle, Sequence Analysis in Molecular Biology (Academic Press 1987); Sequence Analysis Primer (M. Gribskov and J. Devereux, eds., M. Stockton Press 1991); and Carillo et al., 1988, SIAM J. Applied Math., 48:1073.

Preferred methods to determine sequence identity and/or similarity are designed to give the largest match between the sequences tested. Methods to determine sequence identity are described in publicly available computer programs. Preferred computer program methods to determine identity and similarity between two sequences include, but are not limited to, the GCG program package, including GAP (Devereux et al., 1984, Nucleic Acids Res. 12:387; Genetics Computer Group, University of Wisconsin, Madison, Wis.), BLASTP, BLASTN, and FASTA (Altschul et al., 1990, J. Mol. Biol. 215:403-10). The BLASTX program is publicly available from the National Center for Biotechnology Information (NCBI) and other sources (Altschul et al., BLAST Manual (NCB NLM NIH, Bethesda, Md.); Altschul et al., 1990, supra). The Smith Waterman algorithm may also be used to determine identity.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span,” as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 0.1× the gap opening penalty), as well as a comparison matrix such as PAM 250 or BLOSUM 62 are used in conjunction with the algorithm. A standard comparison matrix is also used by the algorithm (see Dayhoff et al., 5 Atlas of Protein Sequence and Structure (Supp. 3 1978) (PAM250 comparison matrix); Henikoff et al., 1992, Proc. Natl. Acad. Sci. USA 89:10915-19 (BLOSUM 62 comparison matrix)). The particular choices to be made with regard to algorithms, gap opening penalties, gap extension penalties, comparison matrices, and thresholds of similarity will be readily apparent to those of skill in the art and will depend on the specific comparison to be made.

Related peptides also include derivatives of the therapeutic peptides defined and/or disclosed herein, wherein the variant retains some of or all of at least one therapeutic activity of the parent peptide. The derivative may also exhibit an increase in at least one therapeutic activity of the parent peptide. Chemical alterations of therapeutic peptide derivatives include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, T. E. Creighton, Proteins, Structure and Molecular Properties, 2nd ed., W.H. Freeman and Company, New York (1993); Posttranslational Covalent Modification of Proteins, B. C. Johnson, ed., Academic Press, New York, pgs. 1-12 (1983); Seifter et al., Meth. Enzymol, 182:626-46 (1990); Rattan et al., Ann. N.Y. Acad. Sci. 663:48-62, 1992).

Therapeutic peptide derivatives also include molecules formed by the deletion of one or more chemical groups from the parent peptide. Methods for preparing chemically modified derivatives of the therapeutic peptides defined and/or disclosed herein are known to one of skill in the art.

In some embodiments of the invention, the therapeutic peptides may be modified with one or more methyl or other lower alkyl groups at one or more positions of the therapeutic peptide sequence. Examples of such groups include methyl, ethyl, propyl, isopropyl, butyl, isobutyl, pentyl, etc. In certain preferred embodiments, arginine, lysine, and histidine residues of the therapeutic peptides are modified with methyl or other lower alkyl groups.

In other embodiments of the invention, the therapeutic peptides may be modified with one or more glycoside moieties relative to the parent peptide. Although any glycoside can be used, in certain preferred embodiments the therapeutic peptide is modified by introduction of a monosaccharide, a disaccharide, or a trisaccharide or it may contain a glycosylation sequence found in natural peptides or proteins in any mammal. The saccharide may be introduced at any position, and more than one glycoside may be introduced. Glycosylation may occur on a naturally occurring amino acid residue in the therapeutic peptide, or alternatively, an amino acid may be substituted with another for modification with the saccharide.

Glycosylated therapeutic peptides may be prepared using conventional Fmoc chemistry and solid phase peptide synthesis techniques, e.g., on resin, where the desired protected glycoamino acids are prepared prior to peptide synthesis and then introduced into the peptide chain at the desired position during peptide synthesis. Thus, the therapeutic peptide polymer conjugates may be conjugated in vitro. The glycosylation may occur before deprotection. Preparation of aminoacid glycosides is described in U.S. Pat. No. 5,767,254, WO 2005/097158, and Doores, K., et al., Chem. Commun., 1401-1403, 2006, which are incorporated herein by reference in their entireties. For example, alpha and beta selective glycosylations of serine and threonine residues are carried out using the Koenigs-Knorr reaction and Lemieux's in situ anomerization methodology with Schiff base intermediates. Deprotection of the Schiff base glycoside is then carried out using mildly acidic conditions or hydrogenolysis. A composition, comprising a glycosylated therapeutic peptide conjugate made by stepwise solid phase peptide synthesis involving contacting a growing peptide chain with protected amino acids in a stepwise manner, wherein at least one of the protected amino acids is glycosylated, followed by water-soluble polymer conjugation, may have a purity of at least 95%, such as at least 97%, or at least 98%, of a single species of the glycosylated and conjugated therapeutic peptide.

Monosaccharides that may by used for introduction at one or more amino acid residues of the therapeutic peptides defined and/or disclosed herein include glucose (dextrose), fructose, galactose, and ribose. Additional monosaccharides suitable for use include glyceraldehydes, dihydroxyacetone, erythrose, threose, erythrulose, arabinose, lyxose, xylose, ribulose, xylulose, allose, altrose, mannose, N-Acetylneuraminic acid, fucose, N-Acetylgalactosamine, and N-Acetylglucosamine, as well as others. Glycosides, such as mono-, di-, and trisaccharides for use in modifying a therapeutic peptide, may be naturally occurring or may be synthetic. Disaccharides that may by used for introduction at one or more amino acid residues of the therapeutic peptides defined and/or disclosed herein include sucrose, lactose, maltose, trehalose, melibiose, and cellobiose, among others. Trisaccharides include acarbose, raffinose, and melezitose.

In further embodiments of the invention, the therapeutic peptides defined and/or disclosed herein may be chemically coupled to biotin. The biotin/therapeutic peptide molecules can then bind to avidin.

As previously noted, modifications may be made to the therapeutic peptides defined and/or disclosed herein that do not alter, or only partially abrogate, the properties and activities of these therapeutic peptides. In some instances, modifications may be made that result in an increase in therapeutic activity. Thus, included in the scope of the invention are modifications to the therapeutic peptides disclosed herein, including in Table A, that retain at least 1%, at least 5%, at least 10%, at least 20%, at least 30%, at least 40%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, and any range derivable therein, such as, for example, at least 70% to at least 80%, and more preferably at least 81% to at least 90%; or even more preferably, between at least 91% and at least 99%, of the therapeutic activity relative to the unmodified therapeutic peptide. Also included in the scope of the invention are modified therapeutic peptides disclosed herein, including in Table A, that have greater than 100%, greater than 110%, greater than 125%, greater than 150%, greater than 200%, or greater than 300%, or greater than 10-fold or greater than 100-fold, and any range derivable therein, of the therapeutic activity relative to the unmodified therapeutic peptide.

The level of therapeutic activity of a given therapeutic peptide, or a modified therapeutic peptide, may be determined by any suitable in vivo or in vitro assay. For example, therapeutic activity may be assayed in cell culture, or by clinical evaluation, ECₛₒ assays, IC₅₀ assays, or dose response curves. In vitro or cell culture assays, for example, are commonly available and known to one of skill in the art for many therapeutic peptides as disclosed herein, including in Table A. It will be understood by one of skill in the art that the percent activity of a modified therapeutic peptide relative to its unmodified parent can be readily ascertained through a comparison of the activity of each as determined through the assays disclosed herein or as known to one of skill in the art.

One of skill in the art will be able to determine appropriate modifications to the therapeutic peptides defined and/or disclosed herein, including those disclosed herein, including in Table A. For identifying suitable areas of the therapeutic peptides that may be changed without abrogating their therapeutic activities, one of skill in the art may target areas not believed to be essential for activity. For example, when similar peptides with comparable activities exist from the same species or across other species, one of skill in the art may compare those amino acid sequences to identify residues that are conserved among similar peptides. It will be understood that changes in areas of a therapeutic peptide that are not conserved relative to similar peptides would be less likely to adversely affect the therapeutic activity. One skilled in the art would also know that, even in relatively conserved regions, one may substitute chemically similar amino acids while retaining therapeutic activity. Therefore, even areas that may be important for biological activity and/or for structure may be subject to amino acid substitutions without destroying the therapeutic activity or without adversely affecting the peptide structure.

Additionally, as appropriate, one of skill in the art can review structure-function studies identifying residues in similar peptides that are important for activity or structure. In view of such a comparison, one can predict the importance of an amino acid residue in a therapeutic peptide that corresponds to an amino acid residue that is important for activity or structure in similar peptides. One of skill in the art may opt for amino acid substitutions within the same class of amino acids for such predicted important amino acid residues of the therapeutic peptides.

Also, as appropriate, one of skill in the art can also analyze the three-dimensional structure and amino acid sequence in relation to that structure in similar peptides. In view of such information, one of skill in the art may predict the alignment of amino acid residues of a therapeutic peptide with respect to its three dimensional structure. One of skill in the art may choose not to make significant changes to amino acid residues predicted to be on the surface of the peptide, since such residues may be involved in important interactions with other molecules. Moreover, one of skill in the art may generate variants containing a single amino acid substitution at each amino acid residue for test purposes. The variants could be screened using therapeutic activity assays known to those with skill in the art. Such variants could be used to gather information about suitable modifications. For example, where a change to a particular amino acid residue resulted in abrogated, undesirably reduced, or unsuitable activity, variants with such a modification would be avoided. In other words, based on information gathered from routine experimentation, one of skill in the art can readily determine the amino acids where further modifications should be avoided either alone or in combination with other modifications.

One of skill in the art may also select suitable modifications based on secondary structure predication. A number of scientific publications have been devoted to the prediction of secondary structure. See Moult, 1996, Curr. Opin. Biotechnol. 7:422-27; Chou et al., 1974, Biochemistry 13:222-45; Chou et al., 1974, Biochemistry 113:211-22; Chou et al., 1978, Adv. Enzymol. Relat. Areas Mol. Biol. 47:45-48; Chou et al., 1978, Ann. Rev. Biochem. 47:251-276; and Chou et al., 1979, Biophys. J. 26:367-84. Moreover, computer programs are currently available to assist with predicting secondary structure. One method of predicting secondary structure is based upon homology modeling. For example, two peptides or proteins which have a sequence identity of greater than 30%, or similarity greater than 40%, often have similar structural topologies. Recent growth of the protein structural database (PDB, http://www.rcsb.org/pdb/home/home.do) has provided enhanced predictability of secondary, tertiary, and quarternary structure, including the potential number of folds within the structure of a peptide or protein. See Holm et al., 1999, Nucleic Acids Res. 27:244-47. It has been suggested that there are a limited number of folds in a given peptide or protein and that once a critical number of structures have been resolved, structural prediction will become dramatically more accurate (Brenner et al., 1997, Curr. Opin. Struct. Biol. 7:369-76).

Additional methods of predicting secondary structure include “threading” (Jones, 1997, Curr. Opin. Struct. Biol. 7:377-87; Sippl et al., 1996, Structure 4:15-19), “profile analysis” (Bowie et al., 1991, Science, 253:164-70; Gribskov et al., 1990, Methods Enzymol. 183:146-59; Gribskov et al., 1987, Proc. Nat. Acad. Sci. U.S.A. 84:4355-58), and “evolutionary linkage” (See Holm et al., supra, and Brenner et al., supra).

### Therapeutic Peptide Conjugates

As described above, a conjugate of the invention comprises a water-soluble polymer covalently attached (either directly or through a spacer moiety or linker) to a therapeutic peptide. Typically, for any given conjugate, there will be about one to five water-soluble polymers covalently attached to a therapeutic peptide (wherein for each water-soluble polymer, the water-soluble polymer can be attached either directly to the therapeutic peptide or through a spacer moiety).

To elaborate, a therapeutic peptide conjugate of the invention typically has about 1, 2, 3, or 4 water-soluble polymers individually attached to a therapeutic peptide. That is to say, in certain embodiments, a conjugate of the invention will possess about 4 water-soluble polymers individually attached to a therapeutic peptide, or about 3 water-soluble polymers individually attached to a therapeutic peptide, or about 2 water-soluble polymers individually attached to a therapeutic peptide, or about 1 water-soluble polymer attached to a therapeutic peptide. The structure of each of the water-soluble polymers attached to the therapeutic peptide may be the same or different. One therapeutic peptide conjugate in accordance with the invention is one having a water-soluble polymer releasably attached to the therapeutic peptide, particularly at the N-terminus of the therapeutic peptide. Another therapeutic peptide conjugate in accordance with the invention is one having a water-soluble polymer stably attached to the therapeutic peptide, particularly at the N-terminus of the therapeutic peptide. Another therapeutic peptide conjugate is one having a water-soluble polymer releasably attached to the therapeutic peptide, particularly at the C-terminus of the therapeutic peptide. Another therapeutic peptide conjugate in accordance with the invention is one having a water-soluble polymer stably attached to the therapeutic peptide, particularly at the C-terminus of the therapeutic peptide. Other therapeutic peptide conjugates in accordance with the invention are those having a water-soluble polymer releasably or stably attached to an amino acid within the therapeutic peptide. Additional water-soluble polymers may be releasably or stably attached to other sites on the therapeutic peptide, e.g., such as one or more additional sites. For example, a therapeutic peptide conjugate having a water-soluble polymer releasably attached to the N-terminus may additionally possess a water-soluble polymer stably attached to a lysine residue. In one embodiment, one or more amino acids may be inserted, at the N- or C-terminus, or within the peptide to releasably or stably attach a water soluble polymer. One preferred embodiment of the present invention is a mono-therapeutic peptide polymer conjugate, i.e., a therapeutic peptide having one water-soluble polymer covalently attached thereto. In an even more preferred embodiment, the water-soluble polymer is one that is attached to the therapeutic peptide at its N-terminus.

Preferably, a therapeutic peptide polymer conjugate of the invention is absent a metal ion, i.e., the therapeutic peptide is not chelated to a metal ion.

For the therapeutic peptide polymer conjugates described herein, the therapeutic peptide may optionally possess one or more N-methyl substituents. Alternatively, for the therapeutic peptide polymer conjugates described herein, the therapeutic peptide may be glycosylated, e.g., having a mono- or disaccharide, or naturally-occurring amino acid glycosylation covalently attached to one or more sites thereof.

As discussed herein, the compounds of the present invention may be made by various methods and techniques known and available to those skilled in the art.

### Water-Soluble Polymers

A conjugate of the invention comprises a therapeutic peptide attached, stably or releasably, to a water-soluble polymer. The water-soluble polymer is typically hydrophilic, nonpeptidic, and biocompatible. A substance is considered biocompatible if the beneficial effects associated with use of the substance alone or with another substance (e.g., an active agent such a therapeutic peptide) in connection with living tissues (e.g., administration to a patient) outweighs any deleterious effects as evaluated by a clinician, e.g., a physician. A substance is considered nonimmunogenic if the intended use of the substance in vivo does not produce an undesired immune response (e.g., the formation of antibodies) or, if an immune response is produced, that such a response is not deemed clinically significant or important as evaluated by a clinician. Typically, the water-soluble polymer is hydrophilic, biocompatible and nonimmunogenic.

Further the water-soluble polymer is typically characterized as having from 2 to about 300 termini, preferably from 2 to 100 termini, and more preferably from about 2 to 50 termini. Examples of such polymers include, but are not limited to, poly(alkylene glycols) such as polyethylene glycol (PEG), polypropylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and combinations of any of the foregoing, including copolymers and terpolymers thereof.

The water-soluble polymer is not limited to a particular structure and may possess a linear architecture (e.g., alkoxy PEG or bifunctional PEG), or a non-linear architecture, such as branched, forked, multi-armed (e.g., PEGs attached to a polyol core), or dendritic (i.e., having a densely branched structure with numerous end groups). Moreover, the polymer subunits can be organized in any number of different patterns and can be selected, e.g., from homopolymer, alternating copolymer, random copolymer, block copolymer, alternating tripolymer, random tripolymer, and block tripolymer.

One particularly preferred type of water-soluble polymer is a polyalkylene oxide, and in particular, polyethylene glycol (or PEG). Generally, a PEG used to prepare a therapeutic peptide polymer conjugate of the invention is “activated” or reactive. That is to say, the activated PEG (and other activated water-soluble polymers collectively referred to herein as “polymeric reagents”) used to form a therapeutic peptide conjugate comprises an activated functional group suitable for coupling to a desired site or sites on the therapeutic peptide. Thus, a polymeric reagent for use in preparing a therapeutic peptide conjugate includes a functional group for reaction with the therapeutic peptide.

Representative polymeric reagents and methods for conjugating such polymers to an active moiety are known in the art, and are, e.g., described in Harris, J. M. and Zalipsky, S., eds, Poly(ethylene glycol), Chemistry and Biological Applications, ACS, Washington, 1997; Veronese, F., and J .M Harris, eds., Peptide and Protein PEGylation, Advanced Drug Delivery Reviews, 54(4); 453-609 (2002); Zalipsky, S., et al., “Use of Functionalized Poly(Ethylene Glycols) for Modification of Polypeptides” in Polyethylene Glycol Chemistry: Biotechnical and Biomedical Applications, J. M. Harris, ed., Plenus Press, New York (1992); Zalipsky (1995) Advanced Drug Reviews 16:157-182, and in Roberts, et al., Adv. Drug Delivery Reviews, 54, 459-476 (2002).

Additional PEG reagents suitable for use in forming a conjugate of the invention, and methods of conjugation are described in the Pasut. G., et al., Expert Opin. Ther. Patents (2004), 14(5). PEG reagents suitable for use in the present invention also include those available from NOF Corporation, as described generally on the NOF website (http://nofamerica.net/store/). Products listed therein and their chemical structures are expressly incorporated herein by reference. Additional PEGs for use in forming a therapeutic peptide conjugate of the invention include those available from Polypure (Norway) and from QuantaBioDesign LTD (Ohio), where the contents of their online catalogs (2006) with respect to available PEG reagents are expressly incorporated herein by reference. In addition, water soluble polymer reagents useful for preparing peptide conjugates of the invention can be prepared synthetically. Descriptions of the water soluble polymer reagent synthesis can be found in, for example, U.S. Pat. Nos. 5,252,714, 5,650,234, 5,739,208, 5,932,462, 5,629,384, 5,672,662, 5,990,237, 6,448,369, 6,362,254, 6,495,659, 6,413,507, 6,376,604, 6,348,558, 6,602,498, and 7,026,440.

Typically, the weight-average molecular weight of the water-soluble polymer in the conjugate is from about 100 Daltons to about 150,000 Daltons. Exemplary ranges include weight-average molecular weights in the range of from about 250 Daltons to about 80,000 Daltons, from 500 Daltons to about 80,000 Daltons, from about 500 Daltons to about 65,000 Daltons, from about 500 Daltons to about 40,000 Daltons, from about 750 Daltons to about 40,000 Daltons, from about 1000 Daltons to about 30,000 Daltons. In a preferred embodiment, the weight average molecular weight of the water-soluble polymer in the conjugate ranges from about 1000 Daltons to about 10,000 Daltons. In certain other preferred embodiments, the range is from about 1000 Daltons to about 5000 Daltons, from about 5000 Daltons to about 10,000 Daltons, from about 2500 Daltons to about 7500 Daltons, from about 1000 Daltons to about 3000 Daltons, from about 3000 Daltons to about 7000 Daltons, or from about 7000 Daltons to about 10,000 Daltons. In a further preferred embodiment, the weight average molecular weight of the water-soluble polymer in the conjugate ranges from about 20,000 Daltons to about 40,000 Daltons. In other preferred embodiments, the range is from about 20,000 Daltons to about 30,000 Daltons, from about 30,000 Daltons to about 40,000 Daltons, from about 25,000 Daltons to about 35,000 Daltons, from about 20,000 Daltons to about 26,000 Daltons, from about 26,000 Daltons to about 34,000 Daltons, or from about 34,000 Daltons to about 40,000 Daltons.

For any given water-soluble polymer, a molecular weight in one or more of these ranges is typical. Generally, a therapeutic peptide conjugate in accordance with the invention, when intended for subcutaneous or intravenous administration, will comprise a PEG or other suitable water-soluble polymer having a weight average molecular weight of about 20,000 Daltons or greater, while a therapeutic peptide conjugate intended for pulmonary administration will generally, although not necessarily, comprise a PEG polymer having a weight average molecular weight of about 20,000 Daltons or less.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons.

Branched versions of the water-soluble polymer (e.g., a branched 40,000 Dalton water-soluble polymer comprised of two 20,000 Dalton polymers or the like) having a total molecular weight of any of the foregoing can also be used. In one or more particular embodiments, depending upon the other features of the subject therapeutic peptide polymer conjugate, the conjugate is one that does not have one or more attached PEG moieties having a weight-average molecular weight of less than about 6,000 Daltons.

In instances in which the water-soluble polymer is a PEG, the PEG will typically comprise a number of (OCH₂CH₂) monomers. As used herein, the number of repeat units is typically identified by the subscript “n” in, for example, “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. Preferred ranges of n include from about 10 to about 700, and from about 10 to about 1800. For any given polymer in which the molecular weight is known, it is possible to determine the number of repeating units (i.e., “n”) by dividing the total weight-average molecular weight of the polymer by the molecular weight of the repeating monomer.

With regard to the molecular weight of the water-soluble polymer, in one or more embodiments of the invention, depending upon the other features of the particular therapeutic peptide conjugate, the conjugate comprises a therapeutic peptide covalently attached to a water-soluble polymer having a molecular weight greater than about 2,000 Daltons.

A polymer for use in the invention may be end-capped, that is, a polymer having at least one terminus capped with a relatively inert group, such as a lower alkoxy group (i.e., a C₁₋₆ alkoxy group) or a hydroxyl group. One frequently employed end-capped polymer is methoxy-PEG (commonly referred to as mPEG), wherein one terminus of the polymer is a methoxy (—OCH₃) group. The -PEG- symbol used in the foregoing generally represents the following structural unit: —CH₂CH₂O—(CH₂CH₂O)ₙ—CH₂CH₂—, where (n) generally ranges from about zero to about 4,000.

Multi-armed or branched PEG molecules, such as those described in U.S. Pat. No. 5,932,462, are also suitable for use in the present invention. For example, the PEG may be described generally according to the structure:

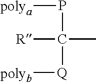

where polyₛ and poly_{b} are PEG backbones (either the same or different), such as methoxy poly(ethylene glycol); R″ is a non-reactive moiety, such as H, methyl or a PEG backbone; and P and Q are non-reactive linkages. In one embodiment, the branched PEG molecule is one that includes a lysine residue, such as the following reactive PEG suitable for use in forming a therapeutic peptide conjugate. Although the branched PEG below is shown with a reactive succinimidyl group, this represents only one of a myriad of reactive functional groups suitable for reacting with a therapeutic peptide.

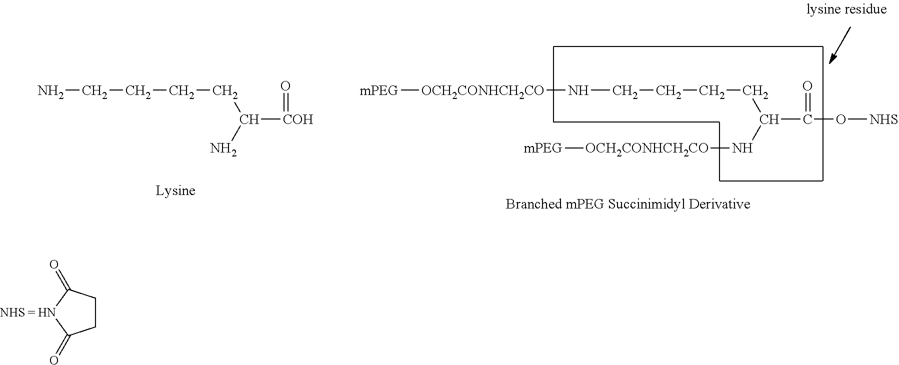

In some instances, the polymeric reagent (as well as the corresponding conjugate prepared from the polymeric reagent) may lack a lysine residue in which the polymeric portions are connected to amine groups of the lysine via a “—OCH₂CONHCH₂CO—” group. In still other instances, the polymeric reagent (as well as the corresponding conjugate prepared from the polymeric reagent) may lack a branched water-soluble polymer that includes a lysine residue (wherein the lysine residue is used to effect branching).

Additional branched-PEGs for use in forming a therapeutic peptide conjugate of the present invention include those described in co-owned U.S. Patent Application Publication No. 2005/0009988. Representative branched polymers described therein include those having the following generalized structure:

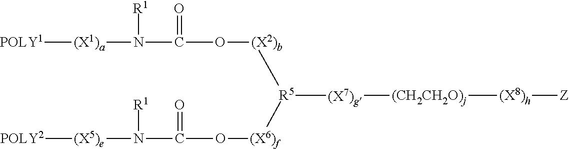

where POLY¹ is a water-soluble polymer; POLY² is a water-soluble polymer; (a) is 0, 1, 2 or 3; (b) is 0, 1, 2 or 3; (e) is 0, 1, 2 or 3; (f) is 0, 1, 2 or 3; (g′) is 0, 1, 2 or 3; (h) is 0, 1, 2 or 3; (j) is 0 to 20; each R¹ is independently H or an organic radical selected from alkyl, substituted alkyl, alkenyl, substituted alkenyl, alkynyl, substituted alkynyl, aryl and substituted aryl; X¹, when present, is a spacer moiety; X², when present, is a spacer moiety; X⁵, when present, is a spacer moiety; X⁶, when present, is a spacer moiety; X⁷, when present, is a spacer moiety; X⁸, when present, is a spacer moiety; R⁵ is a branching moiety; and Z is a reactive group for coupling to a therapeutic peptide, optionally via an intervening spacer. POLY¹ and POLY² in the preceding branched polymer structure may be different or identical, i.e., are of the same polymer type (structure) and molecular weight.

A preferred branched polymer falling into the above classification suitable for use in the present invention is:

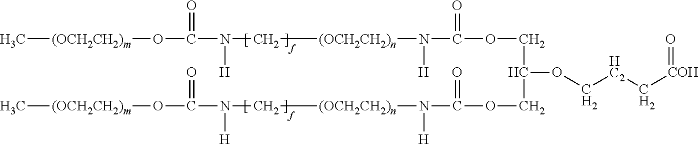

where (m) is 2 to 4000, and (f) is 0 to 6 and (n) is 0 to 20.

Branched polymers suitable for preparing a conjugate of the invention also include those represented more generally by the formula R(POLY)_{y}, where R is a central or core molecule from which extends 2 or more POLY arms such as PEG. The variable y represents the number of POLY arms, where each of the polymer arms can independently be end-capped or alternatively, possess a reactive functional group at its terminus. A more explicit structure in accordance with this embodiment of the invention possesses the structure, R(POLY-Z)_{y}, where each Z is independently an end-capping group or a reactive group, e.g., suitable for reaction with a therapeutic peptide. In yet a further embodiment when Z is a reactive group, upon reaction with a therapeutic peptide, the resulting linkage can be hydrolytically stable, or alternatively, may be degradable, i.e., hydrolyzable. Typically, at least one polymer arm possesses a terminal functional group suitable for reaction with, e.g., a therapeutic peptide. Branched PEGs such as those represented generally by the formula, R(PEG)_{y} above possess 2 polymer arms to about 300 polymer arms (i.e., n ranges from 2 to about 300). Preferably, such branched PEGs typically possess from 2 to about 25 polymer arms, such as from 2 to about 20 polymer arms, from 2 to about 15 polymer arms, or from 3 to about 15 polymer arms. Multi-armed polymers include those having 3, 4, 5, 6, 7 or 8 arms.

Core molecules in branched PEGs as described above include polyols, which are then further functionalized. Such polyols include aliphatic polyols having from 1 to 10 carbon atoms and from 1 to 10 hydroxyl groups, including ethylene glycol, alkane diols, alkyl glycols, alkylidene alkyl diols, alkyl cycloalkane diols, 1,5-decalindiol, 4,8-bis(hydroxymethyl)tricyclodecane, cycloalkylidene diols, dihydroxyalkanes, trihydroxyalkanes, and the like. Cycloaliphatic polyols may also be employed, including straight chained or closed-ring sugars and sugar alcohols, such as mannitol, sorbitol, inositol, xylitol, quebrachitol, threitol, arabitol, erythritol, adonitol, ducitol, facose, ribose, arabinose, xylose, lyxose, rhamnose, galactose, glucose, fructose, sorbose, mannose, pyranose, altrose, talose, tagitose, pyranosides, sucrose, lactose, maltose, and the like. Additional aliphatic polyols include derivatives of glyceraldehyde, glucose, ribose, mannose, galactose, and related stereoisomers. Other core polyols that may be used include crown ether, cyclodextrins, dextrins and other carbohydrates such as starches and amylose. Typical polyols include glycerol, pentaerythritol, sorbitol, and trimethylolpropane.

As will be described in more detail in the linker section below, although any of a number of linkages can be used to covalently attach a polymer to a therapeutic peptide, in certain instances, the linkage is degradable, designated herein as L_{D}, that is to say, contains at least one bond or moiety that hydrolyzes under physiological conditions, e.g., an ester, hydrolyzable carbamate, carbonate, or other such group. In other instances, the linkage is hydrolytically stable.

Illustrative multi-armed PEGs having 3 arms, 4 arms, and 8 arms are known and are available commercially and/or can be prepared following techniques known to those skilled in the art. Multi-armed activated polymers for use in the method of the invention include those corresponding to the following structure, where E represents a reactive group suitable for reaction with a reactive group on the therapeutic peptide. In one or more embodiments, E is an —OH (for reaction with a therapeutic peptide carboxy group or equivalent), a carboxylic acid or equivalent (such as an active ester), a carbonic acid (for reaction with therapeutic peptide —OH groups), or an amino group.

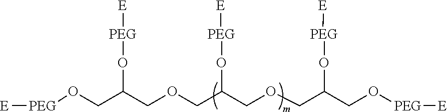

In the structure above, PEG is —(CH₂CH₂O)ₙCH₂CH₂—, and m is selected from 3, 4, 5, 6, 7, and 8. In certain embodiments, typical linkages are ester, carboxyl and hydrolyzable carbamate, such that the polymer-portion of the conjugate is hydrolyzed in vivo to release the therapeutic peptide from the intact polymer conjugate. In such instances, the linker L is designated as L_{D}.

Alternatively, the polymer may possess an overall forked structure as described in U.S. Pat. No. 6,362,254. This type of polymer segment is useful for reaction with two therapeutic peptide moieties, where the two therapeutic peptide moieties are positioned a precise or predetermined distance apart.

In any of the representative structures provided herein, one or more degradable linkages may additionally be contained in the polymer segment, POLY, to allow generation in vivo of a conjugate having a smaller PEG chain than in the initially administered conjugate. Appropriate physiologically cleavable (i.e., releasable) linkages include but are not limited to ester, carbonate ester, carbamate, sulfate, phosphate, acyloxyalkyl ether, acetal, and ketal. Such linkages when contained in a given polymer segment will often be stable upon storage and upon initial administration.

The PEG polymer used to prepare a therapeutic peptide polymer conjugate may comprise a pendant PEG molecule having reactive groups, such as carboxyl or amino, covalently attached along the length of the PEG rather than at the end of the PEG chain(s). The pendant reactive groups can be attached to the PEG directly or through a spacer moiety, such as an alkylene group.

In certain embodiments, a therapeutic peptide polymer conjugate according to one aspect of the invention is one comprising a therapeutic peptide releasably attached, preferably at its N-terminus, to a water-soluble polymer. Hydrolytically degradable linkages, useful not only as a degradable linkage within a polymer backbone, but also, in the case of certain embodiments of the invention, for covalently attaching a water-soluble polymer to a therapeutic peptide, include: carbonate; imine resulting, for example, from reaction of an amine and an aldehyde (see, e.g., Ouchi et al. (1997) Polymer Preprints 38(1):582-3); phosphate ester, formed, for example, by reacting an alcohol with a phosphate group; hydrazone, e.g., formed by reaction of a hydrazide and an aldehyde; acetal, e.g., formed by reaction of an aldehyde and an alcohol; orthoester, formed, for example, by reaction between a formate and an alcohol; and esters, and certain urethane (carbamate) linkages.

Illustrative PEG reagents for use in preparing a releasable therapeutic peptide conjugate in accordance with the invention are described in U.S. Pat. Nos. 6,348,558, 5,612,460, 5,840,900, 5,880,131, and 6,376,470.

Additional PEG reagents for use in the invention include hydrolyzable and/or releasable PEGs and linkers such as those described in U.S. Patent Application Publication No. 2006-0293499. In the resulting conjugate, the therapeutic peptide and the polymer are each covalently attached to different positions of the aromatic scaffold, e.g., Fmoc or FMS, structure, and are releasable under physiological conditions. Generalized structures corresponding to the polymers described therein are provided below.

For example, one such polymeric reagent comprises the following structure:

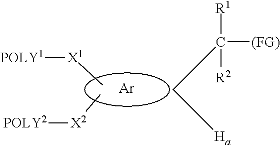

where POLY¹ is a first water-soluble polymer; POLY² is a second water-soluble polymer; X¹ is a first spacer moiety; X² is a second spacer moiety;

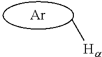

is an aromatic-containing moiety bearing an ionizable hydrogen atom, H_{α}; R¹ is H or an organic radical; R² is H or an organic radical; and (FG) is a functional group capable of reacting with an amino group of an active agent to form a releasable linkage, such as a carbamate linkage (such as N-succinimidyloxy, 1-benzotriazolyloxy, oxycarbonylimidazole, —O—C(O)—Cl, O—C(O)—Br, unsubstituted aromatic carbonate radicals and substituted aromatic carbonate radicals). The polymeric reagent can include one, two, three, four or more electron altering groups attached to the aromatic-containing moiety.

Preferred aromatic-containing moieties are bicyclic and tricyclic aromatic hydrocarbons. Fused bicyclic and tricyclic aromatics include pentalene, indene, naphthalene, azulene, heptalene, biphenylene, as-indacene, s-indacene, acenaphthylene, fluorene, phenalene, phenanthrene, anthracene, and fluoranthene.

A preferred polymer reagent possesses the following structure,

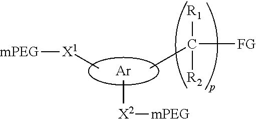

where mPEG corresponds to CH₃O—(CH₂CH₂O)ₙCH₂CH₂—, X¹ and X² are each independently a spacer moiety having an atom length of from about 1 to about 18 atoms, n ranges from 10 to 1800, p is an integer ranging from 1 to 8, R¹ is H or lower alkyl, R² is H or lower alkyl, and Ar is an aromatic hydrocarbon, preferably a bicyclic or tricyclic aromatic hydrocarbon. FG is as defined above. Preferably, FG corresponds to an activated carbonate ester suitable for reaction with an amino group on therapeutic peptide. Preferred spacer moieties, X¹ and X², include —NH—C(O)—CH₂—O—, —NH—C(O)—(CH₂)_{q}—O—, —NH—C(O)—(CH₂)_{q}—C(O)—NH—, —NH—C(O)—(CH₂)_{q}—, and —C(O)—NH—, where q is selected from 2, 3, 4, and 5. Preferably, although not necessarily, the nitrogen in the preceding spacers is proximal to the PEG rather than to the aromatic moiety.

Another such branched (2-armed) polymeric reagent comprised of two electron altering groups comprises the following structure:

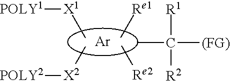

wherein each of POLY¹, POLY², X¹, X², R¹, R²,

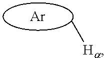

and (FG) is as defined immediately above, and R^{e1} is a first electron altering group; and R^{e2} is a second electron altering group. An electron altering group is a group that is either electron donating (and therefore referred to as an “electron donating group”), or electron withdrawing (and therefore referred to as an “electron withdrawing group”). When attached to the aromatic-containing moiety bearing an ionizable hydrogen atom, an electron donating group is a group having the ability to position electrons away from itself and closer to or within the aromatic-containing moiety. When attached to the aromatic-containing moiety bearing an ionizable hydrogen atom, an electron withdrawing group is a group having the ability to position electrons toward itself and away from the aromatic-containing moiety. Hydrogen is used as the standard for comparison in the determination of whether a given group positions electrons away or toward itself. Preferred electron altering groups include, but are not limited to, —CF₃, —CH₂CF₃, —CH₂C₆F₅, —CN, —NO₂, —S(O)R, —S(O)Aryl, —S(O₂)R, —S(O₂)Aryl, —S(O₂)OR, —S(O₂)OAryl, —S(O₂)NHR, —S(O₂)NHAryl, —C(O)R, —C(O)Aryl, —C(O)OR, —C(O)NHR, and the like, wherein R is H or an organic radical.

An additional branched polymeric reagent suitable for use in the present invention comprises the following structure:

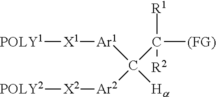

where POLY¹ is a first water-soluble polymer; POLY² is a second water-soluble polymer; X¹ is a first spacer moiety; X² is a second spacer moiety; Ar¹ is a first aromatic moiety; Ar² is a second aromatic moiety; H_{α} is an ionizable hydrogen atom; R¹ is H or an organic radical; R² is H or an organic radical; and (FG) is a functional group capable of reacting with an amino group of therapeutic peptide to form a releasable linkage, such as carbamate linkage.

Another exemplary polymeric reagent comprises the following structure:

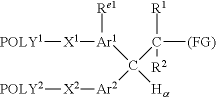

wherein each of POLY¹, POLY², X¹, X², Ar¹, Ar², H_{α}, R¹, R², and (FG) is as previously defined, and R^{e1} is a first electron altering group. While stereochemistry is not specifically shown in any structure provided herein, the provided structures contemplate both enantiomers, as well as compositions comprising mixtures of each enantiomer in equal amounts (i.e., a racemic mixture) and unequal amounts.

Yet an additional polymeric reagent for use in preparing a therapeutic peptide conjugate possesses the following structure:

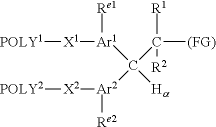

wherein each of POLY¹, POLY², X¹, X², Ar¹, Ar², H_{α}, R¹, R², and (FG) is as previously defined, and R^{e1} is a first electron altering group; and R^{e2} is a second electron altering group.

A preferred polymeric reagent comprises the following structure:

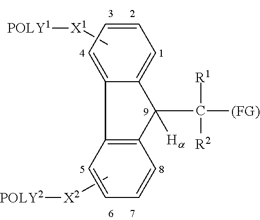

wherein each of POLY¹, POLY², X¹, X², R¹, R², H_{α} and (FG) is as previously defined, and, as can be seen from the structure above, the aromatic moiety is a fluorene. The POLY arms substituted on the fluorene can be in any position in each of their respective phenyl rings, i.e., POLY¹-X¹— can be positioned at any one of carbons 1, 2, 3, and 4, and POLY²-X²— can be in any one of positions 5, 6, 7, and 8.

Yet another preferred fluorene-based polymeric reagent comprises the following structure:

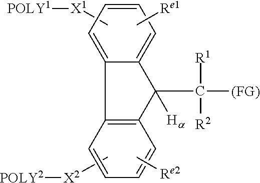

wherein each of POLY¹, POLY², X¹, X², R¹, R², H_{α} and (FG) is as previously defined, and R^{e1} is a first electron altering group; and R^{e2} is a second electron altering group as described above.

Yet another exemplary polymeric reagent for conjugating to a therapeutic peptide comprises the following fluorene-based structure:

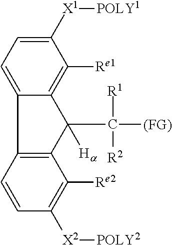

wherein each of POLY¹, POLY², X¹, X², R¹, R², H_{α} and (FG) is as previously defined, and R^{e1} is a first electron altering group; and R^{e2} is a second electron altering group.

Particular fluorene-based polymeric reagents for forming a releasable therapeutic peptide polymer conjugate in accordance with the invention include the following:

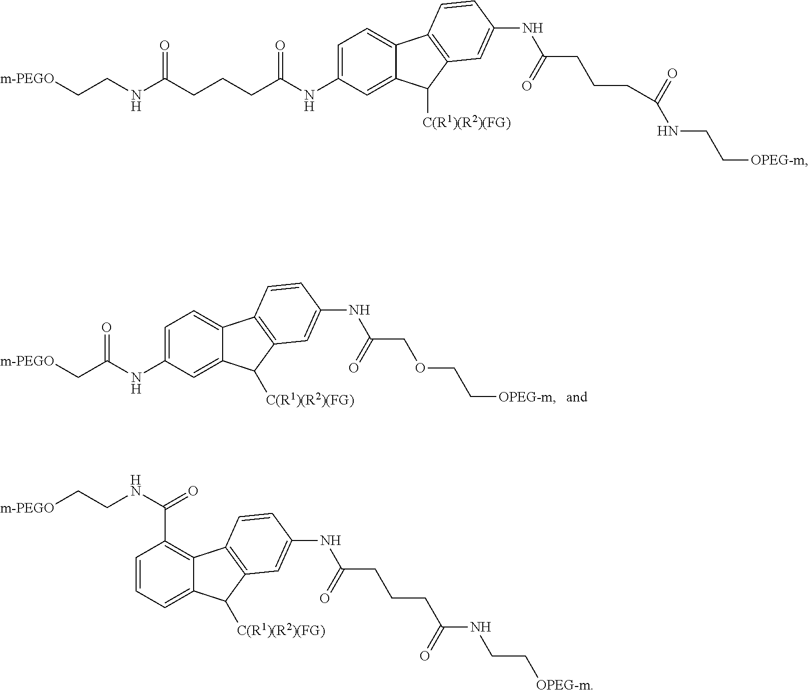

Still another exemplary polymeric reagent comprises the following structure:

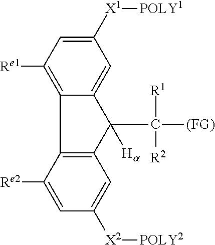

wherein each of POLY¹, POLY², X¹, X², R¹, R², H_{α} and (FG) is as previously defined, and R^{e1} is a first electron altering group; and R^{e2} is a second electron altering group. Branched reagents suitable for preparing a releasable therapeutic peptide conjugate include N-{di(mPEG(20,000)oxymethylcarbonylamino)fluoren-9-ylmethoxycarbonyloxy}succinimide, N-[2,7 di (4mPEG(10,000)aminocarbonylbutyrylamino)fluoren-9 ylmethoxycarbonyloxy]-succinimide (“G2PEG2Fmoc₂₀ₖ-NHS”), and PEG2-CAC-Fmoc₄ₖ-BTC. Of course, PEGs of any molecular weight as set forth herein may be employed in the above structures, and the particular activating groups described above are not meant to be limiting in any respect, and may be substituted by any other suitable activating group suitable for reaction with a reactive group present on the therapeutic peptide.

Those of ordinary skill in the art will recognize that the foregoing discussion describing water-soluble polymers for use in forming a therapeutic peptide conjugate is by no means exhaustive and is merely illustrative, and that all polymeric materials having the qualities described above are contemplated. As used herein, the term “polymeric reagent” generally refers to an entire molecule, which can comprise a water-soluble polymer segment, as well as additional spacers and functional groups.

### Linkages

The particular linkage between the therapeutic peptide and the water-soluble polymer depends on a number of factors. Such factors include, for example, the particular linkage chemistry employed, the particular spacer moieties utilized, if any, the particular therapeutic peptide, the available functional groups within the therapeutic peptide (either for attachment to a polymer or conversion to a suitable attachment site), and the possible presence of additional reactive functional groups or absence of functional groups within the therapeutic peptide due to modifications made to the peptide such as methylation and/or glycosylation, and the like.

In one or more embodiments of the invention, the linkage between the therapeutic peptide and the water-soluble polymer is a releasable linkage. That is, the water-soluble polymer is cleaved (either through hydrolysis, an enzymatic processes, or otherwise), thereby resulting in an unconjugated therapeutic peptide. Preferably, the releasable linkage is a hydrolytically degradable linkage, where upon hydrolysis, the therapeutic peptide, or a slightly modified version thereof, is released. The releasable linkage may result in the water-soluble polymer (and any spacer moiety) detaching from the therapeutic peptide in vivo (and in vitro) without leaving any fragment of the water-soluble polymer (and/or any spacer moiety or linker) attached to the therapeutic peptide. Exemplary releasable linkages include carbonate, carboxylate ester, phosphate ester, thiolester, anhydrides, acetals, ketals, acyloxyalkyl ether, imines, carbamates, and orthoesters. Such linkages can be readily formed by reaction of the therapeutic peptide and/or the polymeric reagent using coupling methods commonly employed in the art. Hydrolyzable linkages are often readily formed by reaction of a suitably activated polymer with a non-modified functional group contained within the therapeutic peptide. Preferred positions for covalent attachment of a water-soluble polymer induce the N-terminal, the C-terminal, as well as the internal lysines. Preferred releasable linkages include carbamate and ester.

Generally speaking, a preferred therapeutic peptide conjugate of the invention will possess the following generalized structure:

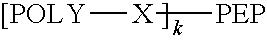

where POLY is a water-soluble polymer such as any of the illustrative polymeric reagents provided in Tables B-D herein, X is a linker, and in some embodiments a hydrolyzable linkage (L_{D}), and k is an integer selected from 1, 2, and 3, and in some instances 4, 5, 6, 7, 8, 9 and 10. In the generalized structure above, where X is L_{D}, L_{D} refers to the hydrolyzable linkage per se (e.g., a carbamate or an ester linkage), while “POLY” is meant to include the polymer repeat units, e.g., CH₃(OCH₂CH₂)ₙ,—. In a preferred embodiment of the invention, at least one of the water-soluble polymer molecules is covalently attached to the N-terminus of therapeutic peptide. In one embodiment of the invention, k equals 1 and X is —O—C(O)—NH—, where the —NH— is part of the therapeutic peptide residue and represents an amino group thereof.

Although releasable linkages are exemplary, the linkage between the therapeutic peptide and the water-soluble polymer (or the linker moiety that is attached to the polymer) may be a hydrolytically stable linkage, such as an amide, a urethane (also known as carbamate), amine, thioether (also known as sulfide), or urea (also known as carbamide). One such embodiment of the invention comprises a therapeutic peptide having a water-soluble polymer such as PEG covalently attached at the N-terminus of therapeutic peptide. In such instances, alkylation of the N-terminal residue permits retention of the charge on the N-terminal nitrogen.

With regard to linkages, in one or more embodiments of the invention, a conjugate is provided that comprises a therapeutic peptide covalently attached at an amino acid residue, either directly or through a linker comprised of one or more atoms, to a water-soluble polymer.

The conjugates (as opposed to an unconjugated therapeutic peptide) may or may not possess a measurable degree of therapeutic peptide activity. That is to say, a conjugate in accordance with the invention will typically possess anywhere from about 0% to about 100% or more of the therapeutic activity of the unmodified parent therapeutic peptide. Typically, compounds possessing little or no therapeutic activity contain a releasable linkage connecting the polymer to the therapeutic peptide, so that regardless of the lack of therapeutic activity in the conjugate, the active parent molecule (or a derivative thereof having therapeutic activity) is released by cleavage of the linkage (e.g., hydrolysis upon aqueous-induced cleavage of the linkage). Such activity may be determined using a suitable in vivo or in vitro model, depending upon the known activity of the particular moiety having therapeutic peptide activity employed.

Optimally, cleavage of a linkage is facilitated through the use of hydrolytically cleavable and/or enzymatically cleavable linkages such as urethane, amide, certain carbamate, carbonate or ester-containing linkages. In this way, clearance of the conjugate via cleavage of individual water-soluble polymer(s) can be modulated by selecting the polymer molecular size and the type of functional group for providing the desired clearance properties. In certain instances, a mixture of polymer conjugates is employed where the polymers possess structural or other differences effective to alter the release (e.g., hydrolysis rate) of the therapeutic peptide, such that one can achieve a desired sustained delivery profile.

One of ordinary skill in the art can determine the proper molecular size of the polymer as well as the cleavable functional group, depending upon several factors including the mode of administration. For example, one of ordinary skill in the art, using routine experimentation, can determine a proper molecular size and cleavable functional group by first preparing a variety of polymer-(therapeutic peptide) conjugates with different weight-average molecular weights, degradable functional groups, and chemical structures, and then obtaining the clearance profile for each conjugate by administering the conjugate to a patient and taking periodic blood and/or urine samples. Once a series of clearance profiles has been obtained for each tested conjugate, a conjugate or mixture of conjugates having the desired clearance profile(s) can be determined.

For conjugates possessing a hydrolytically stable linkage that couples the therapeutic peptide to the water-soluble polymer, the conjugate will typically possess a measurable degree of therapeutic activity. For instance, such conjugates are typically characterized as having a therapeutic activity satisfying one or more of the following percentages relative to that of the unconjugated therapeutic peptide: at least 2%, at least 5%, at least 10%, at least 15%, at least 25%, at least 30%, at least 40%, at least 50%, at least 60%, at least 80%, at least 85%, at least 90%, at least 95%, at least 97%, at least 100%, more than 105%, more than 10-fold, or more than 100-fold (when measured in a suitable model, such as those presented here and/or known in the art). Often, conjugates having a hydrolytically stable linkage (e.g., an amide linkage) will possess at least some degree of the therapeutic activity of the unmodified parent therapeutic peptide.

Exemplary conjugates in accordance with the invention will now be described. Amino groups on a therapeutic peptide provide a point of attachment between the therapeutic peptide and the water-soluble polymer. For example, a therapeutic peptide may comprise one or more lysine residues, each lysine residue containing an ε-amino group that may be available for conjugation, as well as one amino terminus.

There are a number of examples of suitable water-soluble polymeric reagents useful for forming covalent linkages with available amines of a therapeutic peptide. Certain specific examples, along with the corresponding conjugates, are provided in Table B below. In the table, the variable (n) represents the number of repeating monomeric units and “PEP” represents a therapeutic peptide following conjugation to the water-soluble polymer. While each polymeric portion (e.g., (OCH₂CH₂)ₙ or (CH₂CH₂O)ₙ) presented in Table B terminates in a “CH₃” group, other groups (such as H and benzyl) can be substituted therefore.

As will be clearly understood by one skilled in the art, for conjugates such as those set forth below resulting from reaction with a therapeutic peptide amino group, the amino group extending from the therapeutic peptide designation “˜NH-PEP” represents the residue of the therapeutic peptide itself in which the ˜NH— is an amino group of the therapeutic peptide. One preferred site of attachment for the polymeric reagents shown below is the N-terminus. Further, although the conjugates in Tables B-D herein illustrate a single water-soluble polymer covalently attached to a therapeutic peptide, it will be understood that the conjugate structures on the right are meant to also encompass conjugates having more than one of such water-soluble polymer molecules covalently attached to therapeutic peptide, e.g., 2, 3, or 4 water-soluble polymer molecules.

TABLE B
Amine-Specific Polymeric Reagents and the Therapeutic Peptide Conjugates Formed
Therefrom

| Polymeric Reagent | Corresponding Conjugate |
|---|---|
| 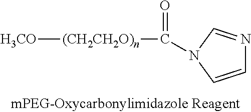 | 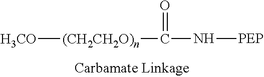 |
| 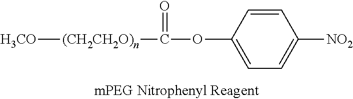 | 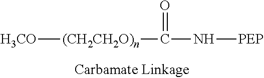 |
| 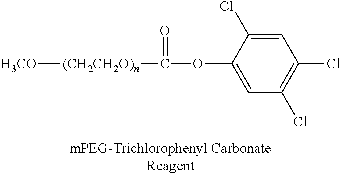 | 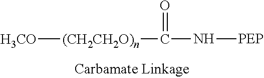 |
| 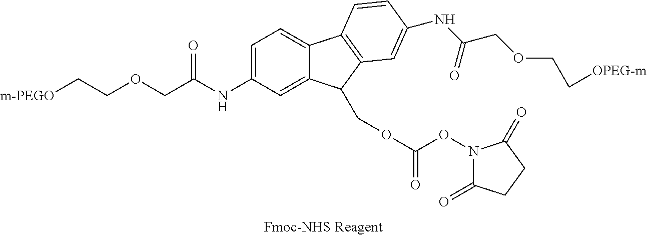 | 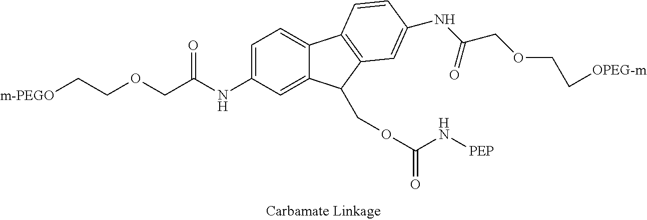 |
| 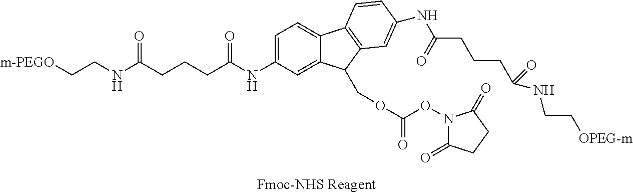 | 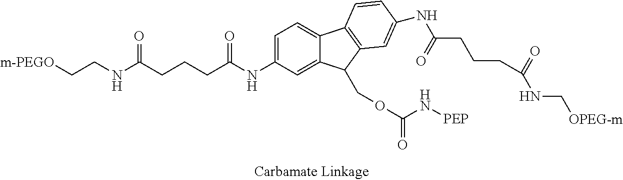 |
| 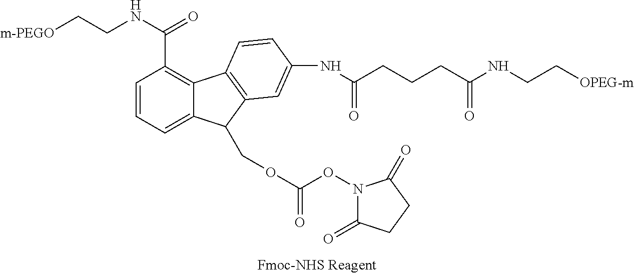 | 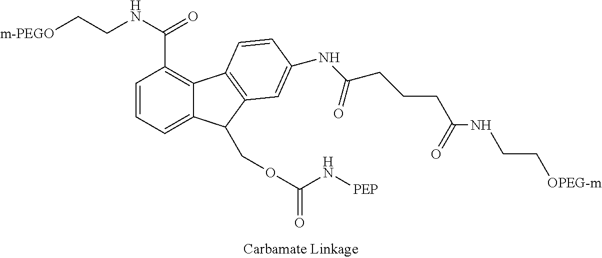 |
| 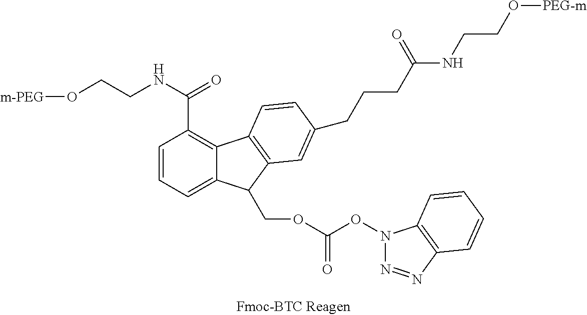 | 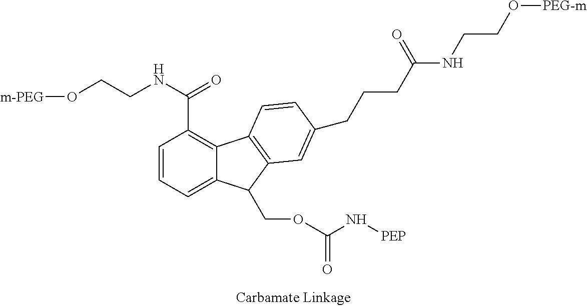 |
| 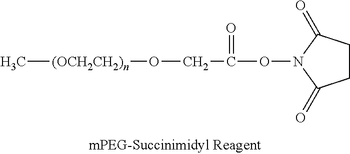 | 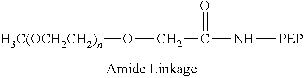 |
| 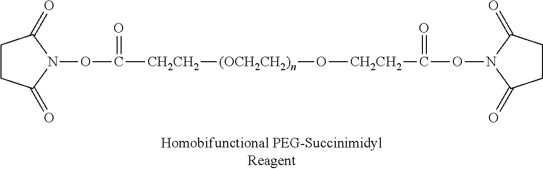 | 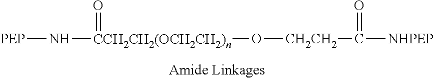 |
| 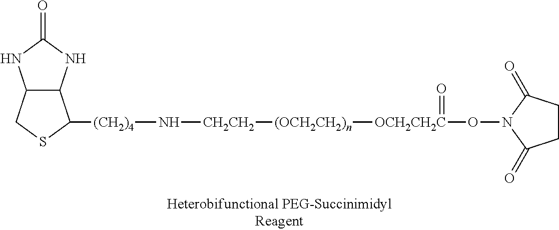 | 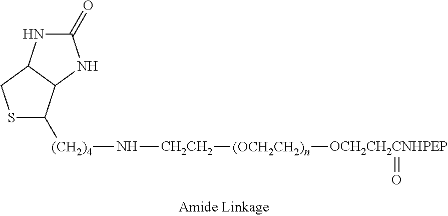 |
| 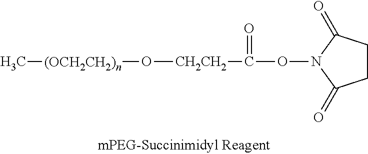 | 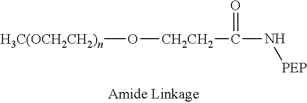 |
| 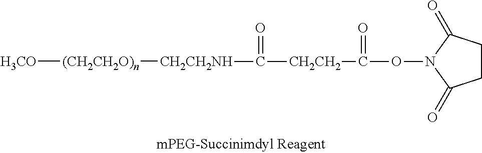 | 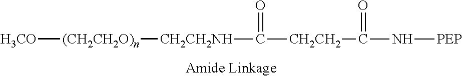 |
| 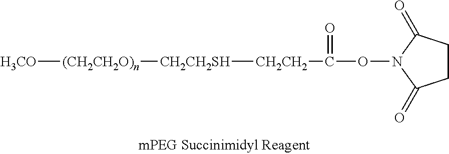 | 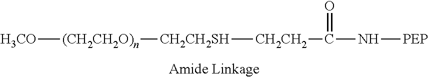 |
| 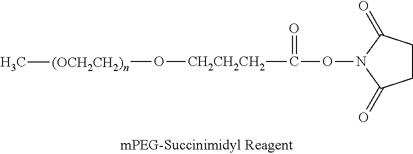 | 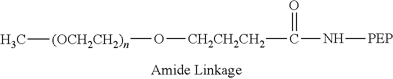 |
| 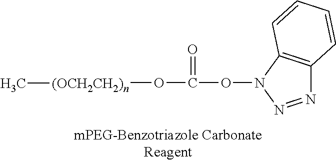 | 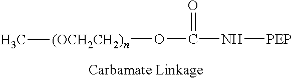 |
| 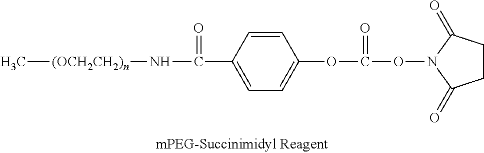 | 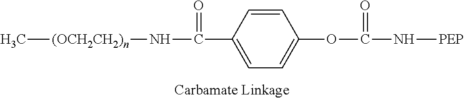 |
| 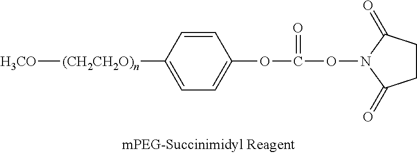 | 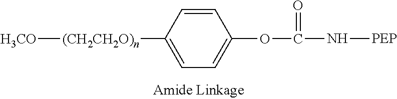 |
| 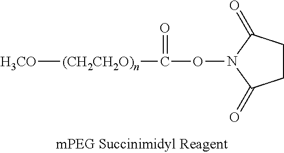 | 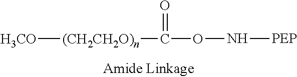 |
| 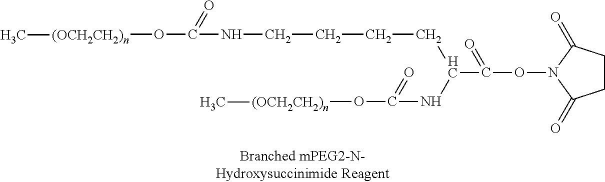 | 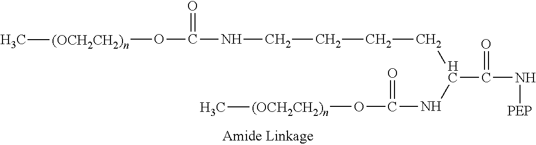 |
| 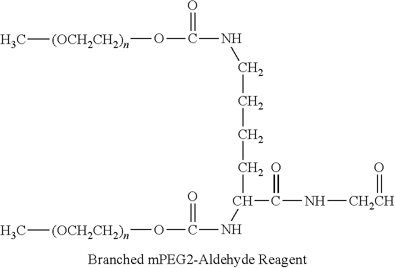 | 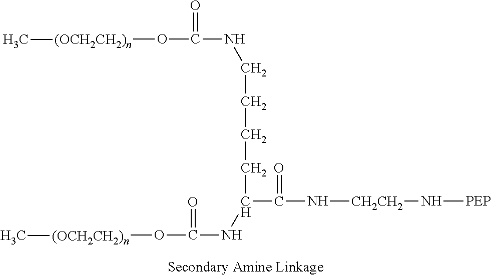 |
| 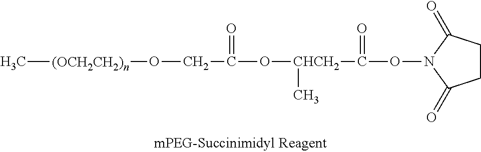 | 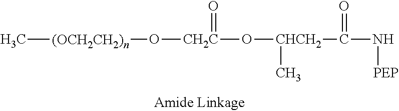 |
| 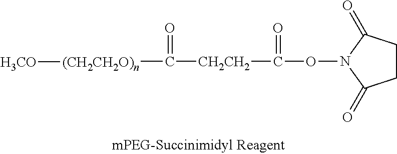 | 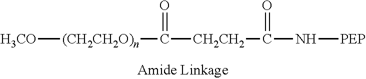 |
| 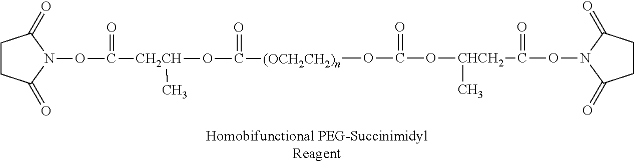 | 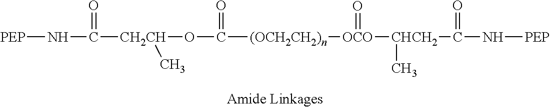 |
| 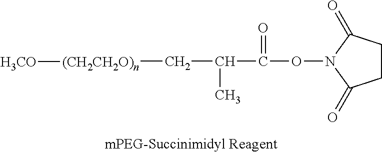 | 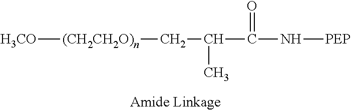 |
| 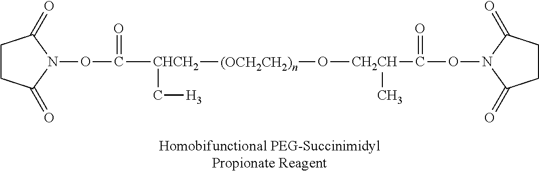 | 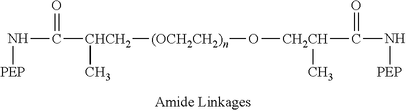 |
| 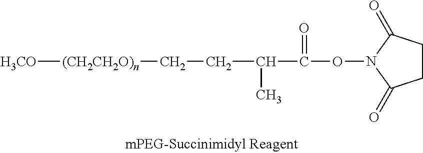 | 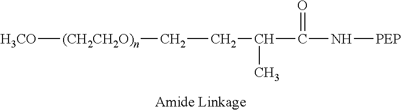 |
| 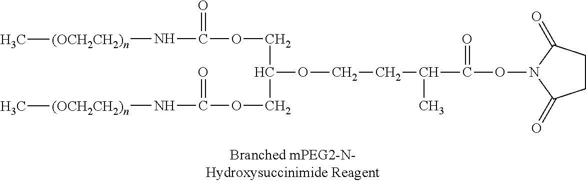 | 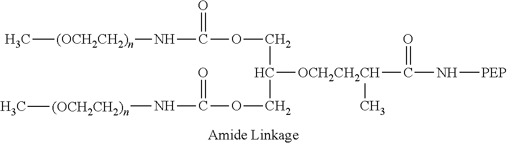 |
| 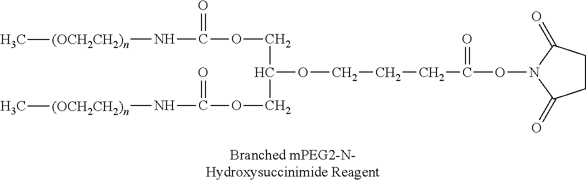 | 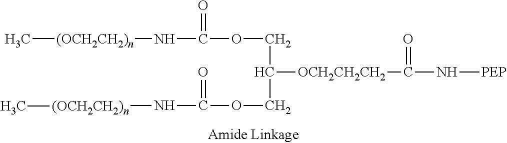 |
| 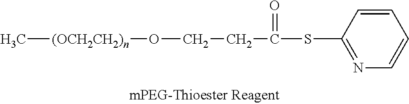 | 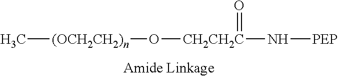 |
| 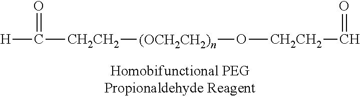 | 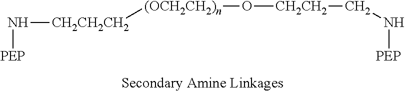 |
| 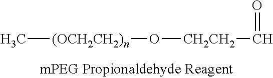 | 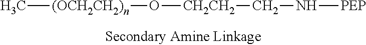 |
| 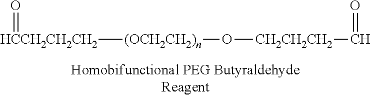 | 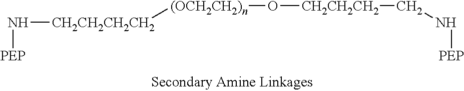 |
| 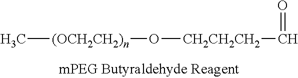 | 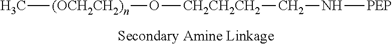 |
| 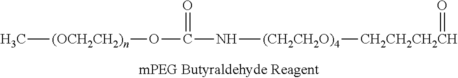 | 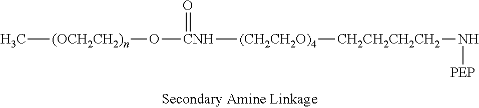 |
| 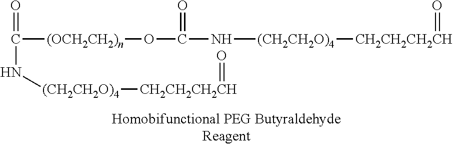 | 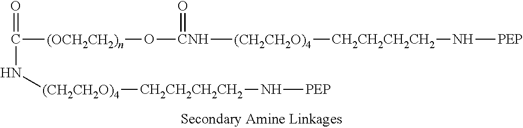 |
| 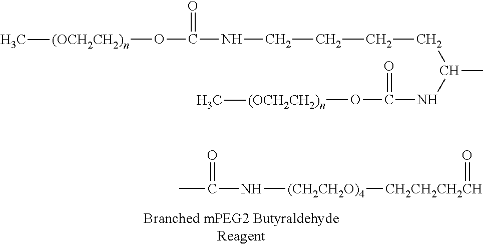 | 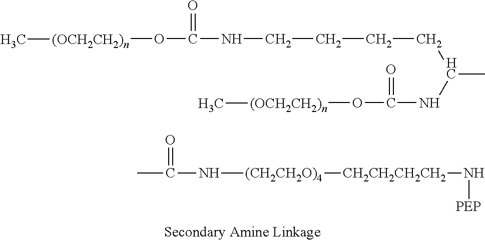 |
| 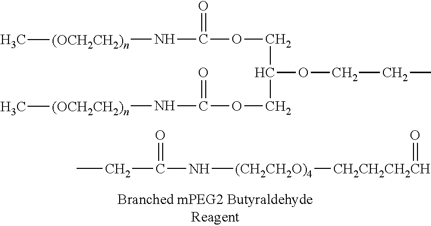 | 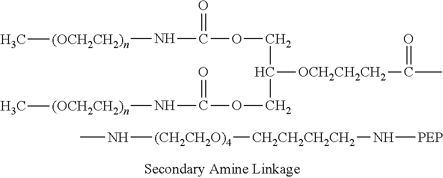 |
| 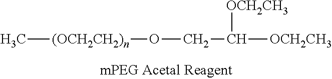 | 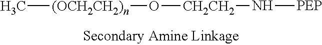 |
| 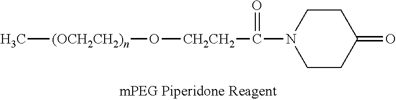 | 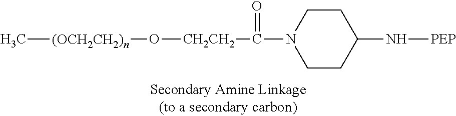 |
| 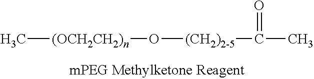 | 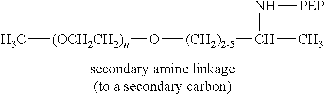 |
| 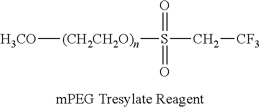 | 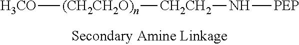 |
| 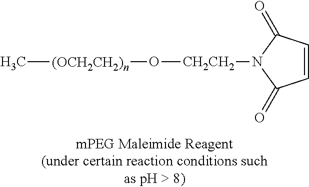 | 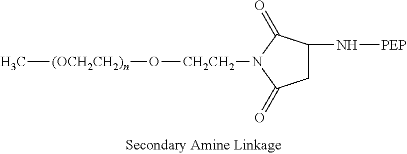 |
| 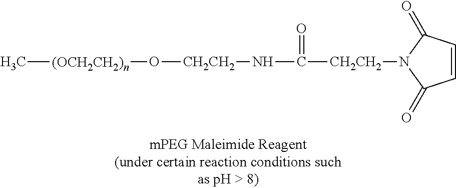 | 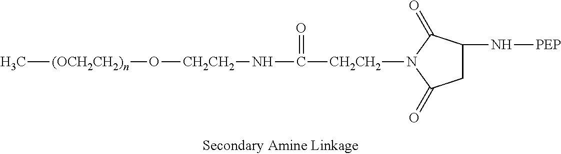 |
| 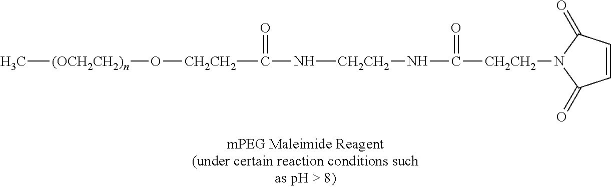 | 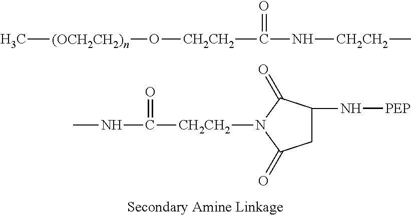 |
| 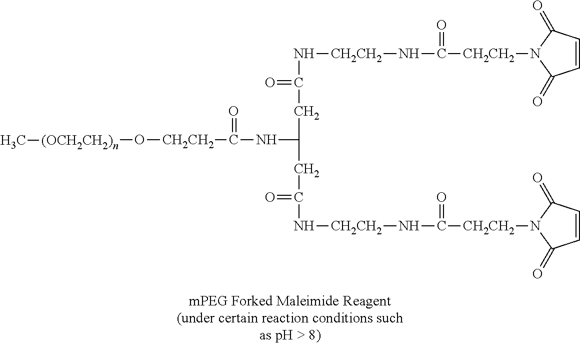 | 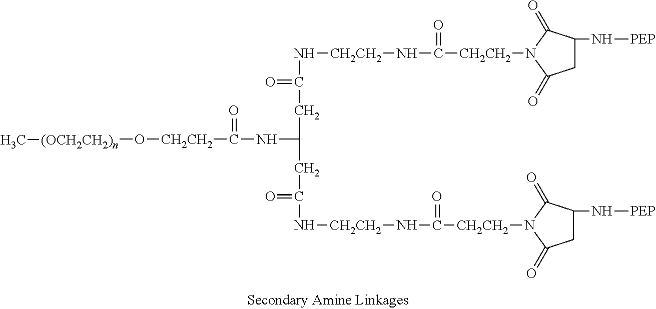 |
| 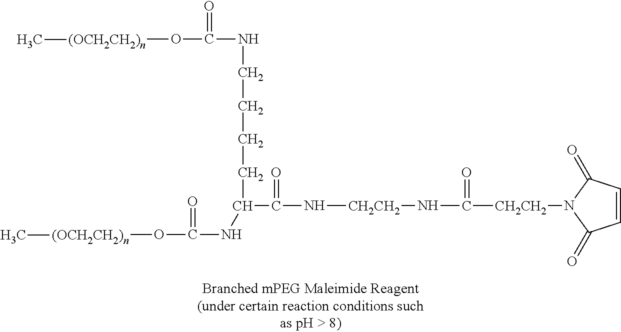 | 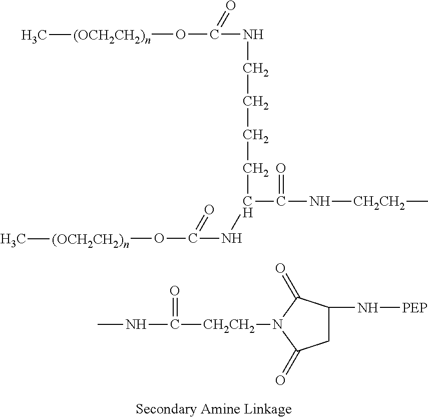 |

### Amine Conjugation and Resulting Conjugates

Conjugation of a polymeric reagent to an amine group of a therapeutic peptide can be accomplished by a variety of techniques. In one approach, a therapeutic peptide is conjugated to a polymeric reagent functionalized with an active ester such as a succinimidyl derivative (e.g., an N-hydroxysuccinimide ester). In this approach, the polymeric reagent bearing the reactive ester is reacted with the therapeutic peptide in aqueous media under appropriate pH conditions, e.g., from pHs ranging from about 3 to about 8, about 3 to about 7, or about 4 to about 6.5. Most polymer active esters can couple to a target peptide such as therapeutic peptide at physiological pH, e.g., at 7.0. However, less reactive derivatives may require a different pH. Typically, activated PEGs can be attached to a peptide such as therapeutic peptide at pHs from about 7.0 to about 11.5, e.g., about 7.0 to about 10.0, for covalent attachment to an internal lysine. Typically, lower pHs are used, e.g., 4 to about 5.75, for preferential covalent attachment to the N-terminus. Thus, different reaction conditions (e.g., different pHs or different temperatures) can result in the attachment of a water-soluble polymer such as PEG to different locations on the therapeutic peptide (e.g., internal lysines versus the N-terminus). Coupling reactions can often be carried out at room temperature, although lower temperatures may be required for particularly labile therapeutic peptide moieties. Reaction times are typically on the order of minutes, e.g., 30 minutes, to hours, e.g., from about 1 to about 36 hours), depending upon the pH and temperature of the reaction. N-terminal PEGylation, e.g., with a PEG reagent bearing an aldehyde group, is typically conducted under mild conditions, pHs from about 5-10, for about 6 to 48 hours. Varying ratios of polymeric reagent to therapeutic peptide may be employed, e.g., from an equimolar ratio up to a 10-fold molar excess of polymer reagent. Typically, up to a 5-fold molar excess of polymer reagent will suffice.

In certain instances, it may be preferable to protect certain amino acids from reaction with a particular polymeric reagent if site specific or site selective covalent attachment is desired using commonly employed protection/deprotection methodologies such as those well known in the art.

In an alternative approach to direct coupling reactions, the PEG reagent may be incorporated at a desired position of the therapeutic peptide during peptide synthesis. In this way, site-selective introduction of one or more PEGs can be achieved. See, e.g., International Patent Publication No. WO 95/00162, which describes the site selective synthesis of conjugated peptides.

Exemplary conjugates that can be prepared using, for example, polymeric reagents containing a reactive ester for coupling to an amino group of therapeutic peptide, comprise the following alpha-branched structure:

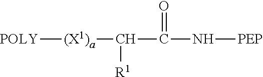

where POLY is a water-soluble polymer, (a) is either zero or one; X¹, when present, is a spacer moiety comprised of one or more atoms; R¹ is hydrogen an organic radical; and “˜NH-PEP” represents a residue of a therapeutic peptide, where the underlined amino group represents an amino group of the therapeutic peptide.

With respect to the structure corresponding to that referred to in the immediately preceding paragraph, any of the water-soluble polymers provided herein can be defined as POLY, any of the spacer moieties provided herein can be defined as X¹ (when present), any of the organic radicals provided herein can be defined as R¹ (in instances where R¹ is not hydrogen), and any of the therapeutic peptides provided herein can be employed. In one or more embodiments corresponding to the structure referred to in the immediately preceding paragraph, POLY is a poly(ethylene glycol) such as H₃CO(CH₂CH₂O)ₙ—, wherein (n) is an integer having a value of from 3 to 4000, more preferably from 10 to about 1800; (a) is one; X¹ is a C₁₋₆ alkylene, such as one selected from methylene (i.e., —CH₂—), ethylene (i.e., —CH₂—CH₂—) and propylene (i.e., —CH₂—CH₂—CH₂—); R¹ is H or lower alkyl such as methyl or ethyl; and PEP corresponds to any therapeutic peptide disclosed herein, including in Table A.

Typical of another approach for conjugating a therapeutic peptide to a polymeric reagent is reductive amination. Typically, reductive amination is employed to conjugate a primary amine of a therapeutic peptide with a polymeric reagent functionalized with a ketone, aldehyde or a hydrated form thereof (e.g., ketone hydrate and aldehyde hydrate). In this approach, the primary amine from the therapeutic peptide (e.g., the N-terminus) reacts with the carbonyl group of the aldehyde or ketone (or the corresponding hydroxy-containing group of a hydrated aldehyde or ketone), thereby forming a Schiff base. The Schiff base, in turn, is then reductively converted to a stable conjugate through use of a reducing agent such as sodium borohydride or any other suitable reducing agent. Selective reactions (e.g., at the N-terminus) are possible, particularly with a polymer functionalized with a ketone or an alpha-methyl branched aldehyde and/or under specific reaction conditions (e.g., reduced pH).

Exemplary conjugates that can be prepared using, for example, polymeric reagents containing an aldehyde (or aldehyde hydrate) or ketone or (ketone hydrate) possess the following structure:

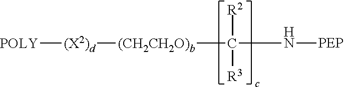

where POLY is a water-soluble polymer; (d) is either zero or one; X², when present, is a spacer moiety comprised of one or more atoms; (b) is an integer having a value of one through ten; (c) is an integer having a value of one through ten; R², in each occurrence, is independently H or an organic radical; R³, in each occurrence, is independently H or an organic radical; and “˜NH-PEP” represents a residue of a therapeutic peptide, where the underlined amino group represents an amino group of the therapeutic peptide.

Yet another illustrative conjugate of the invention possesses the structure:

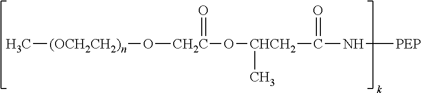

where k ranges from 1 to 3, and n ranges from 10 to about 1800.

With respect to the structure corresponding to that referred to in immediately preceding paragraph, any of the water-soluble polymers provided herein can be defined as POLY, any of the spacer moieties provided herein can be defined as X² (when present), any of the organic radicals provided herein can be independently defined as R² and R³ (in instances where R² and R³ are independently not hydrogen), and any of the PEP moieties provided herein can be defined as a therapeutic peptide. In one or more embodiments of the structure referred to in the immediately preceding paragraph, POLY is a poly(ethylene glycol) such as H₃CO(CH₂CH₂O)ₙ—, wherein (n) is an integer having a value of from 3 to 4000, more preferably from 10 to about 1800; (d) is one; X¹ is amide (e.g., —C(O)NH—); (b) is 2 through 6, such as 4; (c) is 2 through 6, such as 4; each of R² and R³ are independently H or lower alkyl, such as methyl when lower alkyl; and PEP is therapeutic peptide.

Another example of a therapeutic peptide conjugate in accordance with the invention has the following structure:

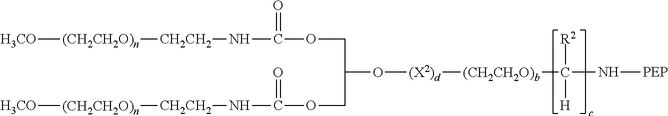

wherein each (n) is independently an integer having a value of from 3 to 4000, preferably from 10 to 1800; X² is as previously defined; (b) is 2 through 6; (c) is 2 through 6; R², in each occurrence, is independently H or lower alkyl; and “˜NH-PEP” represents a residue of a therapeutic peptide, where the underlined amino group represents an amino group of the therapeutic peptide.

Additional therapeutic peptide polymer conjugates resulting from reaction of a water-soluble polymer with an amino group of therapeutic peptide are provided below. The following conjugate structures are releasable. One such structure corresponds to:

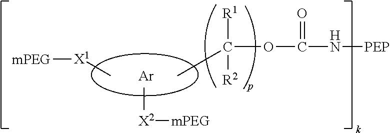

where mPEG is CH₃O—(CH₂CH₂O)ₙCH₂CH₂—, n ranges from 10 to 1800, p is an integer ranging from 1 to 8, R¹ is H or lower alkyl, R² is H or lower alkyl, Ar is an aromatic hydrocarbon, such as a fused bicyclic or tricyclic aromatic hydrocarbon, X¹ and X² are each independently a spacer moiety having an atom length of from about 1 to about 18 atoms, ˜NH-PEP is as previously described, and k is an integer selected from 1, 2, and 3. The value of k indicates the number of water-soluble polymer molecules attached to different sites on the therapeutic peptide. In a preferred embodiment, R¹ and R² are both H. The spacer moieties, X¹ and X², preferably each contain one amide bond. In a preferred embodiment, X¹ and X² are the same. Preferred spacers, i.e., X¹ and X², include —NH—C(O)—CH₂—O—, —NH—C(O)—(CH₂)_{q}—O—, —NH—C(O)—(CH₂)_{q}—C(O)—NH—, —NH—C(O)—(CH₂)_{q}—, and —C(O)—NH—, where q is selected from 2, 3, 4; and 5. Although the spacers can be in either orientation, preferably, the nitrogen is proximal to the PEG rather than to the aromatic moiety. Illustrative aromatic moieties include pentalene, indene, naphthalene, indacene, acenaphthylene, and fluorene.

Particularly preferred conjugates of this type are provided below.

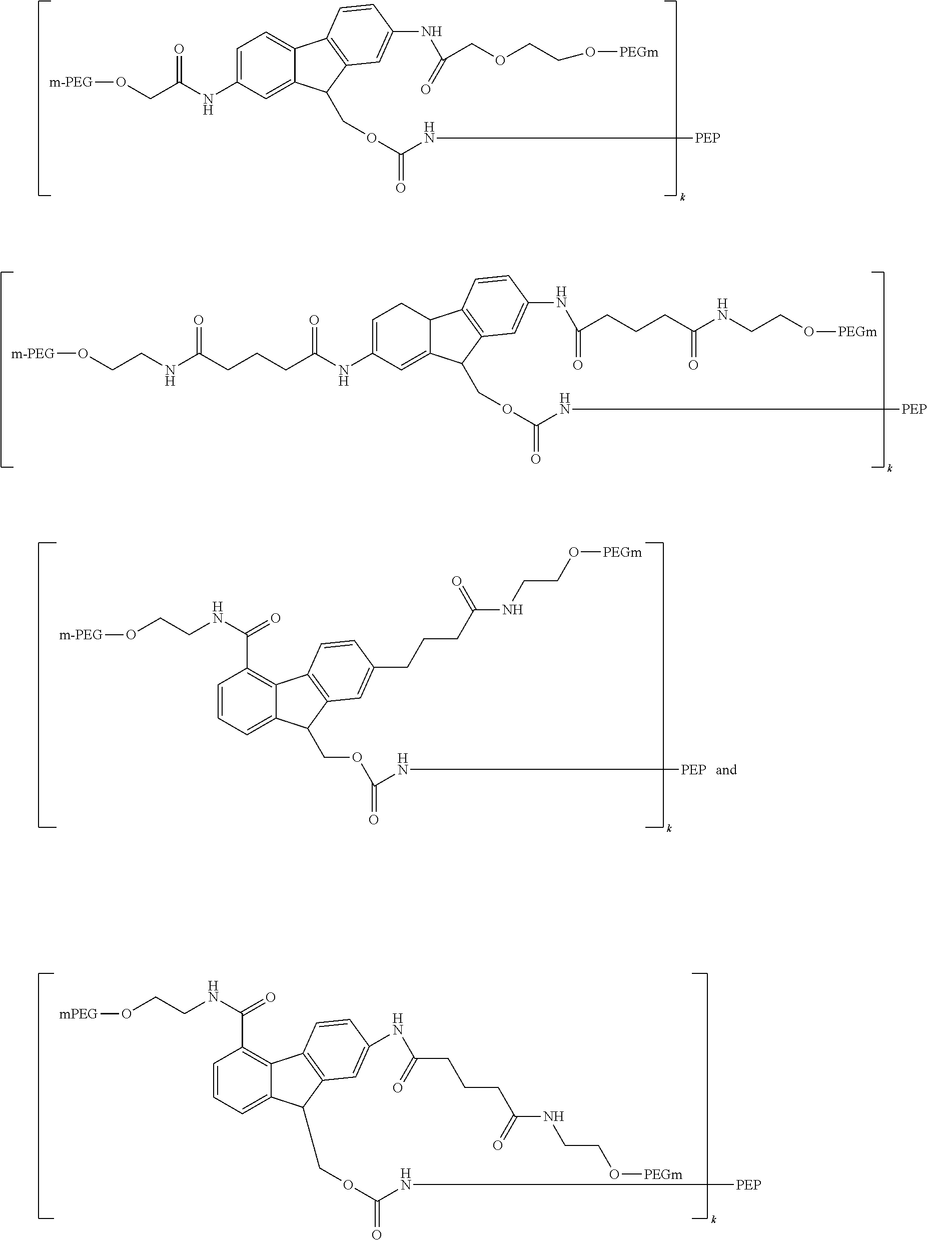

Additional therapeutic peptide conjugates resulting from covalent attachment to amino groups of therapeutic peptide that are also releasable include the following:

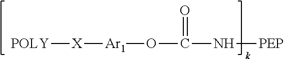

where X is either —O— or —NH—C(O)—, Ar₁ is an aromatic group, e.g., ortho, meta, or para-substituted phenyl, and k is an integer selected from 1, 2, and 3. Particular conjugates of this type include:

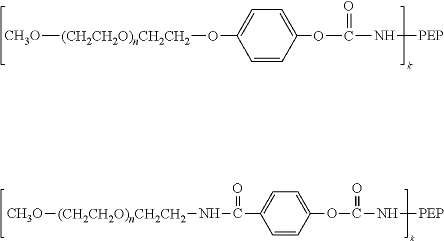

where n ranges from about 10 to about 1800.

Additional releasable conjugates in accordance with the invention are prepared using water-soluble polymer reagents such as those described in U.S. Pat. No. 6,214,966. Such water-soluble polymers result in a releasable linkage following conjugation, and possess at least one releasable ester linkage close to the covalent attachment to the active agent. The polymers generally possess the following structure, PEG-W—CO₂—NHS or an equivalent activated ester, where

W=—O₂C—(CH₂)_{b}—O— b=1-5

—O—(CH₂)_{b}CO₂—(CH₂)_{c}— b=1-5, c=2-5

—O—(CH₂)_{b}—CO₂—(CH₂)_{c}—O— b=1-5, c=2-5

and NHS is N-hydroxysuccinimidyl. Upon hydrolysis, the resulting released active agent, e.g., therapeutic peptide, will possess a short tag resulting from hydrolysis of the ester functionality of the polymer reagent. Illustrative releasable conjugates of this type include: mPEG-O—(CH₂)_{b}—COOCH₂C(O)—NH-therapeutic peptide, and mPEG-O—(CH₂)_{b}—COO—CH(CH₃)—CH₂—C(O)—NH-therapeutic peptide, where the number of water-soluble polymers attached to therapeutic peptide can be anywhere from 1 to 4, or more preferably, from 1 to 3.

### Carboxyl Coupling and Resulting Conjugates

Carboxyl groups represent another functional group that can serve as a point of attachment to the therapeutic peptide. The conjugate will have the following structure:

PEP-C(O)—X-POLY

where PEP-C(O)˜ corresponds to a residue of a therapeutic peptide where the carbonyl is a carbonyl (derived from the carboxy group) of the therapeutic peptide, X is a spacer moiety, such as a heteroatom selected from O, N(H), and S, and POLY is a water-soluble polymer such as PEG, optionally terminating in an end-capping moiety.

The C(O)—X linkage results from the reaction between a polymeric derivative bearing a terminal functional group and a carboxyl-containing therapeutic peptide. As discussed above, the specific linkage will depend on the type of functional group utilized. If the polymer is end-functionalized or “activated” with a hydroxyl group, the resulting linkage will be a carboxylic acid ester and X will be O. If the polymer backbone is functionalized with a thiol group, the resulting linkage will be a thioester and X will be S. When certain multi-arm, branched or forked polymers are employed, the C(O)X moiety, and in particular the X moiety, may be relatively more complex and may include a longer linker structure.

Polymeric reagents containing a hydrazide moiety are also suitable for conjugation at a carbonyl. To the extent that the therapeutic peptide does not contain a carbonyl moiety, a carbonyl moiety can be introduced by reducing any carboxylic acid functionality (e.g., the C-terminal carboxylic acid). Specific examples of polymeric reagents comprising a hydrazide moiety, along with the corresponding conjugates, are provided in Table C, below. In addition, any polymeric reagent comprising an activated ester (e.g., a succinimidyl group) can be converted to contain a hydrazide moiety by reacting the polymer activated ester with hydrazine (NH₂—NH₂) or tert-butyl carbamate (NH₂NHCO₂C(CH₃)₃). In the table, the variable (n) represents the number of repeating monomeric units and “═C-(PEP)” represents a residue of a therapeutic peptide following conjugation to the polymeric reagent were the underlined C is part of the therapeutic peptide. Optionally, the hydrazone linkage can be reduced using a suitable reducing agent. While each polymeric portion (e.g., (OCH₂CH₂)ₙ or (CH₂CH₂O)ₙ) presented in Table C terminates in a “CH₃” group, other groups (such as H and benzyl) can be substituted therefor.

TABLE C
Carboxyl-Specific Polymeric Reagents and the GM-Therapeutic Peptide Conjugates Formed
Therefrom

| Polymeric Reagent | Corresponding Conjugate |
|---|---|
| 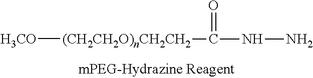 | 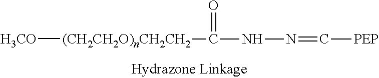 |
| 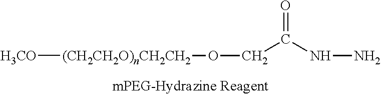 | 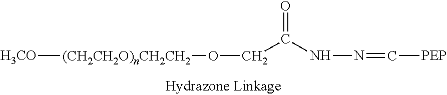 |
| 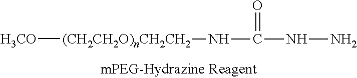 | 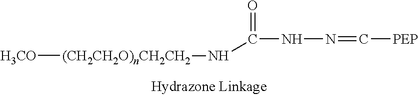 |
| 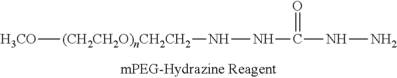 | 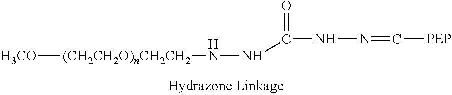 |
| 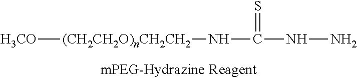 | 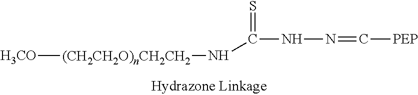 |
| 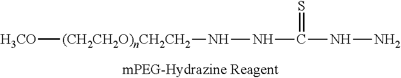 | 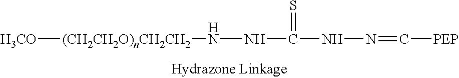 |
| 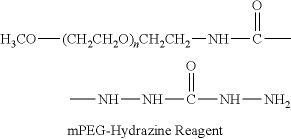 | 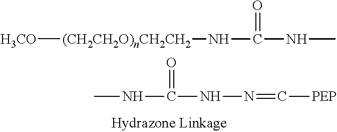 |
| 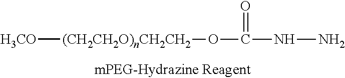 | 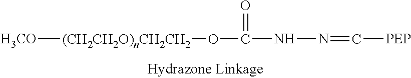 |

### Thiol Coupling and Resulting Conjugates

Thiol groups contained within the therapeutic peptide can serve as effective sites of attachment for the water-soluble polymer. The thiol groups contained in cysteine residues of the therapeutic peptide can be reacted with an activated PEG that is specific for reaction with thiol groups, e.g., an N-maleimidyl polymer or other derivative, as described in, for example, U.S. Pat. No. 5,739,208, WO 01/62827, and in Table D below. In certain embodiments, cysteine residues may be introduced in the therapeutic peptide and may be used to attach a water-soluble polymer.

Specific examples of the reagents themselves, along with the corresponding conjugates, are provided in Table D below. In the table, the variable (n) represents the number of repeating monomeric units and “—S-(PEP)” represents a residue of a therapeutic peptide following conjugation to the water-soluble polymer, where the S represents the residue of a therapeutic peptide thiol group. While each polymeric portion (e.g., (OCH₂CH₂)ₙ or (CH₂CH₂O)ₙ) presented in Table D terminates in a “CH₃” group, other end-capping groups (such as H and benzyl) or reactive groups may be used as well.

TABLE D
Thiol-Specific Polymeric Reagents and the Therapeutic peptide Conjugates Formed
Therefrom

| Polymeric Reagent | Corresponding Conjugate |
|---|---|
| 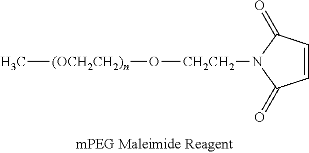 | 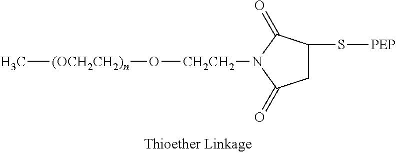 |
| 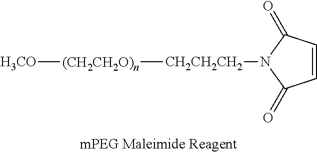 | 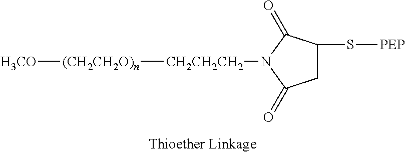 |
| 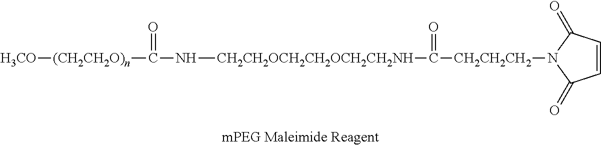 | 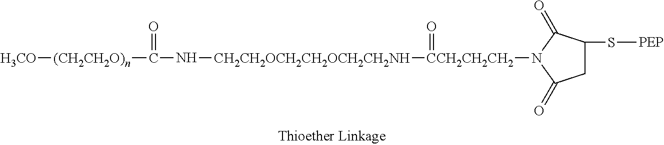 |
| 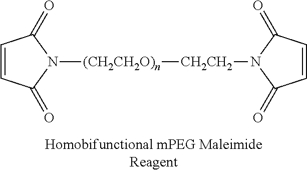 | 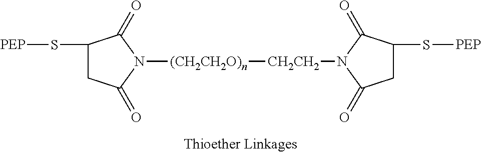 |
| 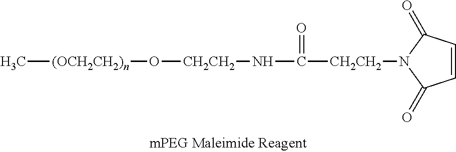 | 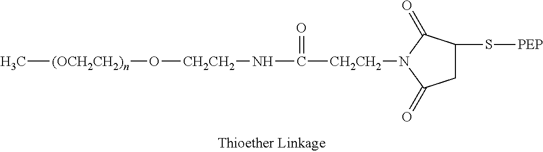 |
| 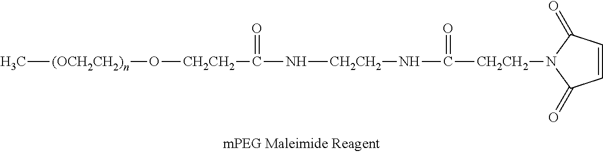 | 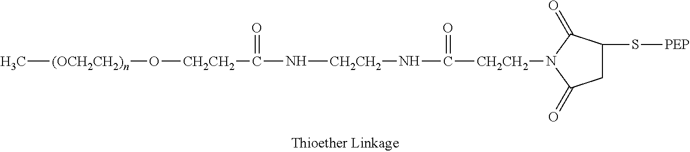 |
| 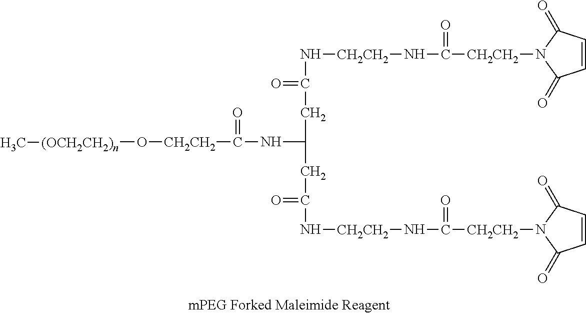 | 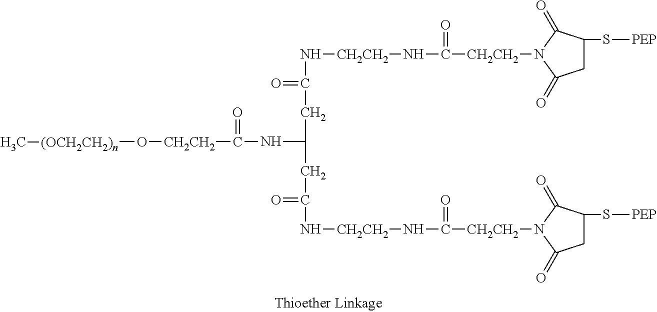 |
| 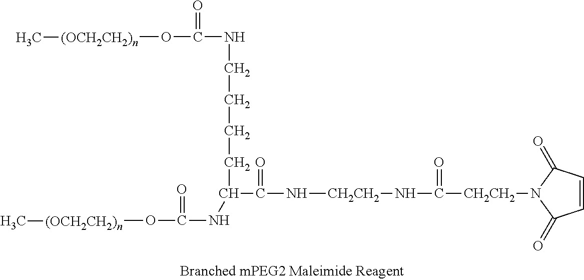 | 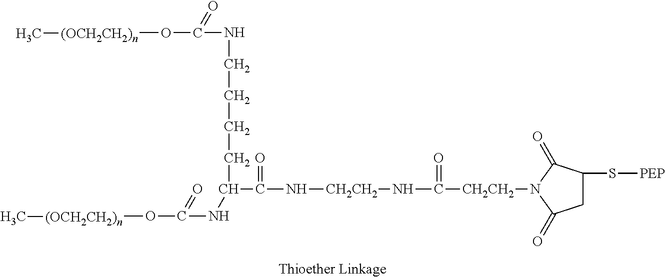 |
| 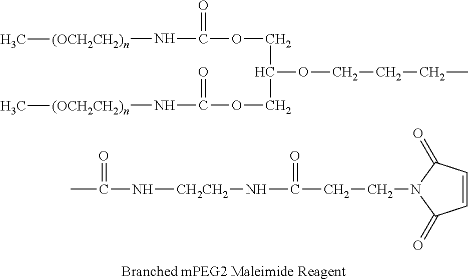 | 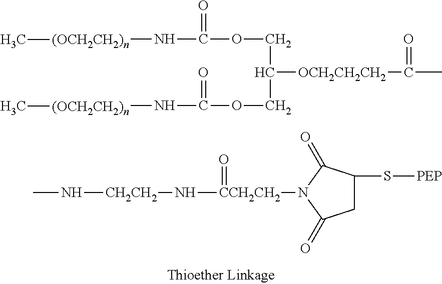 |
| 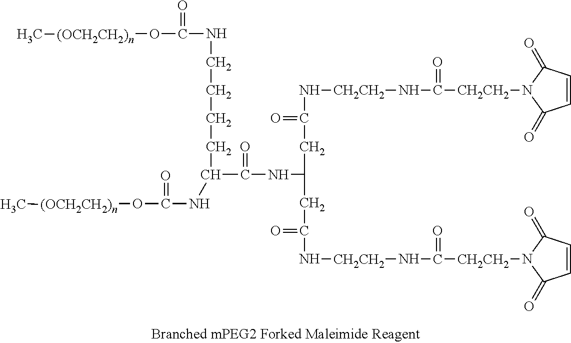 | 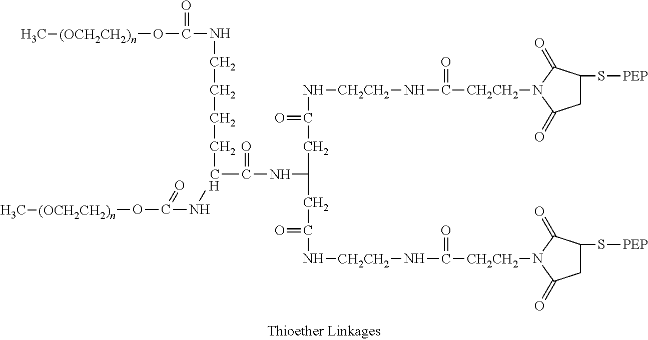 |
| 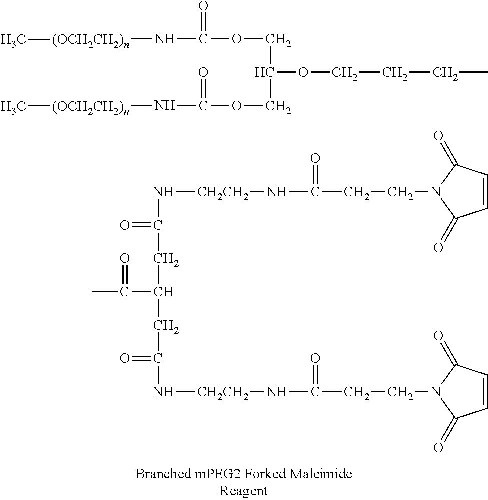 | 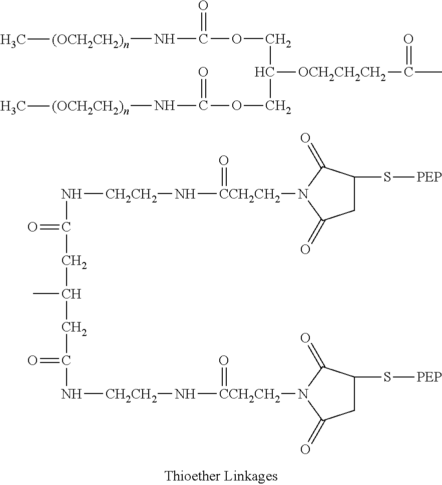 |
| 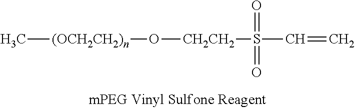 | 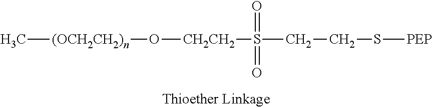 |
| 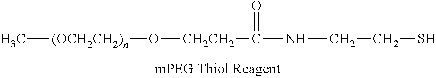 | 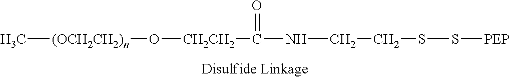 |
| 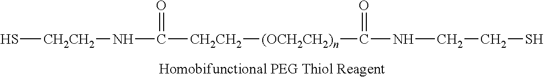 | 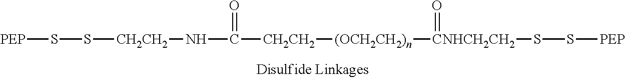 |
| 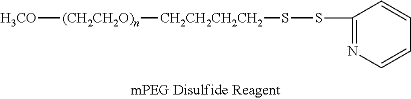 | 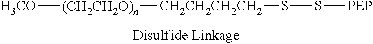 |
| 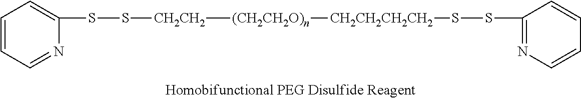 | 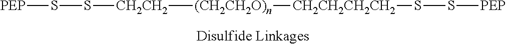 |

With respect to conjugates formed from water-soluble polymers bearing one or more maleimide functional groups (regardless of whether the maleimide reacts with an amine or thiol group on the therapeutic peptide), the corresponding maleamic acid form(s) of the water-soluble polymer can also react with the therapeutic peptide. Under certain conditions (e.g., a pH of about 7-9 and in the presence of water), the maleimide ring will “open” to form the corresponding maleamic acid. The maleamic acid, in turn, can react with an amine or thiol group of a therapeutic peptide. Exemplary maleamic acid-based reactions are schematically shown below. POLY represents the water-soluble polymer, and ˜S-PEP represents a residue of a therapeutic peptide, where the S is derived from a thiol group of the therapeutic peptide.

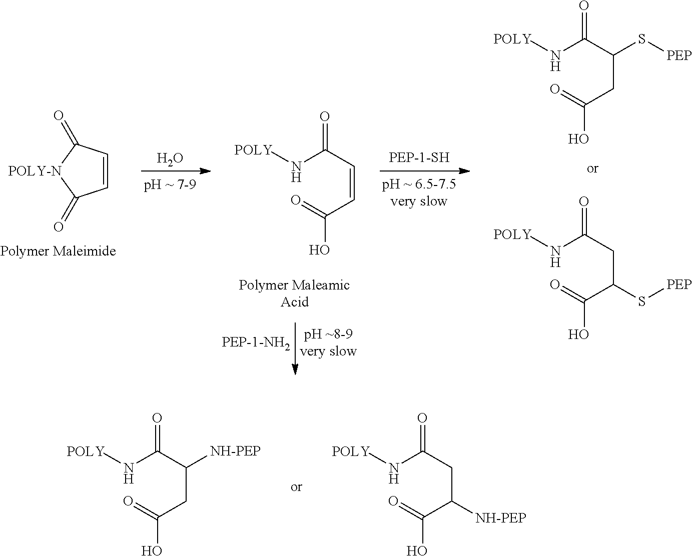

Thiol PEGylation is specific for free thiol groups on the therapeutic peptide. Typically, a polymer maleimide is conjugated to a sulfhydryl-containing therapeutic peptide at pHs ranging from about 6-9 (e.g., at 6, 6.5, 7, 7.5, 8, 8.5, or 9), more preferably at pHs from about 7-9, and even more preferably at pHs from about 7 to 8. Generally, a slight molar excess of polymer maleimide is employed, for example, a 1.5 to 15-fold molar excess, preferably a 2-fold to 10 fold molar excess. Reaction times generally range from about 15 minutes to several hours, e.g., 8 or more hours, at room temperature. For sterically hindered sulfhydryl groups, required reaction times may be significantly longer. Thiol-selective conjugation is preferably conducted at pHs around 7. Temperatures for conjugation reactions are typically, although not necessarily, in the range of from about 0° C. to about 40° C.; conjugation is often carried out at room temperature or less. Conjugation reactions are often carried out in a buffer such as a phosphate or acetate buffer or similar system.

With respect to reagent concentration, an excess of the polymeric reagent is typically combined with the therapeutic peptide. The conjugation reaction is allowed to proceed until substantially no further conjugation occurs, which can generally be determined by monitoring the progress of the reaction over time.

Progress of the reaction can be monitored by withdrawing aliquots from the reaction mixture at various time points and analyzing the reaction mixture by SDS-PAGE or MALDI-TOF mass spectrometry or any other suitable analytical method. Once a plateau is reached with respect to the amount of conjugate formed or the amount of unconjugated polymer remaining, the reaction is assumed to be complete. Typically, the conjugation reaction takes anywhere from minutes to several hours (e.g., from 5 minutes to 24 hours or more). The resulting product mixture is preferably, but not necessarily purified, to separate out excess reagents, unconjugated reactants (e.g., therapeutic peptide) undesired multi-conjugated species, and free or unreacted polymer. The resulting conjugates can then be further characterized using analytical methods such as MALDI, capillary electrophoresis, gel electrophoresis, and/or chromatography.

An illustrative therapeutic peptide conjugate formed by reaction with one or more therapeutic peptide thiol groups may possess the following structure:

POLY-X_{0,1}—C(O)Z—Y—S—S-(PEP)

where POLY is a water-soluble polymer, X is an optional linker, Z is a heteroatom selected from the group consisting of O, NH, and S, and Y is selected from the group consisting of C₂₋₁₀ alkyl, C₂₋₁₀ substituted alkyl, aryl, and substituted aryl, and —S-PEP is a residue of a therapeutic peptide, where the S represents the residue of a therapeutic peptide thiol group. Such polymeric reagents suitable for reaction with a therapeutic peptide to result in this type of conjugate are described in U.S. Patent Application Publication No. 2005/0014903, which is incorporated herein by reference.

With respect to polymeric reagents suitable for reacting with a therapeutic peptide thiol group, those described here and elsewhere can be obtained from commercial sources. In addition, methods for preparing polymeric reagents are described in the literature.

### Additional Conjugates and Features Thereof

As is the case for any therapeutic peptide polymer conjugate of the invention, the attachment between the therapeutic peptide and water-soluble polymer can be direct, wherein no intervening atoms are located between the therapeutic peptide and the polymer, or indirect, wherein one or more atoms are located between the therapeutic peptide and polymer. With respect to the indirect attachment, a “spacer moiety or linker” serves as a link between the therapeutic peptide and the water-soluble polymer. The one or more atoms making up the spacer moiety can include one or more of carbon atoms, nitrogen atoms, sulfur atoms, oxygen atoms, and combinations thereof. The spacer moiety can comprise an amide, secondary amine, carbamate, thioether, and/or disulfide group. Nonlimiting examples of specific spacer moieties (including “X”, X¹, X², and X³) include those selected from the group consisting of —O—, —S—, —S—S—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—(CH₂)ₕ—(OCH2CH2)ⱼ-, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (j) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes. Additionally, any of the above spacer moieties may further include an ethylene oxide oligomer chain comprising 1 to 20 ethylene oxide monomer units (i.e., —(CH₂CH₂O)₁₋₂₀). That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment.

As indicated above, in some instances the water-soluble polymer-(PEP) conjugate will include a non-linear water-soluble polymer. Such a non-linear water-soluble polymer encompasses a branched water-soluble polymer (although other non linear water-soluble polymers are also contemplated). Thus, in one or more embodiments of the invention, the conjugate comprises a therapeutic peptide covalently attached, either directly or through a spacer moiety comprised of one or more atoms, to a branched water-soluble polymer, at in a non-limiting example, an internal or N-terminal amine. As used herein, an internal amine is an amine that is not part of the N-terminal amino acid (meaning not only the N-terminal amine, but any amine on the side chain of the N-terminal amino acid).

Although such conjugates include a branched water-soluble polymer attached (either directly or through a spacer moiety) to a therapeutic peptide at an internal amino acid of the therapeutic peptide, additional branched water-soluble polymers can also be attached to the same therapeutic peptide at other locations as well. Thus, for example, a conjugate including a branched water-soluble polymer attached (either directly or through a spacer moiety) to a therapeutic peptide at an internal amino acid of the therapeutic peptide, can further include an additional branched water-soluble polymer covalently attached, either directly or through a spacer moiety comprised of one or more atoms, to the N-terminal amino acid residue, such as at the N-terminal amine.

One preferred branched water-soluble polymer comprises the following structure:

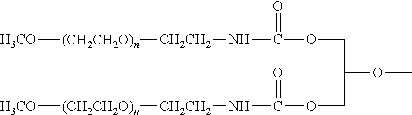

wherein each (n) is independently an integer having a value of from 3 to 4000, or more preferably, from about 10 to 1800.

Also forming part of the invention are multi-armed polymer conjugates comprising a polymer scaffold having 3 or more polymer arms each suitable for capable of covalent attachment of a therapeutic peptide.

Exemplary conjugates in accordance with this embodiment of the invention will generally comprise the following structure:

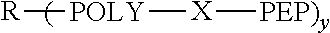

wherein R is a core molecule as previously described, POLY is a water-soluble polymer, X is a cleavable, e.g., hydrolyzable linkage, and y ranges from about 3 to 15.

More particularly, such a conjugate may comprise the structure:

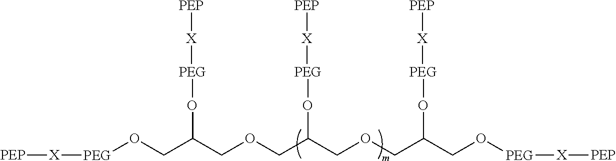

where m is selected from 3, 4, 5, 6, 7, and 8.

In yet a related embodiment, the therapeutic peptide conjugate may correspond to the structure:

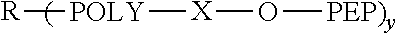

where R is a core molecule as previously described, X is —NH—P—Z—C(O)P is a spacer, Z is —O—, —NH—, or —CH₂—, —O-PEP is a hydroxyl residue of a therapeutic peptide, and y is 3 to 15. Preferably, X is a residue of an amino acid.

In one embodiment, the modified insulin is shown as follows:

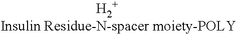

wherein Insulin Residue along with the secondary amine (—NH₂⁺—) comprises an insulin residue optionally including at least one moiety having carbon atoms as described above, spacer moiety is a spacer moiety, and POLY comprises a hydrophilic polymer as discussed above. It should be noted that the (—NH₂⁺—) in the above and below structures may be —NH-depending on the pH, e.g., at pH's greater than 7. In a preferred embodiment, the insulin is substituted at the B1 and/or B29 amino acid residue(s).

The spacer moiety or linker of the invention may be a single atom, such as an oxygen or a sulfur, two atoms, or a number of atoms. A linker is typically but is not necessarily linear in nature. The overall length of the linker will typically range between 1 to about 40 atoms, wherein “length” means the number of atoms in a single chain, not counting substituents. For instance, —CH₂— counts as one atom with respect to overall linker length, —CH₂CH₂O— counts as 3 atoms in length. Preferably, a linker will have a length of about 1 to about 20 atoms or from about 2 to about 15 atoms.

The linker of the invention can be a single functional group such as an amide, an ester, a urethane, or a urea, or may contain methylene or other alkylene groups flanking either side of the single functional group. Alternatively, a linker may contain a combination of functional groups that can be the same or different. Additionally, a linker of the invention can be an alkylene chain, optionally containing one or more oxygen or sulfur atoms (i.e., an ether or thioether). Preferred linkers are those that are hydrolytically stable.

In one or more embodiments, the modified insulin molecule comprises at least one amino acid residue covalently attached to a hydrophilic polymer via a spacer moiety comprising at least 4 carbon atoms, wherein the spacer moiety is attached to the at least one amino acid residue via a secondary amine, is shown as follows:

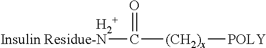

wherein Insulin Residue is as defined above; x is at least 3, such as at least 4 or at least 5, and may, e.g., range from 3 to 20, such as 3 to 10, or 4 to 8; and POLY is a hydrophilic polymer as discussed above. In certain embodiments, x is 3.

In one or more embodiments, the modified insulin molecule comprises at least one amino acid residue covalently attached to a hydrophilic polymer via a spacer moiety comprising at least 4 carbon atoms, wherein the spacer moiety is attached to the at least one amino acid residue via a secondary amine, is shown as follows:

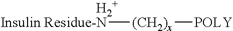

wherein Insulin Residue is as defined above; x is at least 4, such as at least 5 or at least 6, and may, e.g., range from 4 to 20, such as 4 to 10, or 5 to 8; and POLY is a hydrophilic polymer as discussed above. In certain embodiments, x is 4.

In certain embodiments, the modified insulin molecule is shown as follows:

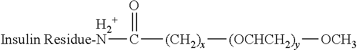

wherein Insulin Residue is as defined above; x is at least 3, such as at least 4 or at least 5, and may, e.g., range from 3 to 20, such as 3 to 10, or 4 to 8; and y is at least 3, such as at least 10, or at least 20, and may range, e.g., from 5 to 400, or 10 to 200. In certain embodiments, x is 3.

In yet other embodiments, the modified insulin is shown as follows:

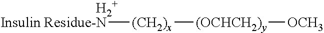

wherein Insulin Residue is as defined above; x is at least 4, such as at least 5 or at least 6, and may, e.g., range from 4 to 20, such as 4 to 10, or 5 to 8; and y is at least 3, such as at least 10, or at least 20, and may range, e.g., from 5 to 400, or 10 to 200. In certain embodiments, x is 4.

In the above embodiments, the modified insulin molecule comprises at least one amino acid residue covalently attached to a hydrophilic polymer via a spacer moiety comprising at least 4 carbon atoms, wherein the spacer moiety is attached to the at least one amino acid residue via a secondary amine. It is believed that the secondary amine may provide advantages over other groups, e.g., amides. For instance, while not wishing to be bound by theory, the secondary amine is believed to result in a more stable conjugate than other groups. For example, the secondary amine may be less prone to aggregation than amides. In this regard, the secondary amine allows the modified insulin to keep its charge at this position.

In one embodiment, the modified insulin comprises a polyethylene glycol butyrl covalently bonded to insulin at A1 and/or B29.

### Moieties Having Carbon Atoms

In some embodiments, the modified therapeutic peptide includes a moiety having carbon atoms, such as a moiety having one to ten carbon atoms, such as one to three carbon atoms. While not wishing to be bound by theory, it is believed that these moieties protect the modified therapeutic peptide from enzymatic degradation, e.g., aminopeptidase and/or trypsin degradation.

In some cases, the moiety having carbon atoms is irreversibly attached to the insulin. In this regard, the moiety cannot be removed without affecting the amino acid sequence of the insulin. In some embodiments, less than 50% of the moiety is removed at pH 2 to 10 or pH 3 to 8, after 24 hours at room temperature. For instance, in some embodiments, less than 50% of the moiety is removed when the modified insulin is subjected to any one of the following conditions or any one of any sub-group of the following conditions:

(1) 5 mg/ml of the modified insulin is placed in trifluoroacetic acid (TFA) for 2 hours at 20° C.;

(2) 5 mg/ml of the modified insulin is placed in water containing 2 M acetic acid for 24 hours at 20° C.;

(3) 5 mg/ml of the modified insulin is placed in water containing 50 mM acetic acid for 24 hours at 20° C.;

(4) 5 mg/ml of the modified insulin is placed in water containing 50 mM Tris at pH 8.5 for 24 hours at 40° C.;

(5) 5 mg/ml of the modified insulin is placed in DMSO containing 20 (w/v) % piperidine for 5 minutes at 20° C.;

(6) 5 mg/ml of the modified insulin is placed in a water/acetonitrile (1:1) mixture solution; the solution is bubbled with N₂ for at least 15 min; Pd/C catalyst (10 wt % on activated carbon) is then added slowly to 10 wt % of modified insulin; then the reaction mixture is agitated; the system is evacuated and recharged with hydrogen gas under 50 psi three times (agitation is stopped during evacuation and recharging); the reaction mixture is then kept at room temperature under 50 psi for 16 hrs;

(7) 5 mg/ml of the modified insulin is placed in ethylene glycol; 10 molar equivalents of hydrazine monohydrate 8 molar equivalents of KOH are added; the reaction mixture is heated to 100° C., under nitrogen for 30 minutes; and

(8) 5 mg/ml of the modified insulin is placed in anhydrous HF at 0° C. for 30 minutes.

As used herein, “moieties having one to ten carbon atoms” include any moiety having one to ten carbons. One to ten means 10, 9, 8, 7, 6, 5, 4, 3, 2, or 1 carbon atom(s). The range of carbon atoms may be from 1 to 10, or 2 to 8, or 3 to 6, or from any integer from 1 to 10 and to any other integer from 1 to 10. Thus, all possible ranges of integers involving the numbers 1, 2, 3, 4, 5, 6, 7, 8, 9, and 10, are expressly contemplated. Similarly, moieties having one to three carbon atoms include any moiety having one to three carbons.

Although the moiety should be large enough to protect the modified insulin from enzymatic degradation, the moiety is typically small enough to avoid substantially interfering with the activity of the modified insulin. For instance, the total number of atoms in the moiety is usually less than 20 atoms, such as less than 19, 18, 17, 16, 15, 14, 13, 12, 11, 10, 9, or 8 atoms, and typically ranges from 4 to 20 atoms, such as 6 to 12 atoms.

The moiety may comprise solely carbons and hydrogens, or may additionally comprise heteroatoms, such as oxygen, nitrogen, or sulfur. Other atoms, such as halogens, are also expressly contemplated as well. Indeed, any atom can be included in the moiety so long as the modified insulin is at least substantially biocompatible and nonimmunogenic.

The moiety may comprise a straight chain, branched chain, or a ring. For instance, the moiety may comprise a straight chain having one to ten carbons, as defined above. The moiety may be added before or after the hydrophilic polymer.

The moiety may comprise a group as represented in the following formula:

-A-D-Q-X

wherein:

A is selected from methyl, —CR₂—, —C(O)—, —O—, —S(O)(O)—, and —S—;

D, if present, is selected from —CR₂—, —C(O)—, —O—, pyridinyl, substituted pyridinyl, phenyl, substituted phenyl, cycloalkyl, —CY₃ where Y is independently selected from hydrogen and lower alkyl;

Q, if present, is selected from —CR₃, phenyl, and substituted phenyl; and

X, if present, is phenyl;

wherein R is selected from hydrogen and lower alkyl.

In certain embodiments, A is —CH₂—, D is —CY₃, and Y is selected from hydrogen and lower alkyl, e.g., —CY₃ may be a lower alkyl, such as methyl.

In certain embodiments, A is —C(O)—, D is CY₃, and Y is selected from hydrogen and lower alkyl, e.g., —CY₃ may be a lower alkyl, such as methyl.

Examples of such moieties include, but are not limited to, methyl, ethyl, propyl, butyl, pentyl, hexyl, t-butoxycarbonyl, fluorenylmethyloxycarbonyl, nicotinyl, t-butyl, benzoyl, acetyl, carbobenzoxyl, methyl ester, ethyl ester, propyl ester, butyl ester, pentyl ester, hexyl ester, N-methyl anthranilyl, amide, 9-fluoreneacetyl, 1-fluorenecarboxyl, 9-fluorenecarboxyl, 9-fluorenone-1-carboxylic group, xanthyl, trityl, 4-methyltrityl, 4-methoxytrityl, 4-methoxy-2,3,6-trimethyl-benzenesulphonyl, mesitylene-2-sulphonyl, 4,4-dimethoxybenzhydryl, tosyl, 2,2,5,7,8-pentamethylchroman-6-sulphonyl, 4-methylbenzyl, 4-methoxybenzyl, benzyloxy, benzyl, 3-nitro-2-pyridinesulphenyl, 1-(4,4-dimethyl-2,6-dioxocyclohexylidene)ethyl, 2,6-dichlorobenzyl, 2-chlorobenzyloxycarbonyl, 2-bromobenzyloxycarbonyl, benzyloxymethyl, cyclohexyloxy, t-butoxymethyl, trifluoroacetyl, 4[N-{1-(4,4-dimethyl-2,6-dioxocyclohexylidene)-3-methyldibutyl}-amino]benzyl ester, α-allyl ester, 2-phenylisopropyl ester, and 1-[4,4-dimethyl-2,6-dioxycyclohex-1-yl-idene]ethyl. The foregoing list is limited for brevity; any “blocking group” or “protecting group,” examples of which are well known in the art, may be used.

It is expressly contemplated that moieties having one to ten carbons, as referred to herein, may optionally exclude any amino acids, including non-naturally occurring amino acids, phosphates, glutathione, as well as sugars, carbohydrates, and any other form of glycosylation. The possible exclusion of any or all of these compounds is expressly contemplated.

### Modification of Insulins

The insulin molecule possesses several sites suitable for modification by addition of a hydrophilic polymer or other moiety, with amino sites generally but not necessarily being most preferred. Specific insulin amino groups suitable for modification include the two N-termini, GlyA1 and PheB1, as well as LysB29. These sites on the insulin molecule are also referred to herein simply as A1, B1, and B29, respectively. In the case of addition and deletion variants, B29 Lys and amino termini may appear at different positions than wild-type insulin. Such analogs are considered within the scope of the present invention. For instance, “B29” as defined herein includes other positions when shifted by addition or deletion of amino acids. Moreover, the insulin molecule can be provided in any manner.

A composition of the invention may, in some embodiments, contain predominantly (greater than 90%) mono-modified insulin, e.g., mono-A1 insulin, mono-B1 insulin, or mono-B29 insulin. Such compositions may contain: i) mono-A1 insulin, ii) a mixture of mono-A1 insulin and mono-B1 insulin, or iii) a mixture of mono-A1, mono-B1, and mono-B29 insulin. Alternatively, a composition of the invention may contain predominantly di-substituted insulin, e.g., di-A1,B1-insulin, or di-A1,B29-insulin, or di-B1,B29-insulin, or any of the various combinations thereof. Still further, modified insulins of the invention may be modified in three positions, such as at each of A1, B1, and B29, or at other positions as well.

Alternatively, a composition in accordance with the invention may contain a mixture of various modified insulins (i.e., hydrophilic polymer or other moiety attached to any one or more of a combination of possible attachment sites). Using the amino sites on insulin as an example, a composition of the invention may contain any one or more of the following modified insulins: monoA1-modified insulin, mono-B1-insulin, mono-B-29 insulin, di-A1,B1-insulin, di-A1,B29-insulin, di-B1,B29-insulin, and tri-A1,B-1,B29-insulin. Such compositions can be described as exhibiting inter-molecule heterogeneity.

Still further, it is noted that the modified insulins of the invention may include different kinds of modifications. Thus, a modified insulin may include multiple modifications that are of different types. A modified insulin of the invention may include modifications with hydrophilic polymers at two positions and a moiety having one to ten carbons at one position. Similarly, a modified insulin may have a hydrophilic polymer at one position and also have a moiety with one to ten carbon atoms at one or two positions. One embodiment, for example, includes a hydrophilic polymer at the B1 position and a moiety having one to ten carbon atoms at the A1 and B29 positions. Such molecules can be described as exhibiting intra-molecule heterogeneity.

It should be noted that the aforementioned A1, B1, and B29 sites are preferred examples of sites that can be modified in accordance with the invention. Alternative sites in the native insulin molecule that can be chemically modified by covalent attachment of hydrophilic polymer or other moiety include, but are not limited to, the C-termini, ArgB22, HisB10, HisA5, GluA4, GluA17, GluB13, and GluB21.

In addition to native insulin, non-native insulins having one or more amino acid substitutions, insertions, or deletions may be utilized, such that additional sites become available for chemical modification by attachment of one or more hydrophilic polymers or other moieties. This embodiment of the invention is particularly useful for introducing additional, customized modification sites within the insulin molecule, for example, for forming a modified insulin having improved resistance to enzymatic degradation. Such an approach provides greater flexibility in the design of a modified insulin having the desired balance of activity, stability, solubility, and pharmacological properties.

One example of a non-native insulin is a substitution variant in which any one of the first four amino acids in the B-chain is replaced with a cysteine residue. Such cysteine residues can then be reacted with an activated PEG that is specific for reaction with thiol groups, e.g., an N-maleimidyl polymer or other derivative, as described in U.S. Pat. No. 5,739,208 and in International Patent Publication No. WO 01/62827. Exemplary sulfhydryl-selective PEGs for use in this particular embodiment of the invention include mPEG-forked maleimide (mPEG(MAL)₂), mPEG2-forked maleimide (mPEG2(MAL)₂), mPEG-maleimide (mPEG-MAL), and mPEG2-maleimide (mPEG2-MAL) (Shearwater Corporation).

Electrophilically activated PEGs for use in coupling to reactive amino groups on insulin include mPEG2-ALD, mPEG-succinimidyl propionate, mPEG-succinimidyl butanoate, mPEG-butyraldehyde, mPEG-CM-HBA-NHA, mPEG-benzotriazole carbonate, mPEG-acetaldehyde diethyl acetal, etc. (Shearwater Corporation, Huntsville, Ala.).

Polyethylene glycols usable in accordance with the present invention are described, for example, in U.S. Pat. No. 6,890,518, which is incorporated herein by reference.

### Methods of Producing Modified Peptides

In view of the present description, the compounds of the present invention may be made by any of the various methods and techniques known and available to those skilled in the art. In this regard, the modified therapeutic peptides, e.g., modified insulins, of the invention can be prepared in any number of ways. Consequently, the modified therapeutic peptides provided herein are not limited to the specific technique or approach used in their preparation. Exemplary approaches for preparing the presently described modified insulins, however, will be discussed in detail below.

Modified therapeutic peptides of the present invention include at least one amino acid residue modified with a hydrophilic polymer and/or at least one amino acid residue modified with a moiety having one to ten carbon atoms. If the two modifications are both present, they can be performed in any order. That being said, adding the “moiety” first, followed by adding the polymer, may have practical advantages. For example, the “moiety” may act as a blocking or protecting group for more reactive sites, thereby allowing for greater targeting of the hydrophilic polymer. Details are provided below for examples of polyethylene glycol, “PEG,” as the hydrophilic polymer and acetyl as the moiety having fewer than ten carbon atoms. Of course, other polymers or moieties can be used and the synthesis procedures modified accordingly.

### Inclusion of Hydrophilic Polymer

Representative polymeric reagents and methods for conjugating such polymers to an active moiety are known in the art, and are, e.g., described in Harris, J. M. and Zalipsky, S., eds, Poly(ethylene glycol), Chemistry and Biological Applications, ACS, Washington, 1997; Veronese, F., and J. M Harris, eds., Peptide and Protein PEGylation, Advanced Drug Delivery Reviews, 54(4); 453-609 (2002); Zalipsky, S., et al., “Use of Functionalized Poly(Ethylene Glycols) for Modification of Polypeptides” in Polyethylene Glycol Chemistry: Biotechnical and Biomedical Applications, J. M. Harris, ed., Plenus Press, New York (1992); Zalipsky (1995) Advanced Drug Reviews 16:157-182, and in Roberts, et al., Adv. Drug Delivery Reviews, 54, 459-476 (2002).

Additional PEG reagents suitable for use in forming a modified insulin of the invention, and methods of conjugation, are described in the Nektar Advanced PEGylation Catalogs, 2005-2006; 2004; 2003; and in Shearwater Corporation, Catalog 2001; Shearwater Polymers, Inc., Catalogs, 2000 and 1997-1998; U.S. Published Application No. 20040116649; and in Pasut, G., et al., Expert Opin. Ther. Patents (2004), 14(5). PEG reagents suitable for use in the present invention also include those available from NOF Corporation, as described generally on the NOF website (2007) under Products, High Purity PEGs and Activated PEGs. Products listed therein and their chemical structures are expressly incorporated herein by reference. Additional PEGs for use in forming a modified insulin of the invention include those available from Polypure (Norway) and from QuantaBioDesign LTD (Ohio), where the contents of their online catalogs (2007) with respect to available PEG reagents are expressly incorporated herein by reference.

The particular linkage between the insulin and the hydrophilic polymer (or the spacer moiety that is attached to the hydrophilic polymer) depends on a number of factors. Such factors include, for example, the particular linkage chemistry employed, the particular insulin amino acid, the available functional groups within the insulin amino acid (either for attachment to a polymer or conversion to a suitable attachment site), the possible presence of additional reactive functional groups on the insulin.

In one or more embodiments of the invention, the linkage between the insulin and the hydrophilic polymer is a releasable linkage. That is, the hydrophilic polymer is released (either through hydrolysis, an enzymatic processes, or otherwise), thereby resulting in the otherwise modified insulin. In some cases, the releasable linkage results in the hydrophilic polymer (and any spacer moiety) detaching from the insulin in vivo (and in vitro) without leaving any fragment of the polymer (and/or any spacer or linker moiety) attached to the insulin. Exemplary releasable linkages include carbonates, carboxylate esters, phosphate esters, thiolesters, anhydrides, acetals, ketals, acyloxyalkyl ethers, imines, carbamates, and orthoesters. Such linkages can be readily formed by reaction of the insulin and/or the polymeric reagent using coupling methods commonly employed in the art. Hydrolyzable linkages are often readily formed by reaction of a suitably activated polymer with a non-modified functional group contained within the insulin. Exemplary hydrolyzable linkages are disclosed in U.S. Pat. Nos. 6,515,100; 6,864,350; and 6,899,867, which are incorporated herein by reference in their entireties.

The linkage between the insulin and the hydrophilic polymer (or the linker moiety that is attached to the polymer) may alternatively be a hydrolytically stable linkage, such as an amide, urethane (also known as carbamate), amine, secondary amine, thioether (also known as sulfide), or urea (also known as carbamide).

Conjugation of a polymeric reagent to an amine group of insulin can be accomplished by a variety of techniques. In one approach, insulin is conjugated to a polymeric reagent functionalized with an active ester such as a succinimidyl derivative (e.g., an N-hydroxysuccinimide ester). In this approach, the polymeric reagent bearing the reactive ester is reacted with the insulin in aqueous media under appropriate pH conditions, e.g., from pHs ranging from about 3 to about 11, about 3 to about 8, about 3.5 to about 7, or about 4 to about 6.5. Most polymer active esters can couple to a target protein such as insulin at physiological pH, e.g., at 7.0. However, less reactive derivatives may require a higher pH. Typically, activated PEGs can be attached to a protein at pHs from about 7.0 to about 10.0 for covalent attachment to an internal lysine. Typically, lower pHs are used, e.g., 4 to about 5.75, for preferential covalent attachment to the N-terminus. Thus, different reaction conditions (e.g., different pHs or different temperatures) can result in the attachment of a hydrophilic polymer such as PEG to different locations on the insulin (e.g., internal lysines versus the N-terminus).

In some embodiments, insulin is reacted with a reactive hydrophilic polymer by contacting insulin with a hydrophilic polymer in an organic solvent. Examples of the organic solvent include, but art not limited to, DMSO, C₁-C₄ alcohols, acetone, dioxane, NMP, THF, DMF, triethylamine, amphiphilic agents, acetonitrile, and mixtures thereof. The reactive hydrophilic polymer may be dissolved in the organic solvent at a temperature above 25° C., the reactive hydrophilic polymer in the organic solvent may be cooled to a temperature below 25° C., and the reactive hydrophilic polymer may be contacted with insulin. For instance, for PEGs having a molecular weight above 5000, the PEG may be dissolved in DMSO/DIPEA at 30° C. The PEG solution may then be cooled back to room temp after dissolution.

Coupling reactions can often be carried out at room temperature, although lower temperatures may be desired. Examples of reaction temperatures include, but are not limited to, 4 to 40° C., 15 to 30° C., or 20 to 25° C. For instance, diacetylated insulin in DMSO/DIPEA may be reacted with PEG at room temperature.

Reaction times are typically on the order of minutes, e.g., 30 minutes or hours, e.g., from about 1 to about 36 hours), depending upon the pH and temperature of the reaction. N-terminal PEGylation, e.g., with a PEG reagent bearing an aldehyde group, is typically conducted under mild conditions, pHs from about 5-10, for about 6 to 36 hours. That being said, certain embodiments of the invention involve conjugation with PEG aldehyde at pH less than 5, as discussed in more detail below.

Varying ratios of polymer reagent to insulin may be employed, e.g., from an equimolar ratio up to a 10-fold molar excess of polymer reagent. Typically, up to a 5-fold molar excess of polymer reagent will suffice.

In an alternative approach to direct coupling reactions, the PEG reagent may be incorporated at a desired position of the insulin during peptide synthesis. In this way, site-selective introduction of one or more PEGs can be achieved. See, e.g., International Patent Publication No. WO 95/00162, which describes the site selective synthesis of conjugated peptides.

In view of the teachings provided herein, optimized experimental conditions for a specific conjugate can readily be determined, typically by routine experimentation, by one skilled in the art. Reactive groups suitable for activating a PEG-polymer for attachment to a thiol (sulfhydryl) group on insulin include vinylsulfones, iodoacetamide, maleimide, and dithio-orthopyridine. Particular reagents include PEG vinylsulfones and PEG-maleimide. Additional representative vinylsulfones for use in the present invention are described in U.S. Pat. No. 5,739,208, which is incorporated herein by reference.

### Inclusion of Blocking or Protecting Moiety

The chemistry for attaching the moiety having one to ten carbon atoms will depend upon the particular moiety used and the desired site of modification. Examples of desirable reaction conditions are known in the art. The choice of reaction conditions will also depend upon the order in which the “moiety” and hydrophilic polymer are attached to the insulin molecule.

As with the chemistry for attachment of the hydrophilic polymer, reaction conditions, such as temperature, pH, and molar ratios of reactants, can affect the end product. Thus, the extent of modification—mono, di, tri, etc. —can be varied; the positions of modification—e.g., A1, B1, B29 of insulin, etc. —can be varied; and the heterogeneity—intramolecule and/or intermolecule—can be varied, simply by varying the reaction conditions. The particular choice will depend on the desired end product; variations and modifications of the methods described herein can be made by those of ordinary skill through nothing more than routine experimentation. The following discussion provides some considerations for the modification of some amino acids.

The order of modification of A1, B1, and B29 can be affected by the exposure of the nucleophilic amino groups of these residues to the solvent and by the accessibility of the blocking or protecting agent to the nucleophilic amino group. Generally, the more an amino group is exposed to the solvent and the less hindrance the remainder of the protein provides, the faster an amino group is blocked or protected. Thus, the ideal reaction conditions depend on the reactants and the targeted site. In view of the present disclosure, a skilled artisan would be able to add blocking or protecting moieties.

For lysine B29, its ε-amino group is predominately exposed to the solvent, while the amino groups of A1 or B1 may have less exposure to the solvent due to their orientation within the three-dimensional structure of the protein. Therefore, under high pH reaction conditions (e.g., pH 9 to 11, such as 10.5) in regular aqueous medium, B29 is modified first, followed by A1. This result is surprising to the extent that the pKₐ's of A1 (about 8.0), B1 (<7.0), and B29 (8.5) would suggest that the order of reaction would be B1, A1, and B29. It appears that steric hindrances dominate over pKₐ. As a result, the ability to block at A1 and B29 while leaving B1 unblocked with great specificity is a great improvement. In other words, the high yield and rate of the present reaction, as discussed in more detail below, is a great improvement.

Changes in the local environment around the protein also can affect the reactivity of the amino groups. For example, if the pH is lowered (e.g., to less than pH 10.5, such as 5 to 10.5), the amino group at B29 becomes protonated. Protonated amino groups are not nucleophilic and therefore do not react as quickly with blocking or protecting agents. In such a case, A1, which is not protonated at this point, is the most reactive group. Still further, if the pH is lowered more (e.g., to less than pH 5), B29 and A1 are protonated, and B1 becomes the only reactive amino acid residue. In view of the above, examples of the pH for protecting or blocking A1 and B29 include, but are not limited to, 8 to 12, such as 8.5 to 11.5 or 9 to 11.

Besides pH as a condition to adjust reactivity and location of the blocking or protecting of amino groups A1, B1, and B29 in insulin, other conditions that may be changed are polarity and dipole property of the solvent. For example, in an organic solvent with organic base (e.g., dimethyl sulfoxide (DMSO)/triethylamine (TEA) or DMSO/N,N′-diisopropylethylamine (DIPEA)), the order of reactivity is A1>B29>B1. In an alkaline aqueous solution, the reactivity rearranges to B29>A1>>B1 (see Uchio et al., Adv. Drug Del. Rev., Vol. 35, pp. 289-306 (1999)). In general, if the polarity of an aqueous solvent is changed by adding less polar co-solvents, e.g., a water-miscible organic solvent such as C₁-C₄ alcohols, acetone, THF, DMSO, DMF, then a conformational adjustment of the protein may be the result. Such a conformational adjustment of the protein may expose residues that were previously less accessible to a greater extent resulting in increased reactivity. Similarly, a conformational adjustment of proteins can be induced by addition of amphiphilic agents, i.e., agents that contain hydrophilic and hydrophobic groups, such as detergents, surfactants, and emulsifiers, e.g., sodium dodecyl sulfate, fatty acids, or fatty alcohols. In view of the above, the solvent system may be aqueous or non-aqueous. Examples of components of solvent systems include, but are not limited to, water, C₁-C₄ alcohols, acetone, dioxane, NMP, THF, DMSO, DMF, triethylamine, amphiphilic agents, and acetonitrile.

The polarity of a solvent can also be changed by the addition of salts, such as sodium chloride, sodium bromide, potassium chloride, potassium bromide, magnesium chloride, magnesium bromide, ammonium chloride or higher organic analogs thereof, e.g., tetra ethyl ammonium chloride. An increase in the polarity of solvent system may also induce a conformational change of the protein resulting in an improved exposure of previously hindered amino acid residues to the solvent. These better accessible residues may exhibit greater reactivity toward blocking or protecting agents.

The solvent also affects the range of feasible concentrations of the reactants. In general, the concentration of insulin should be as high as possible and depends on the solvent. Considering solubility limits in aqueous systems, the concentration of insulin may, e.g., range from 1 mg/ml to 25 mg/ml, such as from 2 mg/ml to 10 mg/ml, or 2.5 mg/ml to 7.5 mg/ml, such as 2.5 mg/ml. For non-aqueous systems, the concentration of insulin may, e.g., range from 1 mg/ml to 250 mg/ml, such as 2 mg/ml to 200 mg/ml or 3 mg/ml to 150 mg/ml.

The molar ratio of modifying agent to insulin depends on the reactants and solvent system. For instance, in aqueous systems, water may compete with amine groups for reaction with the modifying agent. Examples of the molar ratio of modifying agent to insulin for aqueous solvent systems include, but are not limited to, 1.5:1 to 4:1, such as 2:1 to 3:1 or 2.5:1 to 2.9:1. Examples of the molar ratio of modifying agent to insulin for non-aqueous solvent systems include, but are not limited to, 1:1 to 4:1, such as 1.5:1 to 3:1 or 1.7:1 to 2.1:1.

In another aspect, the reactivity of a specific amino acid residue may be adjusted by the choice of the blocking or protecting agent. Blocking or protecting agents generally fall into two groups depending on the mechanism of the nucleophilic amino residue with the blocking or protecting agent. The first group comprises agents that modify by substitution. Here, the nucleophilic amino group of a protein residue P—NH₂ reacts with a blocking or protecting agent AX, forming a modified protein P—NHA and the by-product HX. An example for such compounds is acetyl chloride or more moderate acetylation agents as described below. The second group comprises agents that modify by addition. Here, the amino group of a protein simply adds to the blocking or protecting agent. For example, aldehydes are known to add to primary amines to form imines, also called a Schiff base. In view of the above, examples of blocking or protecting agents include, but are not limited to, acetylation agents, such as acetic acid-N-hydroxysuccinimide, acetic acid anhydride, and citraconic anhydride, and formylation agents.

Nature and structure of the blocking or protecting agent may have various affinities towards different residues in a protein. For example, while modification of B1 may be more preferred under more harsh conditions when a substitution blocking or protecting agent (e.g., an acetylation agent) is being used, i.e., low pH and longer reaction times, the very same amino group at B1 may be selectively modified under mild conditions when an addition blocking or protecting agent is being used, e.g., an aldehyde, such as benzaldehyde. The reaction time of the blocking reaction is often fast. Examples of reaction time include, but are not limited to, 3 minutes to 4 hours, such as 4 minutes to 3 hours, or 5 minutes to 50 minutes.

The temperature of the reaction depends on the solvent and the stability of insulin. Examples of temperature ranges include, but are not limited to, 5° C. to 40° C., such as 7° C. to 30° C. or 10° C. to 25° C. For instance, a solution of insulin in DMSO/DIPEA may be heated to 25° C. to dissolve the insulin. An acetylation reaction may take place at 25° C. to keep insulin and the acetylated products soluble in the DMSO/DIPEA.

Thus, blocking or protecting of the amino groups A1, B1, and B29 in insulin can be achieved by any number of factors, including, for example, adjustment of pH, solvent polarity, conformational adjustment of the protein, and/or choice of blocking or protecting agent. The yield of the blocking or protecting reaction typically ranges from 50% to 90%, such as 60% to 90%, 70% to 85%, or 80% to 85%, e.g., without any purification, e.g., using acetic acid-N-hydroxysuccinimide as the acetylation agent.

In certain embodiments, an N^{ε}-acylated insulin is produced in a one-step synthesis. The reaction permits the preparation of N^{ε}-acylated proteins without the use of amino-protecting groups. The acylation is carried out by reacting an activated ester, e.g., mPEG-succinimidyl propionate, mPEG-succinimidyl butanoate, and mPEG2-N-hydroxysuccinimide, with the ε-amino group of the protein under basic conditions in a polar solvent. Under weakly basic conditions, all the free amino groups are not deprotonated and significant acylation of the N-terminal amino groups results. In an aqueous solvent or semi-aqueous solvent mixture, basic conditions means the reaction is carried out at a pH greater than 9.0. Because protein degradation results at a pH range exceeding 12.0, the pH of the reaction mixture is preferably pH 9.5 to 11.5, and most preferably 10.5. The pH measurement of the reaction mixture in a mixed organic and aqueous solvent is the pH of the aqueous phase prior to mixing.

The acylation of the ε-amino group is often dependent on the basicity of the reaction. At a pH greater than 9.0, the reaction may selectively acylate the ε-amino group of B29-lysine.

In a non-aqueous solvent, the reaction may, e.g., be carried out in the presence of a base with basicity equivalent to a pKₐ around 10 or greater in water in order to sufficiently deprotonate the ε-amino group(s). For instance, the base may be at least as strong as triethylamine, such as tetramethylguanidine (TMG), diisopropylethylamine, or tetrabutylammonium hydroxide.

The choice of polar solvent is dependent largely on the solubility of the insulin and the ester. Most significantly, the solvent may be wholly organic. Generally acceptable organic solvents include DMSO, DMF and the like. Aqueous solvent and mixtures of aqueous and organic solvents are also operable. The selection of the polar solvents depends on the solubility of the reagents. Preferred solvents and solvent systems are DMSO; DMF; acetonitrile and water; acetone and water; ethanol and water; isopropyl alcohol and water; isopropyl alcohol, ethanol and water; and ethanol, propanol and water. Preferably, the solvent is acetonitrile and water; most preferably 50% acetonitrile. One skilled in the art would recognize that other polar solvents are also operable.

Generally it is preferred that the activated fatty acid ester be in molar excess. Preferably, the reaction is carried out with 1 to 4 molar equivalents, most preferably 1 to 2 molar equivalents, of the ester. However, one skilled in the art would recognize that at very high levels of activated ester, bis or tri-acylated product will be produced in significant quantity.

The temperature of the reaction affects reaction time. As an example, the reaction may be carried out at 0° C. to 40° C. and is generally complete in 15 minutes to 24 hours.

The following description is provided as just one example of one manner in which the modification can be achieved using acetyl as the “moiety” and PEG as the hydrophilic polymer. Insulin is first acetylated with acetic acid-N-hydroxysuccinimide (NHSAA), which acetylates at the A1, B29, and B1 positions of insulin. The molar ratio of NHSAA to insulin and the pH of the reaction can be varied to achieve different reaction products. For example, reacting 2:1 NHSAA:insulin at pH 10 produces primarily mono-acetylated B29 species. Reacting 3:1 NHSAA:insulin at pH 9.5 produces primarily di-acetylated A1, B29 species. The acetylated insulin can then be PEGylated by reacting it with methoxyPEG succinimidyl propionic acid.

In one embodiment, insulin is reacted with NHSAA in a molar ratio of 3:1 NHSAA:insulin at pH 9.5, to produce primarily di-acetylated A1, B29 species. These species would then be purified from other modified insulins, and the di-acetylated A1, B29 insulin would be PEGylated at the B1 position as described hereinbefore.

In view of the above, in one embodiment, the present invention relies on the protection of the A1 and B29 sites with a small and non-reversible group, e.g., acetyl group, which minimizes the loss of insulin activity due to derivatization at A1 site. After acetylation, the PEG reagent is added to yield up to about 50% B1-PEG-A 1, B29-di-acetyl-insulin analogs in the DMSO/TEA solvent reaction system. In general, the yields of the present invention are up to 80%, such as up to 70%, up to 60%, including ranges of 50% to 90%, 60% to 80%, or 65% to 75%, depending on many factors, including the size of the hydrophilic polymer. In some embodiments, the synthesis of the present invention is simple, which is advantageous in not requiring purification of the intermediate or de-protection.

After adding the blocking or protecting group, the reaction may be quenched, and the product may be purified by standard methods such as reverse phase, ion exchange chromatography, or hydrophobic chromatography. Thereafter, the product may be recovered by standard methods such as freeze drying or crystallization. Alternatively, the reaction mixture of insulin modified with the carbon containing moiety may be reacted with the hydrophilic polymer. In other words, blocking or protecting the insulin and conjugating the insulin with hydrophilic polymer may occur as a one-pot synthesis.

### Direct PEGylation of Insulin

In one aspect, the present invention is directed to a method comprising contacting a hydrophilic polymer with insulin at a pH less than 5 to form a modified insulin molecule having at least one amino acid residue covalently attached to the hydrophilic polymer via a spacer moiety comprising at least 4 carbon atoms, wherein the spacer moiety is attached to the at least one amino acid residue via a secondary amine. In one aspect, the insulin is not protected with a blocking or protecting group. Thus, in some aspects, the synthesis occurs with few steps, e.g., without a blocking or protecting step. When no blocking or protecting is necessary, the synthesis usually results in few impurities. In some embodiments, the synthesis does not result in any acrolein degradation product.

In certain embodiments, polymer alkanals, such as those described in U.S. Published Application No. 20040116649, which is incorporated herein by reference, are useful for conjugation to at least one amino group available for reaction. Typically, a PEG aldehyde is coupled to an amino group by reductive amination, resulting in formation of a secondary amine linkage between the polymer segment and insulin. In conjugating a polymer alkanal with an amino-bearing insulin, the polymer alkanal is reacted with the target amino-bearing insulin in a suitable solvent to form the corresponding imine-linked intermediate, which is then reduced to form a secondary amine linkage between the polymer and insulin. Reduction of the imine to the corresponding amine is accomplished by addition of a reducing agent as described below.

In certain embodiments, polymer aldehydes can be used to selectively target the modification of the N-terminus under conditions that differentiate the reactivity of the alpha amine at the N-terminal amino acid. Certain polymer alkanals, e.g., PEG butyraldehyde, appear to demonstrate a greater selectivity than other aldehyde derivatives, e.g., PEG propionaldehyde, and, thus, are more suitable for applications where selective N-terminus protein modification is desired.

Exemplary reaction conditions for preparing an N-terminally modified protein or peptide include (1) dissolving the protein or peptide to be modified in a non-amine-containing buffer; (2) adding to the insulin solution a polymer alkanal; (3) allowing the insulin and polymer alkanal to react to form the imine-coupled polymer conjugate; followed by (4) addition of a reducing agent to form the corresponding secondary amine coupled polymer conjugate.

The pH affects the product of the reaction. If the pH is high, the conjugation site tends to be the A1 and B29 sites. In this regard, although the B1 site has a lower pKₐ, it is more sterically hindered than the A1 and B29 sites. If the pH is low, the conjugation site tends to be B1. In general, the pH typically ranges from 3 to 11. To selectively conjugate at B1, examples of pH for this reaction include, but are not limited to, less than pH 5, less than pH 4.5, less than pH 4, less than pH 3.5, such as 3 to 5, 3.2 to 4.7, or 3.5 to 4.5. In this regard, the B1 amine, which has a lower pKa than A1 and B29, is reactive at pH less than 5. Nonetheless, using lower pH's is counterintuitive because such pH's can degrade insulin. For random attachment, the pH is higher, e.g., pH greater than 5, such as 5 to 11.

In general, the concentration of insulin should be as high as possible. Considering solubility limits in aqueous systems, the concentration of insulin may range from 0.5 mg/ml to 25 mg/ml, such as from 1 mg/ml to 10 mg/ml, or 2 mg/ml to 8 mg/ml, such as 2.5 mg/ml.

The ratio of hydrophilic polymer to insulin influences the product. Typically, the polymer alkanal is added to the insulin-containing solution at an equimolar amount or at a molar excess relative to insulin. This ratio (polymer:insulin) may range from 1:0.5 to 1:20, such as from 1:0.8 to 1:10, or 1:1 to 1:2. For instance, 1:1 insulin:PEG generally yields mono PEG insulin. Higher ratios of insulin:PEG (1:5 and 1:10) generally yield other PEG insulin conjugates in addition to the mono PEG insulin.

As noted above, the contacting may occur in the presence of a reducing agent. Examples of reducing agents include, but are not limited to, sodium cyanoborohydride, sodium borohydride, lithium aluminum hydride, tertiary butyl amine borane, sodium triacetyl borohydride, dimethylamine borate, trimethylamine borate, and pyridine borate. The reducing agent may be added in excess, e.g., in amounts ranging from about a 2-fold to a 30-fold molar excess relative to insulin. Preferred is to add the reducing agent in a 10-fold to 20-fold molar excess relative to insulin. For example, the reducing agent may be present at a concentration ranging from 5 mM to 50 mM, such as from 10 mM to 40 mM, or 15 mM to 30 mM, such as 20 mM.

Another variable is the solvent. Examples of solvents include, but are not limited to, at least one member selected from water, acetic acid, dimethylsulfoxide, dimethylformamide, acetonitrile, and mixtures thereof. Examples of mixed solvent systems include, but are not limited to, dimethylsulfoxide/aqueous, acetonitrile/aqueous buffer, ethanol/aqueous buffer, isopropylalcohol/aqueous buffer, and aqueous sodium phosphate with acetonitrile. Suitable buffers for conducting conjugation include sodium phosphate, sodium acetate, sodium carbonate, and phosphate buffered saline (PBS). For example, the solvent system may be 100 mM acetic acid at pH 4. Thus, in some embodiments, an advantage is that aqueous solvent systems may be used.

Still another variable is the reaction temperature. Examples of the reaction temperature include, but are not limited to 4° C. to 50° C., such as 10° C. to 40° C., or 15° C. to 30° C., such as 20° C.

Yet another variable is the reaction time. Examples of the reaction time include, but are not limited to, 1 hour to 48 hours, such as 2 hours to 24 hours, or 3 hours to 20 hours, such as 16 hours. The exact reaction time may be determined by monitoring the progress of the reaction over time. Progress of the reaction is typically monitored by withdrawing aliquots from the reaction mixture at various time points and analyzing the reaction mixture by SDS-PAGE or MALDI-TOF mass spectrometry or any other suitable analytical method. The resulting PEGylated conjugates may be further characterized using analytical methods such as MALDI, capillary electrophoresis, gel electrophoresis, and/or chromatography.

The yield of the direct PEGylation is typically greater than 45%, such as greater than 50%, or greater than 55%, and typically ranges from 40% to 70%, such as 45% to 65%, or 50% to 65%, such as 60%.

An advantage of the present invention is a reduced amount of by-products. In contrast, coupling PEG propionaldehyde to insulin at basic pHs can be problematic due to formation of significant amounts of acrolein (resulting from a retro-Michael type side reaction), which is quite difficult to remove. Formation of such undesirable side products necessitates extensive purification to obtain a pharmaceutical grade product. In view of the above, the present reaction typically results in less than about 40%, such as less than 30%, less than 20%, or less than 10%, of by-products.

In some cases, the resulting composition is absent detectable amounts of iodine-containing species or retro-Michael type reaction products. This is particularly advantageous, since iodine-containing species can lead to degradation of poly(ethylene glycol) chains due to chain cleavage, resulting in a polymer product having a high polydispersity value, e.g., greater than around 1.2. Preferably, the polymer of the invention will possess a polydispersity value of less than about 1.2, preferably less than about 1.1, and even more preferably less than about 1.05. Even more preferred are polymers such as those described herein characterized by a polydispersity of 1.04, 1.03, or less.

More specifically, to couple an aldehyde polymer derivative to insulin, a number of different approaches may be employed. One approach (i.e., a random PEGylation approach) is to covalently attach PEG to any number of lysine residues that are surface accessible. To conduct such a reaction, insulin is typically reacted with a polymer alkanal in a non amine-containing buffer at mild pHs generally ranging from about 5 to 8 (non-amine containing buffers are preferred since the amino-groups in the buffer can compete with protein amino groups for coupling to the polymer alkanal). A suitable non-amine containing buffer is selected having an appropriate pK for the desired pH range for conducting the conjugation chemistry. The coupling reaction generally takes anywhere from minutes to several hours (e.g., from 5 minutes to 24 hours or more), and on average, coupling is achieved between about 0.2 and 4 hours to form the imine-coupled conjugate. To the reaction mixture is then added any one of a number of suitable reducing agents as described above (e.g., sodium cyanoborohydride). The resulting mixture is then generally allowed to react under low to ambient temperature conditions, e.g., 4° C. to 37° C. for about one hour to 48 hrs. Preferably, the reduction reaction is complete in less than about 24 hours. Random coupling is favored at pHs around 7 to 7.5 or so, while coupling at the N-terminal is favored at low pHs (e.g., less than 5).

To increase the degree of modification, that is, to promote an increase in the number of PEGs that are covalently attached at available sites on the insulin, any one or more of the above described conditions (e.g., molar ratio of polymer alkanal to insulin, temperature, reaction time, pH, etc.) can be increased, either independently or simultaneously.

These and other approaches for preparing the modified insulins described herein can be used.

### Purification and Storage of Modified Peptides

The therapeutic peptide polymer conjugates described herein can be purified to obtain/isolate different conjugate species. Specifically, a product mixture can be purified to obtain an average of anywhere from one, two, or three or even more PEGs per therapeutic peptide. In one embodiment of the invention, preferred therapeutic peptide conjugates are mono-conjugates. The strategy for purification of the final conjugate reaction mixture will depend upon a number of factors, including, for example, the molecular weight of the polymeric reagent employed, the therapeutic peptide, and the desired characteristics of the product—e.g., monomer, dimer, particular positional isomers, etc.

For instance, once prepared, the modified insulins can be isolated. Known methods can be used to isolate the product, but it is particularly preferred to use chromatography, e.g., ion-exchange chromatography or size exclusion chromatography. Thus, the method optionally includes the step of purifying the modified insulin once it is formed. Again, standard art-known purification methods can be used to purify the modified insulin.

If desired, conjugates having different molecular weights can be isolated using gel filtration chromatography and/or ion exchange chromatography. Gel filtration chromatography may be used to fractionate different therapeutic peptide conjugates (e.g., 1-mer, 2-mer, 3-mer, and so forth, wherein “1-mer” indicates one polymer molecule per therapeutic peptide, “2-mer” indicates two polymers attached to therapeutic peptide, and so on) on the basis of their differing molecular weights (where the difference corresponds essentially to the average molecular weight of the water-soluble polymer). While this approach can be used to separate PEG and other therapeutic peptide polymer conjugates having different molecular weights, this approach is generally ineffective for separating positional isomers having different polymer attachment sites within the therapeutic peptide. For example, gel filtration chromatography can be used to separate from each other mixtures of PEG 1-mers, 2-mers, 3-mers, and so forth, although each of the recovered PEG-mer compositions may contain PEGs attached to different reactive amino groups (e.g., lysine residues) or other functional groups of the therapeutic peptide. Alternatively, reversed phase separations may be used, especially for analytical analysis requiring large numbers of samples. Ion exchange methods are most preferred for large scale preparation.

For instance, in certain embodiments, conjugates may be purified to obtain/isolate different PEGylated species. Alternatively, and more preferably for lower molecular weight PEGs, e.g., having molecular weights less than about 20 kiloDaltons, such as less than about 10 kiloDaltons, the product mixture can be purified to obtain a distribution around a certain number of PEGs per insulin molecule. The strategy for purification of the final conjugate reaction mixture will depend upon a number of factors—the molecular weight of the polymer employed, the desired dosing regimen, and the residual activity and in vivo properties of the individual conjugate(s) species.

For example, in an exemplary reaction where insulin is randomly conjugated to a polymeric reagent having a molecular weight of about 20 kDa, the resulting reaction mixture will likely contain unmodified insulin (MW 6 kDa), mono-PEGylated insulin (MW 26 kDa), di-PEGylated insulin (MW 46 kDa), and so forth. While this approach can be used to separate PEG and other polymer conjugates having different molecular weights, this approach is generally ineffective for separating positional isomers having different polymer attachment sites within the protein. For example, gel filtration chromatography can be used to separate from each other mixtures of PEG 1-mers, 2-mers, 3-mers, and so forth, although each of the recovered PEG-mer compositions may contain PEGs attached to different reactive amino groups (e.g., lysine residues) within the active agent.

Gel filtration columns suitable for carrying out this type of separation include Superdex™ and Sephadex™ columns available from Amersham Biosciences (Piscataway, N.J.). Selection of a particular column will depend upon the desired fractionation range desired. Elution is generally carried out using a suitable buffer, such as phosphate, acetate, or the like. The collected fractions may be analyzed by a number of different methods, for example, (i) optical density (OD) at 280 nm for protein content, (ii) bovine serum albumin (BSA) protein analysis, (iii) iodine testing for PEG content (Sims et al. (1980) Anal. Biochem, 107:60-63), and (iv) sodium dodecyl sulfate polyacrylamide gel electrophoresis (SDS PAGE), followed by staining with barium iodide.

Separation of positional isomers is typically carried out by reverse phase chromatography using a reverse phase-high performance liquid chromatography (RP-HPLC) C18 column (Amersham Biosciences or Vydac) or by ion exchange chromatography using an ion exchange column, e.g., a DEAE- or CM-Sepharose™ ion exchange column available from Amersham Biosciences. Either approach can be used to separate polymer-therapeutic peptide isomers having the same molecular weight (positional isomers).

When column chromatography is performed on modified insulins, one of the impurities, triacetyl insulin, often sticks to the resin until it is washed with sodium hydroxide, thereby improving our impurity profile.

Following conjugation, and optionally additional separation steps, the conjugate mixture can be concentrated, sterile filtered, and stored at a low temperature, typically from about −80° C. to about −20° C. For instance, chromatography fractions may be pooled and then concentrated and diafiltered against water using TFF (tangential flow filtration).

Alternatively, the conjugate may be lyophilized, either with or without residual buffer and stored as a lyophilized powder. In some instances, it is preferable to exchange a buffer used for conjugation, such as sodium acetate, for a volatile buffer, such as ammonium carbonate or ammonium acetate, which can be readily removed during lyophilization, so that the lyophilized powder is absent residual buffer. Alternatively, a buffer exchange step may be used employing a formulation buffer, so that the lyophilized conjugate is in a form suitable for reconstitution into a formulation buffer and ultimately for administration to a mammal.

The resulting purified compositions are preferably substantially free of the non-conjugated therapeutic peptide. In addition, the compositions preferably are substantially free of all other non-covalently attached water-soluble polymers.

### Compositions

### Compositions of Conjugate Isomers

Also provided herein are compositions comprising any one or more of the therapeutic peptide polymer conjugates described herein. In certain instances, the composition will comprise a plurality of therapeutic peptide polymer conjugates. For instance, such a composition may comprise a mixture of therapeutic peptide polymer conjugates having one, two, three and/or even four water-soluble polymer molecules covalently attached to sites on the therapeutic peptide. That is to say, a composition of the invention may comprise a mixture of monomer, dimer, and possibly even trimer or 4-mer. Alternatively, the composition may possess only mono-conjugates, or only di-conjugates, etc. A mono-conjugate therapeutic peptide composition will typically comprise therapeutic peptide moieties having only a single polymer covalently attached thereto, e.g., preferably releasably attached. A mono-conjugate composition may comprise only a single positional isomer, or may comprise a mixture of different positional isomers having polymer covalently attached to different sites within the therapeutic peptide.

In yet another embodiment, a therapeutic peptide conjugate may possess multiple therapeutic peptides covalently attached to a single multi-armed polymer having 3 or more polymer arms. Typically, the therapeutic peptide moieties are each attached at the same therapeutic peptide amino acid site, e.g., the N-terminus.

With respect to the conjugates in the composition, the composition will typically satisfy one or more of the following characteristics: at least about 85% of the conjugates in the composition will have from one to four polymers attached to the therapeutic peptide; at least about 85% of the conjugates in the composition will have from one to three polymers attached to the therapeutic peptide; at least about 85% of the conjugates in the composition will have from one to two polymers attached to the therapeutic peptide; or at least about 85% of the conjugates in the composition will have one polymer attached to the therapeutic peptide (e.g., be monoPEGylated); at least about 95% of the conjugates in the composition will have from one to four polymers attached to the therapeutic peptide; at least about 95% of the conjugates in the composition will have from one to three polymers attached to the therapeutic peptide; at least about 95% of the conjugates in the composition will have from one to two polymers attached to the therapeutic peptide; at least about 95% of the conjugates in the composition will have one polymers attached to the therapeutic peptide; at least about 99% of the conjugates in the composition will have from one to four polymers attached to the therapeutic peptide; at least about 99% of the conjugates in the composition will have from one to three polymers attached to the therapeutic peptide; at least about 99% of the conjugates in the composition will have from one to two polymers attached to the therapeutic peptide; and at least about 99% of the conjugates in the composition will have one polymer attached to the therapeutic peptide (e.g., be monoPEGylated).

In one or more embodiments, the conjugate-containing composition is free or substantially free of albumin.

Control of the desired number of polymers for covalent attachment to therapeutic peptide is achieved by selecting the proper polymeric reagent, the ratio of polymeric reagent to the Therapeutic peptide, temperature, pH conditions, and other aspects of the conjugation reaction. In addition, reduction or elimination of the undesired conjugates (e.g., those conjugates having four or more attached polymers) can be achieved through purification mean as previously described.

For example, the water-soluble polymer-(therapeutic peptide) conjugates can be purified to obtain/isolate different conjugated species. Specifically, the product mixture can be purified to obtain an average of anywhere from one, two, three, or four PEGs per therapeutic peptide, typically one, two or three PEGs per therapeutic peptide. In one or more embodiments, the product comprises one PEG per therapeutic peptide, where PEG is releasably (via hydrolysis) attached to PEG polymer, e.g., a branched or straight chain PEG polymer.

The present invention includes formulations comprising mixtures of modified insulins having different hydrophilic polymers. The mixtures may also have different blocking groups or moieties. The mixtures may also involve differences in modification sites and differences in the number of hydrophilic polymers and/or blocking groups or moieties. For the sake of clarity, the modified insulins in these mixtures may or may not have one or more blocking groups, e.g., acyl group(s).

The modified insulin mixtures may be optimized to provide the desired pharmacodynamic/pharmacokinetic profile. For instance, the smaller the ratio of blocking group conjugation, e.g., acetyl conjugation, to hydrophilic polymer conjugation, e.g., PEG conjugation, the more the PK/PD profile is extended. In this regard, the ratio of blocking group to hydrophilic polymer for one component may, e.g., range from 0:1 to 1:1, while the ratio of blocking group to hydrophilic polymer for a second component may, e.g., range from 1:1 to 2:1.

As another example of such mixtures, the formulation might contain a first modified insulin comprising a hydrophilic polymer, e.g., PEG, having a molecular weight of less than 1000 Dalton, e.g., less than 800 Dalton, and a second modified insulin comprising a hydrophilic polymer, e.g., PEG, having a molecular weight of greater than 1000 Dalton, e.g., greater than 2000 Dalton. By combining different lots of insulin with different acetyl to PEG ratios as well as different PEG chain lengths, the desired action profile may be obtained.

### Formulations

In view of the present description, the compounds of the present invention may be formulated by various methods and techniques known and available to those skilled in the art. In this regard, the therapeutic peptides of the invention can be formulated in any number of ways. Consequently, the therapeutic peptides provided herein are not limited to the specific technique or approach used in their formulation. Exemplary approaches for formulating the presently described modified insulins, however, will be discussed in detail below.

A composition of the invention may also comprise a mixture of water-soluble polymer-(therapeutic peptide) conjugates and unconjugated therapeutic peptide, to thereby provide a mixture of fast-acting and long-acting therapeutic peptide.

Additional pharmaceutical compositions in accordance with the invention include those comprising, in addition to an extended-action therapeutic peptide water-soluble polymer conjugate as described herein, a rapid acting therapeutic peptide polymer conjugate where the water-soluble polymer is releasably attached to a suitable location on the therapeutic peptide.

In addition to the modified insulin (and other insulins), the formulations may contain any compound useful as a therapeutic agent. These pharmaceutically useful compounds (“pharmaceutical compounds”) of the present invention may include drugs which act on blood glucose levels.

Examples of pharmaceutical compounds suitable for use in combination with the modified insulin include but are not limited to amylin, an antioxidant such as vitamin E, vitamin C, an isoflavone, zinc, selenium, ebselen, a carotenoid; an insulin or insulin analogue such as regular insulin, lente insulin, semilente insulin, ultralente insulin, NPH, Humalog®, or Novolog®; an α-adrenergic receptor antagonist such as prazosin, doxazocin, phenoxybenzamine, terazosin, phentolamine, rauwolscine, yohimine, tolazoline, tamsulosin, or terazosin; a β-adrenergic receptor antagonist such as acebutolol, atenolol, betaxolol, bisoprolol, carteolol, esmolol, metoprolol, nadolol, penbutolol, pindolol, propanolol, timolol, dobutamine hydrochloride, alprenolol, bunolol, bupranolol, carazolol, epanolol, moloprolol, oxprenolol, pamatolol, talinolol, tiprenolol, tolamolol, or toliprolol; a non-selective adrenergic receptor antagonist such as carvedilol or labetolol; a first generation sulphonylurea such as tolazamide, tolubtuamide, chlorpropamide, acetohexamide; a second generation sulphonylurea such as glyburide, glipizide, and glimepiride; a biguanide agent such as is metformin; a benzoic acid derivative such as replaglinide; an α-glucosidase inhibitor such as acarbose and miglitol; a thiazolidinedione such as rosiglitazone, pioglitazone, or troglitazone; a phosphodiesterase inhibitor such as anagrelide, tadalfil, dipyridamole, dyphylline, vardenafil, cilostazol, milrinone, theophylline, or caffeine; a DPP-IV inhibitor, such as sitagliptin or vildagliptin; a cholineresterase antagonist such as donepezil, tacrine, edrophonium, demecarium, pyridostigmine, zanapezil, phospholine, metrifonate, neostigmine, or galathamine; a glutathione increasing compound such as N-acetylcysteine, a cysteine ester, L-2-oxothiazolidine-4-carboxolate (OTC), gamma glutamylcysteine and its ethyl ester, glytathtione ethyl ester, glutathione isopropyl ester, lipoic acid, cysteine, methionine, bucillamine or S-adenosylmethionine; and GLP-1 and glucagon like peptide analogues, such as exenatide, DAC:GLP-1 (CJC-1131), Liraglutide, ZP1O, BIM51077, LY315902, LY307161 (SR), and where applicable, analogues, agonists, antagonists, and inhibitors of the above, including the synthetic, native, glycosylated, unglycosylated, PEGylated forms, and biologically active fragments and analogs thereof.

Optionally, a therapeutic peptide conjugate composition of the invention will comprise, in addition to the therapeutic peptide conjugate, a pharmaceutically acceptable excipient. More specifically, the composition may further comprise excipients, solvents, stabilizers, membrane penetration enhancers, etc., depending upon the particular mode of administration and dosage form.

In one or more embodiments of the invention, a pharmaceutical composition is provided comprising a conjugate comprising a therapeutic peptide covalently attached, e.g., releasably, to a water-soluble polymer, wherein the water-soluble polymer has a weight-average molecular weight of greater than about 2,000 Daltons; and a pharmaceutically acceptable excipient.

Pharmaceutical compositions of the invention encompass all types of formulations and in particular those that are suited for injection, e.g., powders or lyophilates that can be reconstituted as well as liquids, as well as for inhalation. Examples of suitable diluents for reconstituting solid compositions prior to injection include bacteriostatic endotoxin-free water for injection, dextrose 5% in water, phosphate-buffered saline, Ringer's solution, saline, sterile water, deionized water, and combinations thereof. With respect to liquid pharmaceutical compositions, solutions and suspensions are envisioned.

The present invention also includes pharmaceutical preparations comprising a modified insulin as provided herein in combination with a pharmaceutical excipient. Generally, the modified insulin itself will be in a solid form (e.g., a precipitate), which can be combined with a suitable pharmaceutical excipient that can be in either solid or liquid form. The pharmaceutical preparations encompass all types of formulations, including those that are suited for inhalation or injection, e.g., powders that can be reconstituted as well as suspensions and solutions.

Exemplary excipients include, without limitation, those selected from carbohydrates, inorganic salts, antimicrobial agents, antioxidants, surfactants, buffers, acids, bases, and combinations thereof.

Representative carbohydrates for use in the compositions of the present invention include sugars, derivatized sugars such as alditols, aldonic acids, esterified sugars, and sugar polymers. Exemplary carbohydrate excipients suitable for use in the present invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like. Preferred, in particular for formulations intended for inhalation, are non-reducing sugars, sugars that can form a substantially dry amorphous or glassy phase when combined with the composition of the present invention, and sugars possessing relatively high glass transition temperatures, or Tgs (e.g., Tgs greater than 40° C., or greater than 50° C., or greater than 60° C., or greater than 70° C., or having Tgs of 80° C. and above). Such excipients may be considered glass-forming excipients.

The excipient can also be an inorganic salt or buffer such as citric acid, sodium chloride, potassium chloride, sodium sulfate, potassium nitrate, sodium phosphate monobasic, sodium phosphate dibasic, and combinations thereof.

The preparation may also have an antimicrobial agent for preventing or deterring microbial growth. Nonlimiting examples of antimicrobial agents suitable for the present invention include, but are not limited to, benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids, and fatty esters; steroids, such as cholesterol; and chelating agents, such as EDTA, zinc, and other such suitable cations.

Acids or bases may be present as an excipient in the preparation. Nonlimiting examples of acids that can be used include those acids selected from hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Additional excipients include amino acids, peptides and particularly oligomers comprising 2-9 amino acids, or 2-5 mers, and polypeptides, all of which may be homo or hetero species.

Exemplary protein excipients include albumins such as human serum albumin (HSA), recombinant human albumin (rHA), gelatin, casein, hemoglobin, and the like. The compositions may also include a buffer or a pH-adjusting agent, typically but not necessarily a salt prepared from an organic acid or base. Representative buffers include organic acid salts of citric acid, ascorbic acid, gluconic acid, carbonic acid, tartaric acid, succinic acid, acetic acid, or phthalic acid. Other suitable buffers include Tris, tromethamine hydrochloride, borate, glycerol phosphate, and phosphate. Amino acids such as glycine are also suitable.

The compositions of the present invention may also include one or more additional polymeric excipients/additives, e.g., polyvinylpyrrolidones, derivatized celluloses such as hydroxymethylcellulose, hydroxyethylcellulose, and hydroxypropylmethylcellulose, FICOLLs (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin.

The compositions may further include flavoring agents, taste-masking agents, inorganic salts (e.g., sodium chloride), antimicrobial agents (e.g., benzalkonium chloride), sweeteners, antioxidants, antistatic agents, surfactants (e.g., polysorbates such as “TWEEN 20” and “TWEEN 80,” and pluronics such as F68 and F88, available from BASF), sorbitan esters, lipids (e.g., phospholipids such as lecithin and other phosphatidylcholines, phosphatidylethanolamines, although preferably not in liposomal form), fatty acids and fatty esters, steroids (e.g., cholesterol), and chelating agents (e.g., zinc and other such suitable cations). The use of certain di-substituted phosphatidylcholines for producing perforated microstructures (i.e., hollow, porous microspheres) may also be employed.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5%-98% by weight, more preferably from about 15-95% by weight of the excipient, with concentrations less than 30% by weight most preferred. In some embodiments, spray-dried formulations will contain from about 0-50% by weight excipient or from 0-40% by weight excipient.

These foregoing pharmaceutical excipients along with other excipients are described in “Remington: The Science & Practice of Pharmacy”, 21^{St} ed., Williams & Williams, (2005), the “Physician's Desk Reference”, 61^{st} ed., Medical Economics, Montvale, N.J. (2007), and Kibbe, A. H., Handbook of Pharmaceutical Excipients, 3^{rd} Edition, American Pharmaceutical Association, Washington, D.C., 2000.

The amount of the therapeutic peptide conjugate (i.e., the conjugate formed between the active agent and the polymeric reagent) in the composition will vary depending on a number of factors, but will optimally be a therapeutically effective amount when the composition is stored in a unit dose container (e.g., a vial). In addition, a pharmaceutical preparation, if in solution form, can be housed in a syringe. A therapeutically effective amount can be determined experimentally by repeated administration of increasing amounts of the conjugate in order to determine which amount produces a clinically desired endpoint.

Formulations and corresponding doses of the modified insulins of the invention will vary with the bioactivity of the modified insulin employed. The amount of the modified insulin in the composition will vary depending on a number of factors, but will optimally be a therapeutically effective dose when the composition is stored in a unit dose container. A therapeutically effective dose can be determined experimentally by repeated administration of increasing amounts of the conjugate in order to determine which amount produces a clinically desired endpoint. The precise dosages can be determined by one skilled in the art when coupled with the pharmacodynamics and pharmacokinetics of the precise modified insulin composition employed for a particular route of administration, and can readily be adjusted in response to periodic glucose monitoring.

Individual dosages (on a per inhalation basis) for inhalable modified insulin formulations will typically be in the range of from about 0.1 mg to about 50 mg modified insulin (based on equivalent insulin mass), where the desired overall dosage is typically achieved in about 1-10 breaths, such as about 1 to 4 breaths. On average, the overall dose of modified insulin administered by inhalation per day will range from about 0.1 U to about 20 U. The actual dose can be determined by a physician, based upon the need of the patient, whether insulin-naïve or existing insulin user, and based upon response to administration.

When the present invention is used to deliver modified insulin by inhalation to the lung, the amount of modified insulin in the composition will be that amount necessary to deliver a therapeutically effective amount of modified insulin per unit dose to achieve at least one of the therapeutic effects of native insulin, i.e., the ability to control blood glucose levels to near normoglycemia. In practice, this will vary widely depending upon the severity of the diabetic condition to be treated, the patient population, the stability of the composition, and the like.

The composition will generally contain, in terms of solid weight, anywhere from about 1% to about 99%, such as from about 2% to about 95%, from about 5% to about 85%, or from about 70% to about 95%, of pharmaceutical protein, such as the modified insulin. The percentage of the pharmaceutical protein in the composition will also depend upon the relative amounts of excipients/additives contained in the composition. More specifically, the composition will typically contain at least about one of the following solid weight percentages of the pharmaceutical protein: 10%, 20%, 30%, 40%, 50%, 60%, 70%, 80%, 90% or more. In some embodiments, powder compositions will contain at least about 60%, e.g., about 60-100% by weight of the pharmaceutical protein. For liquid formulations, the amount of modified insulin in the pharmaceutical composition may vary. The amount of modified insulin typically ranges from 2 mg/ml to 55 mg/ml, such as from 5 mg/ml to 55 mg/ml, 10 mg/ml to 50 mg/ml, 20 mg/ml to 45 mg/ml, or 30 mg/ml to 40 mg/ml.

It is to be understood that more than one pharmaceutical protein may be incorporated into the compositions described herein. Furthermore, the composition may also contain more than one form of the pharmaceutical protein, for example, modified insulin according to the invention, and another type of insulin, such as one that exhibits a shorter duration of action.

One embodiment of the present invention provides compositions that contain no protamine. Protamines are a group of proteins isolated from fish, and are commonly used in insulin formulations to prolong duration (see, e.g., Vanbever R. et al., “Sustained release of insulin from insoluble inhaled particles,” Drug Dev. Res. 48, 178-185, 1999). However, protamines, as well as protamine-insulin complexes, have been shown to be potentially immunogenic (Samuel T. et al., “Studies on the immunogenicity of protamines in humans and experimental animals by means of a micro-complement fixation test,” Clin. Exp. Immunol. 33(2), 252-260 (1978); Kurtz A. B. et al., “Circulating IgG antibody to protamine in patients treated with protamine-insulins,” Diabetologia. 25(4), 322-324 (1983)). Since the compositions of the present invention are capable of sustained release in the absence of protamine, the present invention provides the option of including no protamine, thereby avoiding the adverse reactions that may be caused by protamine. Protamine, however, is optionally present in the composition of the present invention.

While the use of liposomes is also commonly employed to sustain duration of drug effect, the present invention does not require the use of liposomes. Accordingly, other embodiments of the present invention provide compositions that contain no liposome, no lipid, and/or no polymers in addition to the pharmaceutical protein. However, having noted the possibility that the present compositions exclude lipids or the use of liposomes, it is also noted that the primary particles of the present invention can include lipids or be included into liposomal formulations, described in more detail below.

As discussed herein, in some embodiments, a modified insulin is administered in a formulation to the lungs. In this embodiment, the formulations are generally liquid or solid formulations. Liquid formulations may be solutions or suspensions of the modified insulin, together with excipients. Solid formulations may be powders of the modified insulins, together with excipients. The following groups of excipients may be used in some embodiments of the formulations of the present invention.

The compositions of the invention may include one or more buffering, or pH-adjusting or -controlling, agents. These agents are generally a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers. Suitable amino acids, which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like.

These agents, if present, are generally present in amounts of from about 0.01% to about 10%, by weight, of the composition. In some embodiments, the amount ranges from about 0.02% to about 9%, or from about 0.03% to about 8%, or from about 0.04% to about 7%, or from about 0.05% to about 6% by weight, of the composition. The amount chosen will depend upon its desired effect on the composition and can be varied as needed.

Some embodiments of the invention are dry formulations designed for pulmonary delivery. Some embodiments of the invention include excipients that are designed to impart desired physical characteristics to the end product, which may further impart desired or improved actions on the treated subject. Thus, the inventive compositions may comprise a pharmaceutically acceptable excipient or carrier, which may be taken into the lungs with no significant adverse toxicological effects to the subject, and particularly to the lungs of the subject.

Generally, such excipients will, if present, at least in part, serve to further improve the features of the modified insulin composition, for example by providing more efficient and/or reproducible delivery of the modified insulin, improve the handling characteristics of powders, such as flowability and consistency, and/or facilitate manufacturing and/or filling of unit dosage forms. In particular, excipient materials can often function to further improve the physical and chemical stability of the modified insulin, minimize the residual moisture content and hinder moisture uptake, and to enhance particle size, degree of aggregation, particle surface properties, such as rugosity, ease of inhalation, and the targeting of particles to the lung. One or more excipients may also be provided to serve as bulking agents when it is desired to reduce the concentration of modified insulin in the formulation.

One particular type of dry formulation-enhancing excipient that may be included in the formulation is the dispersibility-enhancing excipient. This excipient generally provides more efficient and/or reproducible delivery of the modified insulin, by improving the physical characteristics of the dry formulation. Dispersibility-enhancing agents include, but are not limited to, amino acids and polypeptides that function as dispersing agents. Amino acids falling into this category include, but are not limited to, hydrophobic amino acids such as leucine, norleucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above. Examples include, but are not limited to, dimers, trimers, tetramers, and pentamers having at least two leucines in any position, such as dileucine and trileucine, as disclosed in U.S. Pat. No. 6,518,239, which is incorporated herein by reference.

These excipients, if present, are generally present in the composition in amounts ranging from about 0.01% to about 95%, such as about 0.5% to about 80%, or about 1% to about 70%, by weight. Thus, the amount may, e.g., range from about 10% to about 90%, or from about 20% to about 80%, or from about 30% to about 70%, or about 60%, by weight of the composition. The amount chosen will depend upon its desired effect on the composition and can be varied as needed. The ideal amount and type of dry formulation-enhancing excipient is an amount and type that furthers dispersibility and deliverability of the modified insulin.

Some embodiments of the invention are dry formulations, and some may benefit from the addition of a component that stabilizes the glass transition temperature of the composition. In some embodiments, this component will have a higher glass transition temperature than the modified insulin of the invention. In some embodiments, the excipient may have a glass transition temperature (Tg) above about 35° C., such as above about 40° C., above about 45° C., above about 55° C., above about 60° C., above about 65° C., above about 70° C., above about 75° C., above about 80° C., above about 85° C., or above about 90° C., as measured by differential scanning calorimetry (DSC).

Glass transition stabilizing excipients, also known as glass stabilizers, glass transition stabilizers, and glass formers, include, but are not limited to, carbohydrates. Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol, and the like. Other examples of glass formers are described in U.S. Pat. No. 6,309,671, which is incorporated herein by reference.

The glass transition stabilizing excipients, if present, will generally be present in an amount by weight of from about 10% to about 90% of the composition. In some embodiments, they are present in an amount from about 20% to about 80%, or from about 30% to about 70%, or from about 40% to about 60%, or about 50%, by weight of the composition. The amount may be 10, 20, 30, 40, 50, 60, 70, 80, or 90% by weight of the composition. The amount chosen will depend upon its desired effect on the composition and can be varied as needed. The ideal amount and type of glass transition stabilizing excipient is an amount and type that furthers dispersibility and deliverability of the modified insulin.

Other pharmaceutical excipients and additives useful in the present pharmaceutical formulation include, but are not limited to, amino acids, peptides, proteins, non-biological polymers, biological polymers, carbohydrates, such as sugars, derivatized sugars such as alditols, aldonic acids, esterified sugars, and sugar polymers, which may be present singly or in combination. Exemplary protein excipients include, but are not limited to, albumins such as human serum albumin (HSA), recombinant human albumin (rHA), gelatin, casein, hemoglobin, and the like. Suitable excipients include those provided in U.S. Pat. No. 6,136,346 and WO 96/32096, which are incorporated herein by reference.

The inventive compositions may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin. In one or more embodiments, the formulation is polymer free (except for the covalent modification to the insulin).

The inventive compositions may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations).

For metered-dose inhaler (MDI) applications, the compositions may also be treated so as to have even greater stability. Several attempts have dealt with improving suspension stability by increasing the solubility of surface-active agents in the HFA propellants. To this end, U.S. Pat. No. 5,118,494, WO 91/11173, and WO 92/00107 disclose the use of HFA soluble fluorinated surfactants to improve suspension stability. Mixtures of HFA propellants with other perfluorinated cosolvents have also been disclosed as in WO 91/04011. Other attempts at stabilization involve the inclusion of nonfluorinated surfactants. In this respect, U.S. Pat. No. 5,492,688 discloses that some hydrophilic surfactants (with a hydrophilic/lipophilic balance greater than or equal to 9.6) have sufficient solubility in HFAs to stabilize medicament suspensions. Increases in the solubility of conventional nonfluorinated MDI surfactants (e.g., oleic acid, lecithin) can also reportedly be achieved with the use of co-solvents such as alcohols, as set forth in U.S. Pat. Nos. 5,683,677 and 5,605,674, as well as in WO 95/17195. All of the aforementioned references are incorporated herein by reference.

Some embodiments of the compositions in accordance with the present invention may exclude penetration enhancers, which can cause irritation and are toxic at the high levels often necessary to provide substantial enhancement of absorption. Specific enhancers, which are typically absent from the compositions of the present invention, are the detergent-like enhancers such as deoxycholate, laureth-9, DDPC, glycocholate, and the fusidates. Certain enhancers, however, such as those that protect the pharmaceutical protein from enzyme degradation, e.g., protease and peptidase inhibitors such as alpha-1 antiprotease, captropril, thiorphan, and the HIV protease inhibitors, may, in certain embodiments of the present invention, be incorporated in the composition of the present invention.

In some embodiments, the modified insulins can be administered parenterally by intravenous, intramuscular, or by subcutaneous injection. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

The modified insulins may be encapsulated in biodegradable polymer-based drug delivery formulations. In some cases, the modified insulin may be encapsulated at higher concentration in the drug delivery formulation than unmodified insulin.

In certain embodiments, the release of modified insulin from biodegradable polymer drug delivery formulations shows less burst than unmodified insulin. The physical and chemical stability of modified insulin in biodegradable polymer drug delivery formulations may be greater, and the antigenicity and immunogenicity may be lower than for unmodified insulin.

Biodegradable polymers for this application include, but are not limited to, poly(lactide)s, poly(glycolide)s, poly(d,l-lactide-co-glycolide)s, poly(caprolactone)s, poly(orthoester)s, copolymers of poly(esters) and poly(ethers), copolymers of poly(lactide) and poly(ethylene glycol), and the like.

Accordingly, the modified insulins can be incorporated into biodegradable polymer drug delivery formulations including, for example, poly(d,l-lactide-co-glycolide) (PLGA) microparticles. This may achieve higher encapsulation of the protein conjugate as compared to unmodified insulin and may also reduce burst (release over the first 24 hours).

Moreover, conjugation with hydrophilic polymers, such as PEG, renders the conjugate soluble in certain organic solvents, simplifying the process of forming PLGA microspheres.

While much of the present discussion has related to excipients that can be included in the invention, the present invention also expressly contemplates the exclusion of certain elements. Generally, the description of any group or list of compounds, elements, components, etc., is to be taken as an express contemplation of the exclusion of any member of such group or list.

### Physical Characteristics of Formulations

The compositions described herein may be in powdered form (e.g., including modified insulins of the invention) or may be flowable liquids.

Some embodiments of the invention include particles that have physical characteristics that allow for their delivery to the deep lung. In one embodiment, a powdered or liquid formulation for use in the present invention includes an aerosol having a particle size selected to permit penetration into the alveoli of the lungs. Dry powders of the present invention are composed of aerosolizable particles effective to penetrate into the lungs. The dry or aerosolized liquid particles of the present invention may generally have a mass median diameter (MMD), or volume median geometric diameter (VMGD), or mass median envelope diameter (MMED), or a mass median geometric diameter (MMGD), of less than about 30 μm, or less than about 20 μm, or less than about 10 μm, or less than about 7.5 μm, or less than about 4 μm, or less than about 3.3 μm, and usually are in the range of 0.1 μm to 5 μm in diameter. Preferred powders or aerosolized liquids are composed of particles having an MMD, VMGD, MMED, or MMGD from about 1 to 5 μm. In some cases, the powder will also contain non-respirable carrier particles such as lactose, where the non-respirable particles are typically greater than about 40 microns in size. Generally dry or liquid particles having an MMD of 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 gm are contemplated, as are values less than any of these discrete values, as well as ranges from any of these discrete values to any of these discrete values, such as from 1-30 μm, or from 7-16 μm, or from 11-29 μm, etc.

The powders or aerosolized liquids of the present invention may also be characterized by an aerosol particle size distribution—mass median aerodynamic diameter (MMAD)—having MMADs less than about 10, 9, 8, 7, 6, or 5 μm, or less than 4.0 μm, even more preferably less than 3.3 μm, and most preferably less than 3 μm. The mass median aerodynamic diameters of the powders or liquid particles will characteristically range from about 0.1-5.0 μm, or from about 0.2-5.0 μm MMAD, or from about 1.0-4.0 μm MMAD, or from about 1.5 to 3.0 μm. Small aerodynamic diameters may be achieved by a combination of optimized spray drying conditions and choice and concentration of excipients. Generally particles having an MMAD of 1, 2, 3, 4, 5, 6, 7, 8, 9, 10, 11, 12, 13, 14, 15, 16, 17, 18, 19, and 20 are contemplated, as are values less than any of these discrete values, and ranges from any of these discrete values to any of these discrete values, such as from 1-20 μm, or from 7-16 μm, or from 11-19 μm, etc.

The powders of the present invention may also be characterized by their densities. The powder will generally possess a bulk density from about 0.1 to 10 g/cubic centimeter, or from about 0.1-2 g/cubic centimeter, or from about 0.15-1.5 g/cubic centimeter. In one embodiment of the present invention, the powders have big and fluffy particles with a density of less than about 0.4 g/cubic centimeter and an MMD between 5 and 30 microns.

When in a dry powder form, the pharmaceutical formulation may have a moisture content below about 10 wt %, such as below about 5 wt %, or below about 3 wt %. Such powders are described in WO 95/24183, WO 96/32149, WO 99/16419, and WO 99/16422, all of which are incorporated herein by reference.

Some particles according to the invention are formed in such a manner that the modified insulins are uniformly distributed throughout the particle. That is, the modified insulin, as well as other elements of the composition, which may include precipitating agent, buffer, dispersibility-enhancing agent, and/or glass-stabilizing agent, is uniformly distributed throughout the particle.

In other embodiments of the invention, particles are formed in such a way as to enrich particular elements of the formulation in particular sections of the particle. For example, a particle may generally be described as including a core at its center and a surface around its periphery. With regard to the heterogeneity within the particle, the transition from core to surface may be gradual or abrupt, or any variation thereof. Particles may be manufactured such that a core is enriched with one element and a surface is enriched with another.

This heterogeneity may be achieved by forming the core and surface in separate preparation steps, using different compositional elements during the different steps. Alternatively, the heterogeneity may be achieved by introducing into a homogeneous mixture a component that has an affinity for a particular section of a particle or which migrates during a drying phase, for example. Examples of such methods are described in U.S. Pat. No. 6,518,239, the entire disclosure of which is incorporated herein by reference.

In one embodiment of the invention, heterogeneous particles are formed by forming a liquid composition comprising modified insulin and one or more excipients. The liquid composition may additionally include at least one surface excipient, which is an agent that has a tendency to migrate to the surface of the particle. Such surface excipients may be “surface active agents,” as described in U.S. Pat. No. 6,518,239. Examples of such agents include, but are not limited to, di- and tripeptides containing at least two leucines.

A particular characteristic that usually relates to improved dispersibility and handling characteristics is the product rugosity. Rugosity is the ratio of the specific area (e.g., as measured by nitrogen surface adsorption and then calculated by BET) and the surface area calculated from the particle size distribution (e.g., as measured by centrifugal sedimentary particle size analyzer, Horiba Capa 700) and particle density (e.g., as measured by pycnometry), assuming non-porous spherical particles. Rugosity may also be measured by air permeametry. If the particles are known to be generally nodular in shape, as is the case in spray drying, rugosity is a measure of the degree of convolution or folding of the surface. This may be verified for powders made by the present invention by SEM analysis. A rugosity of 1 indicates that the particle surface is spherical and non-porous. Rugosity values greater than 1 indicate that the particle surface is non-uniform and convoluted to at least some extent, with higher numbers indicating a higher degree of non-uniformity. For the powders of the present invention, it has been found that particles may have a rugosity of at least about 2, such as at least about 3, at least about 4, or at least about 5, and may range from 2 to 10, such as from 4 to 8, or from 4 to 6, as measured and determined by a combination of (1) nitrogen surface adsorption; (2) centrifugal sedimentary particle size analysis; and (3) pycnometry.

The drying operation may be controlled to provide dried particles having particular characteristics, such as a rugosity above 2, as discussed above. Rugosities above 2 may be obtained by controlling the drying rate so that a viscous layer of material is rapidly formed on the exterior of the droplet. Thereafter, the drying rate should be sufficiently rapid so that the moisture is removed through the exterior layer of material, resulting in collapse and convolution of the outer layer to provide a highly irregular outer surface. The drying should not be so rapid, however, that the outer layer of material is ruptured. The drying rate may be controlled based on a number of variables, including the droplet size distribution, the inlet temperature of the gas stream, the outlet temperature of the gas stream, the inlet temperature of the liquid droplets, and the manner in which the atomized spray and hot drying gas are mixed.

Powder surface area, measured by nitrogen adsorption, typically range from about 6 m²/g to about 13 m²/g, such as from about 7 m²/g to about 10 m²/g. The particles often have a convoluted “raisin” structure rather than a smooth spherical surface.

The powder compositions typically have glass transition temperatures higher than room temperature, e.g., greater than 50° C., such as greater than 60° C., with exemplary ranges from 30° C. to 150° C., 40° C. to 120° C., or 50° C. to 100° C., as measured by DSC. In certain embodiments, the powder compositions have a melting point above 30° C., such as above 40° C. or above 50° C., and may, e.g., range from 40° C. to 60° C.

Liquid formulations are preferably solutions in which the active drug is dissolved in a solvent (e.g., water, ethanol, ethanol-water, saline) and less preferably are colloidal suspensions. The liquid formulation may also be a solution or suspension of the modified insulin in a low boiling point propellant. Liquid formulations containing dileucyl-containing peptides, including, but not limited to dileucine and trileucine, are also highly dispersible, possessing high ED values.

In some embodiments, the pharmaceutical preparations of the present invention may be administered via injection and are therefore generally liquid solutions or suspensions immediately prior to administration. The pharmaceutical preparation can also take other forms such as syrups, creams, ointments, tablets, powders, and the like. Other modes of administration are also included, such as rectal, transdermal, transmucosal, oral, intrathecal, subcutaneous, intra-arterial, and so forth.

### Preparing Formulations

The compositions of one or more embodiments of the present invention may be made by various methods and techniques, examples of which are known and available to those skilled in the art.

Dry powder formulations may be prepared, for example, by spray drying (or freeze drying or spray-freeze drying). Spray drying of the formulations is carried out, for example, as described generally in the “Spray Drying Handbook”, 5′ ed., K. Masters, John Wiley & Sons, Inc., NY, N.Y. (1991), and in WO 97/41833, which are incorporated herein by reference.

The modified insulin compositions of the invention can be spray-dried from a solvent, e.g., an aqueous solution. Utilizing this approach, the modified insulin is first dissolved in water, generally containing a physiologically acceptable buffer or other excipient as described above. The pH range of modified insulin-containing solutions is generally between about 4 and 6. The aqueous formulation may optionally contain additional water-miscible solvents, such as acetone, alcohols, and the like. Representative alcohols are lower alcohols such as methanol, ethanol, propanol, isopropanol, and the like. The pre-spray dried solutions will generally contain solids dissolved at a concentration from 0.01% (weight/volume) to about 20% (weight/volume), usually from 0.1% to 3% (weight/volume). Dispersibility-enhancing agent, glass-stabilizing agent, and/or precipitating agent may be included in the solution.

The solutions are then spray dried in a spray drier, such as those available from commercial suppliers such as Niro A/S (Denmark), Büchi (Switzerland) and the like, resulting in a dispersible, dry powder. Optimal conditions for spray drying the solutions will vary depending upon the formulation components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition of the modified insulin in the sprayed material. Such temperatures are typically determined experimentally, although generally, the inlet temperature will range from about 50° C. to about 200° C. while the outlet temperature will range from about 30° C. to about 150° C.

Variations of the above may be utilized. One such process is described in U.S. Pat. No. 5,985,248, assigned to Nektar Therapeutics, which document is incorporated herein by reference. In this method, a modified insulin is dissolved in an organic solvent or co-solvent system, and any hydrophilic components (e.g., the leucyl-containing peptides and optional other excipients) are at least partially dissolved in the same organic solvent or co-solvent system. The resulting solution is then spray-dried to form particles. Typically, the solubility of the modified insulin and the hydrophilic component will govern the selection of the organic solvent system. The organic solvent is selected to provide a solubility for the hydrophilic component of at least 1 mg/ml, and preferably at least 5 mg/ml, and a solubility for the modified insulin of at least 0.01 mg/ml, preferably at least 0.05 mg/ml.

Alternatively, the composition may be prepared by spray-drying a suspension, as described in U.S. Pat. No. 5,976,574, assigned to Nektar Therapeutics, which document is incorporated herein by reference. In this method, the modified insulin is dissolved in an organic solvent, e.g., methanol, ethanol, isopropanol, acetone, heptane, hexane chloroform, ether, followed by suspension of the hydrophilic excipient in the organic solvent to form a suspension. The suspension is then spray-dried to form particles. Preferred solvents, for both of the above spray-drying methods include alcohols, ethers, ketones, hydrocarbons, polar aprotic solvents, and mixtures thereof.

The dry powders of the invention may also be prepared by combining aqueous solutions or suspensions of the formulation components and spray-drying them simultaneously in a spray-dryer, as described in U.S. Pat. No. 6,001,336, assigned to Nektar Therapeutics, which document is incorporated herein by reference. Alternatively, the dry powders may be prepared by preparing an aqueous solution of a hydrophilic excipient or additive, preparing an organic solution of a modified insulin, and spray drying the aqueous solution and the organic solution simultaneously through a nozzle, e.g., a coaxial nozzle, to form a dry powder, as described in WO 98/29096, which is incorporated herein by reference.

Alternatively, powders may be prepared by lyophilization, vacuum drying, spray-freeze drying, super critical fluid processing, air drying, or other forms of evaporative drying. In some instances, it may be desirable to provide the dry powder formulation in a form that possesses improved handling/processing characteristics, e.g., reduced static, better flowability, low caking, and the like, by preparing compositions composed of fine particle aggregates, that is, aggregates or agglomerates of the above-described dry powder particles, where the aggregates are readily broken back down to the fine powder components for pulmonary delivery, as described, e.g., in U.S. Pat. No. 5,654,007, which is incorporated herein by reference.

In another approach, dry powders may be prepared by agglomerating the powder components, sieving the materials to obtain agglomerates, spheronizing to provide a more spherical agglomerate, and sizing to obtain a uniformly-sized product, as described, e.g., in WO 95/09616, which is incorporated herein by reference.

Dry powders may also be prepared by blending, grinding, sieving or jet milling formulation components in dry powder form.

Once formed, the dry powder compositions are preferably maintained under dry (i.e., relatively low humidity) conditions during manufacture, processing, and storage. Irrespective of the drying process employed, the process will preferably result in respirable, highly dispersible particles comprising the modified insulin, and any other desired excipients.

The liquid formulations of the invention may be prepared by combining (i) the modified insulin; (ii) the fluid or propellant, e.g., in an amount sufficient to propel a plurality of doses, e.g., from an aerosol canister; and (iii) any further optional components; and dispersing the components. The components may be dispersed using a conventional mixer or homogenizer, by shaking, or by ultrasonic energy as well as by the use of a beadmill or a microfluidizer. Bulk formulations can be transferred to smaller individual aerosol vials by using valve transfer methods, pressure filling, or by using known cold-fill methods.

### Packaging and Containers

Unit dose pharmaceutical compositions may be contained in a container. Examples of containers include, but are not limited to, capsules, blisters, vials, ampoules, syringes, or container closure systems made of metal, polymer (e.g., plastic, elastomer), glass, or the like.

The container may be inserted into an aerosolization device. The container may be of a suitable shape, size, and material to contain the pharmaceutical composition and to provide the pharmaceutical composition in a usable condition. For example, the capsule or blister may comprise a wall, which comprises a material that does not adversely react with the pharmaceutical composition. In addition, the wall may comprise a material that allows the capsule to be opened to allow the pharmaceutical composition to be aerosolized. In one version, the wall comprises one or more of gelatin, hydroxypropyl methylcellulose (HPMC), polyethyleneglycol-compounded HPMC, hydroxypropylcellulose, agar, aluminum foil, or the like. In one version, the capsule may comprise telescopically adjoining sections, as described for example in U.S. Pat. No. 4,247,066, which is incorporated herein by reference. The size of the capsule may be selected to adequately contain the dose of the pharmaceutical composition. The sizes generally range from size 5 to size 000 with the outer diameters ranging from about 4.91 mm to 9.97 mm, the heights ranging from about 11.10 mm to about 26.14 mm, and the volumes ranging from about 0.13 mL to about 1.37 mL, respectively. Suitable capsules are available commercially from, for example, Shionogi Qualicaps Co. in Nara, Japan and Capsugel in Greenwood, S.C. After filling, a top portion may be placed over the bottom portion to form a capsule shape and to contain the powder within the capsule, as described in U.S. Pat. Nos. 4,846,876 and 6,357,490, and in WO 00/07572, which are incorporated herein by reference. After the top portion is placed over the bottom portion, the capsule can optionally be banded.

Prior to use, dry powders are generally stored under ambient conditions, and preferably are stored at temperatures at or below about 25° C., and relative humidities (RH) ranging from about 30 to 60%. More preferred relative humidity conditions, e.g., less than about 30%, may be achieved by the incorporation of a desiccating agent in the secondary packaging of the dosage form.

### Administration

The compositions of one or more embodiments of the present invention may be administered by various methods and techniques known and available to those skilled in the art.

The therapeutic peptide conjugates of the invention can be administered by any of a number of routes including without limitation, oral, rectal, nasal, topical (including transdermal, aerosol, buccal and sublingual), vaginal, parenteral (including subcutaneous, intramuscular, intravenous and intradermal), intrathecal, and pulmonary. Preferred forms of administration include parenteral and pulmonary. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

In one or more embodiments of the invention, a method is provided, the method comprising delivering a conjugate to a patient, the method comprising the step of administering to the patient a pharmaceutical composition comprising a therapeutic peptide polymer conjugate as provided herein. Administration can be effected by any of the routes herein described. The method may be used to treat a patient suffering from a condition that is responsive to treatment with therapeutic peptide by administering a therapeutically effective amount of the pharmaceutical composition.

For example, in one or more embodiments, the compositions described herein may be delivered using any suitable dry powder inhaler (DPI), i.e., an inhaler device that utilizes the patient's inhaled breath as a vehicle to transport the dry powder drug to the lungs. Preferred are Nektar Therapeutics' dry powder inhalation devices as described in U.S. Pat. Nos. 5,458,135; 5,740,794; and 5,785,049, which are incorporated herein by reference.

When administered using a device of this type, the powder is contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder medicament are described, e.g., in WO 97/41031 (1997), which is incorporated herein by reference.

Also suitable for delivering the powders described herein are dry powder inhalers of the type described, for example, in U.S. Pat. Nos. 3,906,950 and 4,013,075, which are incorporated herein by reference, wherein a premeasured dose of dry powder for delivery to a subject is contained within a hard gelatin capsule.

Other dry powder dispersion devices for pulmonarily administering dry powders include those described, for example, in EP 129985; EP 472598; EP 467172; and U.S. Pat. No. 5,522,385, which are incorporated herein by reference. Also suitable for delivering the dry powders of the invention are inhalation devices such as the Astra-Draco “TURBOHALER”. This type of device is described in detail in U.S. Pat. Nos. 4,668,281; 4,667,668; and 4,805,811, all of which are incorporated herein by reference. Other suitable devices include dry powder inhalers such as the ROTAHALER™ (Glaxo), Discus™ (Glaxo), Spiros™ inhaler (Dura Pharmaceuticals), and the Spinhaler™ (Fisons). Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572, which is incorporated herein by reference. Another class of dry powder inhalers, which may be used, is disclosed in U.S. Provisional Application Nos. 60/854,601 and 60/906,977, which are incorporated herein by reference, and which are owned by Nektar Therapeutics.

Dry powders may also be delivered using a pressurized, metered dose inhaler (MDI), e.g., the Ventolin™ metered dose inhaler, containing a solution or suspension of drug in a pharmaceutically inert liquid propellant, e.g., a chlorofluorocarbon or fluorocarbon, as described in U.S. Pat. Nos. 5,320,094 and 5,672,581, which are both incorporated herein by reference.

Alternatively, the compositions described herein may be administered by nebulization. For example, a dry powder may be dissolved or suspended in a solvent, e.g., water, ethanol, or saline. Nebulizers for delivering an aerosolized solution include the AERx™ (Aradigm), the Ultravent™ (Mallinkrodt), and the Acorn II™ (Marquest Medical Products).

Liquid formulations can be atomized by any of a variety of procedures. For example, the liquid can be sprayed through a two-fluid nozzle, a pressure nozzle, or a spinning disc, or atomized with an ultrasonic nebulizer or a vibrating orifice aerosol generator (VOAG). In one or more embodiments, a liquid formulation is atomized with a pressure nozzle, such as a BD AccuSpray nozzle. Thus, aerosolization apparatuses may be based on condensation aerosolization, an impinging jet technique, electrospray techniques, thermal vaporizing, or a Peltier device.

Jet nebulizers involve use of air pressure to break a liquid solution into aerosol droplets. In one or more embodiments, a jet nebulizer (e.g., Aerojet, AeroEclipse, Pari L. C., the Parijet, Whisper Jet, Microneb®, Sidestream®, Acorn II®, Cirrus, Salter, and Upmist®) generates droplets as a mist by shattering a liquid stream with fast moving air supplied by tubing from an air pump. Droplets that are produced by this method typically have a diameter of about 2-5 μm.

In one or more embodiments, an ultrasonic nebulizer that uses a piezoelectric transducer to transform electrical current into mechanical oscillations is used to produce aerosol droplets. Examples of ultrasonic nebulizers include, but are not limited to, the Siemens 345 UltraSonic Nebulizer™ and ones commercially available from, for example, Omron Healthcare, Inc. and DeVilbiss Health Care, Inc. See, e.g., EP 1 066 850, which is incorporated by reference herein. The resulting droplets typically have an MMAD in the range of about 1 to about 5 microns.

Vibrating porous plate nebulizers work by using a sonic vacuum produced by a rapidly vibrating porous plate to extrude a solvent droplet through a porous plate. See, e.g., U.S. Pat. Nos. 5,758,637; 5,938,117; 6,014,970; 6,085,740; and 6,205,999, which are incorporated herein by reference. Vibrating porous plates may be included in nebulizer systems such as those disclosed in U.S. Published Application Nos. 20050217666; 20050229927; and 20050229928.

For example, in one or more embodiments, the aerosol generator is the commercially available Aerogen (now Nektar Therapeutics, San Carlos, Calif.) aerosol generator which comprises a vibrational element and dome-shaped aperture plate with tapered holes. When the plate vibrates several thousand times per second, such as about 100 k/s to about 150 k/s, a micro-pumping action causes liquid to be drawn through the tapered holes, creating a low-velocity aerosol with a precisely defined range of droplet sizes. The Aerogen aerosol generator does not require propellant. An exemplary Aerogen aerosol generator that may be used is disclosed in WO 2006/127181, which is incorporated herein by reference.

In the Aerogen Aeroneb and Pari eFlow (Pari Respiratory Equipment, Germany), a piezoelectric oscillator is placed circumferentially around the vibrating mesh and vibrations shake precisely sized droplets of the nebulizer content through the membrane, to form a respirable mist of medication on the other side. In another vibrating mesh nebulizer, the Omron Micro-air (Omron, Japan), the piezoelectric oscillator is positioned proximal to the vibrating mesh instead of circumferentially around it, pushing rather than shaking droplets of droplets of nebulizer content through the pores in the membrane with a similar result.

In condensation aerosol generators, the aerosol is formed by pumping drug formulation through a small, electrically heated capillary. Upon exiting the capillary, the formulation is rapidly cooled by ambient air, and a gentle aerosol is produced that is relatively invariant to ambient conditions and the user inhalation rate. See, e.g., U.S. Pat. No. 6,701,922 and WO 03/059413, which are incorporated herein by reference. In one or more embodiments, the condensation aerosol generator comprises one disclosed by Alexza Molecular Delivery Corporation. See, e.g., U.S. Published Application No. 2004/0096402, which is incorporated herein by reference.

Another apparatus for delivery of a metered quantity of a liquid pharmaceutical composition for inhalation is described for example in WO 91/14468 and WO 97/12687, which are incorporated herein by reference. The nebulizers described therein are known by the name Respimat®.

One or more electrosprays may be used to nebulize liquid formulations. The term electrostatic spray (also known as electrohydrodynamic spray or electrospray) refers to systems in which the dispersion of the liquid relies on its electric charging, so that nebulization and gas flow processes are relatively uncoupled. Examples of electrospray devices are disclosed in U.S. Pat. Nos. 6,302,331; 6,583,408; and 6,803,565, which are incorporated herein by reference.

In one or more embodiments, the aerosol generator comprises a thermal vaporizing device. Such a device may be based on inkjet technology.

In one or more embodiments, the aerosol generator comprises a Peltier device. An example of such a device is disclosed in U.S. Published Application No. 2004/0262513, which is incorporated herein by reference.

In one or more embodiments, the aerosol generator comprises a vibrating orifice monodisperse aerosol generator (VOAG). This device is an example of one type of monodisperse aerosol generator.

In one or more embodiments, the aerosol generator comprises a thin film, high surface area boiler that relies on capillary force and phase transition. By inducing phase transition in a capillary environment, pressure is imparted onto the expanding gas, which is ejected. This technology has been disclosed by Vapore, Inc., and is known as Vapore-Jet CFV technology. See, e.g., U.S. Pat. Nos. 5,692,095; 5,870,525; 6,162,046; 6,347,936; 6,585,509; and 6,634,864, and U.S. application Ser. No. 10/691,067, which are all incorporated herein by reference.

The time for dosing is typically short. For a single unit dose, the total dosing time is normally less than about 1 minute. Administering two unit doses usually takes about 1 min. A five unit dose may take about 3.5 min to administer. Thus, the time for dosing may be less than about 5 min, such as less than about 4 min, less than about 3 min, less than about 2 min, or less than about 1 min.

As previously stated, the method of delivering a therapeutic peptide polymer conjugate as provided herein may be used to treat a patient having a condition that can be remedied or prevented by administration of therapeutic peptide.

Certain conjugates of the invention, e.g., releasable conjugates, include those effective to release the therapeutic peptide, e.g., by hydrolysis, over a period of several hours or even days (e.g., 2-7 days, 2-6 days, 3-6 days, 3-4 days) when evaluated in a suitable in-vivo model.

The actual dose of the therapeutic peptide conjugate to be administered will vary depending upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a conjugate of the invention will be delivered such that plasma levels of a therapeutic peptide are within a range of about 0.5 picomoles/liter to about 500 picomoles/liter. In certain embodiments the conjugate of the invention will be delivered such that plasma leves of a therapeutic peptide are within a range of about 1 picomoles/liter to about 400 picomoles/liter, a range of about 2.5 picomoles/liter to about 250 picomoles/liter, a range of about 5 picomoles/liter to about 200 picomoles/liter, or a range of about 10 picomoles/liter to about 100 picomoles/liter.

On a weight basis, a therapeutically effective dosage amount of a therapeutic peptide conjugate as described herein will range from about 0.01 mg per day to about 1000 mg per day for an adult. For example, dosages may range from about 0.1 mg per day to about 100 mg per day, or from about 1.0 mg per day to about 10 mg/day. On an activity basis, corresponding doses based on international units of activity can be calculated by one of ordinary skill in the art.

The unit dosage of any given conjugate (again, such as provided as part of a pharmaceutical composition) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

The invention also provides a method for administering a modified insulin as provided herein to a patient suffering from diabetes or insulin deficiency. The method comprises administering, generally via inhalation or injection, a therapeutically effective amount of the modified insulin (preferably provided as part of a pharmaceutical preparation). The method of administering may be used to treat any condition that can be remedied or prevented by administration of the modified insulin. Those of ordinary skill in the art appreciate which conditions modified insulin can effectively treat. The actual dose to be administered will vary depend upon the age, weight, and general condition of the subject as well as the severity of the condition being treated, the judgment of the health care professional, and conjugate being administered. In this regard, animal data suggests that if the dose of di-acetylated 5K-PEGylated insulin is too high, the lung tissue may degrade, which may increase the permeability of the lung. Therapeutically effective amounts are known to those skilled in the art and/or are described in the pertinent reference texts and literature. Generally, a therapeutically effective amount, based on equivalent insulin mass, will range from about 0.001 mg to 100 mg, such as from 0.01 mg/day to 75 mg/day, from 0.10 mg/day to 50 mg/day, or from 1 mg/day to 10 mg/day, and may range from 0.005 mg/kg to 0.3 mg/kg, such as 0.01 mg/kg to 0.15 mg/kg, 0.02 mg/kg to 0.1 mg/kg, or 0.03 mg/kg to 0.07 mg/kg.

The unit dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration twice daily, once daily, three times weekly, twice weekly, once weekly, and any combination thereof. Once the clinical endpoint has been achieved, dosing of the composition may be halted.

If the modified insulin is administered to the lungs, the modified insulin will generally stay in the lungs longer than regular insulin. For instance, the modified insulin typically has a residence time in the lungs ranging from 2 hours to 4 days, such as 2 hours to 3 days, 3 hours to 24 hours, or 4 hours to 12 hours. In certain embodiments, at least about 75% of the administered modified insulin is present in the lungs 2 hours after administration, 3 hours after administration, or 4 hours after administration.

Once the modified insulin reaches the blood, the modified insulin will typically stay in the blood longer than regular insulin. For instance, the blood half-life (corrected for baseline plasma level) will usually range from 2 hours to 4 days, such as 2 hours to 3 days, 3 hours to 24 hours, or 4 hours to 12 hours.

Administration of the modified insulin usually results in a measurable reduction in blood glucose level in less than 1 hour after administration, such as less than 50 minutes, less than 40 minutes, or less than 30 minutes. The administration typically results in a measurable reduction in blood glucose level for a period of at least about 6 hours, such as at least about 7 hours, or at least about 8 hours. The measurable reduction usually ranges from 30 minutes to 7 days, such as 40 minutes to 5 days, 50 minutes to 2 days, or 1 hour to 24 hours.

In view of the above, the present invention includes the balancing of several factors. For instance, the residence time in the lungs is dependent on the ability of the modified insulin to avoid passage through the lung membrane as well as resistance to enzymatic degradation. Once the modified insulin passes through the lung, or if the modified insulin is delivered via a different route, resistance to enzymatic degradation is still a factor. Of course, the modified insulin also needs to be active or be capable of metabolizing to an active form.

While balancing these several factors, the present inventors discovered many surprising results. For instance, the present inventors surprisingly found active modified insulins comprising both a hydrophilic polymer and a moiety having one to ten carbon atoms. When the hydrophilic polymer is attached to either the B1 or B29 sites, preferably at B1, the activity is better than when such a polymer is attached to the A1 site. It is also noted that the moiety should have less than ten carbon atoms to provide protection against enzymatic degradation and yet be small enough to avoid reducing activity. The effect on pharmacodynamic profile of using both a hydrophilic polymer and a moiety is typically synergistic. Thus, the modified insulins of the present invention typically have unexpectedly high bioactivity and extended pharmacodynamic profile.

It is to be understood that while the invention has been described in conjunction with the preferred specific embodiments thereof, the foregoing description as well as the examples that follow are intended to illustrate and not limit the scope of the invention. For instance, the above discussion and present invention may, of course, be extended to therapeutic peptides other than insulin. Further, the Examples and entire disclosure of U.S. Provisional Application No. ______ (Attorney Docket No. SHE0203.PRO), filed concurrently herewith, are incorporated herein by reference. Other aspects, advantages and modifications within the scope of the invention will be apparent to those skilled in the art to which the invention pertains.

### EXPERIMENTAL

The practice of the invention will employ, unless otherwise indicated, conventional techniques of organic synthesis and the like, which are within the skill of the art. Such techniques are fully explained in the literature. Reagents and materials are commercially available unless specifically stated to the contrary. See, for example, J. March, Advanced Organic Chemistry: Reactions Mechanisms and Structure, 4th Ed. (New York: Wiley-Interscience, 1992), supra.

In the following examples, efforts have been made to ensure accuracy with respect to numbers used (e.g., amounts, temperatures, etc.) but some experimental error and deviation should be accounted for. Unless indicated otherwise, temperature is in degrees C. and pressure is at or near atmospheric pressure at sea level.

Although other abbreviations known by one having ordinary skill in the art will be referenced, other reagents and materials will be used, and other methods known by one having ordinary skill in the art will be used, the following list and methods description is provided for the sake of convenience.

### Abbreviations

mPEG-SPA mPEG-succinimidyl propionate
mPEG-SBA mPEG-succinimidyl butanoate mPEG-OPSS mPEG-orthopyridyl-disulfide
mPEG-MAL mPEG-maleimide, CH₃O—(CH₂CH₂O)ₙ—CH₂CH₂-MAL
mPEG-SMB mPEG-succinimidyl α-methylbutanoate, CH₃O—(CH₂CH₂O)ₙ—CH₂CH₂—CH(CH₃)—C(O)—O-succinimide
mPEG-ButyrALD H₃O—(CH₂CH₂O)ₙ—CH₂CH₂—O—C(O)—NH—(CH₂CH₂O)₄—CH₂CH₂CH₂C(O)H
mPEG-PIP CH₃O—(CH₂CH₂O)ₙ—CH₂CH₂—C(O)-piperidin-4-one
mPEG-CM CH₃O—(CH₂CH₂O)ₙ—CH₂CH₂—O—CH₂—C(O)—OH)
anh. Anhydrous
CV column volume
Fmoc 9-fluorenylmethoxycarbonyl
NaCNBH₃ sodium cyanoborohydride
HCl hydrochloric acid
HEPES 4-(2-hydroxyethyl)-1-piperazineethanesulfonic acid
NMR nuclear magnetic resonance
DCC 1,3-dicyclohexylcarbodiimide
DMF dimethylformamide
DMSO dimethyl sulfoxide
DI deionized
MW molecular weight
K or kDa kilodaltons
SEC Size exclusion chromatography
HPLC high performance liquid chromatography
FPLC fast protein liquid chromatography
SDS-PAGE sodium dodecyl sulfate-polyacrylamide gel electrophoresis
MALDI-TOF Matrix Assisted Laser Desorption Ionization Time-of-Flight
TLC Thin Layer Chromatography
THF Tetrahydrofuran

### Materials

All PEG reagents referred to in the appended examples are commercially available unless otherwise indicated.

mPEG Reagent Preparation

Typically, a water-soluble polymer reagent is used in the preparation of peptide conjugates of the invention. For purposes of the present invention, a water-soluble polymer reagent is a water-soluble polymer-containing compound having at least one functional group that can react with a functional group on a peptide (e.g., the N-terminus, the C-terminus, a functional group associated with the side chain of an amino acid located within the peptide) to create a covalent bond. Taking into account the known reactivity of the functional group(s) associated with the water-soluble polymer reagent, it is possible for one of ordinary skill in the art to determine whether a given water-soluble polymer reagent will form a covalent bond with the functional group(s) of a peptide.

Representative polymeric reagents and methods for conjugating such polymers to an active moiety are known in the art, and are, e.g., described in Harris, J. M. and Zalipsky, S., eds, Poly(ethylene glycol), Chemistry and Biological Applications, ACS, Washington, 1997; Veronese, F., and J. M Harris, eds., Peptide and Protein PEGylation, Advanced Drug Delivery Reviews, 54(4); 453-609 (2002); Zalipsky, S., et al., “Use of Functionalized Poly(Ethylene Glycols) for Modification of Polypeptides” in Polyethylene Glycol Chemistry: Biotechnical and Biomedical Applications, J. M. Harris, ed., Plenus Press, New York (1992); Zalipsky (1995) Advanced Drug Reviews 16:157-182, and in Roberts, et al., Adv. Drug Delivery Reviews, 54, 459-476 (2002).

Additional PEG reagents suitable for use in forming a conjugate of the invention, and methods of conjugation are described in Shearwater Corporation, Catalog 2001; Shearwater Polymers, Inc., Catalogs, 2000 and 1997-1998, and in Pasut. G., et al., Expert Opin. Ther. Patents (2004), 14(5). PEG reagents suitable for use in the present invention also include those available from NOF Corporation (Tokyo, Japan), as described generally on the NOF website (2006) under Products, High Purity PEGs and Activated PEGs. Products listed therein and their chemical structures are expressly incorporated herein by reference. Additional PEGs for use in forming a GLP-1 conjugate of the invention include those available from Polypure (Norway) and from QuantaBioDesign LTD (Powell, Ohio), where the contents of their online catalogs (2006) with respect to available PEG reagents are expressly incorporated herein by reference.

In addition, water-soluble polymer reagents useful for preparing peptide conjugates of the invention is prepared synthetically. Descriptions of the water-soluble polymer reagent synthesis can be found in, for example, U.S. Pat. Nos. 5,252,714, 5,650,234, 5,739,208, 5,932,462, 5,629,384, 5,672,662, 5,990,237, 6,448,369, 6,362,254, 6,495,659, 6,413,507, 6,376,604, 6,348,558, 6,602,498, and 7,026,440.

### Utility

The compositions of the invention are useful, when administered in a therapeutically effective amount to a mammalian subject, for treating or preventing any condition responsive to the administration of the pharmacologically active compound in the formulation. For example, in cases in which the pharmacologically active compound is a modified insulin of the invention, the condition being treated may be diabetes. Thus, for example, the present invention finds use in the treatment of diabetes.

The following examples are illustrative of the present invention, and are not to be construed as limiting the scope of the invention. Variations and equivalents of these examples will be apparent to those of skill in the art in light of the present disclosure, the drawings, and the claims herein. Unless otherwise stated, all percentages are by weight of the total composition.

### EXAMPLES

### Nomenclature

The prefix B1 when used in naming a compound means that the substituent indicated is attached to the terminal α-amino atom of amino acid on the B-chain of the insulin molecule, i.e., phenylalanine. The prefix B29 when used in naming a compound means that the substituent indicated is attached to the ε-amino atom on the side chain of amino acid twenty-nine of the insulin molecule, i.e., lysine. The prefix A1 when used in naming a compound means that the substituent indicated is attached to the α-amino atom of the amino acid of the A-chain of the insulin molecule, i.e., glycine. The subscript K outside of a pair of brackets means that the contents within the brackets are multiplied by about one thousand, e.g., the term -(ethoxy)_{2K}- means a polyethylene glycol divalent moiety having a molecular weight of about two thousand. For example, a compound of the following formula:

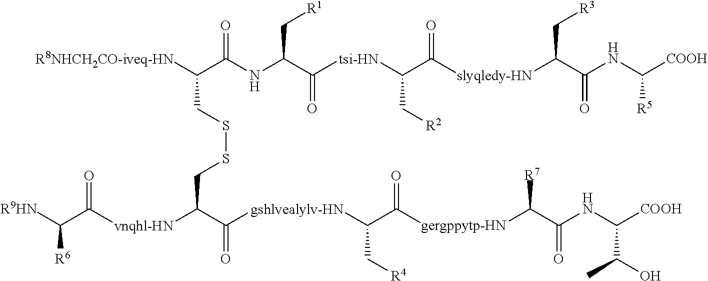

in which each a, d, e, g, h, i, l, n, p, q, r, s, t, v and y is an amino acid residue attached to its adjacent amino acid residue via a peptide linkage, R¹ and R² together form dithio, R³ and R⁴ together form dithio, R⁵ is carbamoylmethyl, R⁶ is benzyl, R⁷ is —(CH₂)₄NHR¹⁰, wherein R¹⁰ is acetyl, R⁸ is 3-[methoxy(ethoxy)_{2K}]propionyl, and R⁹ is acetyl, and is named di-N^{αA1},N^{εB29}-(acetyl)-N^{αB1}-{3-[methoxy(ethoxy)_{2K}]propionyl} insulin.

### Example 1

### Synthesis of N^{αA1},N^{εB29}-{3-[methoxy(ethoxy)₅₅₀]propionyl}insulin (550-lot 1-PEG Insulin)

Insulin (1 g, 172.2 μmol) was added to dry DMSO (4 mL) and the solution was stirred for 10 to 20 minutes until the insulin was dissolved. 2,5-Dioxopyrrolidin-1-yl 3-[methoxy(ethoxy)₅₅₀]propionate (0.28 g, 509.1 μmol) was suspended in dry DMSO (1 mL). The PEG solution was added quickly to the insulin solution. The reaction was stirred at room temperature for 24 hours.

The reaction mixture was purified by semi-prep RP-HPLC using the following conditions: Waters C18 40 mm×100 mm column and UV detection at 277 nm. 0.1% TFA in deionized water was used as mobile phase A, and mobile phase B was 0.1% TFA in acetonitrile. The loading range was 260 mg to 270 mg, and four purifications were performed. After the product was pooled, it was distilled, frozen, and then lyophilized to obtain a dry powder. The product was analyzed by reverse phase HPLC and contained a mixture of 54% mono-PEGylated insulin and 45% di-PEGylated insulin.

### Example 2

### Synthesis of N^{αB1}-{3-[methoxy(ethoxy)₇₅₀]propionyl}insulin (750-lot 1-PEG Insulin and 750-lot 2-PEG Insulin)

### Preparation of Di-tboc-Insulin

Insulin (0.2 g, 34.33 μmol) was added to dry DMSO (1.5 mL) and triethylamine (0.08 mL). The solution was stirred for 10 to 20 minutes until the insulin was dissolved. Di-tert-butyldicarbonate (17 μL, 73.3 μmol) was added to insulin and was reacted at room temperature for 24 hours. The reaction was then precipitated into 200 mL acetone and 8 drops of 6 N HCl. The reaction mixture was dried under vacuum. The product was further isolated from the reaction mixture by purification on a Waters semi-prep C₁₈ column using mobile phases consisting of A: 0.067% TFA in deionized water and B: acetonitrile. The injection volume was 3 mL. The flow rate was set to 3 mL/min, and the UV detector was set at 280 nm. The di-tboc-insulin product was purified using a linear gradient of 5-50% B over 50 minutes. The product was collected and then lyophilized.

### Preparation of 750-Lot 1-PEG Insulin

Di-tboc-insulin (0.07 g, 12.0 μmol) was added to dry DMSO (1.5 mL) and triethylamine (0.05 mL). The solution was stirred for 10 to 20 minutes until the insulin was dissolved. 2,5-Dioxopyrrolidin-1-yl 3-[methoxy(ethoxy)₇₅₀]propionate (0.088 g, 85.0 μmol) was suspended in dry DMSO (1 mL). The PEG solution was added quickly to the insulin solution. The reaction was stirred at room temperature for ˜24 hours. The product was precipitated using acetone and a few drops of 6 N HCl. The product was collected and dried under vacuum. The dry powder (37 mg) was then dissolved into 200 μL trifluoroacetic acid and stirred at room temperature for 1 hour to remove the t-boc protecting groups. The product was precipitated into ethyl ether and dried under vacuum. It was then dissolved in deionized water (15 mL) and lyophilized. The yield of dry powder was 24.5 mg. The insulin content was found to be 29.1% by reverse phase HPLC. The purity was unknown.

### Preparation of 750-Lot 2-PEG Insulin

Di-tboc-insulin (0.063 g, 10.6 μmol) was added to dry DMSO (0.5 mL) and triethylamine (0.2 mL). The solution was stirred for 10 to 20 minutes until the insulin was dissolved. 2,5-Dioxopyrrolidin-1-yl 3-[methoxy(ethoxy)₇₅₀]propionate (0.033 g, 31.8 μmol) was suspended in dry DMSO (1 mL). The PEG solution was added quickly to the insulin solution. The reaction was stirred at room temperature for ˜24 hours. Trifluoroacetic acid (˜300 μL) was added to the reaction mixture and precipitated into ˜100 mL ethyl ether. The product was collected and dried under vacuum. The dry powder was then dissolved into 300 μL trifluoroacetic acid and stirred at room temperature for 1 hour to remove the t-boc protecting groups. The product was precipitated into ethyl ether and dried under vacuum. It was then dissolved in deionized water (15 mL) and lyophilized. The % insulin was found to be 33.6% by reverse phase HPLC. The molecular weight was 6610 Da by MALDI. The purity was unknown.

### Examples 3 and 4

### Synthesis of N^{αA1},N^{εB29}-{3-[methoxy(ethoxy)₇₅₀]propionyl}insulin (750-lot 3- and 750-lot 4-PEG Insulin)

Insulin (1.6 g, 274.6 μmol) was added to dry DMSO (7.5 mL) and triethylamine (0.4 mL). The solution was stirred for 10 to 20 minutes until the insulin was dissolved. 2,5-Dioxopyrrolidin-1-yl 3-[methoxy(ethoxy)₇₅₀]propionate (0.48 g, 475.0 μmol) was suspended in dry DMSO (2.5 mL). The PEG solution was added quickly to the insulin solution. The reaction was stirred at room temperature for ˜24 hours. Trifluoroacetic acid (0.4 mL) was added to the reaction and allowed to stir for approximately 1 hour.

The product was further isolated from the reaction mixture by purification on a Waters semi-prep C₁₈ column using mobile phases consisting of A: 0.1% TFA in deionized water and B: 0.1% TFA in acetonitrile. The UV detector was set at 280 nm. Two pools were made from the purification. The first pool, 750-lot 3, consisted of 50% mono-PEGylated insulin and 50% di-PEGylated insulin. The second pool, 750-lot 4, consisted of 99.2% di-PEGylated insulin. Each pool was distilled and lyophilized separately. Insulin content for 750-lot 3 was determined to be 72.6% and for 750-lot 4 was 82.9%.

### Example 5

### Synthesis of N^{εB29}-{3-[methoxy(ethoxy)₇₅₀]proprionyl}insulin (750-lot 5-PEG Insulin)

Twenty ml 0.1 M Na₂CO₃ pH 10.0 (fresh) and 20 ml of DMSO were combined and stirred to mix. Human insulin (100.9 mg, 17.4 μmol) was dissolved in 4 ml of the sodium carbonate/DMSO solution with stirring. The insulin solution was stirred for an additional 5 minutes after the insulin was dissolved. The pH of the insulin solution was 11.6.

2,5-Dioxopyrrolidin-1-yl 3-[methoxy(ethoxy)₇₅₀]propionate (134.3 mg, 179.1 μmol) was suspended in 1.34 ml of DMSO. Slowly, 0.244 ml of the 2,5-dioxopyrrolidin-1-yl 3-[methoxy(ethoxy)₇₅₀]propionate (24.5 mg, 23.5 μmol) solution was added to the insulin solution while maintaining the pH at 11.87-11.88. At a reaction time of 3 minutes, additional PEG reagent was added (8.2 mg, 10.9 μmol) followed by another addition at 6 minutes (8.2 mg, 10.9 μmol). The PEGylation reaction continued for 5 minutes at pH 11.88 after all of the PEG solution was added. The reaction was quenched by adjusting the pH to 7.58 with 1 M HCl. The solution was filtered through a 0.22 μm filter unit. The reaction mixture was diluted 1:1 with 50 mM Sodium Acetate pH 3.5. The reaction mixture was stored at −20° C. until purification. Analysis of the reaction mixture on an HPLC indicated that the reaction produced 78.5% mono 750 PEG insulin, 13.7% di 750 insulin, and 7.9% unreacted insulin.

The reaction mixture was purified using a chromatography column. Specifically, 186 ml column of SP650M toyopearl resin. Buffer A=50 mM Sodium Acetate pH 3.5, buffer B=buffer A+0.9 M NaCl (pH 3.5). AKTA purifier system used. Sample was diluted with buffer A reaction mixture. Method used was a gradient of 0-20% B over 18 column volumes. Thirteen ml fractions were collected. The pooled fractions were also desalted using an Amberchrom CG71 column with buffers consisting of A: 3% ethanol, 0.5% acetic acid and B: 85% ethanol, 0.5% acetic acid. The product was then lyophilized and yielded 60.6 mg N^{εB29}-{4-[methoxy(ethoxy)₇₅₀]propionyl}insulin with an insulin content of 78.8%.

### Example 6

### Synthesis of N^{εB29}-{3-[methoxy(ethoxy)_{2K}]propionyl}insulin (2K-2-PEG Insulin)

Proceeding as in Example 5, but substituting insulin (50.65 mg, 8.7 μmol) and 2,5-dioxopyrrolidin-1-yl 3-[methoxy(ethoxy)_{2K}]propionate (51.7 mg, 13.96 μmol), gave N^{αA1},N^{εB29}-mPEG₂₀₀₀propionyl-insulin (20.9 mg, 3.6 μmol) and N^{εB29}-mPEG₂₀₀₀propionyl-insulin.

### Example 7

### Synthesis of N^{αA1},N^{εB29}-{3-[methoxy(ethoxy)_{2K}]propionyl}insulin (2K-PEG Insulin)

Proceeding as in Example 3 and 4, but substituting insulin (201.1 mg, 34.5 μmol) and 2,5-dioxopyrrolidin-1-yl 3-[methoxy(ethoxy)_{2K}]propionate (138.3 mg, 69.2 μmol), gave N^{αA1},N^{εB29}-mPEG₂₀₀₀propionyl-insulin (67.5 mg) with an insulin content of 74.8%.

### Example 8

### Synthesis of N^{εB29}-{3-[methoxy(ethoxy)_{5K}]propionyl}insulin (5K-B29 PEG Insulin)

Twenty ml 0.1 M Na₂CO₃ pH 10.0 (fresh) and 20 ml of DMSO were combined and stirred to mix. Five hundred thirty three mg of mSPA-5K were suspended in 5.33 ml of DMSO. Four hundred mg of human insulin was dissolved in 15 ml of the sodium carbonate/DMSO solution with stirring. The insulin solution was stirred for an additional 5 minutes after the insulin was dissolved. The pH of the insulin solution was adjusted to 11.88 with 0.5 N Sodium Hydroxide (˜250 μl). Slowly, 4.8 ml of the mSPA-5K solution was added to the insulin solution while maintaining the pH at 11.87-11.88, the PEG solution was added at ˜1 ml/min. The PEGylation reaction continued for 5 minutes at pH 11.88 after all of the PEG solution was added (the solution was opaque). The reaction was quenched by adjusting the pH to 4.5 with ˜2.7 ml 1 M HCl (the solution was still cloudy). The solution was filtered through a 0.22 μm filter unit. The reaction mixture was diluted 1:1 with 50 mM Sodium Acetate pH 3.5. The reaction mixture was stored at −20° C. until purification. Analysis of the reaction mixture on an HPLC indicated that the reaction produced 62% mono 5K PEG insulin, 6.5% di 5K insulin, and 31% unreacted insulin.

The reaction mixture was purified using a chromatography column. Specifically, eighty-five ml column of SP650S toyopearl resin (2.2 cm i.d.). Buffer A=50 mM Sodium Acetate pH 3.5, buffer B=buffer A+1 M NaCl (pH 3.5). AKTA purifier system used. Sample was the quenched (pH 4.5), filtered, 1:1 dilution with buffer A reaction mixture. Method used was a gradient of 5-25% B over 10 column volumes. Ten ml fractions were collected. The mono 5K PEG insulin eluted at about 11% B (˜88 mM NaCl).

### Example 9

### Synthesis of N^{αB1}-{3-[methoxy(ethoxy)_{5K}]propionyl}insulin (5K-B1 PEG Insulin)

Insulin (700 mg, 120.5 mop was added to dry DMSO (24 mL) and triethylamine (1.2 mL) and the solution was stirred for 10 to 20 minutes until the insulin was dissolved. 3-Methylfuran-2,5-dione (23.7 μL, 132.9 μmol) was added and mixture was stirred at room temperature for 25 minutes to give a solution of di-N^{αA1},N^{εB29}-(3-carboxybut-2-enoyl)insulin.

2,5-Dioxopyrrolidin-1-yl 3-[methoxy(ethoxy)_{5K}]propionate (3 g, 595 μmol) was suspended in dry DMSO (32 mL) and triethylamine (192 μL) and the suspension was added drop-wise quickly to the protected-insulin solution. The reaction was stirred at room temperature for 24 hours and then deionized water (60 mL) was added to the reaction mixture. The mixture was dialyzed in 3500 MWCO tubing (Pierce) vs. 4 L deionized water overnight at room temperature. The remaining mixture was lyophilized to dryness (2 g).

The dry material was dissolved in trifluoroacetic acid (10 mL) with stirring at room temperature for 24 hours. The solution was diluted with deionized water (90 mL) and the pH was adjusted to 3.5 with 1M sodium hydroxide. The solution was dialyzed in 3500 MWCO tubing vs. 4 L water overnight in preparation for purification (200 mL total volume). The pH of the solution was adjusted to 3.5 with 1M hydrochloric acid. Analysis of the reaction mixture on an Agilent 1100 HPLC system indicated that the reaction result contained about 44% mono-substituted product, 26% di-substituted product, and 20% un-substituted product.

An 85 mL column of SP650S toyopearl resin was used with buffer A containing 0.05M sodium acetate at pH 3.5 and buffer B containing 0.05M sodium acetate and 1M sodium chloride at pH 3.5. An AKTA prime system was employed. Seven runs were necessary to purify the dialyzed insulin solution (6×100 mL and 1×91 mL). The purification method used a gradient of 0-17% buffer B over 7 column volumes for all purifications and 12 mL fractions were collected resulting in a mono-substituted product peak at about 10% B (0.1M sodium chloride).

The fractions from each run that contained mono-substituted product were combined, buffer exchanged into deionized water with the PALL Omega 5K Ultrasette and a peristaltic pump with 10-20 psi pressure on the inlet line. The buffer exchanged product (about 140 mL) was 0.2 μm filtered. The solution was frozen overnight at −80° C. (with the bottles lying horizontally) then lyophilized to dryness to give N^{αB1}-{3-[methoxy(ethoxy)_{5K}]propionyl}insulin (104.3 mg, 9.6 μmole) as a dry powder.

### Examples 10 and 11

### Synthesis or N^{αB1}-{3-[methoxy(ethoxy)_{2K}]propionyl}insulin (2K-B1 PEG Insulin); Synthesis of N^{αB1}-{3-[methoxy(ethoxy)_{3K}]propionyl}insulin (3K-B1 PEG Insulin)

Insulin was citraconylated in triethylamine/DMSO at room temperature for about 30 minutes. Five-fold molar excess of 2000- or 3000-dalton methoxyPEG succinimidyl propionic acid was conjugated to the citraconylated insulin. After dialysis into water and lyophilization to dryness, the citraconyl groups were released or cleaved by trifluoroacetic acid. The deprotected PEG-insulins were purified by cation exchange (column packed with SP650S Toyopearl resin) in 50 mM sodium acetate pH 3.5 with a NaCl gradient. The purified conjugate was buffer exchanged into water, 0.2 μm filtered and lyophilized to provide a dry, additive free insulin powder for in vitro and in vivo use. The purity of the PEGylated insulins was determined by C18 RP-HPLC (Betasil) on an Agilent 1100 HPLC system with a 0.1% TFA with an acetonitrile gradient.

The mean MW of the B1-2K-PEG-insulin and B1-3K-PEG-insulin was 8094 and 9019, respectively, as determined by MALDI. The purity of these PEGylated insulin conjugates was >97%.

### Example 12

### Synthesis of N^{αB1}-{4-[methoxy(ethoxy)_{2K}]butyryl}insulin (2K-B1 butyraldehyde PEG Insulin)

PEGylation of Insulin with 2K Butyraldehyde—the Initial Reaction Conditions:

Experiments 1-3 listed below contain derivations of initial conditions.

About 30 mg Insulin was stirred in 25 ml 100 mM Acetic Acid pH 5 for 20 minutes. Then 1.2 ml of 0.5 M Sodium Cyanoborohydride (dissolved in water) was added so that the final concentration in the Insulin solution was 20 mM in the 30 ml reaction. The needed amount of 2K PEG butyraldehyde per reaction was dissolved in 5 ml 100 mM Acetic Acid pH 5. The PEG solution was added dropwise to the Insulin-cyanoborohydride solution. The reaction was stirred overnight at room temperature. A 1:4 dilution of the reaction mixture in 0.1% TFA was made for analytical HPLC investigation and 50 μl per run was injected.

Experiment 1:

All reactions at 1 mg/ml Insulin,
20 mM cyanoborohydride

| | Insulin:PEG ratios: | | |
|---|---|---|---|
| 100 mM Acetic Acid pH 5 | 1:1 | 1:5 | 1:10 |
| 1:1 100 mM Acetic Acid | 1:1 | 1:5 | 1:10 |
| pH 5:Acetonitrile | | | |

Experiment 2:

Control reaction had Insulin
concentration of 1 mg/ml, all reactions
contained 20 mM Cyanoborohydride

| | pH: | | |
|---|---|---|---|
| 100 mM Acetic Acid | pH 4 | pH 4.5 | pH 5 |
| 100 mM Acetic Acid | pH 4 | | pH 5 |
| 2.5 mg/ml Insulin | | | |
| 1:1 100 mM Acetic | | | pH 5 |
| Acid:DMSO | | | |

Experiment 3:

Insulin:PEG ratios of 1:1, 1:1.2, 1:1.5, 1:2

All reactions in 100 mM Acetic Acid pH 4

Control reaction had Insulin concentration
of 2.5 mg/ml, all reactions were in 100 mM
Acetic Acid pH 4 and contained 20 mM
Cyanoborohydride

| | mg/ml Insulin: | | |
|---|---|---|---|
| 1:1 Insulin:PEG | 2.5 mg/ml | 5 mg/ml | 10 mg/ml |
| 1:1.2 Insulin:PEG | 2.5 mg/ml | | |
| 1:1.5 Insulin:PEG | 2.5 mg/ml | | |
| 1:2 Insulin:PEG | 2.5 mg/ml | | |

### Purification of Butyraldehyde PEG Insulin: SP650S

| | Toyopearl |
|---|---|
| Column name | SP650 |
| Column type (millipore) | Vantage L |
| Column diameter (mm) | 22 |
| Column cross area (cm²) | 44 |
| Resin height (cm) | 20 |
| Column volume (ml) | 76.0 |
| Flow rate (mL/min.) | 15 |
| Linear flow rate (cm/h) | 20.5 |
| Total Amount loaded (mg) | 30 |
| Resin load (mg sample/ml resin) | 0.4 |

Buffer conditions:
Buffer A=50 mM Sodium Acetate pH 3.5,
Buffer B=buffer A+1 M NaCl
   System used: AKTA Basic chromatography system (A product of GE Healthcare)
   Fraction collection: 12 ml fractions

### Detection: 280 nm

Flow rate: 15 ml/min

### Method:

| Time (min.) | Injection | % B | CV |
|---|---|---|---|
| 0 | Load | 0 | 2 |
| 10 | Inject | 0 | 1.6 |
| 18 | Load | 0 | 2 |
| 28 | Load | 17 | 10 |
| 79 | Load | 100 | 0 |
| 79 | Load | 100 | 2 |
| 89 | Load | 0 | 0 |
| 89 | Load | 0 | 1 |

The initial PEGylation experiments with m2K butyraldehyde and insulin (with 20 mM cyanoborohydride) all resulted in PEGylated insulin being produced. The 1:1 ratio of insulin to PEG gave mostly a mono PEG insulin product. The higher ratios of insulin:PEG (1:5 and 1:10) led to the production of PEG insulin conjugates other than mono PEG insulin (these could be mono PEG insulin at site A1, di-PEG insulin, and/or tri-PEG insulin). Using 1:2 ratio of insulin to PEG at 2.5 mg/ml insulin in 100 mM acetic acid pH 4 was the best result for producing the most mono PEG insulin and had about 60% mono PEG insulin. The insulin was most soluble at pH 4: up to 5 mg/ml Insulin. All reactions in this Example contained 20 mM cyanoborohydride.

PEGylation of Insulin with m2K PEG Butyraldehyde

Experiment 1

Yield of Mono PEG Insulin in reaction mixture
(HPLC area %)

| | Insulin:PEG ratios: | | |
|---|---|---|---|
| | 1:1 PEG | 1:5 PEG | 1:10 PEG |
| 100 mM Acetic Acid | 31 | 36 | 17 |
| 1:1 100 mM Acetic | 47 | 38 | 28 |
| Acid:acetonitrile | | | |

The best result from this set of experiments was the 1:1 100 mM Acetic Acid, pH 5:Acetonitrile with the production of 47% Mono PEG Insulin. This reaction condition led to precipitate forming in the tube after overnight stirring.

Experiment 2

Yield of mono PEG insulin in reaction
mixture (HPLC area %)

| | pH: | | |
|---|---|---|---|
| | pH 4 | pH 4.5 | pH 5 |
| 100 mM acetic acid | 47 | 46 | 57 |
| 100 mM acetic acid | 47 | | 44 |
| 2.5 mg/ml insulin | | | |
| 1:1 100 mM acetic | | | 24 |
| acid:DMSO | | | |

The best result from this set of experiments was the insulin at pH 5 in 100 mM acetic acid. This is not a good condition to take forward because of the low solubility of insulin at this pH as evidenced by the lower yield of mono PEG insulin at 2.5 mg/ml Insulin in 100 mM Acetic Acid pH 5 (57% vs. 44%). Another good result in this set of experiments was the 1 and 2.5 mg/ml Insulin in 100 mM acetic acid at pH 4. The acetic acid:DMSO solvent system was the worst result in this set of experiments, yielding just 24% mono PEG insulin.

Experiment 3

Yield of Mono PEG Insulin in reaction mixture
(HPLC area %)

| All in 100 mM | mg/ml Insulin: | | |
|---|---|---|---|
| acetic acid pH 4 | 2.5 mg/ml | 5 mg/ml | 10 mg/ml |
| 1:1 insulin:PEG | 47 | 49 | 44 |
| 1:1.2 insulin:PEG | 53 | | |
| 1:1.5 insulin:PEG | 58 | | |
| 1:2 insulin:PEG | 60 | | |

The best result in this set of experiments was the 2.5 mg/ml Insulin in 100 mM acetic acid pH 4 with 1:2 insulin:PEG yielding ˜60% mono PEG insulin.

### Purification of PEG 2K Butyraldehyde: Cation Exchange

Seventy-six ml column of SP650S toyopearl resin was used with buffer A=50 mM Sodium Acetate pH 3.5, buffer B=buffer A+1 M NaCl (pH 3.5). AKTA Basic system was used. One purification run was done after dilution of the PEG Insulin reaction to 125 ml with buffer A and loading the entire sample onto the column. The gradient was 0-17% B over 10 column volumes for this purification. Twelve ml fractions were collected. UV detection at 280 nm was used to visualize the sample elution.

The gradient of 0-17% B was inadequate to elute the Mono PEG Insulin. The gradient should be increased to ˜33%. The Mono PEG Insulin did separate from Insulin in the 100% B column wash portion of the method. Verification of the separation was done by HPLC analysis of fraction 95 (Mono PEG Insulin) and fraction 99 (Insulin).

At pH 5 or lower 2K PEG butyraldehyde was reactive with Insulin. 1:1 Insulin:PEG produced primarily Mono PEG Insulin. Higher ratios of Insulin:PEG (1:5 and 1:10) yielded other PEG Insulin conjugates in addition to the Mono PEG Insulin. Insulin solubility at pH 5 was less than 1 mg/ml. Insulin solubility at pH 4 was ˜5 mg/ml. Mixed solvents such as Acetic Acid/Acetonitrile and Acetic Acid/DMSO did not lead to increased Mono PEG Insulin production compared with Acetic Acid solvent system. The best conditions from all experiments in this report were: 2.5 mg/ml Insulin in 100 mM acetic acid pH 4, 20 mM cyanoborohydride, and 2 fold excess 2K PEG butyraldehyde stirred at room temperature overnight. These reaction conditions led to the production of 60% mono PEG insulin. Purification to separate mono PEG insulin and insulin as presented in this report was sufficient to yield a fraction of mono PEG insulin. Peptide mapping of Insulin and Mono 2K Insulin indicated that site B1 on Insulin was PEGylated.

### Example 13

### Synthesis of Acetylated Insulin

This Example shows the effect of pH and molar ratio of reactants on acetylation.

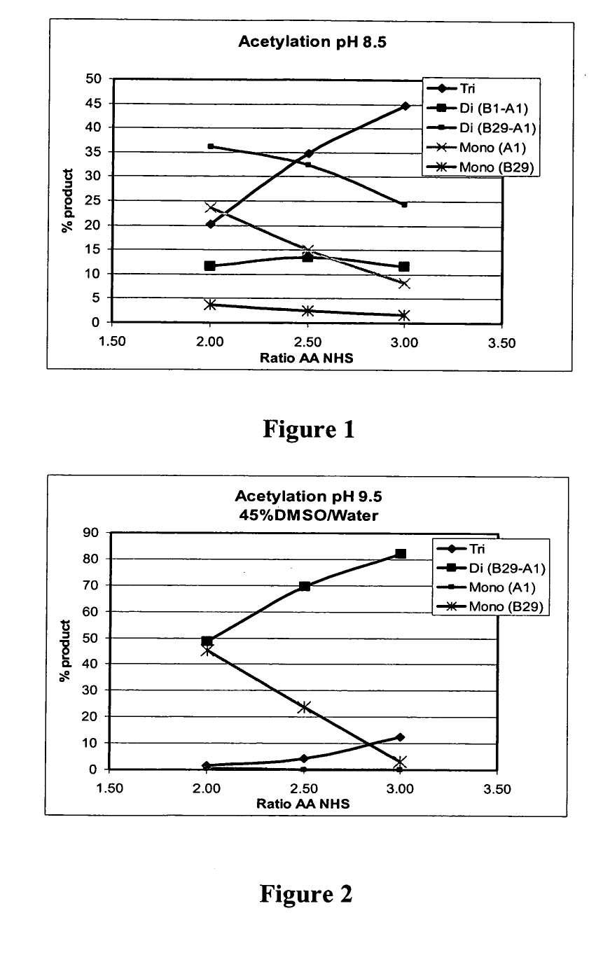

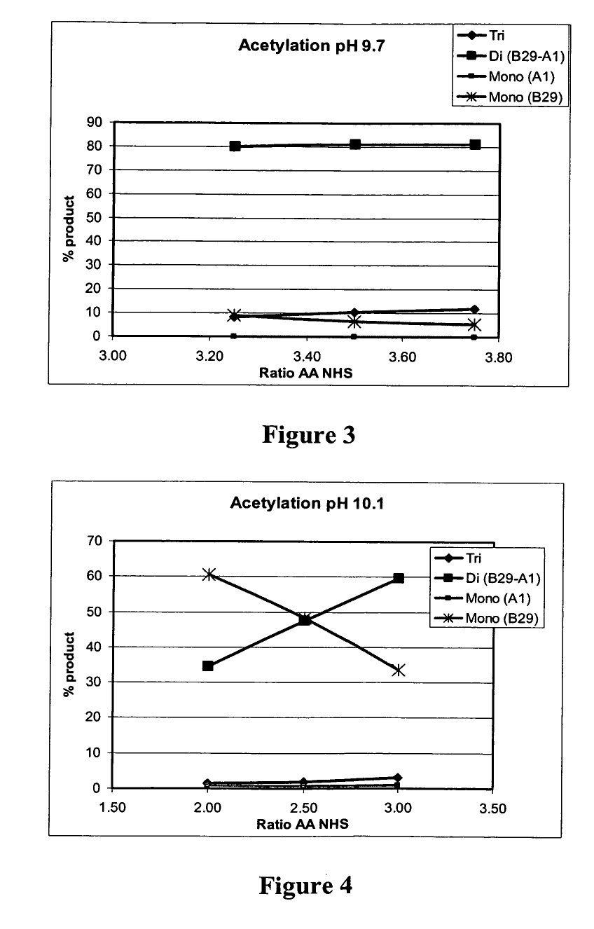

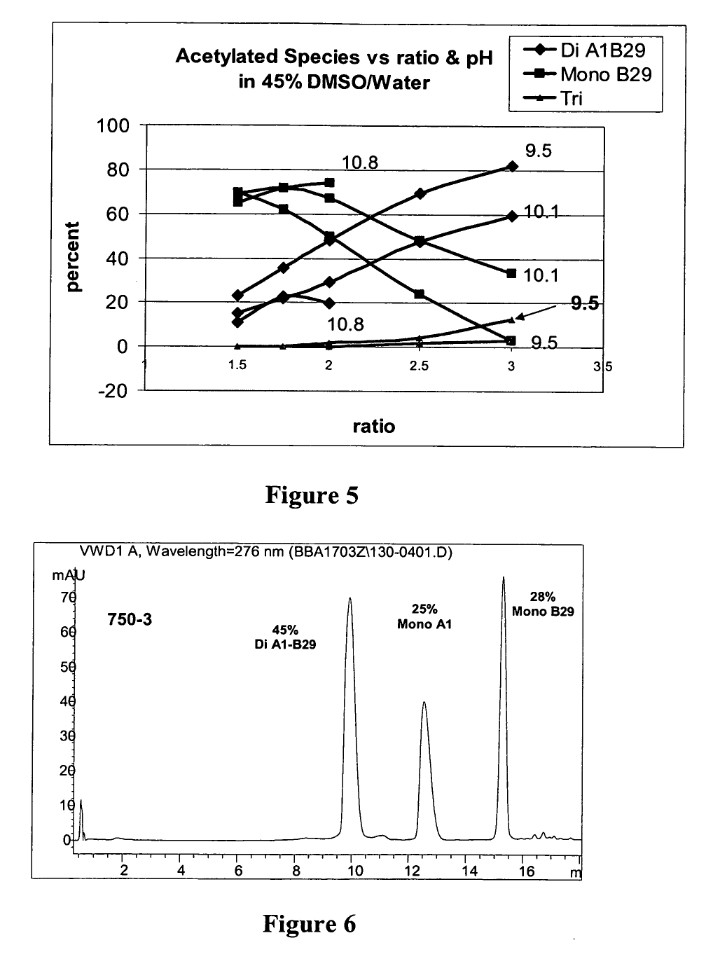

Insulin was dissolved at 40 mg/ml in 50 mM boric acid, and 2 M NaOH was added to adjust the pH to 8.5, 9.5, 9.7, 10.1, or 10.8. DMSO was added to the insulin solutions to yield a ratio of DMSO/water of (45% (v/v)). The added DMSO included acetic acid NHS (23 mg/ml) at a ratio of AANHS:insulin of 2.0, 2.5, or 3.0. The solutions were allowed to react for 30 minutes at room temperature. The products were diluted 10 fold with water and examined by ion exchange chromatography. The results are shown in FIGS. 1-5.

### Example 14

### Synthesis of di-N^{αA1},N^{εB29}-(acetyl)B1-{3-[methoxy(ethoxy)₇₅₀]propyl}insulin (750-lot A1-PEG-Insulin)

Insulin (201.1 mg, 34.6 μmol) was added to 50 mM boric acid at 40 mg/mL, and the solution was stirred for 10 to 20 minutes until the insulin was dissolved. The pH of the insulin solution was adjusted to 9.5 by adding 2 M NaOH. A solution of acetic acid-NHS in dry DMSO (43.9 μmol, 1.0 mL) was prepared. The solution (1.27 fold molar excess of acetic acid-NHS to insulin) was added dropwise to the insulin solution, and the mixture was stirred at room temperature for 40 minutes to give a solution of di(acetyl)insulin.

2,5-Dioxopyrrolidin-1-yl 3-[2-methoxy(ethoxy)₇₅₀]propionate (70.4 mg, 67.7 μmol) was dissolved in dry DMSO (1 ml) and the PEG solution was added drop-wise to the di(acetyl)insulin solution. The mixture was stirred overnight at room temperature under argon.

The reaction was then precipitated by a 100 mL mixture of acetone/ethyl ether (1:2). The product was collected and dried under vacuum. The synthesis yielded 188 mg powder.

### Example 15

### Synthesis of di-N^{αA1},N^{εB29}-(acetyl)B1-{3-[methoxy(ethoxy)₇₅₀]propyl}insulin (750-lot A2-PEG-Insulin)

Insulin (201 mg, 34.6 μmol) was added to 50 mM boric acid at 40 mg/mL, and the solution was stirred for 10 to 20 minutes until the insulin was dissolved. The pH of the insulin solution was adjusted to 9.5 by adding 2 M NaOH. A solution of acetic acid-NHS in dry DMSO (52.2 μmol, 1.0 mL) was prepared. The solution (1.51 fold molar excess of acetic acid-NHS to insulin) was added dropwise to the insulin solution, and the mixture was stirred at room temperature for about 30 minutes to give a solution of di(acetyl)insulin.

2,5-Dioxopyrrolidin-1-yl 3-[2-methoxy(ethoxy)₇₅₀]propionate (59.5 mg, 57.2 μmol) was dissolved in dry DMSO (1 ml) and the PEG solution was added drop-wise to the di(acetyl)insulin solution. The mixture was stirred overnight at room temperature under argon.

The reaction was then precipitated by a 100 mL mixture of acetone/ethyl ether (1:2). The product was collected and dried under vacuum. The synthesis yielded 146 mg powder. The final product was characterized by reverse phase HPLC. It consisted of a mixture of PEGylated insulin conjugates.

In addition to the above procedure for “750-lot A2-PEG-insulin,” the above procedure was repeated with 1.87 and 2.1 molar excesses of acetic acid-NHS to insulin. The amount of tri-acetylated and mono-PEGylated products generally increased with the ratio of acetic acid-NHS to insulin.

### Example 16

### Synthesis of di-N^{αA1},N^{εB29}-(acetyl)-B1-{3-[methoxy(ethoxy)_{2K}]propyl}insulin (2K (B1 PEG Di Ac Insulin)

Insulin (423 mg, 72.8 μmol) was dissolved in dry DMSO (24 mL) and triethylamine (1.2 mL) with stirring for 10 to 20 minutes. A solution of 2,5-dioxopyrrolidin-1-yl acetate in dry DMSO (220 μmol, 1.5 mL) was added to the insulin solution and the mixture was stirred at room temperature for 15 minutes to give a solution of di-N^{αA1},N^{εB29}-(acetyl)insulin

2,5-Dioxopyrrolidin-1-yl 3-[2-methoxy(ethoxy)_{2K}]propionate (798 mg, 352 μmol) was dissolved in dry DMSO (6 ml) and triethylamine (36 μL) and the aldehyde solution was added drop-wise to the di(acetyl)insulin solution. The mixture was stirred overnight at room temperature. Analysis of the reaction mixture on an Agilent 1100 HPLC system indicated a mixture of about 55% di(acetyl)-{3-[methoxy(ethoxy)_{2K}]propionyl}insulin, 27% acetyl-di{3-[methoxy(ethoxy)_{2K}]propionyl}insulin and 18% tri(acetyl)insulin.

The mixture was diluted with deionized water (120 mL) and the pH was adjusted to 3.5 with 2M sodium hydroxide. A pool of the mixture (110 mL) was diluted further to 500 mL with deionized water. Product was purified from the further diluted mixture by cation exchange chromatography as runs 2 and 3. Run 4 was dialyzed flow through from run 1.

A 75 mL column of SP650S toyopearl resin was used with buffer A containing 0.05M sodium acetate at pH 3.5 and buffer B containing 0.05M sodium acetate and 1M sodium chloride at pH 3.5. An AKTA Explorer HPLC system was employed. A gradient of 0-8% buffer B over 15 column volumes for all purification runs was used and 12 mL fractions were collected. The result was a di-N^{αA1},N^{εB29}-(acetyl)-N^{αB1}-{3-[methoxy(ethoxy)_{2K}]propionyl}insulin peak at about 6% buffer B (0.6M sodium chloride).

The fractions from each run that contained product were combined, buffer exchanged into deionized water with the PALL Omega 5K Ultrasette and a peristaltic pump with 10-20 psi pressure on the inlet line. The buffer exchanged product (about 140 mL) was 0.2 μm filtered. The insulin solution was frozen and then lyophilized to dryness to give di-N^{αA1},N^{εB29}-(acetyl)-N^{αB1}-{3-[methoxy(ethoxy)_{2K}]propionyl}insulin (58 mg, 7 μmol) as a dry powder. The molecular weight as determined by matrix assisted laser desorption ionization (MALDI) was 8135.

### Example 17

### Synthesis of di-N^{αA1},N^{εB29}-(acetyl)-B1-{3-[methoxy(ethoxy)_{3K}]propyl}insulin (3K B1 PEG Di Ac Insulin)

Proceeding as in Example 16, but substituting insulin (202.2 mg, 34.8 μmol) and 2,5-dioxopyrrolidin-1-yl 3-[methoxy(ethoxy)_{3K}]propionate (512 mg, 170 μmol), gave di-N^{αA1},N^{εB29}—N^{αB1}-{3-[methoxy(ethoxy)_{3K}]propionyl}insulin (20.6 mg, 2.34 μmol).

### Example 18

### Synthesis of di-N^{αA1},N^{εB29}-(acetyl)-B1-{3-[methoxy(ethoxy)_{5K}]propyl}insulin (5K B1 PEG Di Ac Insulin)

Proceeding as in Example 16, but substituting insulin (506.5 mg, 72.8 μmol) and 2,5-dioxopyrrolidin-1-yl 3-[methoxy(ethoxy)_{5K}]propionate (2.05 g, 410 μmol), gave di-N^{αA1},N^{εB29}-(acetyl)-N^{αB1}-{3-[methoxy(ethoxy)_{5K}]propionyl}insulin (79 mg, 6.9 μmol). Molecular weight as determined by MALDI was 11478.

### Example 19

### Synthesis of di-N^{αA1},N^{εB29}-(acetyl)-B1-{4-[methoxy(ethoxy)_{2K}]butyryl}insulin (2K-B1 butyraldehyde PEG di Ac Insulin)

Insulin, stored at −20° C., was warmed to ambient temperature. The warmed insulin (200 mg) was dissolved in 80 mL of 20 mM sodium acetate buffer at pH 4.0. The pH of the solution was lowered to 3.2 with 1.0 N HCl and readjusted to 4.0 with 1.0 N NaOH to completely dissolve the insulin.

mPEG-butyraldehyde 2K, stored at −20° C. under argon, was warmed to ambient temperature. The warmed mPEG-butyraldehyde (161 mg), dry powder, was added to the insulin solution. The solution was allowed to mix for 15 minutes at room temperature. Sodium cyanoborohydride (106 mg) was then added to the PEG-insulin mixture to form a 20 mM final concentration and was allowed to mix at room temperature over night (˜18 hours). RP-HPLC (C₁₈) analysis was run on the sample, which confirmed the presence of monoconjugated material (“1-mer”). The resulting chromatogram indicated a yield of 58-59% monoPEGylated or “1-mer” species.

The solution was then concentrated to ˜10 mL by ultrafiltration (Millipore YM3 membrane NMWL 3000 at 50 psi), and diluted to 50 mL with 20 mM sodium acetate buffer at pH 4.0 and was again concentrated to 10 mL. This dilution and concentration process was repeated three times. Each filtrate was analyzed by RP-HPLC to confirm no loss of insulin or PEG-insulin conjugate.

The concentrated solution was pHed to 8.5 with 0.5 N NaOH, followed by the addition of 60 mg of acetic acid NHS ester (pre-dissolved in 1 mL of acetonitrile). The reaction was allowed to stir at room temperature for 30 minutes. The pH was monitored and adjusted as needed to maintain a pH of 8.5-9.0. The ester reaction was then quenched with 400 μL of 1 M glycine and the pH was adjusted to 7.0 with 2 N HAc. The solution was allowed to stir at 4° C. for 2 hours. RP-HPLC (C₁₈) was run on the sample, which confirmed the presence of acetylated-PEG-Insulin.

The reaction mixture was diluted in 50 mM acetic acid pH 2.9 and was purified using a 75 mL SP650 (Tosoh) IEX column with 50 mM acetic acid pH 2.9 as purification buffer A and 50 mM acetic acid pH 2.9+1 M NaCl as purification buffer B.

The purified peak was concentrated using a Milli-pore 3K YM membrane and Amicon StirCell. Once concentrated, the sample was buffer exchanged into Milli-A water using a pre-packed DeSalt column from GE (Amersham). The protein peak was collected and lyophilized over night to form the final product.

A Glu-C peptide digest was run on the sample to confirm PEGylation of site B-1 and diacetylation of the other two active sites (A29, B29).

### Example 20

### Synthesis of G2-PEG2-FMOC-carbamate 20K or 40K insulin—random

Insulin (25.5 mg, 4.4 mop was added to dry DMSO (2.5 mL) and the solution was stirred for 30 to 60 minutes until the insulin was dissolved. G2-PEG2-FMOC-NHS 20K (171.9 mg, 8.6 mop was added to dry DMSO (2.5 mL) and was heated to 40° C. until dissolved. The PEG reagent was cooled to room temperature. The PEG solution was added to insulin (1:2 molar ratio of insulin:PEG) via a syringe injection. The stirred reaction continued at room temperature for 1 hour. It was then diluted 1:3 with 20 mM acetic acid, pH 3.1. This reaction scheme is believed to target the A1 and B29 sites on insulin because those 2 sites have much greater reactivity than the B1 site.

A 20 mL column of SP650S toyopearl resin was used to purify the mono-conjugate. Buffer A contained 0.02 M acetic acid at pH 3.1, and buffer B was comprised of 0.02 M acetic acid and 1 M sodium chloride at pH 3.1. An AKTA basic system was employed. The diluted reaction mixture was loaded onto the column. The purification method used a gradient of 0-15% buffer B over 11 column volumes, and 13 mL fractions were collected.

The fractions were analyzed by gel electrophoresis. The fractions that contained mono-substituted product were combined into a pool. The pool was concentrated, and buffer exchanged into 20 mM acetic acid, pH 3.1 using a Tangential Flow Filtration (TFF) apparatus. The TFF membrane was a PALL Omega 5K Ultrasette. The diafiltration buffer, 20 mM acetic acid, pH 3.1, was used to stabilize the releasable conjugate. The peristaltic pump was set at a flow rate of 1 L/min, and backpressure was applied on the retentate line to generate an inlet pressure of 10-20 psi on the inlet line. The pressures on the retentate and filtrate lines were not monitored. The buffer exchanged product (about 140 mL) was 0.22 μm filtered using a 250 mL disposable Nalgene filter unit. The solution was aliquoted into three 250 mL conical tubes. The solution was frozen overnight at −80° C. (with the bottles lying horizontally) then lyophilized to dryness to give G2-PEG2-FMOC-carbamate 20K insulin (104.3 mg).

The above was repeated, but substituting 25.1 mg insulin and 344.5 mg G2-PEG2-FMOC-NHS 40K, yielded G2-PEG2-FMOC-carbamate 40K insulin (44.1 mg).

### Example 21

### Synthesis of G2-PEG2-FMOC-carbamate 20K or 40K insulin—site specific (B1)

Insulin (101.0 mg, 17.4 μmol) was added to 100 mM sodium acetate, pH 4.0 (40 mL) and the solution was stirred for 2 hours until the insulin was dissolved. Solution initially appeared milky. After 2 hours, the solution was clear. G2-PEG2-FMOC-NHS 40K (2.1 g, 105.0 μmol) was added to 100 mM sodium acetate, pH 4.0 (26 mL). The PEG solution was vortexed. The PEG solution was added to insulin (1:3 molar ratio of insulin:PEG). The stirred reaction continued at room temperature for 1 hour. It was then diluted 1:3 with 20 mM acetic acid, pH 3.1. This reaction scheme primarily targets the B1 site because of the majority of A1 and B29 sites are protonated at pH 4 versus B1 whose amine group is unprotonated at low pH. There are other sites on insulin such as histidine side chains that could be modified at this pH as well.

A 77 mL column of SP650S toyopearl resin was used to purify the mono-conjugate. Buffer A contained 0.02 M acetic acid at pH 3.1, and buffer B was comprised of 0.02 M acetic acid and 1 M sodium chloride at pH 3.1. An AKTA basic system was employed. The diluted reaction mixture was loaded onto the column. The purification method used a gradient of 0-16% buffer B over 10 column volumes, and 12 mL fractions were collected.

The fractions were analyzed by gel electrophoresis. The fractions that contained mono-substituted product were combined into a pool. The pool was concentrated, and buffer exchanged into 20 mM acetic acid, pH 3.1 using a Tangential Flow Filtration (TFF) apparatus. The TFF membrane was a PALL Omega 5K Ultrasette. The diafiltration buffer, 20 mM acetic acid, pH 3.1, was used to stabilize the releasable conjugate. The peristaltic pump was set at a flow rate of 1 L/min, and backpressure was applied on the retentate line to generate an inlet pressure of 10-20 psi on the inlet line. The pressures on the retentate and filtrate lines were not monitored. The buffer exchanged product (approximately 150 mL) was 0.22 μm filtered using a 250 mL disposable Nalgene filter unit. The solution was aliquoted into three 250 mL conical tubes. The solution was frozen overnight at −80° C. (with the bottles lying horizontally) then lyophilized to dryness to yield G2-PEG2-FMOC-NHS 40K insulin.

A quick release test on the lyophilized product was performed to determine insulin content and to confirm complete release of insulin from the PEG. G2-PEG2-FMOC-NHS 40K insulin (1.32 mg) was added to 100 mM HEPES, pH 7.5 (1.0 mL). The pH was adjusted to 8.66 with 11.0 μL 2 M NaOH. Incubated solution at 50° C. for approximately 24 hours. Insulin content was determined to be 9.8%. This is slightly lower than the theoretical content of 22.5%. Complete release of insulin from the PEG was also confirmed by gel electrophoresis. Peptide mapping was performed to determine which residue on insulin was modified. The results show that the B1 position was modified, but it was not the only isomer formed under these PEGylation conditions. The purity of the conjugate was 91% mono-conjugate as determined by reverse phase HPLC.

The above was repeated, but substituting 80.0 mg of insulin and 1.1 g of G2-PEG2-FMOC-NHS 20K, yielded G2-PEG2-FMOC-carbamate 20K insulin (68.1 mg).

### Summary of Examples 1-12 and 14-21

The PEGylated insulin analogs synthesized in Examples 1-12 and 14-21 are summarized in Table 1, below.

TABLE 1
PEGylated insulin analog preparations and their characteristics

| | PEG | | | | |
|---|---|---|---|---|---|
| | MW | PEG | PEGylation | Major | |
| Example | (Da) | Target Site | Method | Composition | Batch ID |
| 1 | 550 | A1 & B29 | Random | Mono-A1, | 550-lot |
| | | | PEGylation | Mono-B29, | 1-PEG- |
| | | | | Di-A1/B29 | Insulin |
| 2 | 750 | A1 & B29 | Random | Tri-, Mono-B1, | 750-lot |
| | | | PEGylation | Di-A1/B1 | 1-PEG- |
| | | | | | Insulin |
| 2 | 750 | B1 | Site Specific | Mono-B1, | 750-lot |
| | | | | Mono-B29, | 2-PEG- |
| | | | | Di- | Insulin |
| 3 | 750 | A1 & B29 | Random | Mono-A1, | 750-lot |
| | | | PEGylation | Mono-B29, | 3-PEG- |
| | | | | Di-A1/B29 | Insulin |
| 4 | 750 | A1 & B29 | Random | Di-A1/B29 | 750-lot |
| | | | PEGylation | | 4-PEG- |
| | | | | | Insulin |
| 5 | 750 | B29 | Site Specific | Mono-B29 | 750-lot |
| | | | | | 5-PEG- |
| | | | | | Insulin |
| 6 | 2000 | B29 | Site Specific | Mono-B29 | 2K-lot |
| | | | | | 2 PEG- |
| | | | | | Insulin |
| 7 | 2000 | A1 & B29 | Random | Mono-A1, | 2000- |
| | | | PEGylation | Mono-B29, | PEG- |
| | | | | Di-A1/B29 | Insulin |
| 8 | 5000 | B29 | Site Specific | Mono-B29 | 5K PEG |
| | | | | | Insulin |
| 9 | 5000 | B1 | Site Specific | Mono-B1 | B1-5000- |
| | | | | | PEG |
| | | | | | insulin |
| 10 | 2000 | B1 | Site Specific | Mono-B1 | PEG2000 |
| | | | | | B1 |
| 11 | 3000 | B1 | Site Specific | Mono-B1 | B1-3000- |
| | | | | | PEG |
| | | | | | insulin |
| 12 | 2000 | B1 | Site Specific | Mono-B1 | PEG2K- |
| | | | | | Ald- |
| | | | | | Insulin |
| 14 | 750 | B1 | Random | Di-B1/B29, | 750-lot |
| | | | Acetylation/ | Mono-B1, | A1-PEG- |
| | | | PEGylation | Tri- | Insulin |
| 15 | 750 | B1 | Random | Mono-B1, | 750-lot |
| | | | Acetylation/ | Di-B1/B29, | A2-PEG- |
| | | | PEGylation | Tri- | Insulin |
| 16 | 2000 | B1 | Acetylation/ | Mono-B1, | 2K B1 |
| | | | PEGylation | A1/B29 Di-Ac | PEG Di |
| | | | | | Ac Insulin |
| 17 | 3000 | B1 | Acetylation/ | Mono-B1, | 3K B1 |
| | | | PEGylation | A1/B29 Di-Ac | PEG Di |
| | | | | | Ac Insulin |
| 18 | 5000 | B1 | Acetylation/ | Mono-B1, | 5K B1 |
| | | | PEGylation | A1/B29 Di-Ac | PEG Di |
| | | | | | Ac Insulin |
| 19 | 2000 | B1 | Acetylation/ | Mono-B1, | PEG2K- |
| | | | PEGylation | A1/B29 Di-Ac | Ald-DiAc |
| | | | | | Insulin |
| 20 | 20,000 | A1 & B29 | Random | Mono-B29, | G2-PEG2- |
| | | | Releasable | Mono-A1 | FMOC- |
| | | | PEGylation | | carbamate |
| | | | | | 20K insulin |
| 20 | 40,000 | A1 & B29 | Random | Mono-B29, | G2-PEG2- |
| | | | Releasable | Mono-A1 | FMOC- |
| | | | PEGylation | | carbamate |
| | | | | | 40K insulin |
| 21 | 20,000 | B1 | Site Specific | Mono-B1 | G2-PEG2- |
| | | | | | FMOC- |
| | | | | | carbamate |
| | | | | | 20K insulin |
| 21 | 40,000 | B1 | Site Specific | Mono-B1 | G2-PEG2- |
| | | | | | FMOC- |
| | | | | | carbamate |
| | | | | | 40K insulin |

### PEG-Insulin Analog Characterization

The composition of PEGylated insulin analogs was determined by high resolution cation exchange HPLC. Table 2 summarizes the purity and composition of the PEG-insulin analogs prepared in these studies. Assignments of size were confirmed by MALDI. Assignments of the positional isomers were based on expected outcomes of the literature procedures and were not verified by peptide mapping.

### Specific PEGylation

As shown in Tables 1 and 2, site-specific PEGylation achieved high level of specificity and yielded high purity preparations for B1-5000-PEG-insulin (80%), the B29 modified 2K-lot 2-PEG-insulin (98%), and the 750-5-PEG-insulin (99%) analogs. In contrast, the 750-lot 1-PEG-insulin and 750-lot 2-PEG-insulin analog preparations contained a significant portion of di-PEGylated species (60 and 37%, respectively) and only a small proportion of the targeted (B1) site mono-derivative (27 and 50%, respectively).

### Random PEGylation

As anticipated, random PEGylation resulted in a wide distribution of species, typically at equal percentage between mono-, di- and tri-conjugated analogs (750-lot 3-PEG-insulin, 2000-PEG-insulin and 550-lot 1-PEG-insulin). Even after an RP-HPLC purification step, there was still a significant proportion of A1-B29-PEG-Insulin remaining in these preparations (45-49%). Further, HPLC analysis indicated that the mono-conjugated species were composed of equal amounts of derivatives at the two possible positions (A1 and B29). As an example, the chromatogram of FIG. 6 illustrates the composition of analog 750-3-PEG-insulin, which indicates an almost an equal proportion of mono-conjugates in positions A1 (mono-A1-PEG-Insulin) (25%) and B29 (mono-B29-PEG-Insulin) (28%).

### Non-Reversible Acetylation and PEGylation

The analysis shown in Table 2 (Batch ID numbers 750-lot A1-PEG, 750-lot A2-PEG, 750-lot A3-PEG, and 750-lot A4-PEG) indicates that this approach yielded a broad mixture of mono-, di-, and tri-PEGylated analogs with significant amounts of residual triacetylated insulin. Mono-conjugate refers to a single PEG attached to insulin; Di-conjugate refers to two PEGs attached per insulin molecule and tri-conjugate refers to three PEGs attached per insulin molecule. Assignments of the positional isomers were based on expected outcomes of the literature procedures.

TABLE 2

| | | Mono- | Mono- | Mono- | Di- | Di- | | | |
|---|---|---|---|---|---|---|---|---|---|
| | PEG- | Conjugated | Conjugated | Conjugated | Conjugated | Conjugated | Tri- | Tri- | Residual |
| | Insulin | A1 | B1 | B29 | A1-B29 | A1-B1 | Conjugated | Acetylated | Insulin |
| Ex. | Analog | (%) | (%) | (%) | (%) | (%) | (%) | (%) | (%) |
| 8 | B1-5000- | — | 80 | — | — | 10 | 8 | — | 2 |
| | PEG | | | | | | | | |
| 7 | 2000- | 10 | — | 33 | 44 | — | 12 | — | 0.5 |
| | PEG | | | | | | | | |
| 6 | 2K-2 | — | — | 98 | 1 | — | 1 | — | — |
| | PEG | | | | | | | | |
| 2 | 750-1- | 10 | 27 | 3 | 5 | 20 | 35 | — | 0.5 |
| | PEG | | | | | | | | |
| 2 | 750-2 | 10 | 50 | 3 | 10 | 15 | 12 | — | 0.5 |
| | PEG | | | | | | | | |
| 14 | 750-A1 | 31¹ | | | 42 | | 24 | 3 | N/A |
| | PEG² | | | | | | | | |
| 15 | 750-A2 | 44¹ | | | 32 | | 13 | 10 | N/A |
| | PEG² | | | | | | | | |
| 5 | 750-5 | — | — | 99 | — | — | 1 | — | — |
| | PEG | | | | | | | | |
| 3 | 750-3 | 25 | — | 27 | 44 | — | 12 | — | 0.5 |
| | PEG | | | | | | | | |
| 4 | 750-4 | — | — | — | 95 | — | 5 | — | — |
| | PEG | | | | | | | | |
| 1 | 550-1 | 42 | — | 12 | 42 | — | 4 | — | — |
| | PEG | | | | | | | | |

¹All mono-conjugated species are present - B1 is the dominant.
²Acetylated then PEGylated analogs.

### Example 22

### Formulation and Spray Drying

Aerosol dry powders, as used in Examples 23-27, were prepared by spray drying insulin or PEGylated insulin from aqueous solutions containing trileucine, leucine, and sodium citrate as excipients using a Büchi 190 spray dryer.

More specifically, modified insulin solutions were formed. For example, 2K-PEG-DiAc insulin from Example 16 was dissolved in water along with leucine, sodium citrate, and trileucine, as shown below. The pH was adjusted to 6.7 by adding 1 M NaOH.

| | | | Concentration | |
|---|---|---|---|---|
| Component | Source | Solids (%) | (mg/ml) | Quantity |
| Water | OmniSolv | NA | NA | 200 |
| Leucine | Sigma | 47.00 | 4.70 | 940.0 |
| Sodium citrate | Baker | 8.00 | 0.80 | 182.2 |
| Trileucine | Bachem | 18.0 | 1.80 | 360.0 |
| PEG 2K DiAc | Example 16 | 27.0 | 2.70 | 545.5 |

The modified insulin solutions were spray dried using a laboratory scale Büchi 190 spray dryer (Büchi Labortechnik, Ag., Meierseggstrasse, Switzerland) fitted with a modified cyclone, an atomizer nozzle, and a powder collection vessel. The atomizer of the spray dryer was operated with clean dry air. The liquid flow rate into the spray dryer was 5 ml/min. The inlet temperature was adjusted to achieve the target particle size and morphology (80° C. to 150° C., e.g., 95° C.). The outlet temperature ranged from about 30° C. to 100° C., e.g., 58° C. The atomizer air pressure was set at 48 psi. The yield was 65.5%.

The powders were transferred into a glovebox with a relative humidity less than 5% and placed into unit dosage forms (blister packs, BP's) suitable for use in a dry powder inhaler device as described in U.S. Pat. No. 5,740,794. In other words, dry powders were packaged as premetered doses in individually manufactured blisters to fit Nektar's pulmonary delivery system (PDS), which is commercially available as the Exubera inhaled insulin inhaler.

### Example 23

### Determination of the Effect of PEGylation on Insulin Activity

The impact of PEGylation on insulin activity was determined by administering the PEGylated compounds (synthesized in Example 1) via intravenous injection. Serum insulin concentrations were then analyzed using radioimmunoassay, and results for the PEGylated compounds with varying MW are compared.

### Preparation of Stock Solutions

Appropriate amounts of insulin or PEGylated insulin powder were dissolved in phosphate buffered saline to a final concentration of 1.0 mg/mL (adjusted appropriately to reflect impurities and mass of PEG). The API concentration in the stock solutions was determined post-dosing by UV/VIS spectroscopy. All stock solutions were prepared on the study initiation day.

### Preparation of Dosing Solutions

80 μg/animal of insulin equivalent mass (Insulin, 750-lot 5 PEGylated and 2K-lot 2 PEGylated Insulin dosing solutions) were prepared within 2 hours of dosing by adding 533 μL, of the 1.0 mg/mL stock insulin solution to 1.47 mL of phosphate buffered saline.

### Sample Collection

The heparinized PBS soaked cotton applicator was removed from the hub of the cannula, and a 1 mL syringe was fitted into the cannula hub. The heparinized solution present in the catheter lumen was removed by drawing back on the syringe plunger until blood filled the hub of the syringe. The heparin solution and syringe was discarded and replaced with a new 1 mL syringe for blood sample collection.

Whole blood (0.5 mL) was collected from the jugular vein catheter and placed into serum separator tubes at pre-dose (2 to 0.25 hours prior to dosing) and post-dose time points. After the sample was collected, an equivalent volume of PBS was administered, the catheter lumen was filled with 100 μL of heparinized PBS, and the cannula hub was plugged with a cotton applicator tip soaked in heparinized saline.

A small amount of whole blood was placed on the glucose test strip for blood glucose determination. The remainder of the sample was separated into serum by centrifugation for 10 minutes at 13,000 rpm. The serum was drawn off into a new Eppendorf tubes. The serum samples were stored at −20° C. on dry ice until shipped, and then stored at −80° C. until analysis.

### Sample Analysis

The whole blood samples were analyzed for glucose concentrations at the time of the study and serum samples were analyzed for insulin concentration when applicable.

Effect of PEGylated Insulin Compounds on Insulin Activity following Administration Via Intravenous Injection to Sprague-Dawley Rats

The test and control insulin-containing articles were dissolved in saline (pH 7.4) and injected as an IV bolus into Sprague-Dawley rats. Prior to administration, the rats were lightly anesthetized for approximately 3-5 minutes in a Plexiglas induction chamber by inhaled 3.0-5.0% isoflurane mixed with air. The femoral vein catheter (FVC) was used for IV drug administration and the jugular vein catheter (JVC) was used for venous blood sampling to ensure no cross-contamination occurred.

A 1 mL syringe was fitted into the cannula, and the heparinized phosphate buffered saline solution present in the catheter lumen was removed by drawing back on the syringe plunger until blood filled the hub of the syringe. The heparinized phosphate buffered saline solution and the syringe were discarded and replaced by a new 1 mL syringe containing the appropriate IV dosing volume. The IV dose was then slowly and completely administered to the animal. The cannula was immediately flushed with 300 μL of phosphate buffered saline after dose administration. Blood was withdrawn from an indwelling catheter over a three-hour period. All drug doses refer to equivalent insulin mass delivered to the animals.

### Determination of Serum Insulin Concentrations Using Radioimmunoassays

Serum insulin concentrations were determined by a radioimmunoassay (RIA) performed either by a CRO (Linco Research) or by an in-house method. The in-house method was performed as follows: standard curves were generated for recombinant human (rh) insulin (Akzo Nobel) and the relevant PEG-insulin. Rh-insulin or PEG-insulin standards were prepared in the concentration range 2 μU/mL-1000 μU/mL by serial dilution of the stock (100 U/mL) in assay buffer (0.5 M Na₂HPO₄, 0.02 M Na₂EDTA.2H₂O, 0.2% BSA, 0.01% sodium azide).

Standards or serum samples (100 μL) were incubated with 200 μL [¹²⁵I]insulin (>0.0625 μCi/mL) [¹⁴A]-monoiodinated [¹²⁵I]-insulin, Linco Research) and 100 μL primary antibody (guinea-pig anti-porcine insulin, Linco Research) in 12×75 mm Kimble tubes, overnight at 4° C. Samples were then incubated in secondary antibody (100 μL goat anti-guinea pig IgG, Linco Research) with carrier (100 μL normal goat serum, Linco Research) for 30 min at room temperature, and precipitated by addition of 1 mL wash buffer (0.5 M Na₂HPO₄, 0.02 M Na₂EDTA.2H₂O, 0.2% BSA, 0.01% sodium azide) and 30 min centrifugation at 4° C.

Precipitates were dried by brief inversion of the tubes and radioactivity was counted in a gamma-scintillation counter (Cobra, Packard). [¹²⁵I]insulin and primary antibody were hydrated in assay buffer; secondary antibody and carrier were hydrated in assay buffer containing 3% PEG (Sigma), according to the manufacturer's instructions. Data were analyzed using GraphPad Prism 4.0.

### RIA Insulin Assay

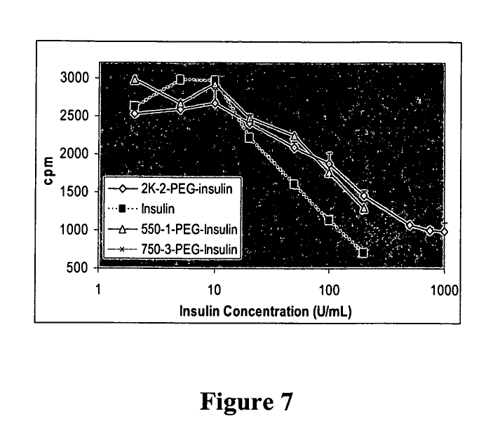

The insulin radioimmunoassay (RIA) is based on an antigen-antibody interaction between radiolabeled insulin (the antigen) and an anti-insulin antibody. Addition of PEG to insulin can alter the antibody binding site, and thus change the quantitative relationship between concentration and binding. Consequently, extrapolation of PEG insulin concentrations from an insulin standard curve can generate inaccurate results. The degree of deviation from accuracy depends on the PEG insulin species and on the concentration in question, as shown in FIG. 7.

### Effect of PEGylation on Insulin Activity

In order to evaluate the impact of PEG molecular weight, conjugation site and PEGylation strategy on insulin bioactivity, PEG derivatives were prepared and their activity was monitored by evaluating plasma serum concentration and blood glucose following intravenous administration to rats. This study was designed to obtain a broad sense of the activity of the compounds rather than serve as an accurate activity measurement. It is important to note that RIA assay does not necessarily determine “activity,” and as such, the results generated from RIA measurements should be carefully interpreted. Thus, in the discussion below, only glucose depression data is used to discuss analog “activity,” except in cases where the correct plasma assays were used, for which we will also report plasma insulin data.

### Effect of PEG Size

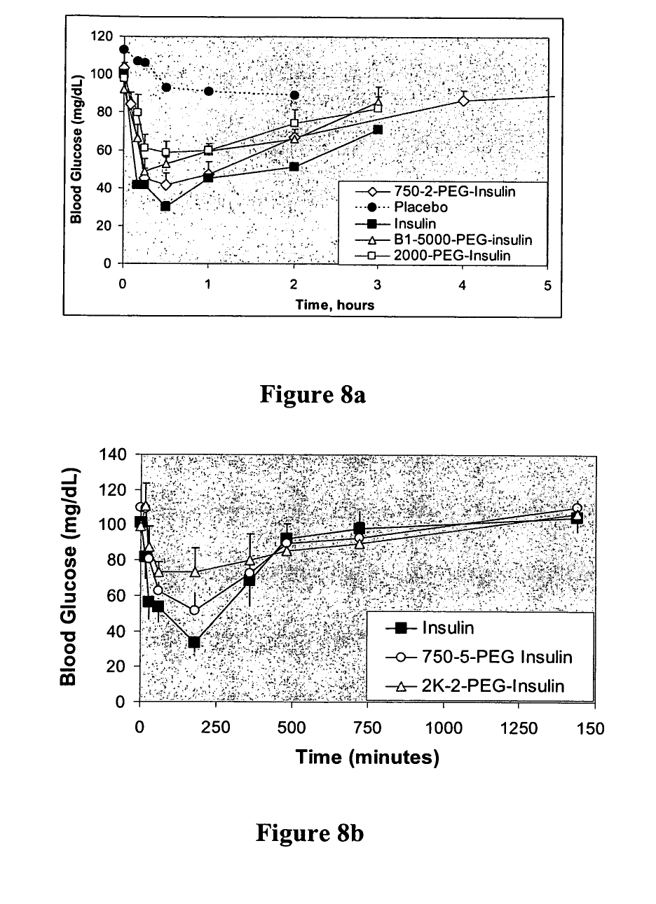

The in vivo activity (as measured by glucose depression) comparison of insulin analogs PEGylated in position B1 is given in FIGS. 8a and 8b.

Both B1-PEGylated analogs (B1-5000-PEG- and 750-lot 2-PEG-insulin) produced a smaller glucose depression in comparison to regular insulin, thus indicating a lower bioactivity. The activity reduction appears to be the least for the lower molecular weight PEG (750-lot 2-PEG-insulin), as indicated by the similar levels of glucose depression with regular insulin. The deviations were significant for B1-5000-PEG-insulin and 2000-PEG-insulin analogs, indicating an inverse proportional relationship between PEG MW and glucose suppressing activity. It should be kept in mind that the 2000-PEG-insulin is a randomly PEGylated species.

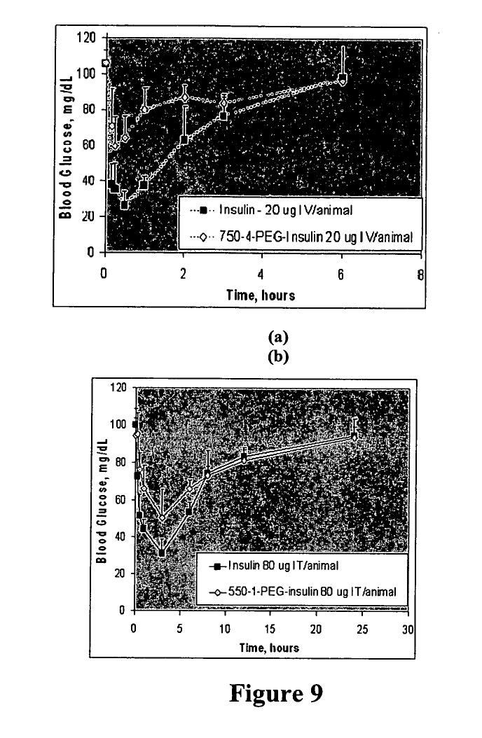

A more direct comparison (due to the purity and specificity of the analogs) is given by B29-conjugated analogs. The results obtained were similar to the B1 analogs. As shown in FIG. 9b, although both the 750 and 2000 PEG derivatives had reduced activity in vivo, the losses were more pronounced with the higher molecular weight PEG. However, it should be noted that the glucose response obtained via intratracheal instillation studies is a convolution of both insulin activity and permeation through the lungs.

### Effect of PEGylation Site

The above studies suggest that, regardless of the site of attachment (B1 or B29), there is significant reduction of insulin activity, which is inversely proportional to the PEG MW.

Di-PEGylation of insulin in positions A1-B29 seems to significantly reduce (by about 50%) insulin activity, as shown in FIG. 9a with the 750-lot 4-PEG-insulin, which is predominantly (95%) a di-(A1, B29)-conjugate. When a 550-lot 1-PEG is used (FIG. 9b), much of the glucose suppression activity of insulin is restored. This may be largely due to the presence of the mono-conjugated species in positions B1 (12%) and A1 (42%).

These results further explain the glucose suppression curve for the 2000-PEG-insulin analog shown in FIG. 8a. The reduced activity of this analog is due to the presence of significant amount (44%) of the relatively inactive di-(A1, B29)-conjugate.

### Effect of Insulin PEGylation on Pulmonary Absorption

In order to evaluate the impact of PEGylation on insulin pulmonary absorption, the appropriate analogs were prepared and their absorption following intratracheal administration to rats was monitored. In the sections below, an effort is made to compare PEG-insulin preparations with respect to PEG molecular weight, PEGylation site, and chemistry. However, an accurate comparison was not always possible due to the presence of multiple PEG-insulin species in the preparations. Also, it could be that observed differences are due to differences in binding affinity to the receptor rather than absorption per se.

### Effect of PEG Size

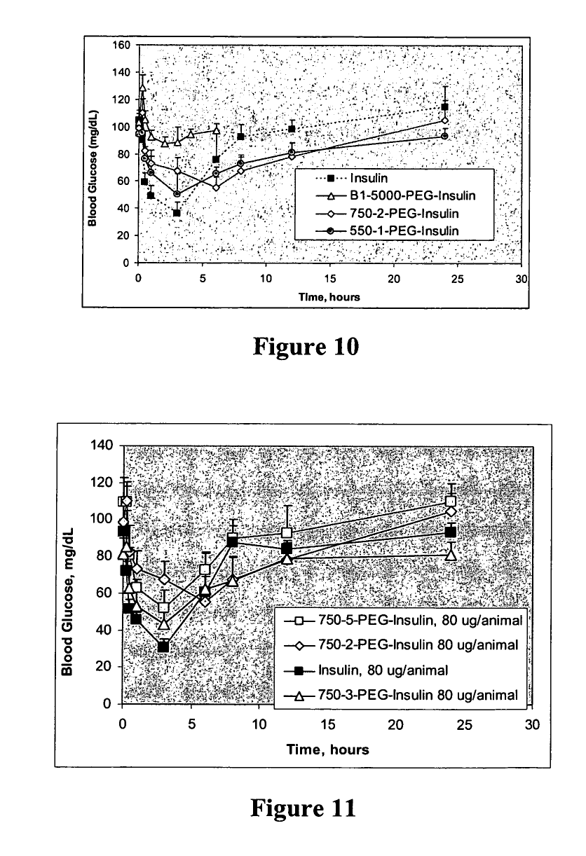

PEGylated insulins were examined that were all derivatized in positions B1 or B29 using the same PEG active agent (SPA). The actual synthetic routes to generate specific B1 or B29 isomers were different. Data for B1-5000-PEG-Insulin, 750-lot 2-PEG-insulin and 2K-lot 2-PEG-Insulin and 750-lot 5-PEG-insulin; their respective glucose suppression curves are shown in FIGS. 10 and 8b. The 750-lot 2-PEG-insulin analog exhibits a prolonged glucose suppression profile, which reaches a nadir at 6 hours and slowly recovers, without however reaching baseline before 12 hours post-administration.

The B1-5000-PEG-insulin analog produced little systemic glucose suppression compared to the insulin control. However, the glucose apparently remains below euglycemic levels for at least 6 hours; albeit reached the nadir much faster than 750-lot 2-PEG-insulin. However, the results for the 5000 PEG analog are not conclusive, as there is no data beyond the 6-hour time point.

In contrast to 750-lot 2-PEG analog, both B29 750-lot 5-PEG- and 550-lot 1-PEG-insulin had a similar glucose profile to regular insulin, indicating that conjugation in position B29 with a small PEG does little to prolong its absorption through the lung (FIG. 8b). However, when the PEG size increased to 2000 D, its absorption appeared prolonged, as indicated by the prolonged glucose suppression.

Overall, both PEG size and site of attachment seem to influence PEG-insulin absorption in rat. When a B1 strategy was utilized (albeit not pure), even a 750 MW PEG appeared capable of producing a prolonged effect, whereas it did little as a B29 derivative. Only B29 conjugates with PEG MW>2000 appear to be efficient in prolonging glucose depression. It is possible that at high molecular weights (>5K) the efficiency of insulin transport through the lung may be limited. However, more experiments are required to confirm these observations. It should be noted however, that the implication of the PEGylation site in transport through the lungs, given a passive diffusion mechanism, is questionable as presumably all mono-750-PEGylated analogs should have identical diffusion coefficients. Alternatively, it is possible that the PEGylation site may confer other changes (such as structure, association state) that may ultimately affect lung permeation kinetics.

### Effect of PEGylation Site

In order to evaluate the impact of PEGylation site on insulin absorption, analogs were examined that were all derivatized with the same MW PEG (750 Da) using site-specific PEGylation: 750-lot 2-PEG-insulin (PEG in position B1), 750-lot 5-PEG-insulin (PEG in position B29) and 750-lot 3-PEG-insulin (random PEGylation in positions A1 and B29) (Table 2). The results are illustrated in FIG. 11.

The data suggest that both 750-lot 2- and 750-lot 3-PEG-insulin analogs produced a prolonged systemic glucose suppression compared to the insulin control. In contrast, the B29 750 analog produced a glucose depression profile similar to the control, thus confirming the above observation that B29 analogs are absorbed faster through the lungs. Finally, the role of the di-PEGylated species is rather unclear, as despite their reduced activity they may contribute in the longer duration of action of the analogs owing to their reduced absorption rate through the lung.

### Effect of Acetylation and PEGylation

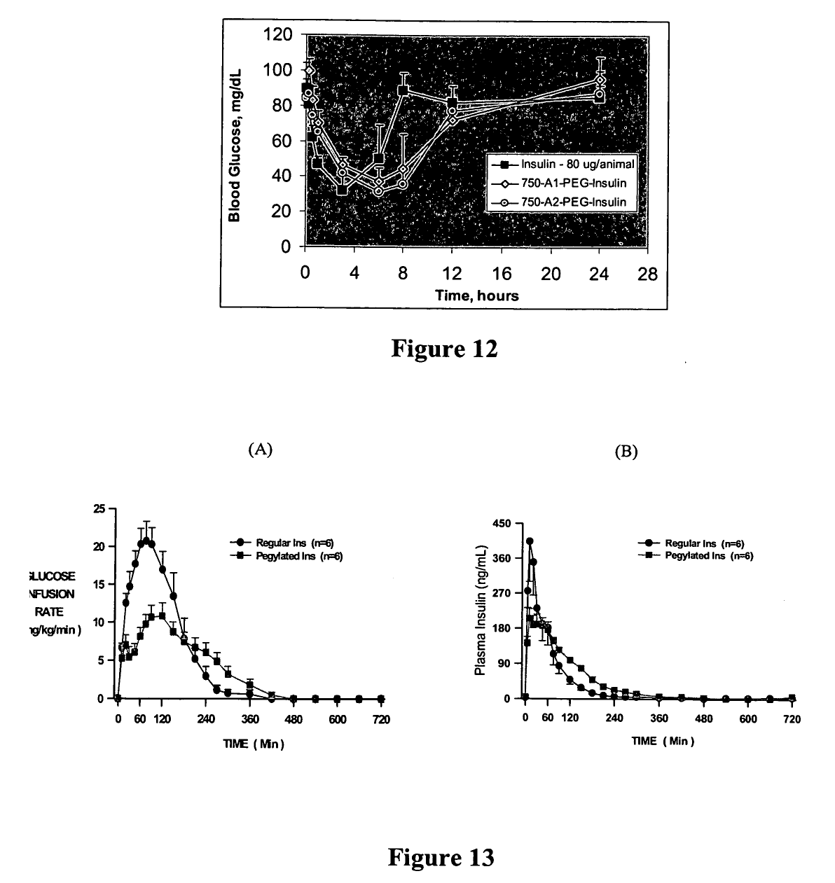

The impact of acetylation with PEGylation on insulin absorption through the lungs was evaluated. Acetylated derivatives were produced at different levels of acetylation and were all PEGylated at the same position (B1) with the same MW PEG (750 Da): 750-lot A1- and 750-lot A2-PEG-insulin (details are given in Table 2). The results are illustrated in FIG. 12.

The results indicate that both acetylated/PEGylated analogs maintained their relative activity, as they produced similar glucose depression levels compared to the insulin control. Further, both analogs prolonged glucose suppression, which reached nadir at 6 hours and returned to baseline by 12 hours. Overall, the glucose suppression profile shows a more pronounced effect, compared to the non-acetylated 750-lot 2- and 750-lot 3-PEG-insulin analogs.

This Example demonstrates that PEGylation of insulin in position B1 or B29 has a minimal impact on its bioactivity when small PEGS are conjugated. At higher PEG MWs, the bioactivity is severely impacted. Further, both PEG MW and site of attachment seem to regulate its transport through the lungs. Although MW had the highest impact, the site of attachment seemed to be important, as B29 attachment seemed to be less efficient in prolonging insulin absorption through the rat lungs. As explained above, assuming that a passive diffusion mechanism is dominant, this effect may be related more to a physical change in the association state of insulin (monomer-dimer-hexamer equilibrium). In order to confirm these findings, the best performing analogs (750-lot 2-, 750-lot 3-, 2K- and 2K-lot 2-PEG-insulin) were evaluated in a dog model as inhaled powders in the following Example. Further, since acetylation/PEGylation appeared to produce a desired glucose suppression prolongation, the impact of B29-acetylated and PEGylated analog with a 2000 MW PEG was also evaluated.

### Example 24

### Determination of Impact of PEGylated Insulin Powder Formulation on Insulin Pulmonary Absorption after Dry Powder Inhalation by Dogs

This Example evaluates the impact of PEGylated insulin powder formulation on pulmonary absorption of insulin after administration via dry powder inhalation in dogs.

### Non-Glucose Clamp Dog Inhalation

Anesthetized beagle dogs were dosed using the formulated dry powder of PEG-insulin (n=2) or regular insulin (n=2) via an endotracheal tube and an animal dry powder dosing system based on the PDS described above. All doses were calculated on the basis of equivalent insulin mass. Blood was withdrawn from an indwelling catheter over a 24-hour period. Resultant serum insulin and glucose concentrations were compared between the two groups.

### Glucose Clamp Dog Inhalation

Anesthetized beagle dogs were dosed using the formulated dry powder of PEG-insulin (n=3) or regular insulin (n=2) via an endotracheal tube and a dry powder dosing system. All doses were calculated on the basis of equivalent insulin mass. The glucose clamp technique involved the continuous infusion of a 50% glucose solution at a rate such that normal serum glucose values were maintained throughout the experiment (e.g., normal range is 80-100 mg/dl). In addition, somatostatin was infused to shut down all endogenous insulin production.

Glucagon was also infused to maintain normal serum physiologic concentrations. Thus, the amount of glucose required to be infused as well as the duration of the glucose infusion was a direct measure of the overall metabolic activity of the inhaled insulin. Blood was withdrawn from an indwelling catheter over a 10-hour period. Resultant serum insulin and glucose concentrations were compared between the two groups.

### Formulation Characterization

The compositions of the dry powder formulations of PEG-insulin analogs are given in Table 3 and their aerosol performance in Table 4.

TABLE 3
Composition of the PEG-Insulin Formulations used in the Dog Inhalation studies

| | PEG- | | Na | | | BP Fill |
|---|---|---|---|---|---|---|
| | insulin | Insulin¹ | Citrate | Trileucine | Leucine | Weight |
| Analog | (% w/w) | (% w/w) | (% w/w) | (% w/w) | (% w/w) | (mg) |
| 750-lot 3 | 26 | — | 15 | 16.6 | 40 | 4 |
| 2K-lot 2- | 26.8 | 72.9 | 8 | 18 | 47.2 | 4 |
| PEG | | | | | | |

¹Mass fraction of insulin in the PEG-Insulin analog

A summary of the delivered doses of the dog inhalation study is given in Table 4. Delivered doses were calculated using the following formula (% insulin purity refers to the purity of the bulk material (99%)):

Delivered·Dose=(# Bps)*(Bp·Fill·Weight)*(% w/w·Insulin)*(% Ins·Purity)*(% FPM)

TABLE 4
PEG-Insulin Aerosol Powder Characteristics
and delivered dose. The latter refers to the
dose delivered in the deep lung, per
the assumption of the calculations above.

| | Delivered |
|---|---|
| PEG-Insulin Analog | Dose (mg) |
| 750-lot 3 | 6.3 |
| 2K-lot 2-PEG | 1.0 |

As can be seen in Table 4, the 750-lot 3-PEG-Insulin was dosed at 6.3 mg insulin, which is unacceptably high, and as such, the results of that study will not be considered.

The B29 2K-2-PEG-insulin analog was evaluated in a glucose clamp study, whose results are shown in FIG. 13. As shown in FIG. 13a, the B29 2K-2-PEG-insulin group required less infused glucose to maintain basal levels, but infusion was necessary for longer duration (420 min) than insulin alone (300 min). The prolonged action of the 2K-2-PEG-insulin was further confirmed by the insulin plasma pharmacokinetic profile illustrated in FIG. 13b. The AUC of the respective insulin serum concentrations for each of the groups was roughly the same, thus indicating that the PEG-insulin analog exhibited equivalent apparent bioavailability to control insulin.

PEG-insulin analogs of the appropriate size can cross the lungs and retain bioactivity.

B1-derivatized insulin with 750 MW PEG seemed to efficiently cross the lung and retain their activity, while producing prolonged glucose suppression. The B29-insulin conjugates produced prolonged glucose depression in rats following instillation administration. However, dog inhalation studies indicated that it exerts a minor only effect on plasma insulin PK.

Furthermore, the B29 2000 Dalton analog appears to have similar pulmonary bioavailability to the insulin control in dog. Insulin combined with acetylation and PEGylation appears to prolong glucose suppression in rat. However, its effect on glucose suppression kinetics may be small.

### Example 25

### Dose Determination and Pharmacokinetic/Pharmacodynamic Evaluation of PEGylated Insulin Compositions

Insulin compound evaluations were conducted in two phases on the compounds synthesized in Examples 9-11, 16, and 18. The first phase established a dose of the compound required to achieve a target blood glucose reduction. This target blood glucose reduction was chosen to be the minimum reduction that is clearly recognizable as different from the resting blood glucose concentration.

The second phase examined the pharmacokinetics/pharmacodynamics of the molecule when administered to rats at the selected dose over an observation time sufficient for the glucose to return to baseline. The sections below provide a synopsis of experimental materials and methods used in these rat in vivo studies.

These experiments were conducted on male adult Sprague-Dawley rats of approximately 0.30-0.40 kg. Animals were obtained pre-cannulated with a jugular vein catheter (JVC) for blood sampling and, in the case of IV administration, and additional femoral vein catheter (FVC).

Stock solutions of the compounds were generally prepared in Dulbecco's PBS using gravimetric measurements. A spectrometric measurement of compound concentration was also made. The optical density (OD) of the stock solution was measured and the concentration of insulin was calculated using an extinction coefficient of 1.05 mL/mg/cm at 276 nm wavelength. Dose calculations and dilutions were based on the spectrophotometrically measured insulin concentrations. Unless specifically noted, compound concentrations and doses throughout this document are calculated with insulin mass, i.e., not including mass of PEG or other derivatives.

### Intratracheal Instillation

Intratracheal Instillation (IT) was conducted as follows. The rats were anesthetized with isoflurane mixed with oxygen and were suspended vertically by their upper incisors on a rubber band which stretched between two burette stands. Compounds were administered in a volume of 300 μl with a gavage needle inserted orally into the rat and descended into the trachea just above the carina. Intravenous administration of compounds was made via a femoral catheter in a volume of 300 μl.

Sample Collection was performed as follows. Blood samples of approximately 0.50 mL were collected from the JVC using a 1 mL syringe at each time point. A new syringe was used for each time point along with precautions to prevent contamination and carry-over.

The glucose monitoring was conducted as follows. Approximately 20 μL of whole blood was touched to the end of the glucose test strip of 2 Glucometer Elite glucose monitors for a duplicate determination of blood glucose concentrations. If there was a discrepancy of greater than 5 mg/dl, a third reading was taken.

The sample for compound quantitation in plasma, which served as basis for the PK analysis, was collected and processed as follows. The volume of blood remaining after the glucose measurement (˜480 μL) was anticoagulated with K2 EDTA and centrifuged to recover plasma. The plasma was collected and stored at −80° C. until analysis. PEGylated insulin derivatives were quantitated in rat plasma using sandwich ELISA assays. The immunoassays used for the PEGylated derivatives are commercially available kits approved for clinical use. These were the Mercodia Human Insulin ELISA kit and the Mercodia Iso-Insulin ELISA kit. The Insulin ELISA kit was used to detect underivatized insulin as well as PEGylated but un-acetylated derivatives. This kit could not detect the acetylated derivatives presumably because the acetylation destroyed an epitope required for the binding of at least one of the kit antibodies. An alternate assay, the Iso-Insulin ELISA kit, was found that could detect the acetylated derivatives. This is thought to be due to the antibodies in this kit being directed to the center portion of the insulin molecule, away from the acetylation sites.

The dose of reference compound AKZO NOBEL Human Insulin required to achieve a target blood glucose reduction when administered to rats via intravenous injection was established. The PD/PK relationship for insulin was measured over a range of doses when administered to rats via intravenous injection. Doses of 1-6 μg/rat of AKZO insulin were administered IV. Blood glucose and insulin concentrations in plasma were monitored for 3 hours after administration.

The PD/PK relationship for insulin was measured over a range of doses when administered to rats via intravenous injection. Doses of 1-6 μg/rat of AKZO insulin were administered via N injection. Blood glucose and insulin concentrations in plasma were monitored for 3 hours after administration.

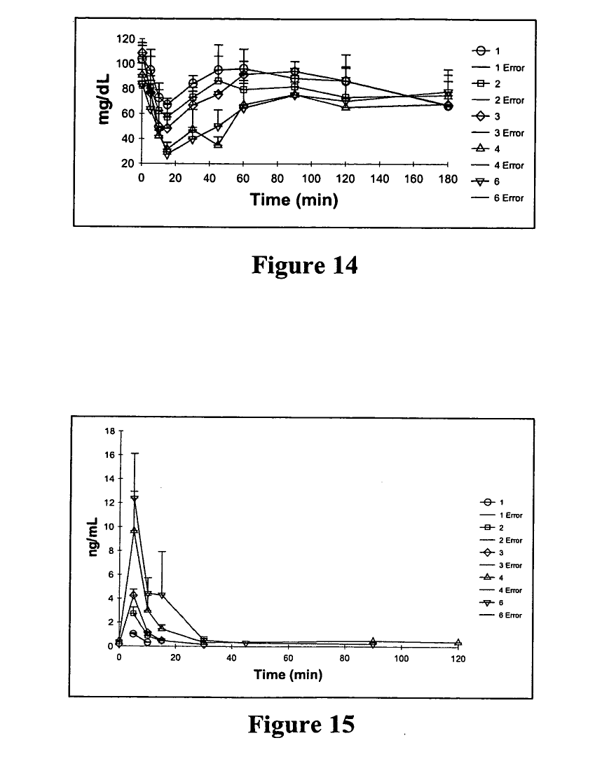

Glucose time concentration curves are given in FIG. 14. Glucose suppression was found to be dose proportional from 1-4 μg/rat with a dose of 3 μg/rat adequate to reduce the blood glucose to the target interval of 40-60 mg/dl. The response to 4 and 6 μg/rat was not distinguishable, owing perhaps to glucose homeostatic mechanisms increasing as blood glucose fell below 40 mg/dl. Blood glucose was back to baseline values by 90 minutes for all doses. Glucose Tₘᵢₙ was 15 minutes for all doses.

Plasma insulin concentrations as a function of time are presented in FIG. 15. Cₘₐₓ values increase with dose administered and were observed at the first sampling post administration. Concentrations decrease rapidly and at or near baseline or below limit of quantitation by 45 minutes.

The dose of reference compound HUMULIN N human insulin required to achieve a target blood glucose reduction when administered to rats via subcutaneous injection was established. In addition, the pharmacokinetics/pharmacodynamics (PK/PD) when HUMULIN N human insulin was administered to rats via subcutaneous injection was evaluated.

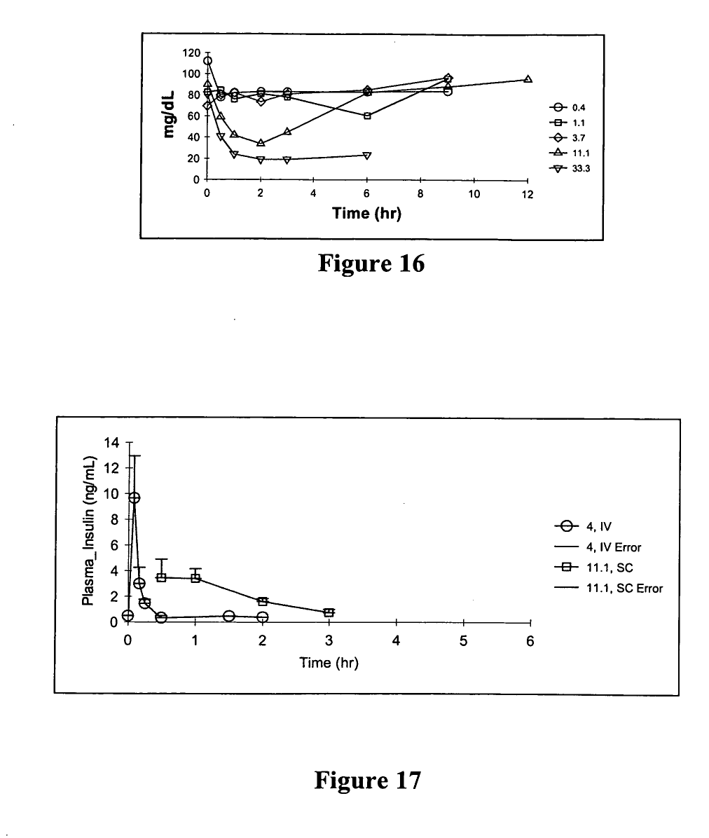

A dose escalation series with HUMULIN N identified 0.3 U as adequate to achieve the target glucose suppression (see FIG. 16). This dose corresponds to 11 μg/rat, assuming a specific activity of 27 U/mg insulin. At a dose of 0.3 U/rat the Tₘᵢₙ was 2 hours and the Cₘᵢₙ was 34 mg/dl. Interestingly, the blood glucose had returned to baseline by 6 hours after administration. This is a substantially shorter duration of action than reported in humans (14-18 hours). It is possible that this difference between activity profiles in the human and the rat is due to the nature of the tissue into which a subcutaneous injection is made in the 2 species.

Plasma insulin concentrations as a function of time following SC administration of HUMULIN N are given in FIG. 17. This profile differs significantly from that resulting from an IV injection of insulin. However, plasma concentrations started decreasing at 1-hour post injection and were approaching the limit of quantitation at 3 hours, the last time point sampled.

The dose of PEG2000-B1, PEG3000-B1, and PEG5000-B1 human insulin required to achieve a target blood glucose reduction was established, and the pharmacokinetics/pharmacodynamics (PK/PD) of each of those PEGylated molecules was evaluated when administered to rats via intravenous injection at the selected dose. A dose of 15 μg/rat of each of these compounds administered IV resulted in a nadir blood glucose concentration in the target range. This suggests that these compounds have similar in vivo potency. The nadir for PEG2000-B1, PEG3000-B1, and PEG5000-B1 were respectively 42, 36, and 42 mg/dl.

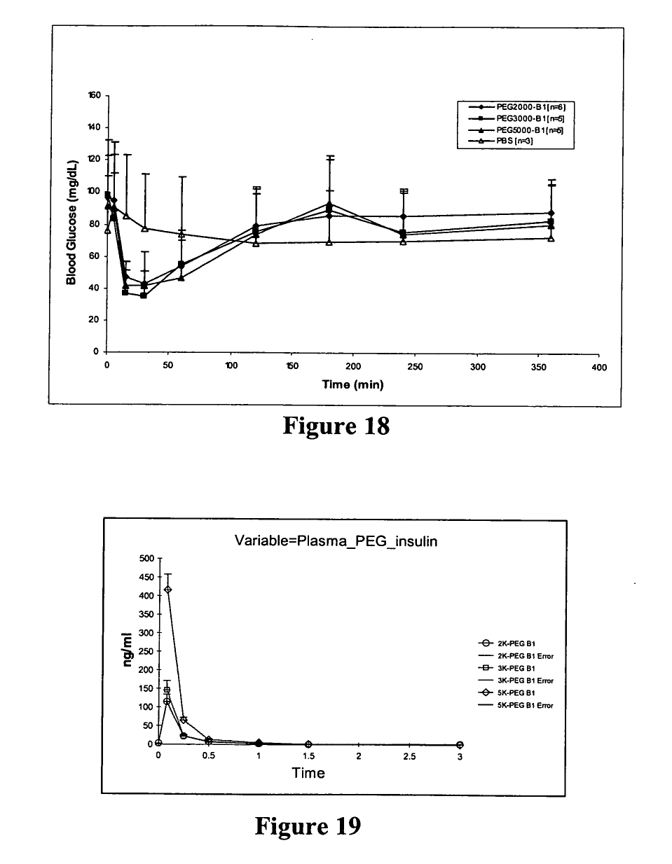

The time taken for the glucose to reach nadir (Tₘᵢₙ) was similar for all 3 compounds. Blood glucose concentrations returned to baseline by 120 minutes following administration. These results are given in Table 5 and FIG. 18.

TABLE 5

| | Dose | Cₘᵢₙ | Tₘᵢₙ |
|---|---|---|---|
| Test Article | (μg/animal) | (mg/dL) | (min) |
| PEG2000-B1 Insulin | 15 | 42 ± 13 | 33 ± 15 |
| PEG3000-B1 Insulin | 15 | 36 ± 11 | 24 ± 12 |
| PEG5000-B1 Insulin | 15 | 42 ± 9 | 24 ± 8 |

Cₘᵢₙ = Minimum blood glucose concentration;
Tₘᵢₙ = Time to Cₘᵢₙ

Plasma compound concentrations are shown in FIG. 19, with time in hours. Compound concentrations are maximal at the first sampled time and decrease rapidly thereafter. The Cₘₐₓ of PEG2000-B1, PEG3000-B1 compounds are similar (114 and 145 ng/ml, respectively). The Cₘₐₓ of PEG5000-B1 is higher at 416 ng/ml. This could indicate a smaller volume of distribution.

The experimental protocol was modified for the analysis of the acetylated compounds. This resulted from the intent to use LC/MS-MS for the quantitation of compound concentrations in plasma. This analytical technique requires a larger volume of plasma to permit concentration of the sample and achieve the requisite increase in sensitivity. The increased requirement for plasma exceeded the permissible amount that can be withdrawn from a rat in the time interval of 24 hours. The protocol was therefore modified to increase the number of animals by two-fold and split the samples between these two cohorts.

The dose ranging leg of this study identified the following doses as adequate to reach the target nadir glucose suppression: 180, 240 and 640 μg/rat for the PEG2000-B1, PEG3000-B1, and PEG5000-B1 molecules, respectively. These doses are different from those required to achieve similar glucose suppression when administered IV. This indicates limited bioavailability following lung administration and may result from low permeation through the lung and/or local metabolism.

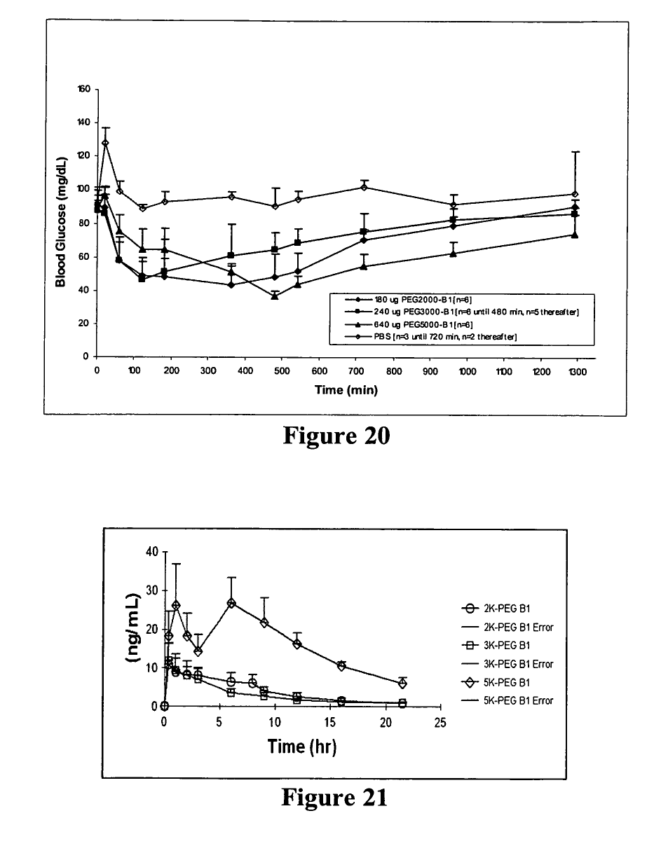

The definitive leg of this study revealed a glucose suppression profile substantially different from that seen after IV administration of these compounds. The Cₘᵢₙ and Tₘᵢₙ are given in Table 6 and the glucose suppression profiles in FIG. 20. The Tₘᵢₙ of all 3 compounds were significantly later after IT administration than after IV administration. The glucose suppression profile of the PEG2000-B1 and PEG3000-B1 molecules are similar for the first 2 hours after which they diverge with the PEG2000-B1 having a prolonged suppressive activity. The glucose suppression profile of the PEG5000-B1 is delayed relative to the 2 other molecules and reaches Cₘᵢₙ at 8 hrs. The duration of glucose suppression for all 3 molecules is extended relative to the IV profile. The blood glucose is still suppressed at 9 hrs after administration of PEG2000-B1 and PEG3000-B1 and as much as 16 hrs after PEG5000-B1 administration.

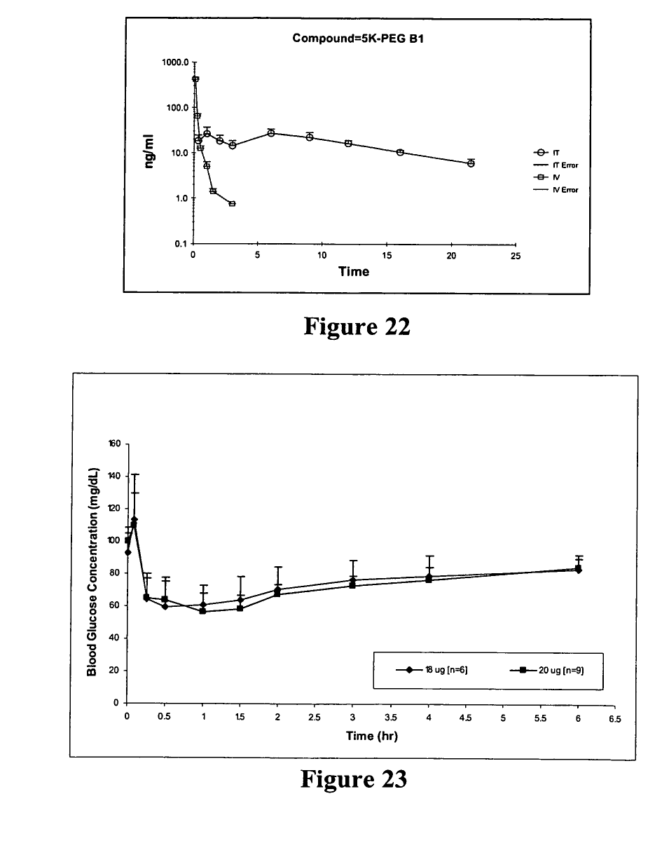

Compound concentrations achieved in plasma as a function of time are given in FIG. 21. The concentration/time profile differs from the IV pattern by the substantially lower Tₘₐₓ, prolonged presence of compound in plasma and flatter aspect of the curve. The difference between the IV and IT patterns are further illustrated in FIG. 22, which shows the IV and IT plasma concentrations as function of time for the PEG5000-B1 molecule on the same graph. The Cₘₐₓ is substantially higher following the IV administration, the decline in compound concentration is much slower after the IT administration leading to a flatter concentration/time curve. The concentrations of PEG5000-B1 remain elevated much longer after an IT administration: they fall below the limit of quantitation by 6 hours post IV while they are still elevated at 21 hours post IT.

TABLE 6

| | Dose | Cₘᵢₙ | Tₘᵢₙ |
|---|---|---|---|
| Test Article | (μg/animal) | (mg/dL) | (min) |
| PEG2000-B1 Insulin | 180 | 38 ± 9 | 290 ± 171 |
| PEG3000-B1 Insulin | 240 | 45 ± 18 | 170 ± 96 |
| PEG5000-B1 Insulin | 640 | 37 ± 3 | 480 ± 0 |

Cmin = Minimum blood glucose concentration;
Tmin = Time to Cmin

This aspect of the example was done twice because in the first series the dose determined in the dose ranging study (18 μg) was found barely sufficient to meet the glucose reduction criteria in the definitive leg. Therefore, the study was repeated at a slightly higher dose of 20 μg/rat. The group's mean Cₘᵢₙ in the second definitive leg was 54 mg/dl. Results for both definitive experiments (18 and 20 μg/rat) are presented in FIG. 23. The return to baseline blood glucose values are later than following IV administration of AKZO insulin (FIG. 23) suggesting a difference in disposition resulting from the addition of PEG.

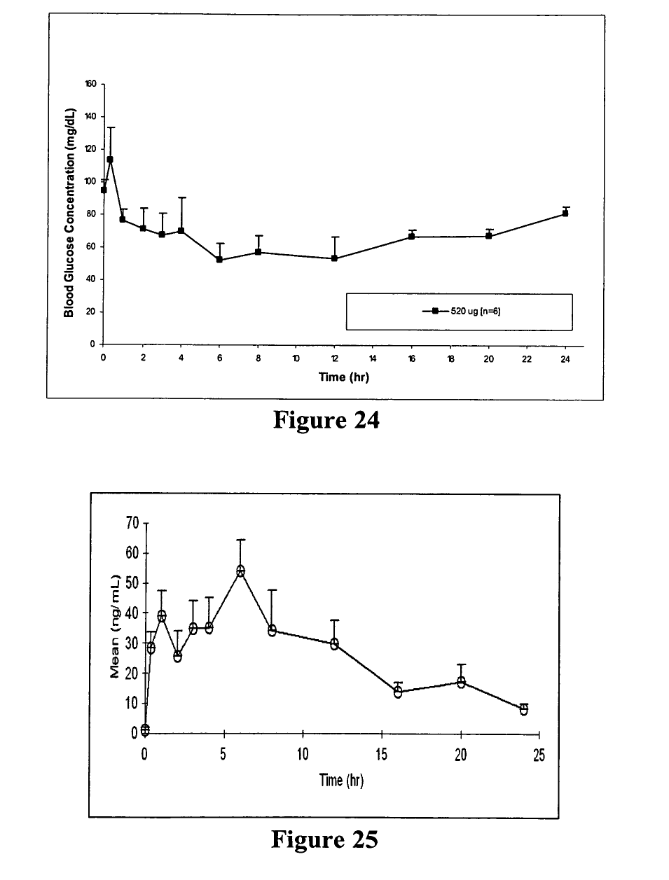

Dose ranging studies indicated that doses from 480 to 780 μg/rat were adequate to achieve the target blood glucose suppression though there was considerable variation between animal and dose groups. A dose of 520 μg/rat dose was used for the definitive leg. Cₘᵢₙ and Tₘᵢₙ values are given in Table 7 and mean glucose values as a function of time are given in FIG. 24. Group mean glucose values suggest the blood glucose is suppressed for at least 12 hours. Group mean plasma compound concentrations as function of time are given in FIG. 25. Compound concentrations are elevated and relatively flat until approximately 12 hours and decline thereafter. However, they remain elevated above pre-dose values at the last sampling point of 24 hours.

TABLE 7

| | Dose | Cₘᵢₙ | Tₘᵢₙ |
|---|---|---|---|
| Test Article | (μg/animal) | (mg/dL) | (hr) |
| PEG5000-B1 Di-acetylated insulin | 520 | 58 ± 11 | 7.1 ± 6.6 |

Cₘᵢₙ = Minimum blood glucose concentration;
Tₘᵢₙ = Time to Cₘᵢₙ

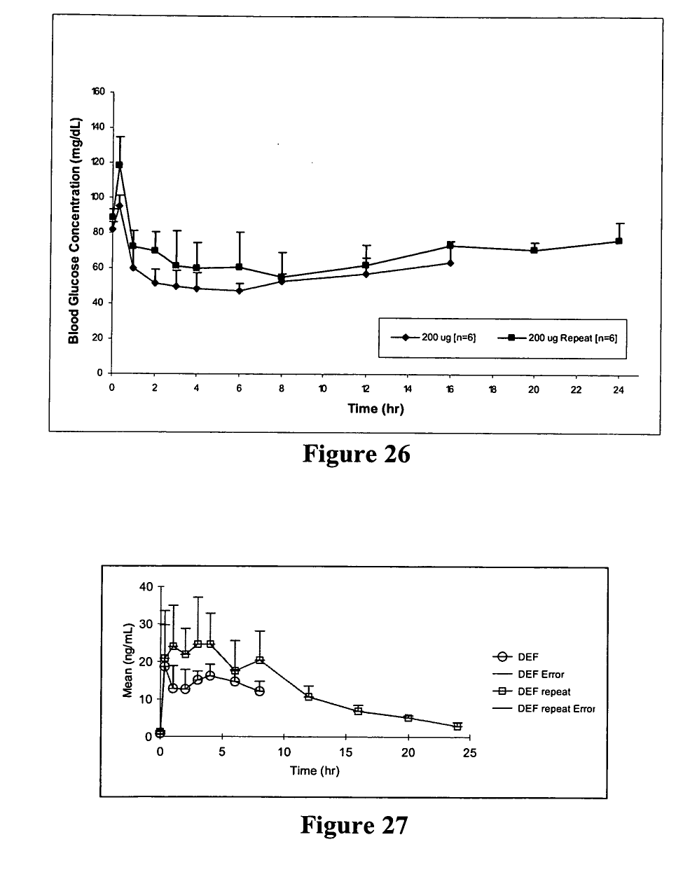

The dose ranging studies identified 240 μg/rat as the most appropriate of the doses tested. At this dose one of 2 rats still went hypoglycemic (</=20 mg/dl) and a dose of 200 μg/rat was chosen for the definitive study. This was the first study designed to use LC/MS-MS for compound quantitation. Blood volume withdrawal regulations limited the observation period to 16 hours in the first definitive study. A second study using split groups was subsequently conducted. Results from both of these studies are given in Table 8 and FIG. 26.

Group mean PEG2000-B1 Di-acetylated insulin concentrations in plasma for these studies are given in FIG. 27. There was considerable variation in concentrations and profiles between individual animals. Overall, the group mean compound concentration was a plateau from the first observation at 20 min to 8 hours and thereafter declines gradually over the next 16 hours. The study for which there was PK data only until 8 hours shows substantially less interanimal variation. The plasma concentration shows a similar plateau throughout the entire observation period (20 min to 8 hrs) and an absolute concentration that trends lower than the companion study.

TABLE 8

| | | Dose | Cₘᵢₙ | Tₘᵢₙ |
|---|---|---|---|---|
| Group | Test Article | (μg/animal) | (mg/dL) | (hr) |
| 6 | PEG2000-B1 Di-acetylated | 200 | 43 ± 5 | 4.3 ± 1.6 |
| | insulin | | | |
| 7 | PEG2000-B1 Di-acetylated | 200 | 54 ± 14 | 9.3 ± 6.9 |
| | insulin | | | |

Cₘᵢₙ = Minimum blood glucose concentration;
Tₘᵢₙ = Time to Cₘᵢₙ

A dose of 10 μg/rat was found adequate to reach the target glucose suppression level. This dose is half of that required for an equivalent suppression with the PEG5000-B1 Di-acetylated derivative. While is it not clear whether this difference is statistically significant, it is compatible with the observation that the affinity of the 5K derivative is lower than that of the 2K derivative as assessed in a competition binding assay using an isolated preparation of the human insulin receptor.

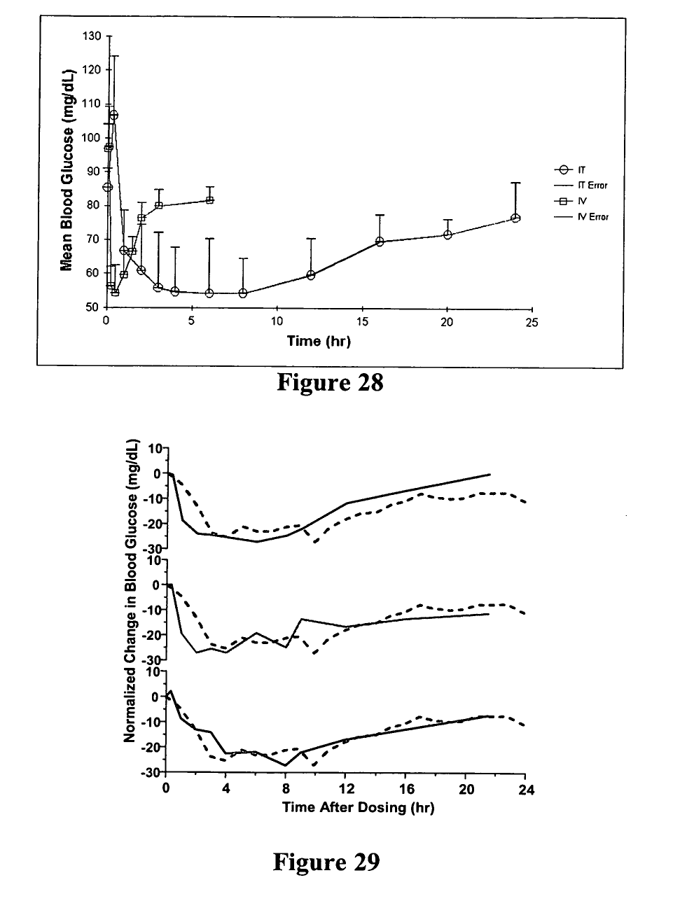

The definitive leg of this study was conducted with a dose of 10 μg/rat. Blood glucose concentration as a function of time is presented in FIG. 28. In this figure, the IV results are graphed along with the results from a corresponding IT study (i.e., the dose ranging and PK/PD studies following IT administration of Di-acetylated PEGylated insulin, described above) to emphasize the differences in pharmacological activity profiles attributable to the different administration routes. Group mean blood glucose nadir concentration after IV administration was 52 mg/dl, and Tₘᵢₙ was approximately 20 minutes post injection. Blood glucose concentrations were back to baseline at approximately 2 hours. By contrast, despite similar nadir concentrations (Cₘᵢₙ 55 mg/dl), Tₘᵢₙ after IT administration was 8 hours and blood glucose values did not return to baseline before 16 hours.

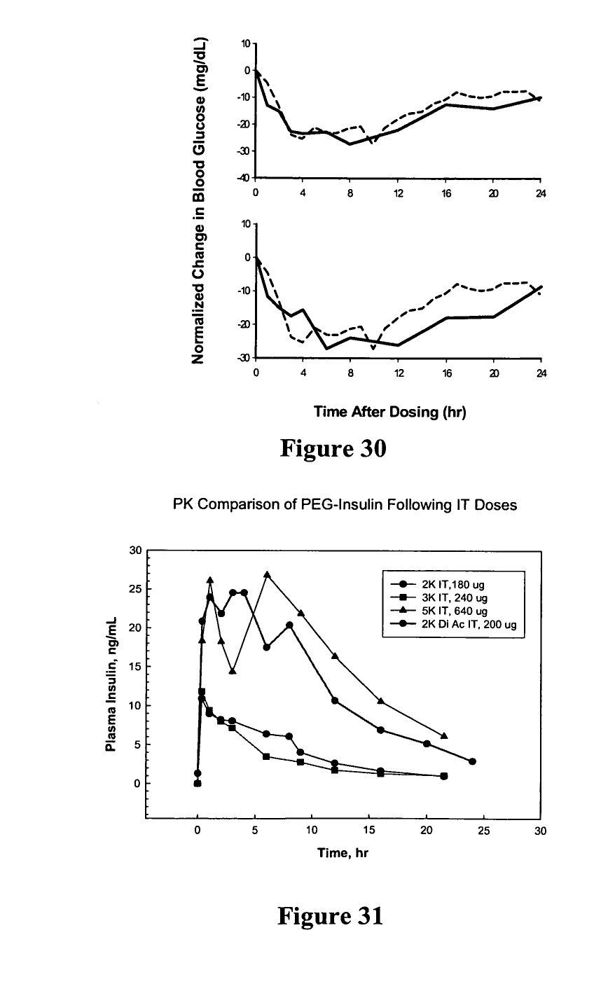

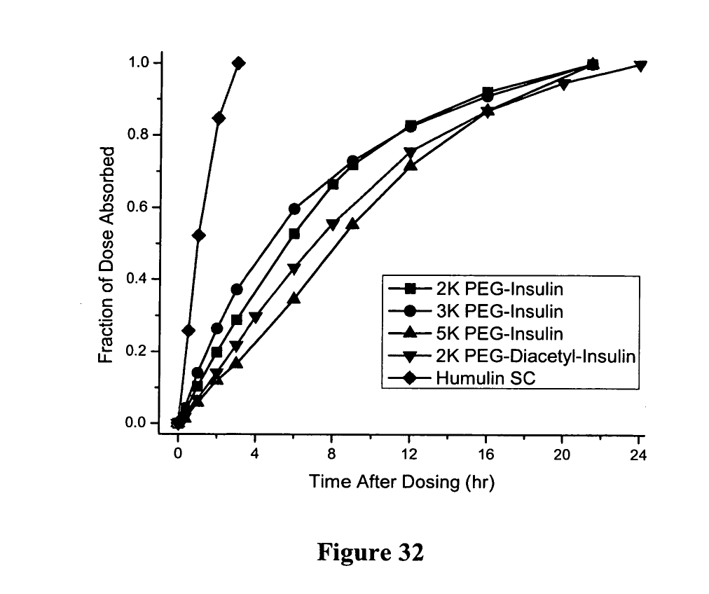

From available PK/PD results in fasting rat model, prolonged glucose suppressions were achieved following a single IT dose of various B1 PEGylated insulins. It is shown in the FIG. 29 that the glucose suppression profiles in fasted rats following IT single dose of B1 PEGylated Insulins compared to NPH Profile in humans following SC administration, whereas FIG. 30 shows the glucose suppression profiles in fasted rats following IT single dose of B1 PEGylated, A1 and B29 di-acetylated Insulins. Both series of compounds showed prolonged glucose suppression in agreement with our target therapeutic profile. Moreover, the PK profiles support the prolonged release of PEGylated Insulin from the lung. FIG. 31 shows the average plasma drug concentrations for the various compounds tested. The terminal t_{1/2} was significantly prolonged as compared to IV doses, indicating sustained release from the lung. To explore the data further, the PK profiles after IT doses were deconvoluted using IV PK parameters using the Wagner Nelson deconvolution procedure, to generate the cumulative absorption profile (FIG. 32). From this figure, we can see that the drug absorption from IT doses was significantly prolonged, with 50% of dose still remaining to be absorbed at around 5-10 hr post IT dosing. In comparison, SC Humulin dose was completely absorbed after 3 hr.

### Example 26

### Evaluation of the Pharmacokinetic and Pharmacodynamic Profiles of B1-PEGylated and A1, B29-Diacetylated Insulins in Comparison to Insulin Following IV and IH Dosing in Dogs

The pharmacokinetic and pharmacodynamic profiles of PEG2000-B1, di-acetylated A1, B29 human insulin (2K di-AC) and PEG5000-B1, di-acetylated A1,B29 human insulin (5K di-AC) following IV and IH dosing in dogs was evaluated. The compounds were prepared in accordance with Examples 16 and 18. The dose required to achieve the target nadir of between 60 and 40 mg/dl was arrived at empirically in the dose ranging studies provided in Example 25.

6 Beagle dogs (˜7-10 kg) were anesthetized in both the IV and IH arms of this study for consistency. Dogs were then given 0.1 mg/kg acepromazine subcutaneously ˜15 minutes prior to 1-5% isoflurane mask induction. Once the appropriate plane of anesthesia was achieved, the pre-shaved neck of the dog was installed with an 18-20 G Abbocath intravenous catheter in the right or left jugular vein and pre-dose sampling was taken. This catheter was used for all blood samplings other than the later time points (16, 20 and 24 hour). Subsequent samples were taken from the cephalic veins.

The test and control articles were administered by bolus intravenous (IV) injection of a volume of 300 μl into the left cephalic vein using a 22 G 1 inch 1 mL tuberculin syringe.

The formulated control and test articles were delivered by inhalation (IH) via an endotracheal (ET) tube. Ventilator stroke volume was set empirically for each dog to achieve a Peak Mouth Pressure (PMP) of 20-25 cm H₂O. A digital manometer (Dwyer Instruments, Michigan City, Ind.) was connected to the ET tube/“Y” connector to monitor PMP. Each blister pack (BP) was delivered with 2 full breathing cycles. No more than 3 BPs were administered during one apneic period. If required to achieve the target administered dose, a second apneic period was induced after an interval of 2 minutes of spontaneous breathing.

Total administered powder as well as estimated inhaled total lung dose were reported. Estimated inhaled total lung dose (mg) was calculated as follows: BP fill weight (mg)×#BPs×F (%)×DE (%) where: BP=Blister pack, F=Formulation (insulin content by weight-% per BP), and DE=Device efficiency.

Whole blood was used for the glucose measurements. EDTA-anticoagulated plasma was used for compound quantitation assays. Sampling time points for the IV treatment arm were: predose, 0.08 (5 min), 0.25, 0.50, 1, 1.5, 2, 3, and 6 hours post-dose, and for the IH arm: predose, 0.33, 1, 2, 3, 4, 6, 8, 12, 16, 20, and 24 hours post-dose.

Quantitation of test and control articles for PK analysis was made with a radio-immuno assay (RIA) conducted at Linco Research, St-Charles, Mo. Prior work selected a guinea-pig antiserum capable of recognizing the 2K and 5K di-AC compounds as well as the 5K unacetylated compound. This antiserum was used as the basis of an RIA assay which was then qualified with respect to accuracy, precision, sensitivity, and dilutional linearity. The working range of this assay was 0.15-5 ng/ml. Samples exceeding the upper boundary of this range were diluted in a pool of charcoal-stripped canine plasma.

C-peptide was measured in one series, 1H-administered insulin. This measurement was also conducted using an RIA assay.

The dose used in the study was primarily based on the maximum glucose suppression achieved from the results of the dose ranging studies in Example 25. The final delivered lung dose (after the ET tube) was slightly different between dogs. As a result, the mean and standard deviation of the dose (mg/dog) were calculated.

TABLE 13
Dose (past ET tube) comparison.

| ROUTE | TEST ARTICLE | N | MEAN | SD | CV % |
|---|---|---|---|---|---|
| IH | INSULIN | 6 | 0.34 | 0.0155 | 4.6 |
| IH | PEG2000-B1 | 12 | 0.60 | 0.0957 | 16.0 |
| | DI-ACETYL INSULIN | | | | |
| IH | PEG5000-B1 | 10 | 2.00 | 0.3667 | 18.4 |
| | DI-ACETYL INSULIN | | | | |
| IV | INSULIN | 6 | 0.027 | 0.0049 | 18.2 |
| IV | PEG2000-B1 | 8 | 0.058 | 0.0136 | 23.3 |
| | DI-ACETYL INSULIN | | | | |
| IV | PEG5000-B1 | 6 | 0.084 | 0.0136 | 16.2 |
| | DI-ACETYL INSULIN | | | | |

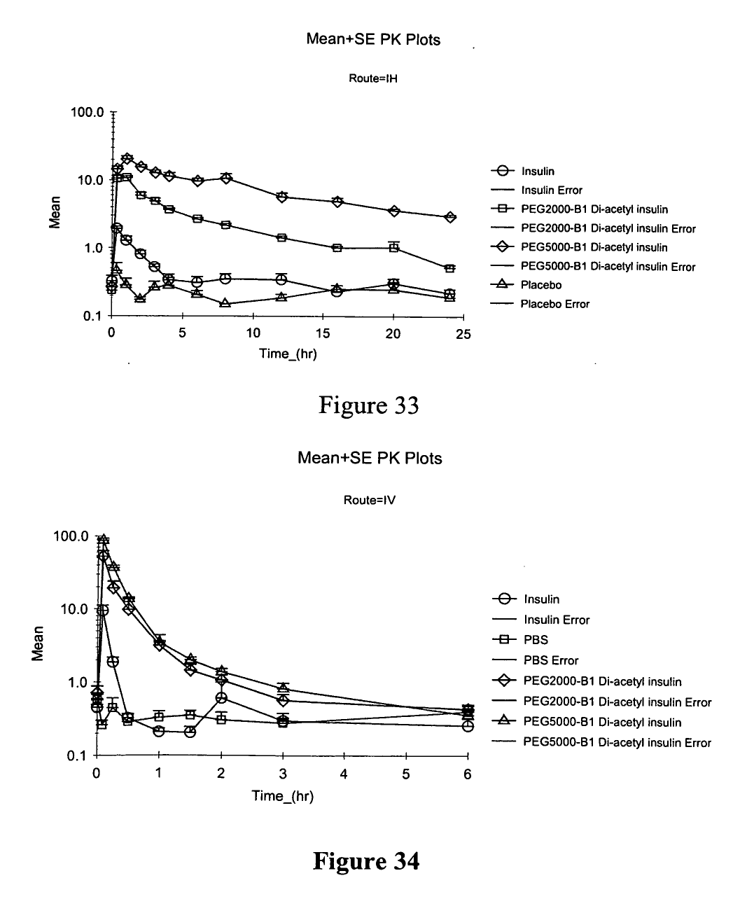

FIGS. 33 and 34 show the measured plasma insulin concentration (mean±SE) following IH and IV dosing in beagle dogs, respectively.

In PBS (IV) and placebo (IH) treated dogs, the baseline insulin level were 0.35±0.20 and 0.26±0.13 ng/mL, respectively. The baseline insulin level is therefore set at 0.35 ng/mL and all measured plasma insulin concentrations were corrected for the baseline (i.e., corrected insulin concentration (ng/mL)=measured insulin concentration (ng/mL)−0.35 (ng/mL)). Furthermore, low concentration samples (with corrected insulin concentration<2*SD of baseline level (i.e., 0.4 ng/mL)) were removed from PK analysis, due to baseline noise.

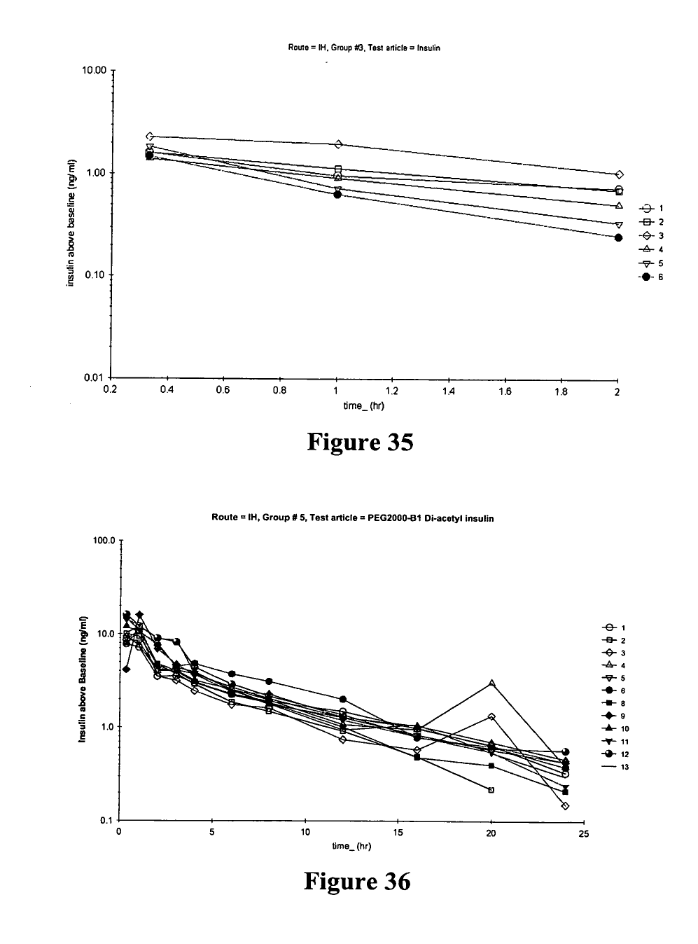

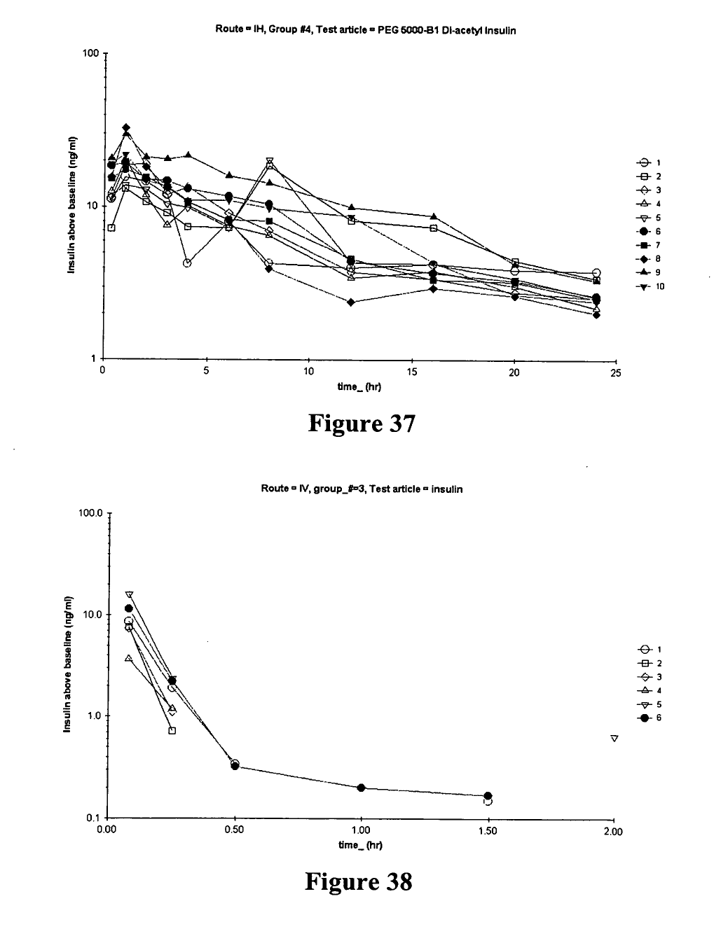

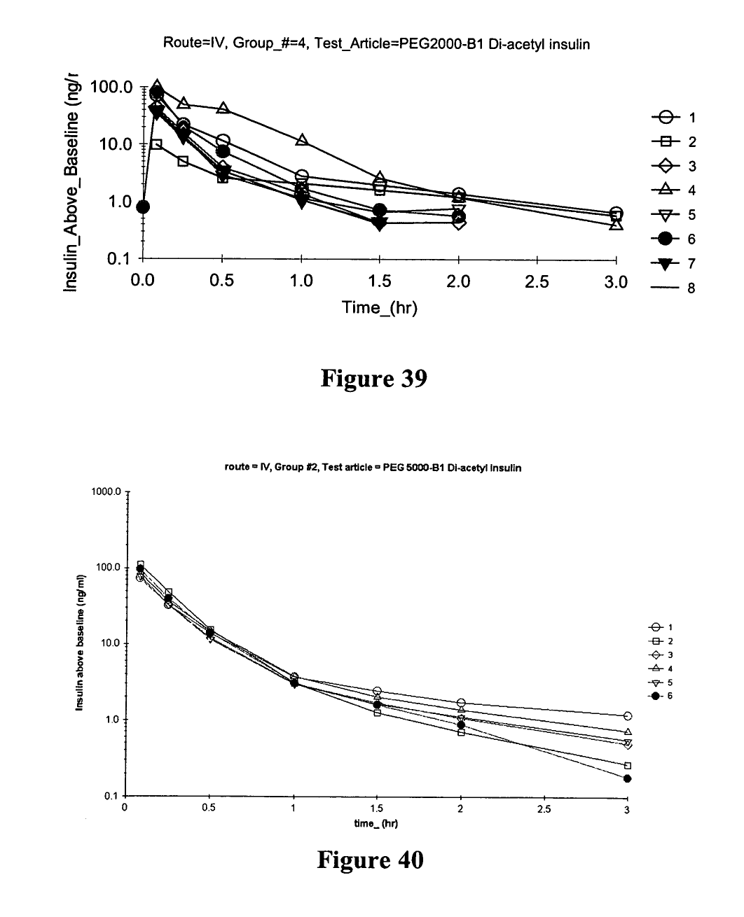

FIGS. 35 to 40 show the individual baseline corrected insulin concentration-time profiles following IH and IV dosing of Insulin, B1-2K-PEG-DiAc-Insulin, and B1-5K-PEG-DiAc-Insulin, respectively.

Non-compartmental pharmacokinetic (PK) model was fitted to individual animal plasma insulin concentration (baseline corrected) curves. The resulting mean PK parameters are summarized in Table 14.

TABLE 14
Non-compartmental PK parameters.

| TEST | | | T_{MAX} | C_{MAX} | T_{1/2} | AUC_{0-LAST} | AUC_{0-INF} |
|---|---|---|---|---|---|---|---|
| ARTICLE | ROUTE | | HR | NG/ML | HR | HR*NG/ML | HR*NG/ML |
| INSULIN | IH | MEAN | 0.33 | 1.567 | | 1.538 | |
| | | SD | 0 | 0.354 | | 0.837 | |
| | | SE | 0 | 0.145 | | 0.342 | |
| INSULIN | IV | MEAN | 0.08 | 9.117 | | 1.822 | |
| | | SD | 0 | 4.34 | | 1.886 | |
| | | SE | 0 | 1.772 | | 0.77 | |
| PEG-2K-B1 | IH | MEAN | 0.553 | 11.767 | 5.771 | 47.15 | 52.223 |
| DI-ACETYL | | SD | 0.33 | 2.777 | 1.202 | 10.289 | 9.839 |
| INSULIN | | SE | 0.095 | 0.802 | 0.347 | 2.97 | 2.84 |
| PEG-2K-B1 | IV | MEAN | 0.08 | 52.275 | 0.525 | 16.546 | 16.968 |
| DI-ACETYL | | SD | 0 | 29.144 | 0.303 | 13.005 | 13.003 |
| INSULIN | | SE | 0 | 10.304 | 0.107 | 4.598 | 4.597 |
| | | MEAN (EXCLUDE | | 58.35 | 0.46 | 18.01 | 18.37 |
| | | DOG 2) | | | | | |
| | | SD | | 25.43 | 0.24 | 13.31 | 13.37 |
| PEG-5K-B1 | IH | MEAN | 2.5 | 21.36 | 15.045 | 174.509 | 236.226 |
| DI-ACETYL | | SD | 2.915 | 5.474 | 13.208 | 44.049 | 71.43 |
| INSULIN | | SE | 0.922 | 1.731 | 4.177 | 13.929 | 22.588 |
| PEG-5K-B1 | IV | MEAN | 0.08 | 86.817 | 0.755 | 26.627 | 27.609 |
| DI-ACETYL | | SD | 0 | 14.485 | 0.404 | 3.743 | 3.754 |
| INSULIN | | SE | 0 | 5.913 | 0.165 | 1.528 | 1.533 |

Following IV and IH dosing in dogs, Insulin concentration increased quickly above baseline with average Tₘₐₓ of 0.08 and 0.33 hr, respectively. The average maximum plasma insulin concentrations above baseline (Cₘₐₓ) are 9.1 and 1.6 ng/mL, following IV and IH dosing, respectively. Since majority of the animals tested had only two quantifiable concentration data points above baseline, T_{1/2} was not calculated for Insulin. Plasma insulin concentrations dropped rapidly following IV and IH dosing, with last quantifiable sample observed at 0.33 hr following IV dosing and 2 hr following IH dosing. The total plasma exposures of insulin were similar between IV (0.027 mg/dog) and IH (0.34 mg/dog) dosing in dogs. The calculated bioavailability was about 6% for Insulin powder after the ET tube.

Following IV dosing of 0.058 mg/dog of B1-2K-PEG-DiAc-Insulin in dogs, Cₘₐₓ appeared at the first sampling time point (i.e., 0.08 hr), with average Cₘₐₓ of 58 ng/mL. The central volume of distribution (V_{c}) was about 111 mL/kg, or roughly twice of plasma volume in dog (i.e., 52 mL/kg). T_{1/2} was about 0.5 hr.

Following IH dosing of 0.60 mg/dog of B1-2K-PEG-DiAc-Insulin in dogs, Tₘₐₓ was about 0.5 hr with average Cₘₐₓ of 12 ng/mL. T_{1/2} was about 5.8 hr.

In summary, the terminal T_{1/2} of B1-2K-PEG-DiAc-Insulin was increased as compared to Insulin. Furthermore, T_{1/2} following IH dosing was significantly longer than after IV dosing. The calculated IH bioavailability was about 28%.

### B1-5K-PEG-DiAc-Insulin PK Following IV and IH Dosing in Dogs

Following IV dosing of 0.084 mg/dog of B1-5K-PEG-DiAc-Insulin in dogs, Cₘₐₓ appeared at the first sampling time point (i.e., 0.08 hr), with average Cₘₐₓ of 87 ng/mL The central volume of distribution (V_{c}) was about 115 mL/kg, or roughly twice of plasma volume in dog (i.e., 52 mL/kg). T₁₁₂ was about 0.76 hr, similar to B1-2K-PEG-DiAc-Insulin following IV dosing.

Following IH dosing of 2.0 mg/dog of B1-5K-PEG-DiAc-Insulin in dogs, Tₘₐₓ was about 2.5 hr (ranging from 1 to 8 hr) with average Cₘₐₓ of 21 ng/mL. Average T_{1/2} was about 15 hr, ranging from 6.6 to 51 hr.

In summary, the terminal T_{1/2} of B1-5K-PEG-DiAc-Insulin was increased as compared to Insulin, but is similar to B1-2K-PEG-DiAc-Insulin. T_{1/2} following IH dosing was significantly longer than after IV dosing. The calculated IH bioavailability was about 36%.

### Pharmacodynamics

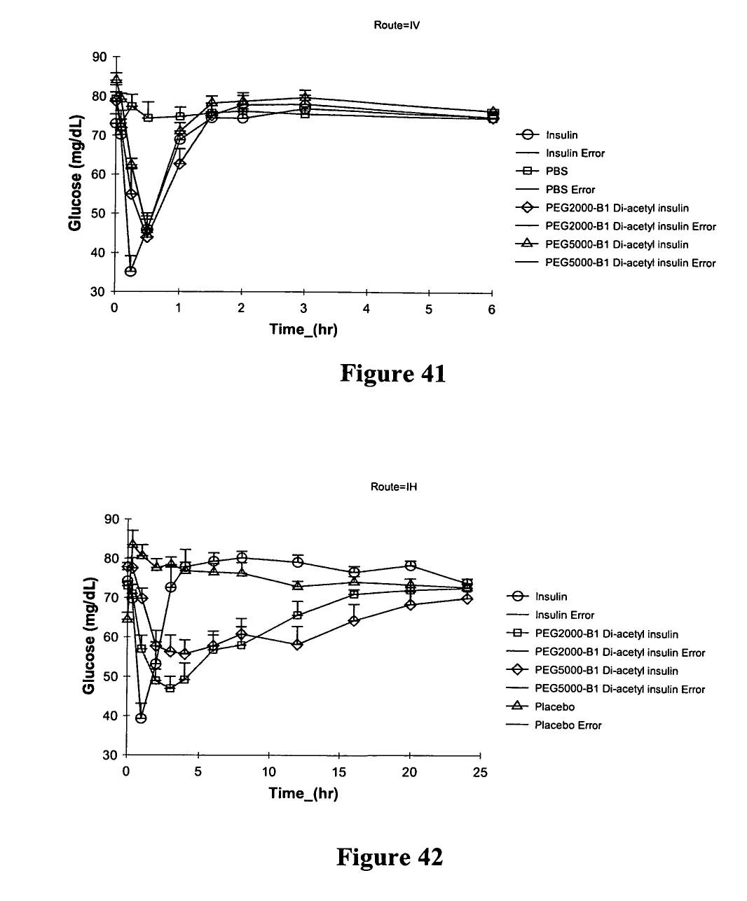

FIGS. 41 and 42 show the blood glucose profiles (mean±SE) following IV and IH dosing in dogs, respectively.

Following IV doses of Insulin, B1-2K-PEG-DiAc-Insulin, and B1-5K-PEG-DiAc-Insulin, the overall glucose suppressions were similar in terms of maximum suppression and duration of action, consistent with the limited increase in terminal T_{1/2} of PEGylated insulin following IV dosing. The time to reach maximum glucose suppression was delayed slightly in PEGylated insulin groups.

Following IH doses of Insulin, B1-2K-PEG-DiAc-Insulin, and B1-5K-PEG-DiAc-Insulin, the duration of glucose suppression was significantly increased in PEGylated insulin groups, in the order of B1-5K-PEG-DiAc-Insulin>B1-2K-PEG-DiAc-Insulin>Insulin. To calculate the overall glucose suppression, glucose levels were normalized by the equation:

GlucoseSuppression(t)=PredoseGlucoseConcentration−Glucose(t)

The glucose suppression-time profiles were integrated using linear-trapezoidal rule to calculate the area under the effect curve (AUEC).

The following table summarizes the overall effect expressed as glucose suppression AUEC and the corresponding IH lung doses of Insulin, B1-2K-PEG-DiAc-Insulin, and B1-5K-PEG-DiAc-Insulin. The specific effect (i.e., effect per mg of IH lung dose) was also calculated for the three test articles. Relatively, with same mg of IH lung dose delivered in dogs, the effect of B1-2K-PEG-DiAc-Insulin was roughly 2-fold of Insulin, whereas the effect of B1-5K-PEG-DiAc-Insulin was similar to insulin.

TABLE 15
Mean PK and PD Parameters
Following IH Administration of Insulin and PEG-Insulins to Dogs

| | | Effect | |
|---|---|---|---|
| | IH Dose | (Glucose Suppression | Effect/mg |
| Drug | (lung dose, mg) | AUEC) | Dosed |
| rh Insulin | 0.34 | 53 | 156 |
| 2K PEG DiAc rh | 0.6 | 220 | 367 |
| Insulin | | | |
| 5K PEG DiAc rh | 2 | 358 | 179 |
| Insulin | | | |

The results from the present study showed that the two analogs tested, B1-2K-PEG-DiAc-Insulin and B1-2K-PEG-DiAc-Insulin, achieved significantly prolonged duration of glucose suppression following powder inhalation in beagle dogs. The duration of suppression is significantly longer than what was observed for the B29-PEGylated insulins. Despite the difference in study design (fasting dog vs. glucose clamp), the conclusion holds that B1-PEGylated, A1, B29-diacetylated insulin analogs are superior in terms of efficacy (i.e., duration of effect). The durations of suppression achieved with B1-PEGylated A1, B29-diacetylated Insulins are likely to be able to provide overnight relief of hyperglycemia.

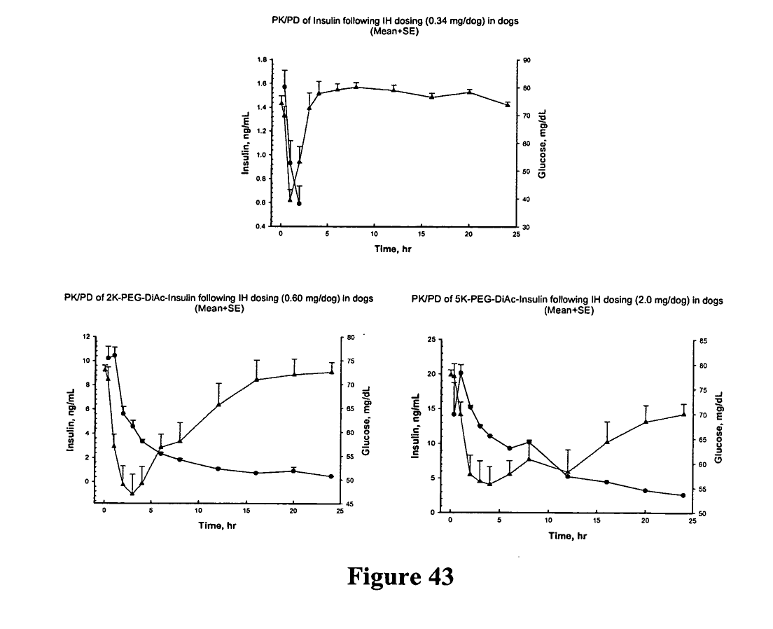

Furthermore, the PK profiles of IH B1-2K-PEG-DiAc-Insulin and B1-2K-PEG-DiAc-Insulin were consistent with their prolonged pharmacodynamic profiles. The duration of glucose suppression effect coincided with the duration of active plasma concentration exposure. FIG. 43 shows the overlay of average insulin and glucose levels following IH dosing in dogs.

### Example 27

### Pharmacokinetic/Pharmacodynamic Evaluation of Mono-PEG2000 Butyraldehyde Di-Acetylated Human Insulin Following Administration Via Intratracheal Instillation to Sprague-Dawley Rats

This Example establishes the dose of mono-PEG2000 butyraldehyde di-acetylated human insulin required to achieve a target blood glucose reduction, and evaluates the pharmacokinetics/pharmacodynamics (PK/PD) of the PEGylated molecule when administered to rats via intratracheal instillation at the selected dose.

Mono-PEG2000 butyraldehyde di-acetylated (PEG2K-Ald-DiAc) insulin molecule (“the modified insulin”) was synthesized and purified in accordance with Example 16.

The liquid formulation was prepared in Dulbecco's phosphate buffered saline (DPBS) for IT administration. Stock solutions of the modified insulin were prepared at insulin equivalent concentrations of 0.90 and 1.27 mg/mL. The dose was administered at a fixed volume of 300 μL; as a result, dosing solutions were prepared within 2 hours of administration by dilution of the stock solution.

The first leg, dose range finding, of this Example established the appropriate IT dose of the modified insulin for the subsequent PK/PD study. Five doses ranging from 80-381 μg per animal were compared in parallel where the target doses of the modified insulin were administered to Group 1 animals on the same day. The lowest dose that met the criteria of achieving nadir blood glucose concentrations between approximately 40 and 60 mg/dL in the first 4 hours after compound administration was chosen for the second leg of the study.

The second leg, the PK/PD study, evaluated the glucose suppression activity and blood concentration kinetics of the modified insulin when administered by IT instillation. During this leg of the study, Group 2 animals received a 270-μg dose of the modified insulin.

The test system included 19 pre-cannulated (jugular vein catheter (JVC)) adult male fasted (˜15-20 hours) Sprague-Dawley rats (Hilltop Lab Animals, Inc., Scottdale, Pa.). Ten animals weighing between 0.285-0.308 kg were used for the dose-ranging leg and 9 animals weighing between 0.288-0.313 kg were used for the PK/PD leg. Prior to dosing, the rats were lightly anesthetized by inhaled Isoflurane (Abbott Laboratories, Chicago, Ill.). Each animal was administered with the target dose of the modified insulin by IT instillation into the lungs. Venous blood samples of approximately 0.45 mL were collected from the JVC at the following time points for the dose-ranging and PK/PD studies, respectively: predose (˜0.16-0.33 hours prior to dosing), 0.33, 1, 2, 3, 4, 6, and 8 hours post-dose and predose (˜0.75 hours prior to dosing), 0.33, 1, 2, 3, 4, 6, 8, 12, 16, and 24 hours post-dose. Two readings of whole blood glucose concentration (mg/dL) per time point were made using a Glucometer Elite glucose monitor (Bayer Corp., Elkhart, Ind.). The remaining portion of the blood sample was processed into plasma and stored at −80° C. for possible analysis for modified insulin concentrations by enzyme-linked immunosorbent assay procedures. Pharmacodynamic analysis was performed using Microsoft Office Excel 2003.

Table 16 summarizes the study design.

TABLE 16

| | | | | Total Dose | Dose | Days |
|---|---|---|---|---|---|---|
| Group | Control/Test | | Animals/ | of Insulin | Volume | of |
| No. | Article | Route | Gender | (μg/animal) | (μL) | Dosing |
| 1 | PEG2K-Ald- | IT | 2/M | 80 | 300 | 1 |
| | DiAc Insulin | IT | 2/M | 160 | 300 | 1 |
| | | IT | 2/M | 200 | 300 | 1 |
| | | IT | 2/M | 270 | 300 | 1 |
| | | IT | 2/M | 381 | 300 | 1 |
| 2 | PEG2K-AW- | IT | 9/M | 270 | 300 | 1 |
| | DiAc Insulin | | | | | |

Dose Ranging = Group 1
PK/PD = Group 2

Table 17 shows a summary of selected individual and mean pharmacodynamic parameters for the dose ranging leg of the study, after mono-PEG2000 butyraldehyde di-acetylated insulin was administered by intratracheal instillation to male rats.

TABLE 17

| | | | Dose | Cₘᵢₙ | Tₘᵢₙ |
|---|---|---|---|---|---|
| Group | Animal | Test Article | (μg/animal) | (mg/dL) | (hr) |
| 1 | 1 | PEG2K-Ald-DiAc | 80 | 78 | 8 |
| | | Insulin | | | |
| 1 | 2 | PEG2K-Ald-DiAc | 80 | 85 | 1 |
| | | Insulin | | | |
| Mean | | | | 81 | 4.50 |
| SD | | | | 5 | 4.95 |
| 1 | 3 | PEG2K-Ald-DiAc | 160 | 86 | 6 |
| | | Insulin | | | |
| 1 | 4 | PEG2K-Ald-DiAc | 160 | 82 | 6 |
| | | Insulin | | | |
| Mean | | | | 84 | 6.00 |
| SD | | | | 6 | 0.00 |
| 1 | 5 | PEG2K-Ald-DiAc | 200 | 109 | 1 |
| | | Insulin | | | |
| 1 | 6 | PEG2K-Ald-DiAc | 200 | 37 | 6 |
| | | Insulin | | | |
| Mean | | | | 73 | 3.50 |
| SD | | | | 42 | 3.54 |
| 1 | 7 | PEG2K-Ald-DiAc | 270 | 31 | 3 |
| | | Insulin | | | |
| 1 | 8 | PEG2K-Ald-DiAc | 270 | 35 | 8 |
| | | Insulin | | | |
| Mean | | | | 33 | 5.50 |
| SD | | | | 3 | 5.54 |
| 1 | 9 | PEG2K-Ald-DiAc | 381 | 20 | 4 |
| | | Insulin | | | |
| 1 | 10 | PEG2K-Ald-DiAc | 381 | 19^{a} | 4 |
| | | Insulin | | | |
| Mean | | | | 20 | 4.00 |
| SD | | | | 1 | 0.00 |

^{a}Below glucometer measurement range, entered as 19 mg/dL for calculation of Mean and SD;
Cₘᵢₙ = Minimum blood glucose concentration;
Tₘᵢₙ = Time to Cₘᵢₙ;
SD = Standard deviation
Animals 1-9 and 1-10 were euthanized at 4 hr due to hypoglycemia symptoms

TABLE 18
Table 18 presents a summary of selected individual and mean
pharmacodynamic parameters for the PK/PD leg of the study,
after mono-PEG2000 butyraldehyde di-acetylated insulin was
administered by intratracheal instillation to male rats.

| | | | Dose | | |
|---|---|---|---|---|---|
| | | | (μg/ | Cₘᵢₙ | Tₘᵢₙ |
| Group | Animal | Test Article | animal) | (mg/dL) | (hr) |
| 2 | 1 | PEG2K-Ald-DiAc Insulin | 270 | 19^{a} | 8 |
| 2 | 2 | PEG2K-Ald-DiAc Insulin | 270 | 89 | 1 |
| 2 | 3 | PEG2K-Ald-DiAc Insulin | 270 | 93 | 1 |
| 2 | 4 | PEG2K-Ald-DiAc Insulin | 270 | 19 | 6 |
| 2 | 5 | PEG2K-Ald-DiAc Insulin | 270 | 46 | 8 |
| 2 | 6 | PEG2K-Ald-DiAc Insulin | 270 | 59 | 6 |
| 2 | 7 | PEG2K-Ald-DiAc Insulin | 270 | 35 | 4 |
| 2 | 8 | PEG2K-Ald-DiAc Insulin | 270 | 83 | 12 |
| 2 | 9 | PEG2K-Ald-DiAc Insulin | 270 | 26 | 6 |
| Mean | | | | 52 | 5.80 |
| SD | | | | 29 | 3.50 |

^{a}Below glucometer measurement range, entered as 19 mg/dL for calculation of Mean and SD;
Cₘᵢₙ = Minimum blood glucose concentration;
Tₘᵢₙ = Time to Cₘᵢₙ;
SD = Standard deviation
Animals 2-1 and 2-4 were either euthanized, due to hypoglycemia, or found dead at 12 hr

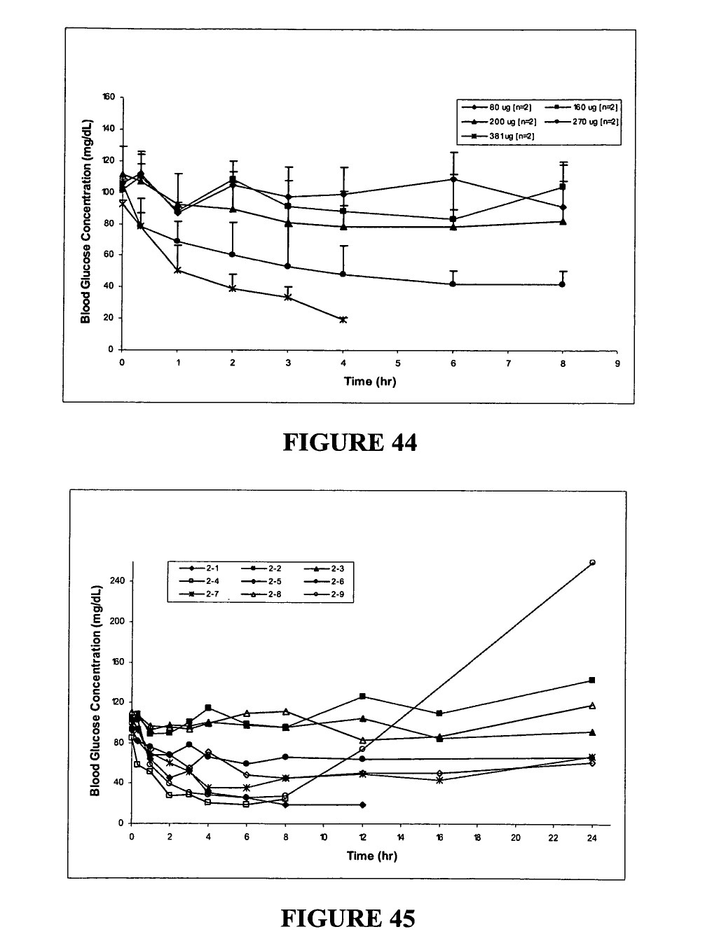

FIG. 44 shows mean blood glucose concentrations following 80-381 μg intratracheal instillation of mono-PEG2000 butyraldehyde di-acetylated insulin to Sprague-Dawley rats (Group 1—dose ranging study). (Note: Error bars indicate standard deviation; 381-μg dose animals were euthanized at 4 hr post-dose due to hypoglycemia symptoms.)

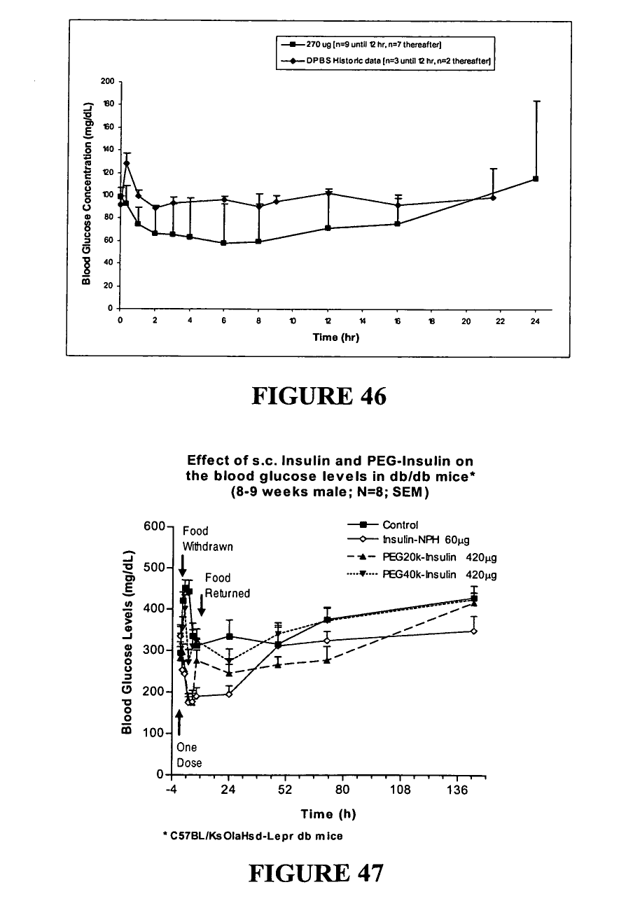

FIG. 45 shows individual blood glucose concentrations following 270 μg intratracheal instillation of mono-PEG2000 butyraldehyde di-acetylated insulin to Sprague-Dawley rats (n=9 until 8 hr, n=8 until 12 hr, n=7 thereafter) (Group 2—PK/PD study). (Note: Animal 2-1 euthanized at 12 hr due to hypoglycemia symptoms; Animal 2-4 found dead at 12 hr; Animals 2-2 and 2-9 sample at 24 hr collected via cardiac puncture due to in-operative catheter; and Animals 2-6 and 2-9 sample at 16 hr unobtainable due to in-operative catheter.) FIG. 46 shows mean blood glucose concentrations following 270 μg intratracheal instillation of mono-PEG2000 butyraldehyde di-acetylated insulin to Sprague-Dawley rats (Group 2—PK/PD study). (Note: Error bars are standard deviation; DPBS data from a previous study.)

### Discussion

Doses ≦270 μg were prepared from a 0.90 mg/mL solution batch, and the 381-μg dose was prepared from a second solution batch of 1.27 mg/mL.

In the dose range finding leg of this study (FIG. 44), doses of up to 160 μg/animal barely met the criteria of achieving nadir blood glucose concentrations between approximately 40 and 60 mg/dL, whereas 381 μg/animal caused hypoglycemia (<20 mg/dL blood glucose concentration). Consequently, Animals 9 and 10 of the highest dose level were euthanized after 4 hours post-dose due to convulsions because of hypoglycemia. Since only one animal responded at the 200 μg dose (FIG. 46), 270 μg was selected as the dose for the PK/PD leg and resulted in the following mean nadir blood glucose levels and times:

| | Insulin Dose | Cₘᵢₙ | Tₘᵢₙ |
|---|---|---|---|
| Test Article | (μg/animal) | (mg/dL) | (hr) |
| PEG2K-Ald-DiAc Insulin | 270 | 52 ± 29 | 5.8 ± 3.5 |

Cₘᵢₙ = Minimum blood glucose concentration;
Tₘᵢₙ = Time to Cₘᵢₙ

In the PK/PD leg, variability was seen in glucose profiles. As seen in FIG. 45, two out of nine animals exceeded the target glucose reduction; at twelve hours post-dose, Animal 2-1 was euthanized after sample collection due to hypoglycemia, and Animal 2-4 was found dead. On the other hand, target glucose suppression was not achieved in three out of nine animals (2-2, 2-3, and 2-8). Glucose measurements were higher at twenty-four hours for Animals 2-2 and 2-9 because samples were collected via terminal cardiac venipuncture due to in-operative JVC. Overall, when comparing the mean blood glucose concentrations of the modified insulin-treated and saline (historic data) treated animals, mono-PEG2000 butyraldehyde di-acetylated insulin does have a pharmacological effect (FIG. 46) at a 200-270 μg/animal dose.

### Example 28

### Delivery of Liquid Formulations of 5K PEG, Di-Ac Insulin

In this Example, liquid formulations (25 mg/ml and 50 mg/ml of 5K PEG, di-Ac Insulin) were delivered by a nebulizer. The delivered dose and VMD were evaluated for consistency from 1 to 30 actuations. The drug solution integrity and concentration were measured pre- and post-nebulization.

Approximately 3 mL of the formulation was dispensed into a Salter jet nebulizer (Salter Lab, Tri-Anim, Sylmar, Calif.). The nebulizer was operated using a driving pressure of ˜60 psig supplied via a Koko dosimeter (Koko, Boulder, Colo.) to give an output flow rate of about 15 LPM. The dosimeter was set at auto tidal volume with a dose delay of 0.3 seconds and to discharge for 3.0 seconds per actuation. Each formulation was tested twice (replicate 1 and replicate 2). The results are shown below, including standard deviation data.

| | | Emitted Mass/Puff | VMD |
|---|---|---|---|
| Formulation | Replicate | (mg/puff) | (μm) |
| 25 mg/ml | 1 | 0.7 ± 0.1 | 3.5 ± 0.3 |
| | 2 | 0.7 ± 0.0 | 3.2 ± 0.2 |
| 50 mg/ml | 1 | NA | 3.6 ± 0.3 |
| | 2 | NA | 3.5 ± 0.2 |

An aliquot (100 μl) of the 50 mg/ml solution was taken pre- and post-nebulization for analysis. Concentration, as measured by UV, increased as the actuation number increased due to evaporation of solution during nebulization. The concentrations at pre- and post-nebulization were measured to be higher than intended (57 mg/ml and 66 mg/ml).

### Example 29

### Delivery of a Liquid Formulation of 5K PEG, Di-Ac Insulin

In this Example, liquid formulations (25 mg/ml, 25 mg/ml, and 45 mg/ml of 5K PEG, di-Ac Insulin) were delivered by a nebulizer. The delivered dose was evaluated for consistency from 1 to 30 actuations.

Approximately 3 mL of the formulation was dispensed into a Salter jet nebulizer (Salter Lab, Tri-Anim, Sylmar, Calif.). The nebulizer was operated using a driving pressure of ˜60 psig supplied via a Koko dosimeter (Koko, Boulder, Colo.) to give an output flow rate of about 15 LPM. The dosimeter was set at auto tidal volume with a dose delay of 0.3 seconds and to discharge for 3.0 seconds per actuation. Each formulation was tested twice (replicate 1 and replicate 2). The results are shown below.

| | | Emitted Mass/Puff | % RSD of |
|---|---|---|---|
| Formulation | Replicate | (mg/puff) | Emitted Mass/Puff |
| 25 mg/ml | 1 | 0.34 | 2.7 |
| | 2 | 0.38 | 8.7 |
| 35 mg/ml | 1 | 0.46 | 4.9 |
| | 2 | 0.46 | 4.9 |
| 45 mg/ml | 1 | 0.64 | 7.3 |
| | 2 | 0.71 | 7.9 |

In view of the above, dose consistency was best at the 35 mg/ml concentration. The above data also allowed estimation of the number of actuations (puffs) for given target doses as follows.

| | Minimum Number of Actuations | | |
|---|---|---|---|
| Target Dose | 25 mg/ml | 35 mg/ml | 45 mg/ml |
| 2.5 mg | 9 | 7 | 5 |
| 5.0 mg | 19 | 14 | 10 |
| 10.0 mg | 37 | 29 | 20 |

### Example 30

### Delivery of Liquid Formulations of 2K PEG, Di-Ac Insulin

In this Example, liquid formulations (20 mg/ml and 25 mg/ml of 2K PEG, di-Ac Insulin) were delivered by a nebulizer. The delivered dose and VMD were evaluated. The drug solution integrity and concentration were measured pre- and post-nebulization.

Approximately 3 mL of the formulation was dispensed into a Salter jet nebulizer (Salter Lab, Tri-Anim, Sylmar, Calif.). The nebulizer was operated using a driving pressure of ˜60 psig supplied via a Koko dosimeter (Koko, Boulder, Colo.) to give an output flow rate of about 13 LPM. The dosimeter was set at auto tidal volume with a dose delay of 0.3 seconds and to discharge for 3.0 seconds per actuation. The results are shown below.

| | Emitted Mass/Puff | VMD |
|---|---|---|
| Formulation | (mg/Puff)¹ | (μm) |
| 20 mg/ml | 0.41 | 3.8² |
| 25 mg/ml | 0.47 | 3.5-3.8³ |

¹Mean values of puffs 8-10
²Measurement taken at >15 puffs
³Measurement taken at about 15 puffs

An aliquot (100 μl) of the solutions was taken pre- and post-nebulization for analysis. Concentration, as measured by UV, increased as the actuation number increased due to evaporation of solution during nebulization. The concentration of the 20 mg/ml solution at pre-nebulization and at puff 15 were measured to be 20.3 mg/ml and 21.3 mg/ml, respectively. The concentration of the 25 mg/ml solution at pre-nebulization and at puff 15 were measured to be 24.9 mg/ml and 26.8 mg/ml, respectively.

### Example 31

### Prophetic Example of Human Administration of Liquid Formulation of 5K PEG, Di-Ac Insulin Combined with Some Actually Done In Vitro Work

5K PEG, di-Ac insulin will be supplied as a lyophilized powder in 30 mL vials containing 140 mg of PEG-Insulin per vial. Upon reconstitution with 4 mL of PBS, the inhalation solution will be a clear, colorless solution formulated as preservative-free solution with a final concentration of 35 mg/mL. Approximately 3 mL of 35 mg/mL will be dispensed into a nebulizer for dosing.

A commercial nebulizer (Salter Lab, Tri-Anim, Sylmar, Calif.) will be used for dosing of each test subject. The nebulizer will operate using a driving pressure of ˜60 psig supplied via a Koko dosimeter (Koko, Boulder, Colo.). The dosimeter enables delivery of a consistent dose of nebulized drug solution (3 mL of 35 mg/mL 5K PEG, di-Ac insulin) from the nebulizer. The dosimeter will automatically discharge the nebulizer each time the subject inhales. The beginning of nebulizer discharge will occur 0.3 seconds after the onset of inhalation, and the duration of nebulizer discharge will be 3.0 seconds followed by a 5-second breath-hold.

The below Table presents in vitro results of the delivered dose per inhalation and volume median diameter (VMD) measured at the mouthpiece. Aerosol performance measurements up to 30 inhalations were characterized. Therefore, the range of insulin delivered at the mouthpiece was 0.43 mg (1 inhalation) to 12.90 mg (30 inhalations).

Delivered dose and VMD at mouthpiece

| | Results |
|---|---|
| Amount of insulin delivered to mouthpiece per | 0.43 ± 0.04 mg |
| inhalation¹ | |
| VMD² | 3.0-4.0 μm |
| Estimated number of inhalations needed to reach a | 8 |
| 2.5 mg insulin lung dose³ | |

¹Mean across 6 nebulizers (30 inhalations each)
²VMD range across 3 nebulizers (30 inhalations each) measured with a light diffraction instrument, Spraytec (Malvern, Inc, USA)
³Derived from: (2.5 mg * 1/CF)/Delivered dose at mouthpiece, where CF is a correction factor of 70% to 80% used in estimating lung dose

The effect of nebulization on the integrity of 5K PEG, di-Ac insulin formulation was evaluated by collecting nebulized aerosols using a modified version of a controlled test tube impactor apparatus described in U.S. Pat. No. 5,457,044, which is incorporated herein by reference. Concentrations of collected samples were determined using UV spectroscopy, while the integrity analysis of the collected protein was evaluated by HPLC. The recovery of the aerosolized protein was estimated to be 94% (see below Table). The results indicated that 5K PEG, di-Ac insulin was stable to nebulization.

Collection and recovery of nebulized NKT-10144 formulation

| | |
|---|---|
| Amount collected as aerosol | 14.3 mg |
| Amount on filter | 0.2 mg |
| % Collected as aerosol | 98%¹ |

¹Calculated using 14.3/(14.3 + 0.2) * 100%

### Example 32

### In-Vivo Study in Mice to Examine the Blood-Glucose Lowering Effects of Illustrative Releasable Polymer Conjugates of Insulin

Male diabetic mice (BKS.Cg-+Lepr db/+Lepr db/01aHsd) were purchased from Harlan Laboratories, Ltd. (Jerusalem, Israel). The 8-9 week old animals (30-40 gm) were placed in mouse cages (two animals per cage), and allowed at least 48 hours of acclimatization before the start of the study.

The preparation of G2-PEG2-FMOC-NHS 20K or 40K insulin—random and G2-PEG2-FMOC-NHS 20K or 40K insulin—site specific (B1) is described in Examples 20 and 21, respectively. Each compound was accurately weighed into a glass vial and dissolved in normal saline in order to prepare a concentration that would accommodate for the dose (based on insulin equivalents) and the injection volume of 100 μl.

The study was divided into two phases: a feasibility phase and an evaluation phase.

In the feasibility phase, the feasibility of using diabetic db/db mice to test the effectiveness of the compounds was first evaluated. In carrying out the feasibility phase, several groups of mice were used wherein four mice were used in each group. Data on the baseline glucose levels were gathered for each mouse for 2-3 days prior to drug dosing. This was performed to identify any outliers in the group of animals. On the day of treatment (Day 0) each animal was weighed. A time 0 day blood sample (5 to 10 μL) was collected from the tail vein. The glucose level (mg/dl) was measured using a glucose analyzer. Each animal was then dosed subcutaneously (SC) below the skin on the back. The amount of test article and the dose (60 and 120 μg/mouse) administered was based on the average body weight of the animal, and the total volume of the dose did not exceed 10 ml/kg. The animals were then allowed to return into their cages. Blood samples of 5 to 10 μl (<0.5% of 2 ml blood volume for a 35 g mouse) were removed through a needle prick/capillary tube at the following time points: −3, −2, −1, 0, 0.04, 0.16, 0.33, 1.0, 1.16 days. Each collected blood sample was tested for its glucose level.

In the evaluation phase, the results from the feasibility phase were used to select the appropriate doses required to attain a sustained delivery of insulin. In carrying out the evaluation phase, eight mice were used in each group. Data on the baseline glucose levels were gathered for each mouse three days prior to drug dosing. On the day of treatment (Day 0) each animal was weighed. A time 0 day blood sample (5 to 10 μl) was collected from the tail vein. The glucose level (mg/dl) was measured using a glucose analyzer. Each animal was then dosed subcutaneously (SC) below the skin on the back. The amount of test article administered was based on the average body weight of the animal, and the total volume of the dose did not exceed 10 ml/kg. The animals were then allowed to return into their cages. Blood samples of 5 to 10 μl (<0.5% of 2 ml blood volume for a 35 g mouse) were removed through a needle prick/capillary tube at the following time points: −3, −2, −1, 0, 0.04, 0.16, 0.33, 0.5, 1, 2, 3, 6 days. Each collected blood sample was for its glucose level. Food was withdrawn from the animals for the first four hours after dosing.

The below Table describes the test compounds and the dose for each group of animals.

Test Compounds and Dose for Each Group of Animals

| | Number of mice per | |
|---|---|---|
| Treatment | group | Dose (in μg) |
| Negative control (saline) | 8 | — |
| Positive control (NPH insulin) | 8 | 60 |
| G2-PEG2-FMOC-NHS 20K or 40K | 8 | 420 |
| insulin - random (A1, B29) | | |
| G2-PEG2-FMOC-NHS 20K or 40K | 8 | 420 |
| insulin - random (A1, B29) | | |
| G2-PEG2-FMOC-NHS 20K or 40K | 8 | 840 |
| insulin - site specific (B1, B29) | | |
| G2-PEG2-FMOC-NHS 20K or 40K | 8 | 840 |
| insulin - site specific (B1, B29) | | |

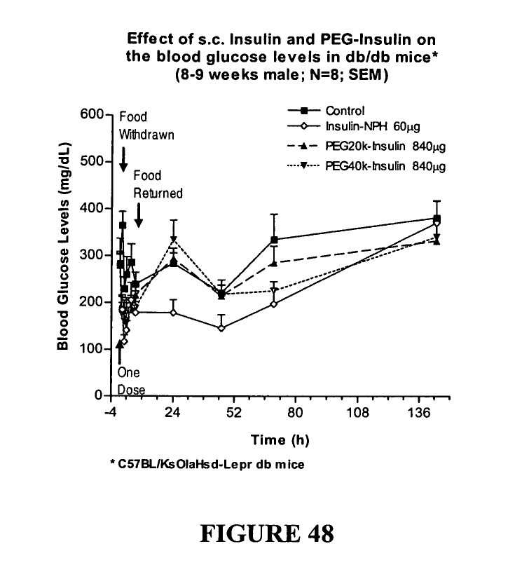

The data from the study was collected and analyzed. It was noted that the animals tolerated the single subcutaneous dose. As illustrated in FIGS. 47 and 48, the blood glucose-lowering effect of G2-PEG2-FMOC-NHS 20K or 40K insulin-random and G2-PEG2-FMOC-NHS 20K or 40K insulin-site specific was confirmed.

### Example 33

### Analytical Ion Exchange Chromatography of PEGylated Insulin Mixtures

The reaction components for the derivitization of insulin were as follows. Humanized porcine insulin by Akzo Nobel (Diosynth, France). An “activated” mPEG-succinimidyl propionate 750 to 5000 Da by Nektar Therapeutics. For acetylation reactions with insulin, acetic acid-NHS (N-hydroxy succinimide), (ICN Biomedical) was used. For some reactions acetylation was done first then the acetylated insulin was further PEGylated.

The reaction conditions were carried out in an aqueous/organic solvent system with specific pH control to produce high yields of specific derivatives. Even so, side reactions occurred for all procedures and produced mixtures that required purification and quantitation. Some derivatives were made at large scale and purified by large scale cation exchange chromatography, while smaller scale procedures used C-18 RP-HPLC for purification.

The matrix used for the Mass Spectrometry samples was sinapinic acid. The Voyager DE-Pro was operated in the linear mode, with positive ions monitored. The 337 nm line of the nitrogen laser was used with about 200 shots averaged for the final spectrum. The spectra were produced by M-Scan Inc. of West Chester, Pa.

The RP-HPLC method used a Superspher (EM Science) C-18, 4 μm, 3 mm×250 mm column. Mobile phase A consisted 7% Acetonitrile 140 mM sodium perchlorate, pH adjusted to 2.3 with phosphoric acid. Mobile phase B 57% acetonitrile 65 mM sodium perchlorate pH adjusted to 2.3 with phosphoric acid. The initial starting condition is isocractic at 55% B for 30 minutes, then a gradient to 80% B at 55 minutes. Flow rate is 0.55 ml/minute. Detector was set at 214 nm.

Cation Exchange: GE Healthcare Mini-PE 4.6×50 mm 0.8 mL/min. Two mobile phase systems were used. For system 1 was mobile phase A consisted of 40% acetonitrile 1 M acetic acid. Mobile phase B consisted 40% acetonitrile 1 M acetic acid and 300 mM sodium perchlorate. Gradient was 1% B at 0 minutes to 80% B at 2 minutes. Flow rate was 0.8 mL/minute, monitored at 276 nm.

System 2 mobile phase A consisted of 30% acetonitrile, and 0.1% TFA, Gradient was 0% B at 0 minutes to 80% B at 20 minutes. Mobile phase B was 30% acetonitrile, 0.1% TFA and 300 mM NaClO4. Monitored at 214 nm, and 276 nm.

Anion Exchange: GE Healthcare Mini-Q PE 4.6×50 mm 0.8 mL/min, Mobile phase A consisted of 20% acetonitrile 50 mM Tris at pH 8.8. Mobile phase B consisted of 20% acetonitrile 50 mM Tris 300 mM NaCl at pH 8.8. Gradient was 0% B at 0 min. to 80% B at 20 min. Flow rate 0.8 mL/minute. Monitored 276 nm. pH was modified as needed.

All chromatography work was performed on Waters 2690 pumps with photo diode array detectors, or Agilent 1200 systems with variable wavelength detectors.

Sample concentrations were approximately 0.2 mM and injection volumes 10 to 50 μl.

One of the most common reactions used to derivatize proteins is an activated ester which reacts with primary amines. The primary amines of insulin are the A-chain N terminus (A1) the B-chain terminus (B1) and the lysine side chain on B29. Acetic acid N-hydroxysuccinimide (acetic acid-NHS) was used for acetylation, and PEG-succinimidyl propionic acid (PEG-SPA) for PEGylation. The NHS was chosen for high specificity and fewer side reactions, as opposed to anhydrides or other moieties.

When these activated esters covalently react with the insulin amines the three Mono, Di, and Tri-derivatives result, in addition the below Table shows how this process creates multiple sub-species of Mono, Di and Tri-derivatives. To assess yields of such reactions requires a reliable method that can quickly separate all of the sub-species of conjugated insulin.

Potential mixture of seven species of insulin
generated from derivitization process

| Insulin | Mono-derivatives | | | Di-derivatives | | | Tri-derivatives |
|---|---|---|---|---|---|---|---|
| Species | | | | | | | |
| Insulin | A1 | B1 | B29 | A1- | A1- | B1- | A1-B1-B29 |
| Sub-species | | | | B1 | B29 | B29 | |

As a model to assess reaction kinetics a mixture of acetylated insulin derivatives were created by reacting 3 mols of acetic-NHS with 1 mol of insulin in an aqueous/organic solution. The reaction conditions produced a mixture of Tri-acetylated insulin, two different Di-acetylated derivatives, and one mono-acetylated derivative (data not shown). The Mono, Di and Tri-acetylated species were confirmed by fraction collection and subsequent analysis by LC-MS. Interestingly, no mono-A1 species were found. The mixture was used to optimize the cation exchange method using the Mini S column.

The mini-S column and a classical cation exchange mobile phase were evaluated. Mobile phase A consisted of 7M urea and 1M acetic acid, and mobile phase B consisted of 7M urea 1M acetic acid, and 300 mM NaCl. The gradient was 0% B to 80% B in 20 minutes. The peak shapes were very poor (data not shown). Acetonitrile was used to replace the urea as a chaotropic agent. The peak shapes and retention were greatly improved (data not shown). The final mobile phase is listed in the previous methods section as mobile phase system 1 for cation exchange, 40% acetonitrile and 300 mM sodium perchlorate, and 1 M acetic acid.

A randomly PEGylated insulin mixture was created by reacting 2.5 mols of PEG-SPA (750 Da) with 1 mol of insulin in a mixed solvent (organic/aqueous) solution. MALDI mass spectra were generated on the final 750 PEG insulin reaction mixture (data not shown). The MALDI confirms only mono-PEG and Di-PEGylated insulin species are present. Insulin at 5808 Da plus 750 Da PEG is 6558 (main peak), and 5808 plus 1500 Da (2 PEG chains) is 7308 (second peak) in addition the spectra shows the expected normal distribution of longer and shorter chains of PEG attached. PEG chains vary by 44 Da for each PEG unit, but the average molecular weight of this sample is a 750 Da PEG chain attached at each conjugation site.

The randomly 750 PEGylated insulin was injected on RP-HPLC and the Mini-S cation exchange method (data not shown). The powerful resolution of cation exchange allows quantitation the sub-species Mono-PEG A1 and Mono-PEG B29, which was not resolved by RP-HPLC or MALDI.

The peak assignments in the cation exchange chromatogram were assigned through a series of peptide mapping experiments using the USP peptide mapping test for human insulin. The experiment is too elaborate for discussion here, but briefly outlined the method essentially cuts the insulin into four fragments, and fortuitously each derivitization site is isolated on one fragment. If the site is derivatized the fragment elutes much later and the degree of site derivitization can be quantitated compared to a reference standard.

As various PEG-insulin derivatization schemes were conceived and synthesized, an insulin derivative acetylated on A1 and B29, and PEGylated on B1 became a molecule of interest. Referred to as (B1PEG Di-Ac), a 5K and 2K version were synthesized and purified. This triply derivatized insulin had poor retention by cation exchange (data not shown). Evaluation of the molecule reveals that there are only 3 of the original 6 positive charges remaining compared to insulin, for an overall net charge of +3 at pH 2.0. Decrease in overall positive charge explains why it eluted very near the void volume at about 1-2 minutes (data not shown).

To proceed rationally to the anion exchange column the theoretical charge state versus the pH of a tri-derivatized insulin, a mono derivative, and underivatized insulin were plotted (data not shown). The plot was created using the pKa values of the free amino acids that compose insulin. The B1PEG Di-Ac insulin, (data not shown) showed that at pH 7.0 to 8.0 it still retained 6 negative charges, and an overall charge of negative 5. Theoretically the derivative should be retained by the anion exchange column more effectively. To leverage this overall more negative state the derivatives behavior was evaluated on the anion exchange column (data not shown).

Three main derivative types were constructed for preclinical experiments (data not included here). Mono PEG insulin on B29, Mono PEG on B1 insulin, and Mono PEG on B1 with di-acetylation (one on B29 and one on A1). The anion exchange method was optimized to ensure purity of each derivative.

When the Mono-PEG B1 was injected on the anion exchange it demonstrated very poor retention and peak shape (data not shown). To optimize the behavior the mono-PEG's the theoretical charge vs. pH curve (data not shown) was referred to again. The graph shows the mono derivatives of insulin can be pushed from net (−3) charge at pH 7 to net (−5) charge at pH 9. The mobile phase was adjusted upward to pH 8.8 to drive the negative charges toward saturation.

The three main types of 5K PEG-insulin derivatives were injected. The improved chromatograms at pH 8.8 vs. pH 8.3 mobile phase were generated (data not shown). Also of note is the resolution of additional peaks in the B1 mono-PEG lots, indicating some residual B29 and tri-derivatized species from the synthesis and purification procedure. Another important finding illustrated in the chromatogram (data not shown) are the same derivative types, but with shorter length (2K) PEG chains. They are notably more retained by the column. This is another important tool for separation.

Pure lyophilized powder of mono-PEG B1 Di-Ac (2K) insulin was put in a 50° C. oven for 20 weeks. The peaks were not specifically characterized but the standard hydrolytic degradation products such as covalent dimers and deamidation products will be the main degradants. The anion exchange resolves these products even with the PEG attached. The C-18 RP-HPLC (not shown) did not have any clearly resolved peaks. For the anion exchange the purity for T=0 was 97.3% main peak. For T=20 weeks the purity was 93.8%. The main degradant at 12.5 minutes is 2.9%, and due to the high charge and high retention is most likely covalent insulin dimer

### Example 34

### Site Specific Acetylation of Brain Natriuretic Peptide (BNP-32)

In the present Example, the pKa difference between the N-terminal amine and the epsilon amines of the lysine residues of BNP-32 was used to specifically acetylate the N-terminus, leaving the lysine amines available for PEGylation.

One milligram of BNP-32 was combined with 2 mol equivalents of acetic acid-NHS (previously dissolved in 2 mM HCl) in a total volume of 1 mL in 20 mM MES buffer at pH 6.0 and incubated at room temperature for 2 h. At this pH, one predominant acetylated product was formed based on RP-HPLC analysis. Based on accepted chemical principles known to those skilled in the art, at pH 6.0 the N-terminal amine group was more reactive than the epsilon amines and acetylation would occur predominantly at this position. Also, at lower pH, all amines were less reactive while at higher pH all amines were more reactive. The reaction above was also performed at other pH levels: At pH 4.5 (20 mM citrate buffer) there was significantly lower acetylation for all amine groups, while at pH 7.5 (20 mM HEPES buffer) and pH 9.0 (20 mM boric acid buffer), all amine groups were more reactive and significant acetylation occurred at all four sites as assessed by RP-HPLC. Site specificity of the purified reaction products may also be confirmed using methods known to the art such as peptide mapping.

The predominant acetylated product from the reaction performed at pH 6.0 can be purified by standard chromatographic methods. The acetylated peptide can then be PEGylated using any of the reagents that are specific for amine reactive groups and standard methods known to the art, again followed by standard chromatographic methods to purify the conjugate of interest.

The following list of documents, some of which may be cited herein, may be referred to in consideration of this specification. All documents, including articles, books, patents, patent publications, and other publications, referenced herein, are incorporated by reference. The citation of any publication is for its disclosure prior to the filing date and should not be construed as an admission that such publication is prior art or that the present invention is not entitled to antedate such publication by virtue of prior invention.
Harris, J. M. and Chess, R. B. (2003) Effect of PEGylation on pharmaceuticals. Nature Reviews 2, 214-221.
Fineberg, S. E., (2002) Antibodies to Insulin and Other Biologics: Implications for Clinicians and Development. Respiratory Drug Delivery VIII, Vol 1, pp 43-50.
Uchio, T., Baudys, M., Liu, F., Song, S. C. and Kim, S. W. (1999) Site-specific insulin conjugates with enhanced stability and extended action profile. Adv. Drug Del. Rev. 35, 289-306.
Murray-Rust, J., McLeod, A. N., Blundell, T. L. and Wood, S. P. (1992) Structure and evolution of insulins: implication for receptor binding. BioEssays 14, 325-331.
Hinds, K., Koh, J. J., Joss, L., Liu, F., Baudys, M. and Kim, S. W. (2000) Synthesis and characterization of poly(ethylene glycol)-insulin conjugates. Bioconjugate Chem. 11, 195-201.
Donovan, M. D., Flynn, G. L. and Amidon, G. L. (1990) Absorption of polyethylene glycols 600 through 2000: The molecular weight dependence of gastrointestinal and nasal absorption. Pharm. Res. (7) 8, 863-868.
Niven, R. W., Whitcomb, K. L., Shaner, L., Ip, A. Y. and Kinstler, O. B. (1995) The pulmonary absorption of aerosolized and intratracheally instilled rhG-CSF and monoPEGylated rhG-CSF. Pharm. Res. (12) 9, 1343-1349.
Brange, J., Havelund, S., Hommel, E., Sorensen, E., and Kuhl, C., (1986) Neutral insulin solutions physically stabilized by addition of Zn²⁺. Diabetic Med. 3, 532-536.
Hinds, K. and Kim, S. W. (2002) Effects of PEG conjugation on insulin properties. Adv. Drug Del. Rev. 54, 505-530.
Wall, D. and Lanutti, A. T. (1993) High levels of exopeptidase activity and are present in rat and canine bronchoalveolar lavage fluid. Int. I Pharm. 97, 171-181.
Forbes, B., Wilson, C. G. and Gumbleton, M. (1999) Temporal dependence of ectopeptidase expression in alveolar epithelial cell culture: implications for study of peptide absorption. Int. J. Pharm. 180, 225-234.
Heise, T., Weyer, C., Serwad, A., Heinrichs, S., Osinga, J., Roach, P., Woodworth, J., Gudatk U., and Heinemann, L. (1998) Time-action profiles of novel premixed preparations of insulin Lispro and NPL insulin. Diabetes Care 21, 800-803.
Myers, S. R., Yakubu-Madus, F. E., Johnson, W. T., Baker, J. E., Cusick, T. S., Williams, V. K., Tinsley, F. C., Kriauciunas, A., Manetta, J., and Chen, V. J. (1997) Acylation of human insulin with palmitic acid extends the time action of human insulin in diabetic dogs. Diabetes, 46, 637-642.
Asada, H., Douen, T., Mizokoshi, Fujita, T., Murakami, M., Yamamoto, A., and Muranishi, S. (1994) Stability of acyl derivatives of insulin in the small intestine: relative importance of insulin association characteristics in aqueous solution. Pharmaceutical Research, 11, 1115-1120.
Berger, S., Davidson, M. H., Landschulz, W. H., Gelfand, R. A. Add-on Therapy with Inhaled Human Insulin in Type 2 Diabetic Patients Failing Oral Agents: Preliminary Results of a Multi-Center Trial, 2000.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

What is claimed is:

1. A modified therapeutic peptide having at least one amino acid residue covalently attached to a hydrophilic polymer and at least one amino acid covalently attached to a moiety having one to three carbon atoms.

2. The modified therapeutic peptide according to claim 1, wherein the hydrophilic polymer comprises a non-naturally occurring polymer.

3. The modified therapeutic peptide according to claim 1, wherein the hydrophilic polymer comprises a non-peptidic polymer.

4. The modified therapeutic peptide according to claim 1, wherein the hydrophilic polymer is attached to the therapeutic peptide via a linker.

5. The modified therapeutic peptide according to claim 1, wherein the hydrophilic polymer comprises a polymer of ethoxy monomers.

6. The modified therapeutic peptide according to claim 1, wherein the hydrophilic polymer comprises polyethylene glycol.

7. The modified therapeutic peptide according to claim 1, wherein the polyethylene glycol has a molecular weight of less than or equal to 10,000 Daltons.

8. The modified therapeutic peptide according to claim 1, wherein the polyethylene glycol has a molecular weight of less than or equal to 5000 Daltons.

9. The modified therapeutic peptide according to claim 1, wherein the modified therapeutic peptide comprises an amino acid sequence of human insulin.

10. The modified therapeutic peptide according to claim 1, wherein the moiety having one to three carbon atoms comprises a substituted or unsubstituted acetyl or formyl moiety.

11. The modified therapeutic peptide according to claim 1, wherein the therapeutic peptide comprises insulin, and wherein the hydrophilic polymer is attached at the B1 amino acid residue and the moiety having one to three carbon atoms is attached at the A1 amino acid residue.

12. The modified therapeutic peptide according to claim 11, further comprising an additional moiety having one to three carbon atoms attached at the B29 amino acid residue.

13. The modified therapeutic peptide according to claim 1, wherein the therapeutic peptide comprises insulin, wherein the hydrophilic polymer is attached at the B29 amino acid residue, and wherein the moiety having one to three carbon atoms is attached at the A1 amino acid residue.

14. The modified therapeutic peptide according to claim 13, further comprising an additional moiety having one to three carbon atoms attached at the B1 amino acid residue.

15. The modified therapeutic peptide according to claim 11, wherein the hydrophilic polymer exhibits a molecular weight of less than or equal to 10,000 Daltons.

16. The modified therapeutic peptide according to claim 15, wherein the hydrophilic polymer exhibits a molecular weight of less than or equal to 5000 Daltons.

17. A modified therapeutic peptide having at least one amino acid residue covalently attached to a hydrophilic polymer and at least one amino acid covalently attached to a moiety, wherein none of the following removal conditions will remove more than 50% of the moiety:
(1) 5 mg/ml of the modified therapeutic peptide is placed in trifluoroacetic acid (TFA) for 2 hours at 20° C.;
(2) 5 mg/ml of the modified therapeutic peptide is placed in water containing 2 M acetic acid for 24 hours at 20° C.;
(3) 5 mg/ml of the modified therapeutic peptide is placed in water containing 50 mM acetic acid for 24 hours at 20° C.;
(4) 5 mg/ml of the modified therapeutic peptide is placed in water containing 50 mM Tris at pH 8.5 for 24 hours at 40° C.;
(5) 5 mg/ml of the modified therapeutic peptide is placed in DMSO containing 20 (w/v) % piperidine for 5 minutes at 20° C.;
(6) 5 mg/ml of the modified therapeutic peptide is placed in a water/acetonitrile (1:1) mixture solution; the solution is bubbled with N₂ for at least 15 min; Pd/C catalyst (10 wt % on activated carbon) is then added slowly to 10 wt % of modified therapeutic peptide; then the reaction mixture is agitated; the system is evacuated and recharged with hydrogen gas under 50 psi three times (agitation is stopped during evacuation and recharging); the reaction mixture is then kept at room temperature under 50 psi for 16 hrs;
(7) 5 mg/ml of the modified therapeutic peptide is placed in ethylene glycol; 10 molar equivalents of hydrazine monohydrate 8 molar equivalents of KOH are added; the reaction mixture is heated to 100° C., under nitrogen for 30 minutes; and
(8) 5 mg/ml of the modified therapeutic peptide is placed in anhydrous HF at 0° C. for 30 minutes.

18. The modified therapeutic peptide according to claim 17, wherein the hydrophilic polymer comprises polyethylene glycol.

19. The modified therapeutic peptide according to claim 17, wherein the hydrophilic polymer has a molecular weight of less than or equal to 10,000 Daltons.

20. The modified therapeutic peptide according to claim 17, wherein the modified therapeutic peptide comprises an amino acid sequence of human insulin.

21. The modified therapeutic peptide according to claim 17, wherein the moiety has one to ten carbon atoms.

22. The modified therapeutic peptide according to claim 17, wherein the therapeutic peptide comprises insulin, and wherein the hydrophilic polymer is attached to the B1 amino acid residue and the moiety is attached to the A1 amino acid residue.

23. The modified therapeutic peptide according to claim 22, wherein the therapeutic peptide comprises insulin, and further comprising an additional moiety none of the removal conditions will remove more than 50% of the additional moiety, wherein the additional moiety is attached to the B29 amino acid residue.

24. A modified therapeutic peptide having at least one amino acid residue covalently attached to a hydrophilic polymer and at least one amino acid covalently attached to a moiety as shown in the following formula:
-A-D-Q-X
wherein:
A is selected from methyl, —CR₂—, —C(O)—, —S(O)(O)—, and —S—;
D, if present, is selected from —CR₂—, —C(O)—, —O—, pyridinyl, substituted pyridinyl, phenyl, substituted phenyl, cycloalkyl, and —CY₃ where Y is independently selected from hydrogen and lower alkyl;
Q, if present, is selected from —CR₃, phenyl, and substituted phenyl; and
X, if present, is phenyl;
wherein R is selected from hydrogen and lower alkyl.

25. The modified therapeutic peptide according to claim 24, wherein A is —CH₂—, D is —CY₃, and Y is selected from hydrogen and lower alkyl.

26. The modified therapeutic peptide according to claim 24, wherein A is —C(O)—, D is CY₃, and Y is selected from hydrogen and lower alkyl.

27. The modified therapeutic peptide according to claim 24, wherein the hydrophilic polymer comprises polyethylene glycol.

28. The modified therapeutic peptide according to claim 24, wherein the hydrophilic polymer has a molecular weight of less than or equal to 10,000 Daltons.

29. The modified therapeutic peptide according to claim 24, wherein the modified therapeutic peptide comprises an amino acid sequence of human insulin.

30. The modified therapeutic peptide according to claim 24, wherein the therapeutic peptide comprises insulin, and wherein the hydrophilic polymer is attached to the B1 amino acid residue and the moiety is attached to the A1 amino acid residue.

31. The modified therapeutic peptide according to claim 30, further comprising an additional moiety of the formula -A-D-Q-X, attached to the B29 amino acid residue.

32. A pharmaceutical composition, comprising: a modified therapeutic peptide having at least one amino acid residue covalently attached to a hydrophilic polymer and at least one amino acid covalently attached to a moiety having one to ten carbon atoms; and at least one pharmaceutically acceptable excipient; wherein the moiety having one to ten carbon atoms is not a hydrophilic polymer; and wherein the composition is pharmaceutically acceptable for administration.

33. The pharmaceutical composition according to claim 32, wherein the moiety having one to ten carbon atoms has two to eight carbon atoms.

34. The pharmaceutical composition according to claim 33, wherein the moiety having one to ten carbon atoms has three to six carbon atoms.

35. The pharmaceutical composition according to claim 34, wherein the moiety having one to ten carbon atoms has one to three carbon atoms.

36. The pharmaceutical composition according to claim 35, wherein the moiety having one to ten carbon atoms comprises an acetyl moiety.

37. The pharmaceutical composition according to claim 32, wherein the therapeutic peptide comprises insulin, wherein the moiety having one to ten carbon atoms is attached at the A1 amino acid residue.

38. The pharmaceutical composition according to claim 37, further comprising a moiety having one to ten carbon atoms attached at the B29 amino acid residue.

39. The pharmaceutical composition according to claim 37, further comprising a moiety having one to ten carbon atoms attached at the B1 amino acid residue.

40. The pharmaceutical composition according to claim 38, wherein the modified insulin comprises a hydrophilic polymer attached at the B1 residue.

41. The pharmaceutical composition according to claim 40, comprising an acetyl group attached at the A1 and B29 amino acid residues and a polyethylene glycol attached at the B1 amino acid residue.

42. The pharmaceutical composition according to claim 39, wherein the modified insulin comprises a hydrophilic polymer attached at the B29 residue.

43. The pharmaceutical composition according to claim 42, comprising an acetyl group attached at the A1 and B1 amino acid residues and a polyethylene glycol attached at the B29 amino acid residue.

44. The pharmaceutical composition according to claim 41, wherein the polyethylene glycol has a molecular weight of less than or equal to 10,000 Daltons.

45. The pharmaceutical composition according to claim 44, wherein the polyethylene glycol has a molecular weight of less than or equal to 5000 Daltons.

46. The pharmaceutical composition according to claim 43, wherein the polyethylene glycol has a molecular weight of less than or equal to 10,000 Daltons.

47. The pharmaceutical composition according to claim 46, wherein the polyethylene glycol has a molecular weight of less than or equal to 6000 Daltons.

48. The pharmaceutical composition of claim 40, wherein the hydrophilic polymer is absent a fatty acid moiety.

49. The pharmaceutical composition of claim 41, wherein the polyethylene glycol is end-capped.

50. The pharmaceutical composition of claim 49, wherein the polyethylene glycol is end-capped with an alkoxy group.

51. The pharmaceutical composition of claim 41, wherein the polyethylene glycol comprises linear polyethylene glycol, branched polyethylene glycol, forked polyethylene glycol, or dumbbell polyethylene glycol.

52. The pharmaceutical composition of claim 51, wherein the polyethylene glycol is linear.

53. The pharmaceutical composition of claim 51, wherein the polyethylene glycol comprises a releasable linkage.

54. The pharmaceutical composition of claim 51, wherein the polyethylene glycol comprises a number of (OCH₂CH₂) subunits of from about 2 to 300 subunits.

55. The pharmaceutical composition of claim 54, wherein the polyethylene glycol comprises a number of (OCH₂CH₂) subunits of from about 4 to 200 subunits.

56. The pharmaceutical composition of claim 55, wherein the polyethylene glycol comprises a number of (OCH₂CH₂) subunits of from about 10 to 100 subunits.

57. The pharmaceutical composition of claim 44, wherein at least about 75% of the therapeutic peptides in the composition are covalently coupled to polyethylene glycol.

58. The pharmaceutical composition of claim 57, wherein at least about 90% of the therapeutic peptides in the composition are covalently coupled to polyethylene glycol.

59. The pharmaceutical composition of claim 41, wherein the insulin is covalently coupled to the polyethylene glycol via a linking moiety positioned at a terminus of said polyethylene glycol.

60. The pharmaceutical composition of claim 59, wherein the polyethylene glycol, prior to coupling with insulin, possesses an activated linking moiety at one terminus suitable for covalent coupling with insulin.

61. The pharmaceutical composition of claim 60, wherein the activated linking moiety is suitable for coupling with reactive insulin amino groups.

62. The pharmaceutical composition of claim 61, wherein said activated linking moiety comprises a reactive functional group selected from N-hydroxysuccinimide active esters, active carbonates, aldehydes, and acetals.

63. The pharmaceutical composition of claim 59, wherein insulin is covalently coupled to polyethylene glycol via an amide linkage.

64. The pharmaceutical composition of claim 32 in aerosolized form.

65. The pharmaceutical composition of claim 32 in liquid form.

66. The pharmaceutical composition of claim 32 in dry form.

67. The pharmaceutical composition of claim 32 in spray-dried form.

68. The pharmaceutical composition of claim 32, having a T_{g} greater than 50° C.

69. The pharmaceutical composition of claim 32, comprising from 2% to 95% by weight modified therapeutic peptide.

70. The pharmaceutical composition of claim 32, wherein the excipient comprises at least one carbohydrate, amino acid, dipeptide, tripeptide, buffer, or combinations thereof.

71. The pharmaceutical composition of claim 70, wherein said excipient is a di- or tripeptide containing two or more leucyl residues.

72. The pharmaceutical composition of claim 32, wherein the composition comprises particles having an MMD (mass median diameter) of less than 15 μm.

73. The pharmaceutical composition of claim 72, wherein the composition comprises particles having an MMD of less than 10 μm.

74. The pharmaceutical composition of claim 73, wherein the composition comprises particles having an MMD of less than 5 μm.

75. The pharmaceutical composition of claim 32, wherein the composition comprises particles having an MMAD (mass median aerodynamic diameter) of less than 10 μm.

76. The pharmaceutical composition of claim 75, wherein the composition comprises particles having an MMAD of less than 5 μm.

77. The pharmaceutical composition of claim 76, wherein the composition comprises particles having an MMAD of less than 3 μm.

78. An inhalable pharmaceutical composition comprising:
a modified therapeutic peptide comprising at least one amino acid residue covalently attached to a moiety having one to ten carbon atoms; and
at least one pharmaceutically acceptable excipient suitable for inhalation;
wherein the moiety is not a hydrophilic polymer.

79. An inhalable composition comprising:
a modified therapeutic peptide having at least one amino acid covalently attached to a hydrophilic polymer and at least one amino acid covalently attached to a moiety having one to ten carbon atoms; and
at least one pharmaceutically acceptable excipient suitable for inhalation;
which upon pulmonary administration to a mammal exhibits a T_{1/2} of greater than or equal to about 4 hours.

80. A method of increasing the bioavailability of a pulmonarily administered modified insulin comprising covalently attaching to at least one of the A1 and B29 amino acid residues a moiety having one to ten carbon atoms, and maintaining the modified insulin in a form suitable for pulmonary administration; wherein the moiety is not a hydrophilic polymer.

81. A method of increasing the bioavailability of a pulmonarily administered modified insulin comprising covalently attaching to at least one of the A1 and B1 amino acid residues a moiety having one to ten carbon atoms, and maintaining the modified insulin in a form suitable for pulmonary administration; wherein the moiety is not a hydrophilic polymer.

82. A method of prolonging the half-life of a pulmonarily administered therapeutic peptide comprising covalently attaching a hydrophilic polymer to at least one amino acid residue and covalently attaching a moiety having one to ten carbon atoms to at least one amino acid residue.

83. An aerosolized formulation, comprising: a modified therapeutic peptide having at least one amino acid covalently attached to a moiety having one to ten carbon atoms; and
at least one pharmaceutically acceptable excipient; wherein the moiety having one to ten carbon atoms is not a hydrophilic polymer.

84. The aerosolized formulation of claim 83, wherein the pharmaceutically acceptable excipient comprises a dry formulation-enhancing excipient.

85. The aerosolizable formulation of claim 84, wherein the dry formulation-enhancing excipient is chosen from leucine, dileucine, and trileucine.

86. The aerosolizable formulation of claim 83, wherein the pharmaceutically acceptable excipient comprises a glass transition stabilizing excipient.

87. The aerosolizable formulation of claim 86, wherein the glass transition stabilizing excipient is chosen from monosaccharides, disaccharides, polysaccharides, and alditols.

88. The aerosolizable formulation of claim 83, wherein the modified therapeutic peptide is present in an amount of from about 20 weight % to about 99 weight %.

89. The aerosolizable formulation according to claim 83, wherein the formulation comprises a dry powder formulation comprising particles having a mass median diameter (MMD) of less than 30 gm.

90. The aerosolizable formulation according to claim 89, wherein the formulation comprises particles having an MMD of less than 15 μm.

91. The aerosolizable formulation according to claim 90, wherein the dry powder formulation comprises particles having an MMD of less than 10 μm.

92. The aerosolizable formulation according to claim 91, wherein the dry powder formulation comprises particles having an MMD of less than 5 μm.

93. The aerosolizable formulation according to claim 83, wherein the formulation comprises particles having a mass median aerodynamic diameter (MMAD) of less than 10 μm.

94. The aerosolizable formulation according to claim 93, wherein the formulation comprises particles having an MMAD of less than 5 μm.

95. The aerosolizable formulation according to claim 94, wherein the formulation comprises particles having an MMAD of less than 3 μm.

96. The aerosolizable formulation according to claim 83, wherein the formulation comprises particles having a powder surface area of ranges from about 6 m²/g to about 25 m²/g.

97. The aerosolizable formulation according to claim 96, wherein the powder surface area ranges from about 7 m²/g to about 10 m²/g.

98. A composition comprising a conjugate of therapeutic peptide covalently coupled to one or more molecules of polyethylene glycol and to one or more moieties having one to three carbon atoms.

99. The composition according to claim 98, wherein the composition comprises a powder, which (i) is characterized by an emitted dose value of at least about 50%; and (ii) when administered to a subject by inhalation, sustains elevated blood levels of therapeutic peptide in said subject for at least about 6 hours post administration.

100. A modified insulin having at least one amino acid residue covalently attached to a hydrophilic polymer via a spacer moiety comprising at least 4 carbon atoms, wherein the spacer moiety is attached to the at least one amino acid residue via a secondary amine, and wherein the at least one amino acid residue comprises at least the B1 or B29 amino acid residue.

101. The modified insulin according to claim 100, wherein the hydrophilic polymer comprises a non-naturally occurring polymer.

102. The modified insulin according to claim 100, wherein the hydrophilic polymer comprises a non-peptidic polymer.

103. The modified insulin according to claim 100, wherein the hydrophilic polymer comprises a polymer of ethoxy monomers.

104. The modified insulin according to claim 100, wherein the hydrophilic polymer comprises a polyethylene glycol.

105. The modified insulin according to claim 104, wherein the polyethylene glycol has a molecular weight of less than or equal to 10,000 Daltons.

106. The modified insulin according to claim 104, wherein the polyethylene glycol has a molecular weight of less than or equal to 5000 Daltons.

107. The modified insulin according to claim 100, wherein the modified therapeutic peptide comprises an amino acid sequence of human insulin.

108. A modified insulin comprising the following structure: 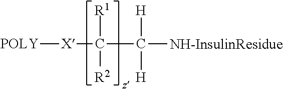
wherein:
POLY is a water-soluble polymer segment;
X′ is a linker moiety;
z′ is an integer from 1 to about 21;
R¹, in each occurrence, is independently H or an organic radical selected from the group consisting of alkyl, substituted alkyl, alkenyl, substituted alkenyl, alkynyl, substituted alkynyl, aryl, and substituted aryl;
R², in each occurrence, is independently H or an organic radical selected from the group consisting of alkyl, substituted alkyl, alkenyl, substituted alkenyl, alkynyl, substituted alkynyl, aryl, and substituted aryl,
“—NH-Insulin Residue” represents an insulin residue comprising an amino group, and wherein the —NH— comprises the amino group of at least one of B1 and B29 amino acid residues,
and further wherein the following apply:
when POLY is linear:
(a) the total number of carbonyls present in said polymer is 0 or 2 or greater except when X′ comprises one or more contiguous (—CH₂CH₂O—) segments,
(b) and X′ is oxygen or comprises at least one (—CH₂CH₂O—) segment and z′ is from 2 to 12, then at least one of R¹ or R² in at least one occurrence is an organic radical as defined above or said polymer is heterobifunctional where POLY comprises a reactive group at one terminus that is not hydroxy, and
when POLY is branched:
(c) then either at least one of R¹ or R² in at least one occurrence is an organic radical as defined above or X′ includes —(CH₂CH₂O)_{b}— where b is from 1 to about 20,
and further in the instance where POLY comprises a lysine residue,
(d) and has 2 polymer arms, then neither polymer arm comprises oxygen as the only heteroatom in the instance where POLY comprises “C—H” as a branch point.

109. The modified insulin of claim 108, wherein POLY is selected from poly(alkylene oxide), poly(vinyl pyrrolidone), poly(vinyl alcohol), polyoxazoline, poly(acryloylmorpholine), and poly(oxyethylated polyol).

110. The modified insulin of claim 108, wherein POLY is a poly(ethylene glycol).

111. The modified insulin of claim 110, wherein the poly(ethylene glycol) is terminally capped with an end-capping moiety.

112. The modified insulin of claim 111, wherein the end-capping moiety is selected from alkoxy, substituted alkoxy, alkenyloxy, substituted alkenyloxy, alkynyloxy, substituted alkynyloxy, aryloxy, substituted aryloxy.

113. The modified insulin of claim 112, wherein the end-capping moiety is selected from methoxy, ethoxy, and benzyloxy.

114. The modified insulin of claim 108, wherein X′ comprises a moiety corresponding to the structure:
—(CH₂)_{c}-Dₑ-(CH₂)_{f}— or —(CH₂)ₚ-Mᵣ-C(O)—Kₛ—(CH₂)_{q}—
wherein:
c is zero to 8,
D is O, NH, or S,
e is 0, 1
f is zero to 8,
p is zero to 8,
M is —NH, O
K is NH, O
q is from zero to 8, and
r and s are each independently 0, 1

115. The modified insulin of claim 108, wherein X′ includes a moiety corresponding to the structure —(CH₂CH₂O)_{b}— or —(CH₂CH₂NH)_{g}—, and b and g are each independently 1 to 20.

116. The modified insulin of claim 115, wherein b and g are each independently 1 to 10.

117. The modified insulin of claim 115, wherein b and g are each independently 1 to 6.

118. The modified insulin of claim 108, wherein X′ comprises a moiety corresponding to the structure:
—(CH₂)_{c}-Dₑ-(CH₂)_{f}—P— or —(CH₂)ₚ-Mᵣ-C(O)—Kₛ—(CH₂)_{q}-T-
wherein:
P and T are each independently —(CH₂CH₂O)_{b}— or —(CH₂CH₂NH)_{g},
b and g are each independently 1 to 20,
and the remaining variables are as defined in claim 18.

119. The modified insulin of claim 108, wherein said X′ comprises —C(O)NH—(CH₂)₁₋₆NH—C(O)— or —NHC(O)NH—(CH₂)₁₋₆NH—C(O)—.

120. The modified insulin of claim 108, wherein X′ is —C(O)—.

121. The modified insulin of claim 108, wherein z′ ranges from 2 to 12.

122. The modified insulin of claim 108, wherein z′ ranges from 4 to about 12.

123. The modified insulin of claim 108, wherein z′ ranges from 4 to about 8.

124. The modified insulin of claim 108, wherein R¹ and R² are both H in each occurrence.

125. A method of reducing blood glucose level in a mammal, comprising pulmonarily administering to the mammal a pharmaceutical formulation comprising particles comprising a modified insulin having at least one amino acid covalently attached to a hydrophilic polymer and at least one amino acid covalently attached to a moiety having one to ten carbon atoms, wherein the moiety having one to ten carbon atoms is not a hydrophilic polymer.

126. The method according to claim 125, wherein administration results in a measurable reduction in blood glucose level in less than 1 hour after administration, and wherein administration results in a measurable reduction in blood glucose level for a period of at least about 6 hours.

127. The method according to claim 126, wherein administration results in a measurable reduction in blood glucose level for a period of at least about 7 hours.

128. The method according to claim 127, wherein administration results in a measurable reduction in blood glucose level for a period of at least about 8 hours.

129. The method according to claim 126, wherein at least about 75% of the administered modified insulin is present in the lungs 2 hours after administration.

130. A pharmaceutical formulation for inhalation, comprising particles having a mass median aerodynamic diameter (MMAD) of less than 10 μm, comprising a modified therapeutic peptide having at least one amino acid covalently attached to a hydrophilic polymer and at least one amino acid covalently attached to a moiety having one to ten carbon atoms, wherein the moiety having one to ten carbon atoms is not a hydrophilic polymer.

131. A method of decreasing enzymatic digestion of insulin, comprising covalently attaching to at least one of the insulin A1 and B29 amino acid residues a moiety having one to ten carbon atoms to create a modified insulin, and pulmonarily administering the modified insulin; wherein the moiety having one to ten carbon atoms is not a hydrophilic polymer.

132. A method of decreasing enzymatic digestion of insulin, comprising covalently attaching to at least one of the insulin A1 and B1 amino acid residues a moiety having one to ten carbon atoms to create a modified insulin, and pulmonarily administering the modified insulin; wherein the moiety having one to ten carbon atoms is not a hydrophilic polymer.

133. A method of making a modified therapeutic peptide composition for administration comprising reacting therapeutic peptide with a blocking agent having one to three carbon atoms, and reacting therapeutic peptide with a reactive hydrophilic polymer to produce a therapeutic peptide-polymer conjugate; wherein a modified, hydrophilic polymer-conjugated, therapeutic peptide is produced by the reactions; and further comprising formulating the modified, hydrophilic polymer-conjugated, therapeutic peptide into a composition for administration.

134. The method according to claim 133, wherein the blocking agent is an acetylating agent.

135. The method according to claim 133, wherein the composition is formulated for pulmonary administration.

136. The method according to claim 133, wherein the composition is formulated for injection.

137. The method according to claim 136, wherein the composition is formulated for subcutaneous injection.

138. The method according to claim 133, wherein the reacting with the acetylating agent is performed prior to the reacting with the reactive hydrophilic polymer.

139. The method according to claim 133, comprising reacting the therapeutic peptide with an acetylating agent in a molar ratio of acetylating agent to therapeutic peptide of at least 1:1.

140. The method according to claim 139, comprising reacting the therapeutic peptide with an acetylating agent in a molar ratio of acetylating agent to therapeutic peptide of at least 2:1 to produce diacetylated therapeutic peptide.

141. The method according to claim 140, comprising reacting the therapeutic peptide with an acetylating agent in a molar ratio of acetylating agent to therapeutic peptide of at least 3:1 to produce diacetylated therapeutic peptide.

142. The method according to claim 140, comprising reacting the diacetylated therapeutic peptide with a reactive hydrophilic polymer, wherein a diacetylated, hydrophilic polymer-conjugated therapeutic peptide is produced.

143. The method according to claim 138, comprising reacting the therapeutic peptide with an acetylating agent in a pH of greater than or equal to about 8.5.

144. The method according to claim 143, comprising reacting the therapeutic peptide with an acetylating agent in a pH of greater than or equal to about 9.0.

145. The method according to claim 144, comprising reacting the therapeutic peptide with an acetylating agent in a pH ranging from 8 to 12.

146. The method according to claim 145, comprising reacting the therapeutic peptide with an acetylating agent in a molar ratio of at least 3:1, in a pH of from about 9.5 to about 10.0.

147. The method according to claim 146, wherein diacetylated therapeutic peptide is produced in a yield of greater than about 75%.

148. The method according to claim 147, wherein the therapeutic peptide comprises insulin, and wherein the insulin is diacetylated at amino acid residues A1 and B29.

149. The method according to claim 147, wherein the therapeutic peptide comprises insulin, and wherein the insulin is diacetylated at amino acid residues A1 and B1.

150. The method according to claim 133, wherein the reacting therapeutic peptide with the blocking agent having one to three carbon atoms comprises contacting therapeutic peptide with the blocking agent in an organic solvent.

151. The method according to claim 150, wherein the contacting occurs at a temperature of at least 25° C.

152. The method according to claim 133, wherein the reacting therapeutic peptide with a reactive hydrophilic polymer comprises contacting therapeutic peptide with the hydrophilic polymer in an organic solvent.

153. The method according to claim 152, wherein the reactive hydrophilic polymer is dissolved in the organic solvent at a temperature above 25° C., the reactive hydrophilic polymer in the organic solvent is cooled to a temperature below 25° C., and the reactive hydrophilic polymer is contacted with therapeutic peptide.

154. The method according to claim 133, further comprising subjecting the modified, hydrophilic polymer-conjugated, therapeutic peptide to column chromatography prior to formulating the modified, hydrophilic polymer-conjugated, therapeutic peptide into the composition for administration.

155. A method of making a modified therapeutic peptide composition, comprising reacting acetylated therapeutic peptide with a hydrophilic polymer to produce the modified therapeutic peptide composition.

156. The method according to claim 155, wherein the hydrophilic polymer comprises polyethylene glycol.

157. A method of making a modified therapeutic peptide composition, comprising reacting an therapeutic peptide-hydrophilic polymer conjugate with an acetylation agent to form the modified therapeutic peptide composition.

158. The method according to claim 157, wherein the acetylation agent comprises acetic acid-N-hydroxysuccinimide.

159. A method comprising, contacting a hydrophilic polymer butyraldehyde with insulin at a pH less than 5 to form a modified insulin having at least one amino acid residue covalently attached to the hydrophilic polymer via a spacer moiety comprising at least 4 carbon atoms, wherein the spacer moiety is attached to the at least one amino acid residue via a secondary amine, and wherein the at least one amino acid residue comprises at least the B1 or B29 amino acid residue.

160. The method of claim 159, wherein the insulin is not protected with a protecting group.

161. The method of claim 159, wherein the contacting occurs in the presence of a reducing agent.

162. The method of claim 159, wherein a yield of the contacting is at least 50%.
